(19) United States
(12) Patent Application Publication
(10) Pub. No.: US 2023/0242624 A1
(43) Pub. Date: Aug. 3, 2023

(54) HUMAN ANTIBODIES AGAINST SEVERE ACUTE RESPIRATORY SYNDROME CORONAVIRUS-2 (SARS-CoV-2)

(71) Applicant: Neurimmune AG, Schlieren (CH)

(72) Inventors: Stefan MOESE, Oberwil-Lieli (CH); Nike KRÄUTLER, Zurich (CH); Cédric CORTIJO, Urdorf (CH); Jan GRIMM, Dübendorf (CH); Benoit COMBALUZIER, Urdorf (CH); Roger NITSCH, Zumikon (CH); Christoph HOCK, Erlenbach (CH)

(21) Appl. No.: 18/007,672

(22) PCT Filed: Jun. 2, 2021
(86) PCT No.: PCT/EP2021/064892

§ 371 Date: Dec. 1, 2022

(30) Foreign Application Priority Data
Jun. 2, 2020 (EP) 20177854.5
Jun. 26, 2020 (EP) 20182480.2
Sep. 7, 2020 (EP) 20194901.3

(51) Int. Cl. C07K 16/10; A61P 31/14; G01N 33/569

(52) U.S. Cl. CPC C07K 16/10; A61P 31/14; G01N 33/56983; C07K 2317/22; C07K 2317/24; C07K 2317/52; C07K 2317/92; C07K 2317/565; C07K 2317/56; C07K 2317/34; C07K 2317/76; C07K 2317/31; C07K 2317/54; C07K 2317/41; C07K 2317/14; A61K 2039/505

(57) ABSTRACT

Provided are novel human-derived monoclonal antibodies as well as antigen-binding fragments thereof which specifically recognize and preferably neutralize severe acute respiratory syndrome coronavirus-2 (SARS-CoV-2). The anti-SARS-CoV-2 antibodies provided herein are useful in treating and diagnosis of COVID-19.

### FIELD OF THE INVENTION

The present invention generally relates to novel human-derived antibodies and antigen-binding fragments thereof which specifically recognize and preferably neutralize severe acute respiratory syndrome coronavirus-2 (SARS-CoV-2).

### BACKGROUND OF THE INVENTION

Coronaviruses are a large family of enveloped viruses with single stranded RNA genomes common across the world. At least seven species are known to cause disease in humans, some causing symptoms of a common cold; others causing Middle East Respiratory Syndrome (MERS) or Severe Acute Respiratory Syndrome (SARS). Coronavirus disease 2019 (COVID-19) is caused by a new strain of coronavirus not previously seen in humans named SARS-CoV-2. Since the outbreak in China end of 2019, infections with SARS-CoV-2 were spreading globally resulting in a COVID-19 pandemic, challenging the global health care systems and economies.

Since SARS-CoV-2 is a new virus, probably originally transmitted from animals to humans, the lack of immunity in the general population results in low barriers to spread extensively. In principle, all human beings are susceptible as long as there is no herd immunity or vaccination available. It is estimated that the vast majority of cases (around 80%) show only a mild-to-moderate self-limiting illness and recover without the need for specific treatments. However, a subpopulation of 15-20% of COVID-19 patients are requiring hospital care, 16-26% of these hospitalized patients are in need of intensive care unit treatment (Wang et al., JAMA 323 (2020), 1061-1069 and Grasselli et al., JAMA (2020), doi:10.1001/jama.2020.4031). Globally, about 3.4% of reported COVID-19 patients have died as reported by the World Health Organization (WHO).

There is currently neither a vaccine against COVID-19, nor any specific, proven, antiviral medication. Most treatments are therefore directed towards managing symptoms and providing support to patients with complications.

Example Even if some of the novel vaccination approaches that are currently in development will prove effective, it will be challenging to vaccinate the entire population and certain subgroups, especially the elderly population, may not develop adequate protective immunity responses. Therefore, for passive immunization and treatment, one approach is to collect convalescent plasma from people who have recovered from COVID-19 or from the 2002-03 SARS since cross-reactivity of plasma from the latter and of individual antibodies has been reported. At the same time, while recent publications report on identifying neutralizing monoclonal anti-SARS-CoV-2 antibodies, the current mindset suggests that either polyclonal antibody or monoclonal antibody cocktails directed against the coronavirus causing COVID-19 are required in order to successfully combat with the disease. Besides, there is still a demand for antibodies that are highly specific for SARS-CoV-2, both as a research tool and COVID-19 test, either as a direct detection means or as positive controls.

Thus, there is an ongoing need for providing coronavirus-targeted antibodies that have distinct properties such as binding specificities towards SARS-CoV-2 and SARS as well as for providing suitable antibody formats and strategies for the treatment of COVID-19 and preferably prevention infection with SARS-CoV-2.

The present invention and its embodiments as characterized in the claims and taught in the description and illustrated in the Examples contributes to the solution of the above-mentioned problem.

### SUMMARY OF THE INVENTION

The present invention generally relates to SARS-CoV-2-specific human-derived monoclonal antibodies and SARS-CoV-2-binding fragments thereof as well as equivalent synthetic variants and biotechnological derivatives of the antibodies exemplified herein, that recognize SARS-CoV-2, in particular the receptor-binding domain (RBD) of the Spike (S) protein of SARS-CoV-2. Thus, a composition of antibodies is provided that bind to the RBD of SARS-CoV-2 S with an EC₅₀ of at least <100 pM and even down to about 1-10 pM, i.e. they show a very high affinity to the RBS of SARS-CoV-2. In this context, in view of the above-mentioned multiple and different challenges by COVID-19 the antibodies provided by the present invention may be grouped in good neutralization and SARS-CoV cross reactive; good neutralization and SARS-CoV-2 specific and highly SARS-CoV-2 specific antibodies. However, since in particular the series of NI-607.53× antibodies have been obtained from the same donor who recovered from COVID-19 and had a mild course of the disease, it might well be that also antibodies that proved less potent in the neutralization assay nevertheless contributed to some extent to the elimination of the virus and the subject's recovery. For example, for influenza it has been described that both neutralizing and non-neutralizing anti-influenza protein antibodies can offer heterosubtypic protection against influenza A virus; see Quirarte et al., Front. Immunol. 10 (2019):1677.doi: 10.3389/fimmu.2019.01677.

Furthermore, in a combined as well as general independent aspect the present invention relates to anti-SARS-CoV-2 antibodies and antigen-binding fragment thereof as well as to polynucleotides encoding the antibody or antigen-binding fragment thereof, wherein the antibody is capable of binding to RBD of the S protein of SARS-CoV-2, wherein the antibody or antigen-binding fragment thereof is characterized to be of the IgG4 isotype, i.e. the antibody or antigen-binding fragment thereof comprises an IgG4 constant domain, preferably including the S228P mutation. In addition, the antibody preferably binds to a conformational epitope of the RBD. Furthermore, in one embodiment, the antibody does not bind to the RBD of SARS-CoV while in another embodiment the antibody binds to the RBD of SARS-CoV.

Coronavirus entry into host cells is mediated by the transmembrane S glycoprotein that forms homotrimers protruding from the viral surface. The S glycoprotein comprises two functional subunits: S1 (divided into A, B, C and D domains) that is responsible for binding to host cell receptors and S2 that promotes fusion of the viral and cellular membranes. Both the novel SARS-CoV-2 causing COVID-19 and SARS-CoV belong to the sarbecovirus subgenus and their S glycoproteins share about 80% amino acid sequence identity. Recently, it has been demonstrated that human-angiotensin converting enzyme 2 (hACE2) is a functional receptor for SARS-CoV-2, as is the case for SARS-CoV. The S domain B (SB) is the RBD and binds to hACE2 with high-affinity, possibly contributing to the current rapid SARS-CoV-2 transmission in humans (Pinot et al., Nature (2020), doi:10.1038/s41586-020-2349-y as well as references cited therein). While this enhanced affinity may explain a much stronger spreading ability of SARS-CoV-2, it also suggests that finding potent neutralization antibodies targeting SARS-CoV-2 RBD could be much more challenging.

As the coronavirus S glycoprotein mediates entry into the host cells, it is the main target for developing therapeutic and vaccine approaches. Human monoclonal antibodies to the viral surface S glycoprotein have already been show to mediate immunity to other betacoronaviruses including SARS-CoV and MERS.

In accordance with the present invention human monoclonal anti-SARS-CoV-2 antibodies have been cloned and identified from memory B cells and plasma cell preparations obtained from patients that have successfully recovered from COVID-19 in a discovery process comprising Reverse Translational Medicine™ (RTM™), Neurimmune's proprietary technology platform and bio-layer interferometry (BLI) technology applied to an antibody discovery platform similar as described and visualized in FIG. S2b of Zost et al., bioRxiv (2020), doi:10.1101/2020.05.12.091462, wherein human B cells producing anti-SARS-CoV-2 antibodies are bound by RBD-coated beads and wherein the anti-SARS-CoV-2 antibodies are detected by secondary fluorescent antibodies; see Examples 1 and 2.

Positive hits were counter-screened to exclude clones cross-reacting with unrelated targets and selective SARS-CoV-2 RBD-reactive B-cells were subjected to cDNA cloning. The amino acid and DNA sequences of the resulting antibodies are provided in Table II. Those antibodies were tested for their binding specificity and binding efficiency on SARS-CoV-2 S RBD and SARS-CoV S RBD. Human SARS-CoV-2-specific antibodies showed high affinity to their targets within the picomolar range. They were either SARS-CoV-2 specific or also cross-reactive towards SARS-CoV; see Table I and Examples 3 and 4.

Antibodies binding with high specificity to the RBD of SARS-CoV-2, but do not substantially bind to SARS-CoV are useful for diagnostics since they can distinguish SARS-CoV-2 from other coronaviruses, in particular from SARS-CoV. Antibodies binding to the RBD of SARS-CoV-2 as well as SARS-CoV with high affinity are useful for the treatment of COVID-19 as well as SARS and probably for the treatment of other diseases caused by related coronaviruses. In this context, it is prudent to expect that those antibodies bind to an epitope which is conserved in coronaviruses and which has a low mutations frequency. Accordingly, those antibodies might be useful for the treatment of future diseases related to coronaviruses, for example in the treatment of a potentially new emerging virus. Indeed, subject antibodies NI-607.531_C8 and NI-607.649_B11 which are demonstrated to have therapeutic utility both in prophylactic and therapeutic treatment settings, see Example 10 and FIG. 8, have been determined to recognize a conformational epitope which might not be as affected by mutations in the primary structure of the RBD domain of the S protein as linear epitopes. Since antibody NI-607.649_B11 also recognizes SARS-CoV with high affinity it seems that both SARS-CoV-2 and SARS-CoV share a common conservative conformational epitope which might be involved in ACE2 receptor binding.

Since safety of antibody-based therapy is highly dependent on target specificity, the cross-reactivity of anti-SARS-CoV-2 antibodies towards a panel of unrelated proteins was evaluated by ELISA or iQue analysis; see Examples 3 and 4. The results showed that the antibodies have no substantial cross-reactivity to unrelated targets like BSA and further non-related proteins.

Specificity of the antibodies was further tested via measuring their binding to the RBD of MERS-CoV. The spike (S) protein of MERS-CoV mediates infection by binding to the cellular receptor dipeptidyl peptidase 4 (DPP4) and the sequence of the RBD of MERS-CoV differs from the one of SARS-CoV-2. As shown in Table I, the tested antibodies only show moderate binding to MERS-CoV and thus are specific for SARS viruses.

As further shown in Table I, the tested antibodies show a quite different neutralization potency which does not directly correlate with the determined EC₅₀ values. For example, antibody NI-607.529_B9 showed a rather high EC₅₀ value, i.e., about 34 pM in comparison to various other antibodies, but surprisingly has a high potency for neutralization, i.e., it has a low IC₅₀ of 116 pM. The EC₅₀ value of antibody NI-607.531_C8 is lower, i.e., 11 pM, but the antibody seems to have a slightly lower neutralization potency with an IC₅₀ of 153 pM. In principle those antibodies showing a low IC₅₀ value and in particular those showing an IC₅₀ value in the picomolar range are of particular interest.

Accordingly, novel anti-SARS-CoV-2 antibodies have been cloned and identified, which can be used for diagnosing and treatment of COVID-19 and possibly other coronavirus related diseases.

TABLE I
Binding properties of anti-SARS-CoV-2 antibodies, n.a. = no IC₅₀ available, n.d. = not
determined. Antibody in italics are originally IgA and *presumably IgA, respectively. First
two digits XX of the second number in NI-607.XXY indicates origin from the same donor, e.g.,
NI-607.531_C8 and NI-607.532_B6. Indices 1, 2 and 3 in superscript indicate the most
suitable candidates for each group of (1) good neutralization and SARS cross reactive;
(2) good neutralization and SARS-Cov-2 specific; and (3) no neutralization but
SARS-Cov-2 specific strong binding.

| | ELISA | iQue | iQue | iQue | RBD |
|---|---|---|---|---|---|
| | SARS-CoV-2 | SARS-CoV-2 | SARS-CoV | MERS-CoV | Neutralization |
| | binding EC₅₀ | binding EC₅₀ | binding | binding | ELISA IC₅₀ |
| Antibody | [pM] | [nM] | [nM] | [nM] | [pM] |
| NI-607.274_B7³ | 21 | 0.67 | 900 | 900 | 7800 |
| NI-607.274_E5³ | 15 | 0.39 | 900 | 900 | 12600 |
| NI-607.275_C5 | 20 | 1.5 | 800 | 800 | 36833 |
| NI-607.426_D4³ | 33 | 0.76 | 1000 | 1000 | 24353 |
| NI-607.426_E2 | 30 | 2.06 | 600 | 600 | 10693 |
| NI-607.426_F11² | 17 | 0.89 | 1000 | 1000 | 1142 |
| NI-607.427_C5 | 14 | 3.2 | 3 | 600 | 2877 |
| NI-607.428_B9 | 26 | 1.3 | 800 | 800 | 15787 |
| NI-607.429_B9² | 14 | 1.87 | 600 | 600 | 74 |
| NI-607.429_E4¹ | 8.6 | 1.9 | 200 | 200 | 275 |
| NI-607.529_B9¹ | 34 | 1 | 20 | 300 | 116 |
| NI-607.529_G4 | 26 | 1.3 | 10 | 1000 | n.a |
| NI-607.531_C8 | 11 | 1.2 | 1000 | 1000 | 153 |
| NI-607.531_D8 | 4.5 | 1.4 | 1000 | 1000 | n.a |
| NI-607.532_B6 | 4.5 | 1.1 | 4.2 | 1000 | n.a |
| NI-607.532_{—}C11² | 13 | 2.4 | 1000 | 1000 | 826 |
| NI-607.532_{—}C8² | 9.9 | 2.3 | 400 | 400 | n.a |
| NI-607.532_{—}D3² | 4.9 | 1 | 1000 | 1000 | 917 |
| NI-607.532_{—}D4 | 5.5 | 1 | 1000 | 1000 | n.a |
| NI-607.532_{—}D8 | 5.1 | 14.4 | 32 | 1000 | n.a |
| NI-607.532_{—}F9*² | 2.5 | 3.3 | 1000 | 1000 | 775 |
| NI-607.649_{—}B11*¹ | 27 | 1.4 | 1.7 | 1000 | 80 |
| NI-607.531_{—}E7* | 27 | 1.3 | 600 | 800 | 3666 |
| NI-607.532_{—}F3¹ | 19 | 1.2 | 1.3 | 1000 | 353 |
| NI-607.649_G7 | 24 | 5.7 | 22.2 | 800 | n.a. |
| NI-607.761_B7 | 1 | 0.44 | 200 | 200 | 56000 |
| NI-607.791_B10 | 62 | 13.7 | 400 | 400 | n.d. |
| NI-607.531_{—}E3* | 20 | 1.4 | 2.6 | 1000 | n.a. |
| NI-607.820_B6 | 29 | 2.4 | 60.2 | 1000 | n.d |
| NI-607.820_B7 | 82 | 9 | 800 | 800 | n.d. |
| PBS | | | | | |
| antiSars-Cov2 AB | 17 | 1.2 | 2.6 | 600 | 100 |
| Isotype Control | >400000 | | | | |

Moreover, as demonstrated in Example 10 and shown in FIG. 8, the anti-SARS-CoV-2 antibodies of the present invention have in vivo efficacy in a recognized animal model for SARs-CoV and SARS-CoV-2 infection when used prophylactically and therapeutically. In particular, the golden Syrian hamster model has been used that was already established for the investigation of anti-SARs-CoV antibodies and found to be a suitable and better model for specific evaluation of therapeutic effects than the mouse; see, e.g., Roberts et. al., “MAb Therapy Reduces SARs-CoV Disease Severity” JID 193 (2006), 685-692.

So far, most approaches of immunotherapy of COVID-19 are based on whole-inactivated virus, live attenuated virus, protein subunit, replicating and non-replicating viral vectors expressing SARS-CoV-2 proteins as well as DNA and RNA technologies delivering gene sequences that encode SARS-CoV-2 proteins that then are produced by host cells. In addition, convalescent plasma (plasma with antibodies from recovered COVID-19 patients) is under investigation for the treatment of patients with COVID-19.

Regarding monoclonal antibodies against SARS-CoV-2 only recently proof of concept for the use of an antibody cocktail of two distinct neutralizing antibodies in the treatment of SARS-CoV-2 also utilizing the golden Syrian hamster model has been provided; see Baum et al. “REGN-COV2 antibody cocktail prevents and treats SARS-CoV-2 infection in rhesus macaques and hamsters”. Preprint at bioRxiv https://doi.org/10.1101/2020.08.02.233320 (2020). As described therein, a cocktail consisting of two neutralizing antibodies (REGN10987+REGN10933) targeting non-overlapping epitopes on the SARS-CoV-2 spike protein have been investigated and seemed to provide evidence that such antibody cocktail may be useful in the prevention and treatment of COVID-19 disease.

Though the origin and nature of the two antibodies do not seem to be specifically disclosed in Baum et al. (2020), Matthews 2020 (Nature Reviews Immunology https://doi.org/10.1038/s41577-020-00431-9 Published online: 17. August 2020) refers the antibodies as “fully humanized antibodies”. Therefore, as described in Hansen et al., 2020 (Science 369 (2020), 1010-1014), REGN10987 and REGN10933 seem to be obtained from VelocImmune® (VI) mice that were immunized using DNA encoding full length (FL) SARS-CoV-2 spike protein and a recombinant protein of spike receptor binding domain (RBD) with an inline fusion of mouse Fc tag on the C-terminus (RBD-mFc); see Hansen et al. 2020 SUPPLEMENTARY MATERIALS; science.sciencemag.org/content/369/6506/1010/suppl/DC1, Materials and Methods. Both antibodies demonstrate strong effector functions and mediate both antibody-dependent cellular cytotoxicity (ADCC), though more pronounced for REGN10987, and antibody-dependent cellular phagocytosis (ADCP). In view of the isotype control used in the studies of Baum et al. 2020 and Hansen et al. 2020 both antibodies seem to be of the IgG1 isotype.

In contrast, the present invention provides truly fully human antibodies which have been isolated from plasma cells (PCs) and memory B cells, respectively, obtained from clinically interesting donors which have successfully recovered from COVID-19, utilizing the proprietary Reverse Translational Medicine™ (RTM™) technology platform by Neurimmune AG. As illustrated in the Examples and Figures, the antibodies of the invention are capable of suppressing and reducing viral infection in cellular assays as well as in an established animal model for COVID-19 when administered as a single antibody.

Furthermore, the experiments performed in accordance with the present invention surprisingly demonstrate that, as illustrated with antibody NI-607.649_B11, antibodies which recognize both SARS-CoV and SARS-CoV-2 are substantially as efficacious in vivo as antibodies, such as NI-607.531_C8, which specifically recognize SARS-CoV-2. These results are unexpected since Hansen et al. 2020 observed that antibodies shown to cross-neutralize SARS-CoV and SARS-CoV-2 spike proteins were weakly neutralizing, for which reason cross-neutralizers had not been pursued in their approach.

Hence, due to the method of their generation REGN10987 and REGN10933 seem to target non-overlapping linear epitopes on the SARS-CoV-2 spike protein. In contrast, in a preferred embodiment of the present invention and as illustrated in the Examples with the two subject antibodies NI-607.531_C8 and NI-607.649_B11, the antibodies of the present invention preferably recognize conformational epitopes which might safeguard against mutational virus escape assuming that the structure of the receptor binding domain (RBD) and thus conformation should be more conserved than the individual amino acids of the spike protein. In this context, in one preferred embodiment, the antibody of the present invention, as mentioned before also recognizes SARS-CoV. This is because when considering a conformational epitope there are sound reasons to believe that due to the cross-reactivity this epitope might even be more conservative and thus less prone to mutations than epitopes that are unique for SARS-CoV-2 or even if mutations in the primary amino acid sequence occur the conformation of the epitope may remain unaffected in kind.

In addition, and in contrast to REGN10987 and REGN10933 as characterized in Hansen et al. 2020, see supra, recombinant human-derived antibodies of the IgG4 type have been used in the in vivo studies performed in accordance with the present invention. As known in the art, the level of ADCC effector function for IgG4 is much lower than for IgG1. Accordingly, the antibodies of the present invention in particular when formatted as IgG4 can be advantageously used with the benefit that adverse events such as the induction of pro-inflammatory cytokines (i.e., cytokines storm) due to unintended effector function can be avoided or reduced. Accordingly, in a particular preferred embodiment in the prophylactic and therapeutic treatment, respectively, of COVID-19, the human antibody of the present invention has attenuated effector function and preferably is of the IgG4 type.

Moreover, the assumption that the antibodies of the present invention will have therapeutic utility in humans is further supported by the following fact: While in the study by Baum et al. 2020 for the therapeutic treatment setting in the animal model the antibody cocktail has been administered already one day after challenge with the virus, in the experiments performed in accordance with the present invention the single dose antibody has been administered two days after virus challenge, i.e. at a time at which viral application and spreading has already advanced and which might better reflect a possible scenario in the treatment of humans. For example, a subject that had been notified to be in contact with SARS-CoV-2 and another subject that was infected, respectively, might not be able to receive a treatment already one day after the incident.

In another publication, Roberts et al., Science 10.1126/science.abc7520 (2020), neutralizing antibodies to epitopes on the receptor binding domain (RBD) and to distinct non-RBD epitopes on the spike protein have been isolated and tested in the golden Syrian hamster model. However, as described therein, here the authors used quite unusual parameters since in the prophylactic treatment SARS-CoV-2 challenge has been made already 12 hours post antibody infusion in contrast to one or two days as described for example in Baum et. al. 2020 and in Example 10 of the present invention, and the read-out for the body weight of the hamsters was already made on day 5 though the highest loss of body weight after infection with SARS-CoV-2 is observed at days 6 and 7 post infection; see e.g., Imai et al., PNAS 117 (2020), 16587-16595 and Baum et. al. (2020), supra, as well as Example 10 below and FIG. 8. Furthermore, only a prophylactic setting has been tested while no data are presented regarding any therapeutic utility of the antibodies. Accordingly, no conclusion can be drawn as to the in vivo efficacy of therapeutic settings.

In summary, the present invention as illustrated in the Examples provides not only novel human-derived anti-SARS-CoV-2 antibodies but also generally novel prophylactic and therapeutic settings for the treatment of COVID-19 by teaching appropriate administration regime and antibody formats, i.e., IgG4 and thus represents an important contribution to immunotherapy in both, prevention and treatment settings of COVID-19 disease.

Of course, though as illustrated in the Example 10 the present invention provides the treatment of COVID-19 with a single recombinant human-derived monoclonal antibody, the prophylactic and therapeutic treatment settings can also be performed with any of the antibody combinations, antibody compositions and antibody cocktails described herein. On the other hand, though the use of any one of the antibodies of the present invention disclosed herein, in particular those illustrated in the Examples, either alone or in combination is always preferred, regarding the general novel findings in accordance with the present invention illustrated in Example 10, i.e. the use of anti-SARS-CoV-2 antibodies which (i) are of the IgG4 type, (ii) recognize a conformational epitope, and optionally (iii) recognize both SARS-CoV and SARS-CoV-2 with high affinity, the present invention is generally directed to such antibodies and to their use in the prophylactic and therapeutic treatment settings disclosed herein.

Furthermore, as illustrated in Example 11 and FIG. 9, antibodies of the present invention could be shown to be capable of binding circulating SARS Cov2 mutants as determined by ELISA. In particular, the antibody of the invention is capable of and thus in alternative or additional embodiment to any of the preceding or following embodiments may be characterized by binding to SARS-CoV-2 variants selected from the group consisting of S1 Mink, B1.351, B1.1.7, P1, B.1.135, RBD N439K, RBD Y453F, RBD N501Y, S1 D614G.

In addition, as illustrated in Example 12 and summarized in FIG. 10 by using a combination of cross-linking and deuterium exchange mass spectrometry the conformational, i.e., discontinuous epitope of antibodies of the present invention, NI-607.531_C8 and NI-607.649_B11 have been demonstrated to be biologically active in the prophylactic and therapeutic treatment settings and capable of binding substantially all circulating SARS Cov2 mutants discovered so far, the antibody of the present invention is preferably characterized by the binding region of either antibody as defined further below and/or by the same functional features, i.e. by binding to one or more, preferably all SARS-CoV-2 variants selected from the group consisting of S1 Mink, B1.351, B1.1.7, P1, B.1.135, RBD N439K, RBD Y453F, RBD N501Y, S1 D614G; binding to at least a first epitope, a second epitope and/or a third epitope of a discontinuous epitope as characterized in Example 12 and shown in FIG. 10, respectively, and/or wherein the binding of the antibody to SARS-CoV-2 and its RBD, respectively, involves interaction with one or more, preferably all amino acid residues identified in Example 12 and shown in FIG. 12, preferably amino acids 112 T, 120 S, 140 K, 144 K and 151 S of the RBD (SEQ ID NO: 301) as determined by Cross-linking Mass Spectrometry, since they are shared by antibody NI-607.531_C8 and NI-607.649_B11 which show a similar profile towards binding to the SARS-CoV-2 variants, for which reason it is tempting to assume that amino acid residues involved in binding of the two antibodies to SARS-CoV-2 RBD that are identical may be important for binding. In addition, or alternatively in one embodiment the antibody of the present invention can be characterized by binding to at least one epitope of the RBD of the SARS-CoV-2 comprising or consisting of the amino acid sequence of SEQ ID NO: 302 and/or to an epitope comprising or consisting of the amino acid sequence of SEQ ID NO: 313, as determined by Cross-linking Mass Spectrometry.

In one embodiment, the antibody of the present invention [NI-607.274_B7] may be characterized by the complementarity determining regions (CDRs) or hypervariable regions of the variable heavy (VH) and variable light (VL) chain comprising the amino acid sequence of SEQ ID: 2 and SEQ ID NO: 7 as shown in Table II and explained in the paragraph below said Table. In another embodiment, the antibody of the present invention [NI-607.274_E5] may be characterized by the CDRs or hypervariable regions of the VH and VL chain comprising the amino acid sequence of SEQ ID NO: 12 and SEQ ID NO: 17 as shown in Table II and explained in the paragraph below said Table. In another embodiment, the antibody of the present invention [NI-607.275_C5] may be characterized by the CDRs or hypervariable regions of the VH and VL chain comprising the amino acid sequence of SEQ ID NO: 22 and SEQ ID NO: 27 as shown in Table II and explained in the paragraph below said Table. In another embodiment, the antibody of the present invention [NI-607.426_D4] may be characterized by the CDRs or hypervariable regions of the VH and VL chain comprising the amino acid sequence of SEQ ID NO: 32 and SEQ ID NO: 37 as shown in Table II and explained in the paragraph below said Table. In another embodiment, the antibody of the present invention [NI-607.426_E2] may be characterized by the CDRs or hypervariable regions of the VH and VL chain comprising the amino acid sequence of SEQ ID NO: 42 and SEQ ID NO: 47 as shown in Table II and explained in the paragraph below said Table. In another embodiment, the antibody of the present invention [NI-607.426_F11] may be characterized by the CDRs or hypervariable regions of the VH and VL chain comprising the amino acid sequence of SEQ ID NO: 52 and SEQ ID NO: 57 as shown in Table II and explained in the paragraph below said Table. In another embodiment, the antibody of the present invention [NI-607.427_C5] may be characterized by the CDRs or hypervariable regions of the VH and VL chain comprising the amino acid sequence of SEQ ID NO: 62 and SEQ ID NO: 67 as shown in Table II and explained in the paragraph below said Table. In another embodiment, the antibody of the present invention [NI-607.428_B9] may be characterized by the CDRs or hypervariable regions of the VH and VL chain comprising the amino acid sequence of SEQ ID NO: 72 and SEQ ID NO: 77 as shown in Table II and explained in the paragraph below said Table. In another embodiment, the antibody of the present invention [NI-607.429_B9] may be characterized by the CDRs or hypervariable regions of the VH and VL chain comprising the amino acid sequence of SEQ ID NO: 82 and SEQ ID NO: 87 as shown in Table II and explained in the paragraph below said Table. In another embodiment, the antibody of the present invention [NI-607.429_E4] may be characterized by the CDRs or hypervariable regions of the VH and VL chain comprising the amino acid sequence of SEQ ID NO: 92 and SEQ ID NO: 97 as shown in Table II and explained in the paragraph below said Table. In another embodiment, the antibody of the present invention [NI-607.529_B9] may be characterized by the CDRs or hypervariable regions of the VH and VL chain comprising the amino acid sequence of SEQ ID NO: 102 and SEQ ID NO: 107 as shown in Table II and explained in the paragraph below said Table. In another embodiment, the antibody of the present invention [NI-607.529_G4] may be characterized by the CDRs or hypervariable regions of the VH and VL chain comprising the amino acid sequence of SEQ ID NO: 112 and SEQ ID NO: 117 as shown in Table II and explained in the paragraph below said Table. In another embodiment, the antibody of the present invention [NI-607.531_C8] may be characterized by the CDRs or hypervariable regions of the VH and VL chain comprising the amino acid sequence of SEQ ID NO: 122 and SEQ ID NO: 127 as shown in Table II and explained in the paragraph below said Table. In another embodiment, the antibody of the present invention [NI-607.531_D8] may be characterized by the CDRs or hypervariable regions of the VH and VL chain comprising the amino acid sequence of SEQ ID NO: 132 and SEQ ID NO: 137 as shown in Table II and explained in the paragraph below said Table. In another embodiment, the antibody of the present invention [NI-607.532_B6] may be characterized by the CDRs or hypervariable regions of the VH and VL chain comprising the amino acid sequence of SEQ ID NO: 142 and SEQ ID NO: 147 as shown in Table II and explained in the paragraph below said Table. In another embodiment, the antibody of the present invention [NI-607.532_C11] may be characterized by the CDRs or hypervariable regions of the VH and VL chain comprising the amino acid sequence of SEQ ID NO: 152 and SEQ ID NO: 157 as shown in Table II and explained in the paragraph below said Table. In another embodiment, the antibody of the present invention [NI-607.532_C8] may be characterized by the CDRs or hypervariable regions of the VH and VL chain comprising the amino acid sequence of SEQ ID NO: 162 and SEQ ID NO: 167 as shown in Table II and explained in the paragraph below said Table. In another embodiment, the antibody of the present invention [NI-607.532_D3] may be characterized by the CDRs or hypervariable regions of the VH and VL chain comprising the amino acid sequence of SEQ ID NO: 172 and SEQ ID NO: 177 as shown in Table II and explained in the paragraph below said Table. In another embodiment, the antibody of the present invention [NI-607.532_D4] may be characterized by the CDRs or hypervariable regions of the VH and VL chain comprising the amino acid sequence of SEQ ID NO: 182 and SEQ ID NO: 187 as shown in Table II and explained in the paragraph below said Table. In another embodiment, the antibody of the present invention [NI-607.532_D8] may be characterized by the CDRs or hypervariable regions of the VH and VL chain comprising the amino acid sequence of SEQ ID NO: 192 and SEQ ID NO: 197 as shown in Table II and explained in the paragraph below said Table. In another embodiment, the antibody of the present invention [NI-607.532_F9] may be characterized by the CDRs or hypervariable regions of the VH and VL chain comprising the amino acid sequence of SEQ ID NO: 202 and SEQ ID NO: 207 as shown in Table II and explained in the paragraph below said Table. In another embodiment, the antibody of the present invention [NI-607.649_B11] may be characterized by the CDRs or hypervariable regions of the VH and VL chain comprising the amino acid sequence of SEQ ID NO: 212 and SEQ ID NO: 217 as shown in Table II and explained in the paragraph below said Table. In another embodiment, the antibody of the present invention [NI-607.531_E7] may be characterized by the CDRs or hypervariable regions of the VH and VL chain comprising the amino acid sequence of SEQ ID NO: 222 and SEQ ID NO: 227 as shown in Table II and explained in the paragraph below said Table. In another embodiment, the antibody of the present invention [NI-607.532_F3] may be characterized by the CDRs or hypervariable regions of the VH and VL chain comprising the amino acid sequence of SEQ ID NO: 232 and SEQ ID NO: 237 as shown in Table II and explained in the paragraph below said Table. In another embodiment, the antibody of the present invention [NI-607.649_G7] may be characterized by the CDRs or hypervariable regions of the VH and VL chain comprising the amino acid sequence of SEQ ID NO: 242 and SEQ ID NO: 247 as shown in Table II and explained in the paragraph below said Table. In another embodiment, the antibody of the present invention [NI-607.761_B7] may be characterized by the CDRs or hypervariable regions of the VH and VL chain comprising the amino acid sequence of SEQ ID NO: 252 and SEQ ID NO: 257 as shown in Table II and explained in the paragraph below said Table. In another embodiment, the antibody of the present invention [NI-607.791_B10] may be characterized by the CDRs or hypervariable regions of the VH and VL chain comprising the amino acid sequence of SEQ ID NO: 262 and SEQ ID NO: 267 as shown in Table II and explained in the paragraph below said Table. In another embodiment, the antibody of the present invention [NI-607.531_E3] may be characterized by the CDRs or hypervariable regions of the VH and VL chain comprising the amino acid sequence of SEQ ID NO: 272 and SEQ ID NO: 277 as shown in Table II and explained in the paragraph below said Table. In another embodiment, the antibody of the present invention [NI-607.820_B6] may be characterized by the CDRs or hypervariable regions of the VH and VL chain comprising the amino acid sequence of SEQ ID NO: 282 and SEQ ID NO: 287 as shown in Table II and explained in the paragraph below said Table. In another embodiment, the antibody of the present invention [NI-607.820_B7] may be characterized by the CDRs or hypervariable regions of the VH and VL chain comprising the amino acid sequence of SEQ ID NO: 292 and SEQ ID NO: 297 as shown in Table II and explained in the paragraph below said Table.

In one embodiment, the present invention provides a composition characterized by comprising said antibodies.

TABLE II
Nucleotide and amino acid sequences of the variable regions (VH, VL)
of the antibodies of the present invention. Underlined amino acids
indicate the CDR coding regions in the variable chain sequence.

| | Nucleotide and amino acid sequence of the variable |
|---|---|
| Antibody | heavy (VH) and variable light (VL) chains. |
| NI-607.274_B7-VH | CAGGTGCAGCTGGTGGAGTCTGGGGGAGGCAGGGTCCAGCCTGGGAGG |
| | TCCCTGAGACTCTCCTGTGCAGCCTCTGGATTCACCTTCAGTACTCAT |
| | GGCATGCACTGGGTCCGCCAGGTTCCAGGCAAGGGTCTGGACTGGATT |
| | ACAGGTATCACATATGATGGAAGTGATCAGTTTTATGCAGACTCCGTG |
| | AAGGGCCGCTTCACCATCTCCAGAGACAATTCAAAGAATATGCTATAT |
| | CTACAAATGAACAGCCTGAGAGCTGAGGACACGGCTGTGTATTTCTGT |
| | GCGAAAGATCTGGAGTGGTTAACAAACTATCTTGACCACTGGGGCCAG |
| | GGAACCCTGGTCACCGTCTCCTCA SEQ ID NO: 1 |

| | |
|---|---|
| NI-607.274_B7-VH | QVQLVESGGGRVQPGRSLRLSCAASGFTFSTHGMHWVRQVPGKGLDWI |
| | TGITYDGSDQFYADSVKGRFTISRDNSKNMLYLQMNSLRAEDTAVYFC |
| | AKDLEWLTNYLDHWGQGTLVTVSS SEQ ID NO: 2 |
| NI-607.274_B7-VL | GATGTTGTGATGACTCAGTCTCCAGGCACCCTGTCTGTGTCTCCAGGG |
| | GAAAGAGCCACCCTCTCCTGCAGGGCCAGTCAGAATATTAACTACAAC |
| | TTAGCCTGGTACCAGCAGAAGCCTGGCCAGGCTCCCAGGCTTCTCATC |
| | TTCGATGCGTCCACCAGGGCCACTGGTATCCCAGCCAGATTCACTGGC |
| | AGTGGGTCTGGGACAGAGTTCACTCTCACCATCAGCAGCCTGCAGTCT |
| | GAAGATTTTGCAGTTTATTACTGTCAGCAGTCTAATGCCTGGCTCACT |
| | TTCGGCGGAGGGACCAAGGTGGAAATCAAA SEQ ID NO: 6 |
| NI-607.274_B7-VL | DVVMTQSPGTLSVSPGERATLSCRASQNINYNLAWYQQKPGQAPRLLI |
| | FDASTRATGIPARFTGSGSGTEFTLTISSLQSEDFAVYYCQQSNAWLT |
| | FGGGTKVEIK SEQ ID NO: 7 |
| NI-607.274_E5-VH | GAGGTGCAGCTGGTGGAGTCTGGGGGAGGCATGGTCCAGCCTGGGAGG |
| | TCCCTGAGACTCTCCTGTGCAGGCTCTGGATTCACCTTCAGCACTCAT |
| | GGCATGCACTGGGTCCGCCAGGCTCCAGGCAAGGGTCTGGAGTGGATT |
| | ACAGGTATCACATATGATGGAAGTGATGAATTTTATGCAGACTCCGTG |
| | AAGGGCCGATTCACCATCGCCAGAGACAATTCAAAGAATATGCTATAT |
| | CTACAAATGAACAGCCTGAGAGCTGAGGACACGGCTGTGTATTTCTGT |
| | GCGAAAGATCTGGAGTGGTTAACAAACTATGTTGACCACTGGGGCCAG |
| | GGAACCCTGGTCACCGTCTCCTCA SEQ ID NO: 11 |
| NI-607.274_E5-VH | EVQLVESGGGMVQPGRSLRLSCAGSGFTFSTHGMHWVRQAPGKGLEWI |
| | TGITYDGSDEFYADSVKGRFTIARDNSKNMLYLQMNSLRAEDTAVYFC |
| | AKDLEWLTNYVDHWGQGTLVTVSS SEQ ID NO: 12 |
| NI-607.274_E5-VL | GATATTGTGATGACTCAGTCTCCAGGCACCCTGTCTGTGTCTCCAGGG |
| | GAAAGAGCCACCCTCTCCTGCAGGGCCAGTCAGAGTGTTAACTACAAC |
| | TTAGCCTGGTACCAGCAGAAGCCTGGCCAGGCTCCCAGGCTTCTCATC |
| | TTCGATGCGTCCACCAGGGCCACTGGTATCCCAGCCAGATTCACTGGC |
| | AGTGGGTCTGGGACAGAGTTCACTCTCACCATCAGCAGCCTGCAGTCT |
| | GAAGATTTTGCAGTTTATTACTGTCAGCAGTCTAATGCCTGGCTCACT |
| | TTCGGCGGAGGGACCAAGCTGGAGATCAAA SEQ ID NO: 16 |
| NI-607.274_E5-VL | DIVMTQSPGTLSVSPGERATLSCRASQSVNYNLAWYQQKPGQAPRLLI |
| | FDASTRATGIPARFTGSGSGTEFTLTISSLQSEDFAVYYCQQSNAWLT |
| | FGGGTKLEIK SEQ ID NO: 17 |
| NI-607.275_C5-VH | CAGGTGCAGCTGGTGGAGTCTGGGGGAGGCTTGGTCCAGCCTGGGGGG |
| | TCCCTGAGACTCTCCTGTGCAGCCTCTGGATTCACCGTCAGTAGCAAC |
| | TACATGAGCTGGGTCCGCCAGGCTCCAGGGAAGGGGCTGGAGTGGGTC |
| | TCAGTTATTTATAGCGGTGGTAGCACATACTACGCAGACTCCGTGAAG |
| | GGCAGATTCACCATCTCCAGAGACAATTCCAAGAACACGCTGTATCTT |
| | CAAATGAACAGCCTGAGAGCCGAGGACACGGCTGTGTATTACTGTGCG |
| | AGAGATTTTGGGGATTACTACTTTGACTACTGGGGCCAGGGAACCCTG |
| | GTCACCGTCTCCTCA SEQ ID NO: 21 |
| NI-607.275_C5-VH | QVQLVESGGGLVQPGGSLRLSCAASGFTVSSNYMSWVRQAPGKGLEWV |
| | SVIYSGGSTYYADSVKGRFTISRDNSKNTLYLQMNSLRAEDTAVYYCA |
| | RDFGDYYFDYWGQGTLVTVSS SEQ ID NO: 22 |
| NI-607.275_C5-VL | GATATTGTGTTGACGCAGTCTCCAGCCACCCTGTCTTTGTCTCCAGGG |
| | GAAAGAGCCACCCTCTCCTGCGGGGCCAGTCAGAGTGTTAGCAGCAGC |
| | TACTTAGCCTGGTACCAGCAGAAACCTGGCCTGGCGCCCAGGCTCCTC |
| | ATCTATGATGCATCCAGCAGGGCCACTGGCATCCCAGACAGGTTCAGT |
| | GGCAGTGGGTCTGGGACAGACTTCACTCTCACCATCAGCAGACTGGAG |
| | CCTGAAGATTTTGCAGTGTATTACTGTCAGCAGTATGGTAGCTCACCT |
| | AGGACTTTTGGCCAGGGGACCAAGCTGGAGATCAAA |
| | SEQ ID NO: 26 |
| NI-607.275_C5-VL | DIVLTQSPATLSLSPGERATLSCGASQSVSSSYLAWYQQKPGLAPRLL |
| | IYDASSRATGIPDRFSGSGSGTDFTLTISRLEPEDFAVYYCQQYGSSP |
| | RTFGQGTKLEIK SEQ ID NO: 27 |
| NI-607.426_D4-VH | GAGGTGCAGCTGGTGCAGTCTGGGGCTGAGGTGAAGAAGCCTGGGGCC |
| | TCAGTGAAGGTCTCCTGCAAGGCTTCTGGATACACCTTCACCGGCTAC |
| | TATATGCACTGGGTGCGACAGGCCCCTGGACAAGGGCTTGAGTGGATG |
| | GGATGGATCAACCCTAACAGTGGTGGCACAAACTATGCACAGAAGTTT |
| | CAGGGCAGGGTCACCATGACCAGGGACACGTCCATCAGCACAGCCTAC |
| | ATGGAGCTGAGCAGGCTGAGATCTGACGACACGGCCATGTATTACTGT |
| | GCGAGAGAGGGGTGTATAGTGTGGGAGATAGCGGGGGCACTTTGCTAC |
| | TACGGTATGGACGTCTGGGGCCAAGGGACCACGGTCACCGTCTCCTCA |
| | SEQ ID NO: 31 |
| NI-607.426_D4-VH | EVQLVQSGAEVKKPGASVKVSCKASGYTFTGYYMHWVRQAPGQGLEWM |
| | GWINPNSGGTNYAQKFQGRVTMTRDTSISTAYMELSRLRSDDTAMYYC |
| | AREGCIVWEIAGALCYYGMDVWGQGTTVTVSS SEQ ID NO: 32 |
| NI-607.426_D4-VL | GAAATTGTGTTGACACAGTCTCCAGCCACCCTGTCTTTGTCTCCAGGG |
| | GAAAGAGCCACCCTCTCCTGCAGGGCCAGTCAGAGTGTTAGCAGCTAC |
| | TTAGCCTGGTACCAACAGAAACCTGGCCAGGCTCCCAGGCTCCTCATC |
| | TATGATGCATCCAACAGGGCCACTGGCATCCCAGCCAGGTTCAGTGGC |
| | AGTGGGTCTGGGACAGACTTCACTCTCACCATCAGCAGCCTAGAGCCT |
| | GAAGATTTTGCAGTTTATTACTGTCAGCAGCGTAGCAACTGGCCTCCG |
| | GTCACCTTCGGCCAAGGGACACGACTGGAGATTAAA |
| | SEQ ID NO: 36 |
| NI-607.426_D4-VL | EIVLTQSPATLSLSPGERATLSCRASQSVSSYLAWYQQKPGQAPRLLI |
| | YDASNRATGIPARFSGSGSGTDFTLTISSLEPEDFAVYYCQQRSNWPP |
| | VTFGQGTRLEIK SEQ ID NO: 37 |
| NI-607.426_E2-VH | GAGGTGCAGCTGGTGGAGTCTGGGGGAGGCTTGGTACAGCCTGGGGGG |
| | TCCCTGAGACTCTCCTGTGCAGCCTCTGGATTCACCTTCAGTAGCTAC |
| | GACATGCACTGGGTCCGCCAAGCTACAGGAAAAGGTCTGGAGTGGGTC |
| | TCAGCTATTGGTACTGCTGGTGACACATACTATCCAGGCTCCGTGAAG |
| | GGCCGATTCACCATCTCCAGAGAAAATGCCAAGAACTCCTTGTATCTT |
| | CAAATGAACAGCCTGAGAGCCGAGGACACAGCCGTGTATTACTGTGCA |
| | AGAGCATCCTTTCTAGACTACTCGTGGCTCTACTTTGACTACTGGGGC |
| | CAGGGAACCCTGGTCACCGTCTCCTCA SEQ ID NO: 41 |
| NI-607.426_E2-VH | EVQLVESGGGLVQPGGSLRLSCAASGFTFSSYDMHWVRQATGKGLEWV |
| | SAIGTAGDTYYPGSVKGRFTISRENAKNSLYLQMNSLRAEDTAVYYCA |
| | RASFLDYSWLYFDYWGQGTLVTVSS SEQ ID NO: 42 |
| NI-607.426_E2-VL | GACATCCAGATGACCCAGTCTCCATCCTCCCTGTCTGCATCTGTAGGA |
| | GACAGAGTCACCATCACTTGCCGGGCAAGTCAGAGCATTAGCAGCTAT |
| | TTAAATTGGTATCAGCAGAAACCAGGGAAAGCCCCTAAGCTCCTGATC |
| | TATGCTGCATCCAGTTTGCAAAGTGGGGTCCCATCAAGGTTCAGTGGC |
| | AGTGGATCTGGGACAGATTTCACTCTCACCATCAGCAGTCTGCAACCT |
| | GAAGATTTTGCAACTTACTACTGTCAACAGAGTTACACTCAACCAGAG |
| | GGGACGTTCGGCCAAGGGACCAAAGTGGATATCAAA |
| | SEQ ID NO: 46 |
| NI-607.426_E2-VL | DIQMTQSPSSLSASVGDRVTITCRASQSISSYLNWYQQKPGKAPKLLI |
| | YAASSLQSGVPSRFSGSGSGTDFTLTISSLQPEDFATYYCQQSYTQPE |
| | GTFGQGTKVDIK SEQ ID NO: 47 |
| NI-607.426_F11-VH | CAGGTGCAGCTGGTGGAGTCTGGGGGAGGCTTGGTCCAGCCTGGGGGG |
| | TCCCTGAGACTCTCCTGTGCAGCCTCTGGATTCACCTTTAGTAGCTAT |
| | TGGATGAGCTGGGTCCGCCAGGCTCCAGGGAAGGGGCTGGAGTGGGTG |
| | GCCAACATAAAGCAAGATGGAAGTGAGAAATACTATGTGGACTCTGTG |
| | AAGGGCCGATTCACCATCTCCAGAGACAACGCCAAGAACTCACTGTAT |
| | CTGCAAATGAACAGCCTGAGAGCCGAGGACACGGCTGTGTATTACTGT |
| | GCGAGAGGACTAGTTGGGCAGTGGCTGGGGGTTGACTACTGGGGCCAG |
| | GGAACCCTGGTCACCGTCTCCTCA SEQ ID NO: 51 |
| NI-607.426_F11-VH | QVQLVESGGGLVQPGGSLRLSCAASGFTFSSYWMSWVRQAPGKGLEWV |
| | ANIKQDGSEKYYVDSVKGRFTISRDNAKNSLYLQMNSLRAEDTAVYYC |
| | ARGLVGQWLGVDYWGQGTLVTVSS SEQ ID NO: 52 |
| NI-607.426_F11-VL | CAGTCTGTGCTGACTCAGCCCCACTCTGTGTCGGAGTCTCCGGGGAAG |
| | ACGGTAACCATCTCCTGCACCGGCAGCAGTGGCAGCATTGCCAGCAAC |
| | TATGTGCAGTGGTACCAGCAGCGCCCGGGCAGTGCCCCCACCACTGTG |
| | ATCTATGAGGATAACCAAAGACCCTCTGGGGTCCCTGATCGGTTCTCT |
| | GGCTCCATCGACAGCTCCTCCAACTCTGCCTCCCTCACCATCTCTGGA |
| | CTGAAGACTGAGGACGAGGCTGACTACTACTGTCAGTCTTATGATAGC |
| | AGCAATTGGGTGTTCGGCGGAGGGACCAAGCTGACCGTCCTA |
| | SEQ ID NO: 56 |
| NI-607.426_Fl 1-VL | QSVLTQPHSVSESPGKTVTISCTGSSGSIASNYVQWYQQRPGSAPTTV |
| | IYEDNQRPSGVPDRFSGSIDSSSNSASLTISGLKTEDEADYYCQSYDS |
| | SNWVFGGGTKLTVL SEQ ID NO: 57 |
| NI-607.427_C5-VH | CAGGTGCAGCTGGTGCAGTCTGGAGCTGAGGTGAAGAAGCCTGGGGCC |
| | TCAGTGAAGGTCTCCTGCAAGGCTTCTGGTTACACCTTTACCAGCTAT |
| | GGTATCAGCTGGGTGCGACAGGCCCCTGGACAAGGGCTTGAGTGGATG |
| | GGATGGATCAGCGCTTACAATGGTAACACAAACTATGCACAGAAGCTC |
| | CAGGGCAGAGTCACCATGACCACAGACACATCCACGAGCACAGCCTAC |
| | ATGGAGCTGAGGAGCCTGAGATCTGACGACACGGCCGTGTATTACTGT |
| | GCGAGAGATAATCGGGCGGGGTGGGACCAACTGGACTACTGGGGCCAG |
| | GGAACCCTGGTCACCGTCTCCTCA SEQ ID NO: 61 |
| NI-607.427_C5-VH | QVQLVQSGAEVKKPGASVKVSCKASGYTFTSYGISWVRQAPGQGLEWM |
| | GWISAYNGNTNYAQKLQGRVTMTTDTSTSTAYMELRSLRSDDTAVYYC |
| | ARDNRAGWDQLDYWGQGTLVTVSS SEQ ID NO: 62 |
| NI-607.427_C5-VL | GACATCCAGATGACCCAGTCTCCTTCCACCCTGTCTGCATCTGTAGGA |
| | GACAGAGTCACCATCACTTGCCGGGCCAGTCAGAGTATTAGTAGCTGG |
| | TTGGCCTGGTATCAGCAGAAACCAGGGAAAGCCCCTAAGCTCCTGATC |
| | TATAAGGCGTCTAGTTTAGAAAGTGGGGTCCCATCAAGGTTCAGCGGC |
| | AGTGGATCTGGGACAGAATTCACTCTCACCATCAGCAGCCTGCAGCCT |
| | GATGATTTTGCAACTTATTACTGCCAACAGTATAATAGTTACCCGTAC |
| | ACTTTTGGCCAGGGGACCAAGCTGGAGATCAAA SEQ ID NO: 66 |
| NI-607.427_C5-VL | DIQMTQSPSTLSASVGDRVTITCRASQSISSWLAWYQQKPGKAPKLLI |
| | YKASSLESGVPSRFSGSGSGTEFTLTISSLQPDDFATYYCQQYNSYPY |
| | TFGQGTKLEIK SEQ ID NO: 67 |
| NI-607.428_B9-VH | CAGGTGCAGCTGGTGCAGTCTGGGGCTGAGGTGAAGAAGCCTGGGGCC |
| | TCAGTGAAGGTCTCCTGCAAGGCTTCTGGATACACCTTCACCGGCTAC |
| | TATATGCACTGGGTGCGACAGGCCCCTGGACAAGGGCTTGAGTGGATG |
| | GGATGGATCAACCCTAACAGTGGTGGCACAAACTATGCACAGAAGTTT |
| | CAGGGCAGGGTCACCATGACCAGGGACACGTCCATCAGCACAGCCTAC |
| | ATGGAGCTGAGCAGGCTGAGATCTGACGACACGGCCGTGTATTACTGT |
| | GCGAGAGAGGGGTGTATAGTGTGGGAGATAGCGGGGGCACTTTGCTAC |
| | TACGGTATGGACGTCTGGGGCCAAGGGACCACGGTCACCGTCTCCTCA |
| | SEQ ID NO: 71 |
| NI-607.428_B9-VH | QVQLVQSGAEVKKPGASVKVSCKASGYTFTGYYMHWVRQAPGQGLEWM |
| | GWINPNSGGTNYAQKFQGRVTMTRDTSISTAYMELSRLRSDDTAVYYC |
| | AREGCIVWEIAGALCYYGMDVWGQGTTVTVSS SEQ ID NO: 72 |
| NI-607.428_B9-VL | GAAATAGTGATGACGCAGTCTCCAGCCACCCTGTCTTTGTCTCCAGGG |
| | GAAAGAGCCACCCTCTCCTGCAGGGCCAGTCAGAGTGTTAGCAGCTAC |
| | TTAGCCTGGTACCAACAGAAACCTGGCCAGGCTCCCAGGCTCCTCATC |
| | TATGATGCATCCAACAGGGCCACTGGCATCCCAGCCAGGTTCAGTGGC |
| | AGTGGGTCTGGGACAGACTTCACTCTCACCATCAGCAGCCTAGAGCCT |
| | GAAGATTTTGCAGTTTATTACTGTCAGCAGCGTAGCAACTGGCCTCCG |
| | GTCACCTTCGGCCAAGGAACACGACTGGAGATTAAA |
| | SEQ ID NO: 76 |
| NI-607.428_B9-VL | EIVMTQSPATLSLSPGERATLSCRASQSVSSYLAWYQQKPGQAPRLLI |
| | YDASNRATGIPARFSGSGSGTDFTLTISSLEPEDFAVYYCQQRSNWPP |
| | VTFGQGTRLEIK SEQ ID NO: 77 |
| NI-607.429_B9-VH | GAGGTGCAGCTGGTGGAATCTGGGGGAGGCTTGGTCCAGCCTGGGGGG |
| | TCCCTGAGACTCTCCTGTGCAGCCTCTGGATTCACCTTTAGTAGCTAT |
| | TGGATGAGCTGGGTCCGCCAGGCTCCAGGGAAGGGGCTGGAGTGGGTG |
| | GCCAACATAAAGCAAGATGGAAGTGAGAAATACTATGTGGACTCTGTG |
| | AAGGGCCGATTCACCATCTCCAGAGACAACGCCAAGAACTCACTGTAT |
| | CTGCAAATGAACAGCCTGCGAGCCGAGGACACGGCCGTGTATTACTGT |
| | GCGAGCTTATTTTGGAGACGGGGCTCGTTCGACCCCTGGGGCCAGGGA |
| | ACCCTGGTCACCGTCTCCTCA SEQ ID NO: 81 |
| NI-607.429_B9-VH | EVQLVESGGGLVQPGGSLRLSCAASGFTFSSYWMSWVRQAPGKGLEWV |
| | ANIKQDGSEKYYVDSVKGRFTISRDNAKNSLYLQMNSLRAEDTAVYYC |
| | ASLFWRRGSFDPWGQGTLVTVSS SEQ ID NO: 82 |
| NI-607.429_B9-VL | CAGTCTGTGCTGACTCAGCCGCACTCTGTGTCGGAGTCTCCGGGGAAG |
| | ACGGTAACCATCTCCTGCACCCGCAGCACTGGCAGCATTGCCAGCAAC |
| | TATGTGCAGTGGTACCAGCAGCGCCCGGGCAGTGCCCCCACCACTGTG |
| | ATCTATGAGGATAACCAAAGACCCTCTGGGGTCCCTGATCGGTTCTCT |
| | GGCTCCATCGACAGCTCCTCCAACTCTGCCTCCCTCACCATCTCTGGA |
| | CTGAAGACTGAGGACGAGGCTGACTACTACTGTCAGTCTTATGATACC |
| | AGCTCCTATTGGGTGTTCGGCGGAGGGACCAAGCTGACCGTCCTA |
| | SEQ ID NO: 86 |
| NI-607.429_B9-VL | QSVLTQPHSVSESPGKTVTISCTRSTGSIASNYVQWYQQRPGSAPTTV |
| | IYEDNQRPSGVPDRFSGSIDSSSNSASLTISGLKTEDEADYYCQSYDT |
| | SSYWVFGGGTKLTVL SEQ ID NO: 87 |
| NI-607.429_E4-VH | CAGGTGCAGCTGGTGGAGTCTGGGGGAGGCTTGGTCCAGCCTGGGGGG |
| | TCCCTGAGACTCTCCTGTGCAGCCTCTGGATTCACCGTCAGTAGCAAC |
| | TACATGAGCTGGGTCCGCCAGGCTCCAGGGAAGGGGCTGGAGTGGGTC |
| | TCAGTTATTTATAGCGGTGGTAGCACATTCTACGCAGACTCCGTGAAG |
| | GGCAGATTCACCATCTCCAGAGACAATTCCAAGAACACGCTGTATCTT |
| | CAAATGAACAGCCTGAGAGCCGAGGACACGGCTGTGTATTACTGTGCG |
| | AGAGATTTGCAGGAGTCCGGTATGGACGTTTGGGGCCAAGGGACCACG |
| | GTCACCGTCTCCTCA SEQ ID NO: 91 |
| NI-607.429_E4-VH | QVQLVESGGGLVQPGGSLRLSCAASGFTVSSNYMSWVRQAPGKGLEWV |
| | SVIYSGGSTFYADSVKGRFTISRDNSKNTLYLQMNSLRAEDTAVYYCA |
| | RDLQESGMDVWGQGTTVTVSS SEQ ID NO: 92 |
| NI-607.429_E4-VL | GACATCCAGTTGACCCAGTCTCCATCTTCCGTGTCTGCATCTGTAGGA |
| | GACAGAGTCACCATCACTTGTCGGGCGAGTCAGGGTATTAGCAGCTGG |
| | TTAGCCTGGTATCAGCAGAAGCCAGGGAAAGCCCCTAAGCTCCTGATC |
| | TATGCTGCATCCAGTTTGCAAAGTGGGGTCCCATCAAGGTTCAGCGGC |
| | AGTGGATCTGGGACAGATTTCACTCTCACCATCAGCAGCCTGCAGCCT |
| | GAAGATTTTGCAACTTACTATTGTCAACAGGCTAACAGTTTCCCCACC |
| | TTCGGCCAAGGGACACGACTGGAGATCAAA SEQ ID NO: 96 |
| NI-607.429_E4-VL | DIQLTQSPSSVSASVGDRVTITCRASQGISSWLAWYQQKPGKAPKLLI |
| | YAASSLQSGVPSRFSGSGSGTDFTLTISSLQPEDFATYYCQQANSFPT |
| | FGQGTRLEIK SEQ ID NO: 97 |
| NI-607.529_B9-VH | CAGGTGCAGCTGCAGGAGTCGGGCCCAGGACTGGTGAAGCCTTCGGAG |
| | ACCCTGTCCCTCACCTGCACTGTCTCTGGTGGCTCCATCAGCAGTAGT |
| | AGTTACTACTGGGGCTGGATCCGCCAGCCCCCAGGGAAGGGGCTGGAG |
| | TGGATTGGGAATATCTATTATAGTGGGAGCACCTACTACAACCCGTCC |
| | CTCAAGAGTCGAGTCACCATATCCGTAGACACGTCCAAGAACCAGTTC |
| | TCCCTGAAGCTGAACTCTGTGACCGCCGCAGACACGGCTGTGTATTTC |
| | TGTGCGAGACTACTATGGTTACGCGGGAGCTACTATTGGGGCCAGGGA |
| | ACCCTGGTCACCGTCTCCTCA SEQ ID NO: 101 |
| NI-607.529_B9-VH | QVQLQESGPGLVKPSETLSLTCTVSGGSISSSSYYWGWIRQPPGKGLE |
| | WIGNIYYSGSTYYNPSLKSRVTISVDTSKNQFSLKLNSVTAADTAVYF |
| | CARLLWLRGSYYWGQGTLVTVSS SEQ ID NO: 102 |
| NI-607.529_B9-VL | TCCTATGAGCTGACTCAGCCCCACTCTGTGTCGGAGTCTCCGGGGAAG |
| | ACGGTAACCATCTCCTGCACCGGCAGCAGTGGCAGCATTGCCAGCAAC |
| | TATGTGCAGTGGTACCAGCAGCGCCCGGGCAGTGCCCCCACCACTGTG |
| | ATCTATGAGGATAACCAAAGACCCTCTGGGGTCCCTGATCGGTTCTCT |
| | GGCTCCATCGACAGCTCCTCCAACTCTGCCTCCCTCACCATCTCTGGA |
| | CTGAAGACTGAGGACGAGGCTGACTACTACTGTCAGTCTTATGATAGC |
| | AGCAATTGGGTGTTCGGCGGAGGGACCAAGCTGACCGTCCTA |
| | SEQ ID NO: 106 |
| NI-607.529_B9-VL | SYELTQPHSVSESPGKTVTISCTGSSGSIASNYVQWYQQRPGSAPTTV |
| | IYEDNQRPSGVPDRFSGSIDSSSNSASLTISGLKTEDEADYYCQSYDS |
| | SNWVFGGGTKLTVL SEQ ID NO: 107 |
| NI-607.529_G4-VH | CAGGTGCAGCTACAGCAGTGGGGCGCAGGACTGTTGAAGCCTTCGGAG |
| | ACCCTGTCCCTCACCTGCGCTGTCTATGGTGGGTCCTTCAGTGGTTAC |
| | TACTGGAGCTGGATCCGCCAGCCCCCAGGGAAGGGGCTGGAGTGGATT |
| | GGGGAAATCAATCATAGTGGAAGCACCAACTACAACCCGTCCCTCAAG |
| | AGTCGAGTCACCATATCAGTAGACACGTCCAAGAACCAGTTCTCCCTG |
| | AAGCTGAGCTCTGTGACCGCCGCGGACACGGCTGTGTATTACTGTGCG |
| | AGAGCCTCCCTCTATTACTCTGATAGTAGTGGTTATTCACACCCGTAC |
| | TACTTTGACTACTGGGGCCAGGGAACCCTGGTCACCGTCTCCTCA |
| | SEQ ID NO: 111 |
| NI-607.529_G4-VH | QVQLQQWGAGLLKPSETLSLTCAVYGGSFSGYYWSWIRQPPGKGLEWI |
| | GEINHSGSTNYNPSLKSRVTISVDTSKNQFSLKLSSVTAADTAVYYCA |
| | RASLYYSDSSGYSHPYYFDYWGQGTLVTVSS SEQ ID NO: 112 |
| NI-607.529_G4-VL | CAGCCTGTGCTGACGCAGCCGCCCTCAGTGTCTGGGGCCCCAGGGCAG |
| | AGGGTCACCATCTCCTGCACTGGGAGCAGCTCCAACATCGGGGCAGGT |
| | TATGATGTACACTGGTACCAGCAGCTTCCAGGAACAGCCCCCAAACTC |
| | CTCATCTATGGTAACAGCAATCGGCCCTCAGGGGTCCCTGACCGATTC |
| | TCTGGCTCCAAGTCTGGCATGTCAGCCTCCCTGGCCATCACTGGGCTC |
| | CAGGCTGAGGATGAGGCTGATTATTACTGCCAGTCCTATGACAGCAGC |
| | CTGAGTGGTTGGGTGTTCGGCGGAGGGACCAAGCTGACCGTCCTA |
| | SEQ ID NO: 116 |
| NI-607.529_G4-VL | QPVLTQPPSVSGAPGQRVTISCTGSSSNIGAGYDVHWYQQLPGTAPKL |
| | LIYGNSNRPSGVPDRFSGSKSGMSASLAITGLQAEDEADYYCQSYDSS |
| | LSGWVFGGGTKLTVL SEQ ID NO: 117 |
| NI-607.531_C8-VH | GAGGTGCAGCTGGTGGAATCTGGGGGAGGCGTGGTCCAGCCTGGGAGG |
| | TCCCTGAGACTCTCCTGTACAGCCTCTGGATTCACCTTCAGTTTCTAT |
| | TCTGTGCACTGGGTCCGCCAGGCTCCAGGCAAGGGGCTGGAGTGGGTG |
| | GCAGTTATTTCATATGATGGAAGCACTAAATACTACGCAGACTCCGTG |
| | AAGGGCCGATTCACCATCTCCAGAGACAATTCCAAGAATACGCTGTAT |
| | CTGCAAATGGACAGCCTGAGAACTGAGGACACGGCTGTCTATTACTGT |
| | GCGAGTCCCCCCATCTCCCCCATACCTGCAGCTGGTACGCCCCTTGCC |
| | TACTGGGGCCAGGGAACCCTGGTCACCGTCTCCTCA |
| | SEQ ID NO: 121 |
| NI-607.531_C8-VH | EVQLVESGGGVVQPGRSLRLSCTASGFTFSFYSVHWVRQAPGKGLEWV |
| | AVISYDGSTKYYADSVKGRFTISRDNSKNTLYLQMDSLRTEDTAVYYC |
| | ASPPISPIPAAGTPLAYWGQGTLVTVSS SEQ ID NO: 122 |
| NI-607.531_C8-VL | GACATCCAGTTGACCCAGTCTCCAGACTCCCTGGCTGTGTCTCTGGGC |
| | GAGAGGGCCACCATCAACTGCAAGTCCAGCCAGAATATTTTATACAGC |
| | TCCAGCAATAAGAACTACTTGGCTTGGTACCAGCAGAAACCAGGACAG |
| | CCTCCTAAGCTGCTCATTGACTGGGCATCTACCCGGGAATCCGGGGTC |
| | CCTGACCGATTCAGTGGCAGCGGGTCTGGGACAGATTTCACTCTCACC |
| | ATCAGCAGCCTGCAGGCTGAAGATGTGGCAGTTTATTACTGTCACCAA |
| | TATTATAGTACTCCATTCACTTTCGGCCCTGGGACCAAGGTGGAAATC |
| | AAA SEQ ID NO: 126 |
| NI-607.531_C8-VL | DIQLTQSPDSLAVSLGERATINCKSSQNILYSSSNKNYLAWYQQKPGQ |
| | PPKLLIDWASTRESGVPDRFSGSGSGTDFTLTISSLQAEDVAVYYCHQ |
| | YYSTPFTFGPGTKVEIK SEQ ID NO: 127 |
| NI-607.531_D8-VH | GAGGTGCAGCTGGTGGAGTCTGGGGGAGGCTTGGTACAGCCTGGCAGG |
| | TCCCTGAGACTCTCCTGTGCAGCCTCTGGATTTATATTTGATGATTAT |
| | GCCATGCACTGGGTCCGGCAAGCTCCAGGGAAGGGCCTGGAGTGGGTC |
| | TCAGGTATTAGTTGGAATAGTGATAGCATAGGCTATGCGGACTCTGTG |
| | AAGGGCCGATTCACCATCTCCAGAGACAACGCCAAGAACTCCCTGTAT |
| | CTGCAAATGAACAGTCTGAGAGCTGAGGACACGGCCTTGTATTACTGT |
| | GCAAAAGGCAGCCGCAGTGACTGGTCAAACCCATCGGACTACTGGGGC |
| | CAGGGAACCTTGGTCACCGTCTCTTCA SEQ ID NO: 131 |
| NI-607.531_D8-VH | EVQLVESGGGLVQPGRSLRLSCAASGFIFDDYAMHWVRQAPGKGLEWV |
| | SGISWNSDSIGYADSVKGRFTISRDNAKNSLYLQMNSLRAEDTALYYC |
| | AKGSRSDWSNPSDYWGQGTLVTVSS SEQ ID NO: 132 |
| NI-607.531_D8-VL | TCCTATGAGCTGACTCAGCCACCCTCGGTGTCAGTGGCCCCAGGAAAG |
| | ACGGCCAGGATTACCTGTGGGGGAAACAACATTGGAAGTAAAAGTGTG |
| | CACTGGTACCAGCAGAAGCCAGGCCAGGCCCCTGTGCTGGTCGTCTAT |
| | GATGATAGCGACCGGCCCTCAGGGATCCCTGAGCGATTCTCTGGCTCC |
| | AACTCTGGGAACACGGCCACCCTGACCATCAGCAGGGTCGAAGCCGGG |
| | GATGAGGCCGACTATTACTGTCAGGTGTGGGATAGTAGTAGTGATCCG |
| | GTGGTTTTCGGCGGAGGGACCAAGCTGACCGTCCTA |
| | SEQ ID NO: 136 |
| NI-607.531_D8-VL | SYELTQPPSVSVAPGKTARITCGGNNIGSKSVHWYQQKPGQAPVLVVY |
| | DDSDRPSGIPERFSGSNSGNTATLTISRVEAGDEADYYCQVWDSSSDP |
| | VVFGGGTKLTVL SEQ ID NO: 137 |
| NI-607.532_B6-VH | CAGGTGCAGCTGGTGCAGTCTGGGGCTGAGGTGAAGAAGCCTGGGGCC |
| | TCAGTGAAGGTCTCCTGCAAGGCTTCTGGATACACCTTCACCAGTTAT |
| | GATATCAACTGGGTGCGACAGGCCACTGGACAAGGGCTTGAGTGGATG |
| | GGATGGATGAACCCTAACAGTGGTAACACAGGCTATGCACAGAAGTTC |
| | CAGGGCAGAGTCACCATGACCAGGAACACCTCCATAAGCACAGCCTAC |
| | ATGGAGCTGAGCAGCCTGAGATCTGAGGACACGGCCGTGTATTACTGT |
| | GCGAGAGTCGTATCTTTGACTGGCTTTTTTGACTACTGGGGCCAGGGA |
| | ACCCTGGTCACCGTCTCCTCA SEQ ID NO: 141 |
| NI-607.532_B6-VH | QVQLVQSGAEVKKPGASVKVSCKASGYTFTSYDINWVRQATGQGLEWM |
| | GWMNPNSGNTGYAQKFQGRVTMTRNTSISTAYMELSSLRSEDTAVYYC |
| | ARVVSLTGFFDYWGQGTLVTVSS SEQ ID NO: 142 |
| NI-607.532_B6-VL | TCCTATGAGCTGACTCAGCCACCCTCGGTGTCAGTGGCCCCAGGAAAG |
| | ACGGCCAGGATTACCTGTGGGGGAAACAACATTGGAAGTAAAAATGTG |
| | CACTGGTACCAGCAGAAGCCAGGCCAGGCCCCTGTGCTGGTCGTCTAT |
| | GATGATAGCGACCGGCCCTCAGGGATCCCTGAGCGATTCTCTGGCTCC |
| | AACTCTGGGAACACGGCCACCCTGACCATCAGCAGGGTCGAAGCCGGG |
| | GATGAGGCCGACTATTACTGTCAGGTGTGGGATAGTAGTAGTGATCCT |
| | TGGGTGTTCGGCGGAGGGACCAAGCTGACCGTCCTA |
| | SEQ ID NO: 146 |
| NI-607.532_B6-VL | SYELTQPPSVSVAPGKTARITCGGNNIGSKNVHWYQQKPGQAPVLVVY |
| | DDSDRPSGIPERFSGSNSGNTATLTISRVEAGDEADYYCQVWDSSSDP |
| | WVFGGGTKLTVL SEQ ID NO: 147 |
| NI-607.532_Cl 1-VH | CAGGTGCAGCTGGTGGAGTCTGGAGGAGGCTTGATCCAGCCTGGGGGG |
| | TCCCTGAGACTCTCCTGTGCAGCCTCTGGGTTTACCGTCAGTAGCAAC |
| | TACATGAGCTGGGTCCGCCAGGCTCCAGGGAAGGGGCTGGAGTGGGTC |
| | TCAGTTATTTATAGCGGTGGTAGCACATACTACGCAGACTCCGTGAAG |
| | GGCCGATTCACCATCTCCAGAGACAATTCCAAGAACACGCTGTTTCTT |
| | CAAATGAACAGCCTGAGAGCCGAGGACACGGCCGTGTATTACTGTGCG |
| | AGAGATCTTCAGTACTACGGTATGGACGTCTGGGGCCAAGGGACCACG |
| | GTCACCGTCTCCTCA SEQ ID NO: 151 |
| NI-607.532_Cl 1-VH | QVQLVESGGGLIQPGGSLRLSCAASGFTVSSNYMSWVRQAPGKGLEWV |
| | SVIYSGGSTYYADSVKGRFTISRDNSKNTLFLQMNSLRAEDTAVYYCA |
| | RDLQYYGMDVWGQGTTVTVSS SEQ ID NO: 152 |
| NI-607.532_Cl 1-VL | GACATCCAGATGACCCAGTCTCCATCCTTCCTGTCTGCATCTGTAGGA |
| | GACAGAGTCACCATCACTTGCCGGGCCAGTCAGGGCATTAGCAGTTAT |
| | TTAGCCTGGTATCAGCAAAAACCAGGGAAAGCCCCTAAGCTCCTGATC |
| | TATGCTGCATCCACTTTGCAAAGTGGGGTCCCATCAAGGTTCAGCGGC |
| | AGTGGATCTGGGACAGAATTCACTCTCACAATCAGCAGCCTGCAGCCT |
| | GAAGATTTTGCAACTTATTACTGTCAACAGCTTGATAGTTACCCTCCG |
| | CGGGACACTTTTGGCCAGGGGACCAAGGTGGAAATCAAA |
| | SEQ ID NO: 156 |
| NI-607.532_C11-VL | DIQMTQSPSFLSASVGDRVTITCRASQGISSYLAWYQQKPGKAPKLLI |
| | YAASTLQSGVPSRFSGSGSGTEFTLTISSLQPEDFATYYCQQLDSYPP |
| | RDTFGQGTKVEIK SEQ ID NO: 157 |
| NI-607.532_C8-VH | CAGGTGCAGCTGGTGGAGTCTGGGGGAGGCTTGGTAAAGCCAGGGCGG |
| | TCCCTGAGACTCTCCTGTACAGCTTCTGGATTCACCTTTGGTGATTAT |
| | GCTATGAGCTGGTTCCGCCAGGCTCCAGGGAAGGGGCTGGAGTGGGTA |
| | GGTTTCATTAGAAGCAAAGCTTATGGTGGGACAACAGAATACGCCGCG |
| | TCTGTGAAAGGCAGATTCAGCATCTCAAGAGATGATTCCAAAAGCATC |
| | GCCTATCTGCAAATGAACAGCCTGAAAACCGAGGACACAGCCGTGTAT |
| | TACTGTACTAGAGGTTGGATAGTAGTGGTTATCGAGGATGCTTTTGAT |
| | ATCTGGGGCCAAGGGACAATGGTCACCGTCTCTTCA |
| | SEQ ID NO: 161 |
| NI-607.532_C8-VH | QVQLVESGGGLVKPGRSLRLSCTASGFTFGDYAMSWFRQAPGKGLEWV |
| | GFIRSKAYGGTTEYAASVKGRFSISRDDSKSIAYLQMNSLKTEDTAVY |
| | YCTRGWIVVVIEDAFDIWGQGTMVTVSS SEQ ID NO: 162 |
| NI-607.532_C8-VL | GAAATTGTGTTGACGCAGTCTCCAGGCACCCTGTCTTTGTCTCCAGGG |
| | GAAAGAGCCACCCTCTCCTGCAGGGCCAGTCAGAGTGTTAGCAGCAGC |
| | TACTTAGCCTGGTACCAGCAGAAACCTGGCCAGGCTCCCAGGCTCCTC |
| | ATCTATGGTGCATCCAGCAGGGCCACTGGCATCCCAGACAGGTTCAGT |
| | GGCAGTGGGTCTGGGACAGACTTCACTCTCACCATCAGCAGACTGGAG |
| | CCTGAAGATTTTGCAGTGTATTACTGTCAGCAGTATGGTAGCTCACCG |
| | CTCACTTTCGGCGGAGGGACCAAGGTGGAAATCAAA |
| | SEQ ID NO: 166 |
| NI-607.532_C8-VL | EIVLTQSPGTLSLSPGERATLSCRASQSVSSSYLAWYQQKPGQAPRLL |
| | IYGASSRATGIPDRFSGSGSGTDFTLTISRLEPEDFAVYYCQQYGSSP |
| | LTFGGGTKVEIK SEQ ID NO: 167 |
| NI-607.532 D3-VH | GAGGTGCAGCTGGTGCAGTCTGGGGCTGAGGTGAAGAGGCCTGGGGCC |
| | TCAGTGAAGGTCTCCTGCAAGGCTTCTGGATACCACTTCACCGACTAC |
| | TATATACACTGGGTGCGACAGGCCCCTGGACAAGGGCTTGAGTGGATG |
| | GGATGGATCAACCCTAACAGTGGTGGCACAAACTATGCACAGAAGTTT |
| | CAGGGCAGGGTCACCATGACCAGGGACACGTCCATCAGCACAGCCTAC |
| | ATGGAGCTGAGCAGGCTGAGATCTGACGACACGGCCGTGTATTACTGT |
| | GCGAGGGTTTATTACTATGATAGTAGTAGTTCTGCTGACTACTGGGGC |
| | CAGGGAACCCTGGTCACCGTCTCCTCA SEQ ID NO: 171 |
| NI-607.532 D3-VH | EVQLVQSGAEVKRPGASVKVSCKASGYHFTDYYIHWVRQAPGQGLEWM |
| | GWINPNSGGTNYAQKFQGRVTMTRDTSISTAYMELSRLRSDDTAVYYC |
| | ARVYYYDSSSSADYWGQGTLVTVSS SEQ ID NO: 172 |
| NI-607.532_D3-VL | GACATCCAGATGACCCAGTCTCCATCCTCCCTGTCTGCATCTGTAGGA |
| | GACAGAGTCACCATCACTTGCCAGGCGAGTCAGGACATTAGCAACTAT |
| | TTAAATTGGTATCAGCAGAAACCAGGGAAAGCCCCTAAGCTCCTGATC |
| | TACGATGCATCCAATTTGGAAACAGGGGTCCCATCAAGGTTCAGTGGA |
| | AGTGGATCTGGGACAGATTTTACTTTCACCATCAGCAGCCTGCAGCCT |
| | GAAGACATTGCAACATATTACTGTCAACAGTATGATAATCTCCCTCCG |
| | GCGCTCACTTTCGGCGGAGGGACCAAGGTGGAAATCAAA |
| | SEQ ID NO: 176 |
| NI-607.532_D3-VL | SEQ ID NO: 177 |
| | DIQMTQSPSSLSASVGDRVTITCQASQDISNYLNWYQQKPGKAPKLLI |
| | YDASNLETGVPSRFSGSGSGTDFTFTISSLQPEDIATYYCQQYDNLPP |
| | ALTFGGGTKVEIK |
| NI-607.532_D4-VH | CAGGTGCAGCTGGTGCAGTCTGGGGCTGAGGTGAAGAGGCCTGGGGCC |
| | TCAGTGAAGGTCTCCTGCAAGGCTTCTGGATACCACTTCACCGACTAC |
| | TATATACACTGGGTGCGACAGGCCCCTGGACAAGGGCTTGAGTGGATG |
| | GGATGGATCAACCCTAACAGTGGTGGCACAAACTATGCACAGAAGTTT |
| | CAGGGCAGGGTCACCATGACCAGGGACACGTCCATCAGCACAGCCTAC |
| | ATGGAGCTGAGCAGGCTGAGATCTGACGACACGGCCGTGTATTACTGT |
| | GCGAGGGTTTATTACTATGATAGTAGTAGTTTTGCTGACTACTGGGGC |
| | CAGGGAACCCTGGTCACCGTCTCCTCA SEQ ID NO: 181 |
| NI-607.532_D4-VH | QVQLVQSGAEVKRPGASVKVSCKASGYHFTDYYIHWVRQAPGQGLEWM |
| | GWINPNSGGTNYAQKFQGRVTMTRDTSISTAYMELSRLRSDDTAVYYC |
| | ARVYYYDSSSFADYWGQGTLVTVSS SEQ ID NO: 182 |
| NI-607.532_D4-VL | GACATCCAGATGACCCAGTCTCCATCCTCCCTGTCTGCATCTGTAGGA |
| | GACAGAGTCACCATCACTTGCCAGGCGAGTCAGGACATTAGCAACTAT |
| | TTAAATTGGTATCAGCAGAAACCAGGGAAAGCCCCTAAGCTCCTGATC |
| | TACGATGCATCCAATTTGGAAACAGGGGTCCCATCAAGGTTCAGTGGA |
| | AGTGGATCTGGGACAGATTTTACTTTCACCATCAGCAGCCTGCAGCCT |
| | GAAGACATTGCAACATATTACTGTCAACAGTATGATAATCTCCCTCCG |
| | GCGCTCACTTTCGGCGGAGGGACCAAGGTGGAAATCAAA |
| | SEQ ID NO: 186 |
| NI-607.532_D4-VL | DIQMTQSPSSLSASVGDRVTITCQASQDISNYLNWYQQKPGKAPKLLI |
| | YDASNLETGVPSRFSGSGSGTDFTFTISSLQPEDIATYYCQQYDNLPP |
| | ALTFGGGTKVEIK SEQ ID NO: 187 |
| NI-607.532_D8-VH | GAGGTGCAGCTGGTGGAGTCTGGGGGAGGCGTGGTCCAGCCTGGGAGG |
| | TCCCTGAGACTCTCCTGTGCAGCCTCTGGATTCACCTTCAGTAGCTAT |
| | GGCATGCACTGGGTCCGCCAGGCTCCAGGCAAGGGGCTGGAGTGGGTG |
| | GCAGTTATATCATATGATGGAAGTAATAAATACTATGCAGACTCCGTG |
| | AAGGGCCGATTCACCATCTCCAGAGACAATTCCAAGAACACGCTGTAT |
| | CTGCAAATGAACAGCCTGAGAGCTGAGGACACGGCTGTGTATTACTGT |
| | GCGAAACAAGGAGGGGGCCAATATTGTAGTGGTGGTAGCTGCTACTCT |
| | GGCTACTTTGACTACTGGGGCCAGGGAACCCTGGTCACCGTCTCCTCA |
| | SEQ ID NO: 191 |
| NI-607.532_D8-VH | EVQLVESGGGVVQPGRSLRLSCAASGFTFSSYGMHWVRQAPGKGLEWV |
| | AVISYDGSNKYYADSVKGRFTISRDNSKNTLYLQMNSLRAEDTAVYYC |
| | AKQGGGQYCSGGSCYSGYFDYWGQGTLVTVSS SEQ ID NO: 192 |
| NI-607.532_D8-VL | GACATCCAGTTGACCCAGTCTCCTTCCACCCTGTCTGCATCTGTAGGA |
| | GACAGAGTCACCATCACTTGCCGGGCCAGTCAGAGTATTAGTAGCTGG |
| | TTGGCCTGGTATCAGCAGAAACCAGGGAAAGCCCCTAAGCTCCTGATC |
| | TATAAGGCGTCTAGTTTAGAAAGTGGGGTCCCATCAAGGTTCAGCGGC |
| | AGTGGATCTGGGACAGAATTCACTCTCACCATCAGCAGCCTGCAGCCT |
| | GATGATTTTGCAACTTATTACTGCCAACAGTATAATAGTTATCCGACG |
| | TTCGGCCAAGGGACCAAAGTGGATATCAAA SEQ ID NO: 196 |
| NI-607.532_D8-VL | DIQLTQSPSTLSASVGDRVTITCRASQSISSWLAWYQQKPGKAPKLLI |
| | YKASSLESGVPSRFSGSGSGTEFTLTISSLQPDDFATYYCQQYNSYPT |
| | FGQGTKVDIK SEQ ID NO: 19 7 |
| NI-607.532_F9-VH | GAGGTGCAGCTGGTGGAGTCTGGAGGAAACTTGATCCAGCCTGGGGGG |
| | TCCCTGAGACTCTCCTGTGCAGCCTCTGGGTTCACCGTCAGTAGCAAC |
| | TACATGAGCTGGGTCCGCCAGGCTCCAGGGAAGGGGCTGGAGTGGGTC |
| | TCAGTTATTTATAGCGGTGGTAGCACATACTACGCAGACTCCGTGAAG |
| | GGCCGATTCACCATCTCCAGAGACAATTCCAGGAACACGCTGTATCTT |
| | CAAATGAACAGCCTGAGAGCCGAGGACACGGCCGTGTATTTCTGTGCG |
| | AGAGATAGGGTGGTTCGGGGAGTTAGAGGGAACGGTATGGACGTCTGG |
| | GGCCAAGGGACCACGGTCACCGTCTCCTCA SEQ ID NO: 201 |
| NI-607.532_F9-VH | EVQLVESGGNLIQPGGSLRLSCAASGFTVSSNYMSWVRQAPGKGLEWV |
| | SVIYSGGSTYYADSVKGRFTISRDNSRNTLYLQMNSLRAEDTAVYFCA |
| | RDRVVRGVRGNGMDVWGQGTTVTVSS SEQ ID NO: 202 |
| NI-607.532_F9-VL | GCCATCCGGATGACCCAGTCTCCATCCTCCCTGTCTGCATCTGTAGGA |
| | GACAGAGTCACCATCACTTGCCAGGCGAGTCAGGACATTAACATCTAT |
| | TTAAATTGGTATCAGCAGAGACCAGGGAAAGCCCCTAAGCTCCTGATC |
| | TACGATGCATCCAATTTGGAAACAGGGGTCCCATCAAGGTTCAGTGGA |
| | AGTGGATCTGGGACAGATTTTACCTTCACCATCAGCAGCCTGCAGCCT |
| | GAAGATATTGCAACATATTACTGTCACCAGTATGATAATCTCCCTCGG |
| | ACGTTCGGCCAAGGGACCAAAGTGGATATCAAA SEQ ID NO: 206 |
| NI-607.532_F9-VL | AIRMTQSPSSLSASVGDRVTITCQASQDINIYLNWYQQRPGKAPKLLI |
| | YDASNLETGVPSRFSGSGSGTDFTFTISSLQPEDIATYYCHQYDNLPR |
| | TFGQGTKVDIK SEQ ID NO: 207 |
| NI-607.649_B11-VH | CAGGTTCAGCTGGTGCAGTCTGGGGCTGAGGTGAAGAAGCCTGGGTCC |
| | TCGGTGAAGGTCTCCTGCAAGGCTTCTGGAGGCACCTTCAGCAACTAT |
| | GCTATCAGCTGGGTGCGACAGGCCCCTGGACAAGGGCTTGAGTGGATG |
| | GGAGGGATCATCCCTATCTTTGGTACAGCAAACTACGCACAGAAGTTC |
| | CAGGGCAGAGTCACGATTACCGCGGACGAATCCACGAGCACAGCCTAC |
| | ATGGAGCTGAGCAGCCTGAGATCTGAGGACACGGCCGTGTATTACTGT |
| | GCGGGAGATAGATGGTACTATGATAGTAGTGGTTATCCAACACCAGAT |
| | GAGTACTTCCAGCACTGGGGCCAGGGCACCCAGGTCACCGTCTCCTCA |
| | SEQ ID NO: 211 |
| NI-607.649_B11-VH | QVQLVQSGAEVKKPGSSVKVSCKASGGTFSNYAISWVRQAPGQGLEWM |
| | GGIIPIFGTANYAQKFQGRVTITADESTSTAYMELSSLRSEDTAVYYC |
| | AGDRWYYDSSGYPTPDEYFQHWGQGTQVTVSS SEQ ID NO: 212 |
| NI-607.649_B11-VL | CAGTCTGTGCTGACGCAGCCGCCCTCAGTGTCTGGGGCCCCAGGGCAG |
| | AGGGTCACCATCTCCTGCACTGGGAGCAGCTCCAACATCGGGGCAGGT |
| | TATGATGTACACTGGTACCAGCAGCTTCCAGGAACAGCCCCCAAACTC |
| | CTCATCTATGGTAACAGCAATCGGCCCTCAGGGGTCCCTGACCGATTC |
| | TCTGGCTCCAAGTCTGGCACCTCAGCCTCCCTGGCCATCACTGGGCTC |
| | CAGGCTGAGGATGAGGCTGATTATTACTGCCAGTCCTATGACAGCAGC |
| | CTGAGTGGTGTGGTATTCGGCGGAGGGACCAAGCTGACCGTCCTA |
| | SEQ ID NO: 216 |
| NI-607.649_B11-VL | QSVLTQPPSVSGAPGQRVTISCTGSSSNIGAGYDVHWYQQLPGTAPKL |
| | LIYGNSNRPSGVPDRFSGSKSGTSASLAITGLQAEDEADYYCQSYDSS |
| | LSGVVFGGGTKLTVL SEQ ID NO: 217 |
| NI-607.531_E7-VH | GAAGTGCAGCTGGTGGAGTCTGGGGGAGGCGTGGTCCAGCCTGGGAGG |
| | TCCCTGAGACTCTCCTGTGCAGCCTCTGGATTCACCTTCAATTTCTAT |
| | TCTATGCACTGGGTCCGCCAGGTTCCAGGCAAGGGGCTGGAATGGGTG |
| | GCAGTTATATCGTTCGATGGGAGCACTAAACACTACGCAGAGTCCCTG |
| | AGGGGCCGATTTGCCGTCTCCAGAGACAATTCCAAGAGTACCCTGTAT |
| | CTCCAAATGAACAGCCTGAGACCTGAGGACACGGCTGTGTATTACTGT |
| | GCGAGTCCCCCCCTCTCCCCCATACCAGCTGCTGGTACGCCCCTTGAC |
| | TACTGGGGCCAGGGAACCCTGGTCACCGTCTCCTCA |
| | SEQ ID NO: 221 |
| NI-607.531_E7-VH | EVQLVESGGGVVQPGRSLRLSCAASGFTFNFYSMHWVRQVPGKGLEWV |
| | AVISFDGSTKHYAESLRGRFAVSRDNSKSTLYLQMNSLRPEDTAVYYC |
| | ASPPLSPIPAAGTPLDYWGQGTLVTVSS SEQ ID NO: 222 |
| NI-607.531_E7-VL | GAAATAGTGATGACGCAGTCTCCAGACTCCCTGGCTGTGTCTCTGGGC |
| | GAGAGGGCCACCATCAACTGCAAGTCCAGCCAGAGTATTTTCTACAGA |
| | TCCGACGATAAGGACTTCTTAGCTTGGTACCAGCAGAAACCAGGACAG |
| | CCTCCTAAGTTGCTCATTTCCTGGGCATCTACCCGGGAATCCGGGGTC |
| | CCTGACCGATTCAGTGGCAGCGGGTCTGGGACAGATTTCACTCTCACC |
| | ATCAGCAGCCTGCAGGCTGAAGATGTGGCAGTTTATTACTGTCAGCAA |
| | TTTTACTATACTCCATTCACTTTCGGCCCTGGGACCAAGCTGGAGATC |
| | AAA SEQ ID NO: 226 |
| NI-607.531_E7-VL | EIVMTQSPDSLAVSLGERATINCKSSQSIFYRSDDKDFLAWYQQKPGQ |
| | PPKLLISWASTRESGVPDRFSGSGSGTDFTLTISSLQAEDVAVYYCQQ |
| | FYYTPFTFGPGTKLEIK SEQ ID NO: 227 |
| NI-607.532_F3-VH | GAGGTGCAGCTGGTGCAGTCTGGAGCAGAGGTGAAAAAGCCCGGGGAA |
| | TCTCTGAAGATCTCATGTAAGGGTTTTGGATACATCTTTAGCAACGAC |
| | TGGATCGGCTGGGTGCGCCAGAAGCCCGGGAAAGGCCTGGAGTGGATG |
| | GGCATTATCTATCCTGGTGACTCTGACACCCGATACAGCCCGTCCTTC |
| | CGAGGCCAGGTCACCATCTCAGCCGACAAGTCCGTCAGCACCGCCTAT |
| | CTGCAGTGGGACAGCCTGAAGGCCTCGGACACCGCCGTCTATTATTGT |
| | ACAAGACCCCGATACACTACAATGATAGTGGGATCGAATCCCTTTGAT |
| | GTCTGGGGCCAGGGGACTACGGTCACCGTCTCTTCA |
| | SEQ ID NO: 231 |
| NI-607.532_F3-VH | EVQLVQSGAEVKKPGESLKISCKGFGYIFSNDWIGWVRQKPGKGLEWM |
| | GIIYPGDSDTRYSPSFRGQVTISADKSVSTAYLQWDSLKASDTAVYYC |
| | TRPRYTTMIVGSNPFDVWGQGTTVTVSS SEQ ID NO: 232 |
| NI-607.532_F3-VL | CAGTCTGTGCTGACGCAGCCGCCCTCAGTGTCTGGGGCCCCAGGGCAG |
| | AGGGTCACCATCTCCTGCACTGGGAGCAGCTCCAACATCGGGGGAGGC |
| | TATGATGTACACTGGTACCAGAAGCTTCCAGGAACAGCCCCCAAACTC |
| | CTCATCTTTGATAATGACAATCGGCCCTCAGGGGTCCCTGACCGATTC |
| | TCTGGCTCCAAGTCTGACACTTCAGCCTCCCTGACCATCACTGGACTC |
| | CAGGCTGAGGATGAGGCTGATTATTTTTGTCAGTCCTATGACAGCAGC |
| | CTGAGTGGCTCGGGAGTGGTATTCGGCGGAGGGACCAAGCTGACCGTC |
| | CTG SEQ ID NO: 236 |
| NI-607.532_F3-VL | QSVLTQPPSVSGAPGQRVTISCTGSSSNIGGGYDVHWYQKLPGTAPKL |
| | LIFDNDNRPSGVPDRFSGSKSDTSASLTITGLQAEDEADYFCQSYDSS |
| | LSGSGVVFGGGTKLTVL SEQ ID NO: 237 |
| NI-607.649_G7-VH | CAGGTGCAGCTGGTGGAGTCTGGGGGAGGCTTGGTACAGCCTGGGGGG |
| | TCCCTGAGACTCTCCTGTGCAGCCTCTGGATTCACCTTTAGCCGCTAT |
| | GCCATGAGCTGGGTCCGCCAGGCTCCAGGGAAGGGGCTGGAGTGGGTC |
| | TCAGCTATTAGTGGTAGTGGTGGTAGCACATACTACGCAGACTCCGTG |
| | AAGGGCCGGTTCACCATCTCCAGAGACAATTCCAAGAACACGCTGTAT |
| | CTGCAAATTAACAGCCTGAGAGCCGAGGACACGGCCGTATATTACTGT |
| | GCGAAAGGGGTTAGGTGGGGAGTCGACTACTTTGACTACTGGGGCCAG |
| | GGAACCCTGGTCACCGTCTCTTCA SEQ ID NO: 241 |
| NI-607.649_G7-VH | QVQLVESGGGLVQPGGSLRLSCAASGFTFSRYAMSWVRQAPGKGLEWV |
| | SAISGSGGSTYYADSVKGRFTISRDNSKNTLYLQINSLRAEDTAVYYC |
| | AKGVRWGVDYFDYWGQGTLVTVSS SEQ ID NO: 242 |
| NI-607.649_G7-VL | GCCATCCGGATGACCCAGTCTCCTTCCACCCTGTCTGCATCTGTAGGA |
| | GACAGAGTCACCATCACTTGCCGGGCCAGTCAGAGTATTAGTAGCTGG |
| | TTGGCCTGGTATCAGCAGAAACCAGGGAAAGCCCCTAAGCTCCTGATC |
| | TATGATGCCTCCAGTTTGGAAAGTGGGGTCCCATCAAGGTTCAGCGGC |
| | AGTGGATCTGGGACAGAATTCACTCTCACCATCAGCAGCCTGCAGCCT |
| | GATGATTTTGCAACTTATTACTGCCAACAGTATAATAGTTATTCTTCG |
| | TGGACGTTCGGCCAAGGGACCAAGCTGGAGATCAAA |
| | SEQ ID NO: 246 |
| NI-607.649_G7-VL | AIRMTQSPSTLSASVGDRVTITCRASQSISSWLAWYQQKPGKAPKLLI |
| | YDASSLESGVPSRFSGSGSGTEFTLTISSLQPDDFATYYCQQYNSYSS |
| | WTFGQGTKLEIK SEQ ID NO: 247 |
| NI-607.761_B7-VH | GAGGTGCAGCTGGTGGAGTCTGGGGGAGGCGTGGTCCAGCCTGGGAGG |
| | TCCCTGAGACTCTCCTGTGCAGCGTCTGGATTCACCTTCACTAGGTAT |
| | GGCATGCACTGGGTCCGCCAGGCTCCAGGCAAGGGGCTGGAGTGGGTG |
| | GCAGTTATATGGTATGATGGAAATAATAAAAACTATGAAGACTCCGTG |
| | AGGGGCCGATTCACCATCTCCAGAGACAATTCCAAGAACACGCTGTTT |
| | CTGCAAATGAACAGCCTGAGAGCCGAAGACACGGCTGTGTATTACTGT |
| | GCGAGAGGCCGCCGCGGTGGATACAGCTATGGCCTCTTTGACTACTGG |
| | GGCCAGGGAATCCTGGTCACCGTCTCCTCA SEQ ID NO: 251 |
| NI-607.761_B7-VH | EVQLVESGGGVVQPGRSLRLSCAASGFTFTRYGMHWVRQAPGKGLEWV |
| | AVIWYDGNNKNYEDSVRGRFTISRDNSKNTLFLQMNSLRAEDTAVYYC |
| | ARGRRGGYSYGLFDYWGQGILVTVSS SEQ ID NO: 252 |
| NI-607.761_B7-VL | GCCATCCGGATGACCCAGTCTCCATCCTCCCTGTCTGCATCTGTAGGG |
| | GACAGAGTCACCATCACTTGCCAGGCGAGTCAGGACATTAGCAACTAT |
| | TTAAATTGGTATCAGCAGAAACCAGGGAAAGCCCCTAAGCTCCTGATC |
| | TACGATGCATCCAATTTGGAGACAGGGGTCCCATCAAGGTTCAGTGGA |
| | AGTGGATCTGGGACAGATTTTACTTTCACCATCAGCAGCCTGCAGCCT |
| | GAAGATGTTGCAACATATTCCTGTCAACAGTATGATAATCTCCCGCTC |
| | ACTTTCGGCGGGGGGACCAAGGTGGAAATCAAA |
| | SEQ ID NO: 256 |
| NI-607.761_B7-VL | AIRMTQSPSSLSASVGDRVTITCQASQDISNYLNWYQQKPGKAPKLLI |
| | YDASNLETGVPSRFSGSGSGTDFTFTISSLQPEDVATYSCQQYDNLPL |
| | TFGGGTKVEIK SEQ ID NO: 257 |
| NI-607.791_B10-VH | CAGGTGCAGCTACAGCAGTGGGGCGCAGGACTGTTGAAGCCTTCGGAG |
| | ACCCTGTCCCGCACCTGCGCTGTCTATGGTGGGTCCTTCAGTGATTAC |
| | TACTGGAGCTGGATCCGCCAGCCCCCAGGGAAGGGACTGGAGTGGATT |
| | GGCGAAGTCAATCATAGGGGTGTCACCAACTACAACCCGTCCCTCAAG |
| | AGTCGAGTCACCATATCAATAGACACGTCTAAGAGCCAGTTCTCCCTG |
| | GAACTGAGGTCTGTGACCGACGCGGACACGGCTCTCTATTTCTGTGCG |
| | AGAAGCCCCTTCTGGACGATAGCGGCTCGTCCGTTTGACTACTGGGGC |
| | CGGGGAACCCTGGTCACCGTCTCCTCA SEQ ID NO: 261 |
| NI-607.791_B10-VH | QVQLQQWGAGLLKPSETLSRTCAVYGGSFSDYYWSWIRQPPGKGLEWI |
| | GEVNHRGVTNYNPSLKSRVTISIDTSKSQFSLELRSVTDADTALYFCA |
| | RSPFWTIAARPFDYWGRGTLVTVSS SEQ ID NO: 262 |
| NI-607.791_B10-VL | GAAATAGTGATGACGCAGTCTCCAGGCACCCTGTCTTTGTCTCCAGGG |
| | GAAAGAGCCACCCTCTCCTGCAGGGCCAGTCAGAGTGTTATCAGCAAC |
| | TACTTAGCCTGGTACCAGCAGAAACCTGGCCAGGCTCCCAGGTTCCTC |
| | ATCTATGCTGCATCCACCAGGGCCAGAGGCATCCCAGACAGGTTCAGT |
| | GGCAGTGGGTCTGGGACAGACTTCACTCTCACCATCAGCAGACTGGAG |
| | GCTGAAGATTTTGCAGTTTATTACTGTCAGCAATATGGTAACTCACCG |
| | CTCACTTTCGGCGGAGGGACCAAGCTGGAGATCAAA |
| | SEQ ID NO: 266 |
| NI-607.791_B10-VL | EIVMTQSPGTLSLSPGERATLSCRASQSVISNYLAWYQQKPGQAPRFL |
| | IYAASTRARGIPDRFSGSGSGTDFTLTISRLEAEDFAVYYCQQYGNSP |
| | LTFGGGTKLEIK SEQ ID NO: 267 |
| NI-607.531_E3-VH | GAGGTGCAGCTGGTGGAGTCTGGGGGAGGCGTGGTCCAGCCTGGGAGG |
| | TCCCTGAGACTCTCCTGTGCAGCCTCTGGATTCACCTTCAGTAGCTAT |
| | GGCATGCACTGGGTCCGCCAGGCTCCAGGCAAGGGGCTGGAGTGGGTG |
| | GCAGTTATATCATATGATGGAAGTAATAAATACTATGCAGACTCCGTG |
| | AAGGGCCGATTCACCATCTCCAGAGACAATTCCAAGAACACGCTGTAT |
| | CTGCAAATGAACAGCCTGAGAGCTGAGGACACGGCTGTGTATTACTGT |
| | GCGAAACAAATCAGTGGCTACAATTACCCCTACTACTTTGACTACTGG |
| | GGCCAGGGAACCCTGGTCACCGTCTCCTCA SEQ ID NO: 271 |
| NI-607.531_E3-VH | EVQLVESGGGVVQPGRSLRLSCAASGFTFSSYGMHWVRQAPGKGLEWV |
| | AVISYDGSNKYYADSVKGRFTISRDNSKNTLYLQMNSLRAEDTAVYYC |
| | AKQISGYNYPYYFDYWGQGTLVTVSS SEQ ID NO: 272 |
| NI-607.531_E3-VL | GACATCCAGTTGACCCAGTCTCCATCCTCCCTGTCTGCATCTGTAGGA |
| | GACAGAGTCACCATCACTTGCCAGGCGAGTCAGGACATTAGCAACTAT |
| | TTAAATTGGTATCAGCAGAAACCAGGGAAAGCCCCTAAGCTCCTGATC |
| | TACGATGCATCCAATTTGGAAACAGGGGTCCCATCAAGGTTCAGTGGA |
| | AGTGGATCTGGGACAGATTTTACTTTCACCATCAGCAGCCTGCAGCCT |
| | GAAGATATTGCAACATATTACTGTCAACAGTATGATAATCTCCCGCTC |
| | ACTTTCGGCGGAGGGACCAAGCTGGAGATCAAA SEQ ID NO: 276 |
| NI-607.531_E3-VL | DIQLTQSPSSLSASVGDRVTITCQASQDISNYLNWYQQKPGKAPKLLI |
| | YDASNLETGVPSRFSGSGSGTDFTFTISSLQPEDIATYYCQQYDNLPL |
| | TFGGGTKLEIK SEQ ID NO: 277 |
| NI-607.820_B6-VH | GAGGTGCAGCTGCAGGAGTCGGGCCCAGGACTGGTGAAGCCTTCGGAG |
| | ACCCTGTCCCTCACCTGCACTGTCTCTGGTGGCTCCGTCAGCAGTGGT |
| | AGTTATTACTGGAGCTGGATCCGGCAGCCCCCAGGGAAGGGACTGGAG |
| | TGGATTGGGTATATCTATTACAGTGGGAGCACCAACTACAACCCCTCC |
| | CTCAAGAGTCGAGTCACCATATCAGTAGACACGTCCAAGAACCAGTTC |
| | TCCCTGAAGGTGAGCTCTGTGACCGCTGCGGACACGGCCGTGTATTAC |
| | TGTGCGAGAGATTCTATTGTAGTAGTACCAGCTGCTAGACCGCTTTAC |
| | TATGGTATGGACGTCTGGGGCCAAGGGACCACGGTCACCGTCTCCTCA |
| | SEQ ID NO: 281 |
| NI-607.820_B6-VH | EVQLQESGPGLVKPSETLSLTCTVSGGSVSSGSYYWSWIRQPPGKGLE |
| | WIGYIYYSGSTNYNPSLKSRVTISVDTSKNQFSLKVSSVTAADTAVYY |
| | CARDSIVVVPAARPLYYGMDVWGQGTTVTVSS SEQ ID NO: 282 |
| NI-607.820_B6-VL | CAGTCTGTGCTGACGCAGCCACCCTCAGCGTCTGGGACCCCCGGGCAG |
| | AGGGTCACCATCTCTTGTTCTGGAAGCAGCTCCAACATCGGAAGTAAT |
| | TCTGTAGACTGGTACCAGCAGCTCCCAGGAACGGCCCCCAAACTCCTC |
| | ATCTATAGTACTAATCAGCGGCCCTCAGGGGTCCCTGACCGATTCTCT |
| | GGCTCCAAGTCTGGCACCTCAGCCTCCCTGGCCATCAGTGGGCTCCAG |
| | TCTGAGGATGAGGCTGATTATTACTGTGCAGCATGGGATGACAGCCTG |
| | AATGTCCGTTATGTCTTCGGAACTGGGACCAAGGTCACCGTCCTA |
| | SEQ ID NO: 286 |
| NI-607.820_B6-VL | QSVLTQPPSASGTPGQRVTISCSGSSSNIGSNSVDWYQQLPGTAPKLL |
| | IYSTNQRPSGVPDRFSGSKSGTSASLAISGLQSEDEADYYCAAWDDSL |
| | NVRYVFGTGTKVTVL SEQ ID NO: 287 |
| NI-607.820_B7-VH | GAGGTGCAGCTGGTGGAGTCTGGGGGAGGCTTGGTACAGCCTGGCAGG |
| | TCCCTGAGACTCTCCTGTGCAGCCTCCGGATTTACTTTTGATGATTTT |
| | GACATGCACTGGGTCCGGCAAGCTCCAGGGAAGGGCCTGGAATGGGTC |
| | TCAGGCGTTACTTGGAACAGTGGAATCATAGGGTATGCGGACTCTGTG |
| | AAGGGCCGATTCACCATCTCCAGAGACAACGCCAAGAACTCCCTGTAT |
| | CTGCAAATGAACAGTCTGAGAGCTGAGGACACGGCCTTGTATTACTGT |
| | GCAAAAGAATTCTATCCCCGTTATGGTTCGGGTATTAGGTCCCACTAC |
| | TTCGGTATGGACGTCTGGGGCCAAGGGACCACGGTCACCGTCTCCTCA |
| | SEQ ID NO: 291 |
| NI-607.820_B7-VH | EVQLVESGGGLVQPGRSLRLSCAASGFTFDDFDMHWVRQAPGKGLEWV |
| | SGVTWNSGIIGYADSVKGRFTISRDNAKNSLYLQMNSLRAEDTALYYC |
| | AKEFYPRYGSGIRSHYFGMDVWGQGTTVTVSS SEQ ID NO: 292 |
| NI-607.820_B7-VL | CAGTCTGTGCTGACTCAGTCACCCTCAGCGTCTGGGACCCCCGGGCAG |
| | AGGGTCACCATCTCTTGTTCTGGCAGCAGCTCCAACGTCGGAAGTAAT |
| | GCTGTAAACTGGTACCAGCAGCTCCCAGGAACGGCCCCCAAACTCCTC |
| | ATCTATAGTAATAATCGGCGGCCCTCAGGGGTCCCGAACCGATTCTCT |
| | GGCTCCAAGTCTGGCACCTCAGCCTCCCTGGCCATCAGTGGACTACAG |
| | TCTGACGATGAGGCTGATTATTACTGTGCAGCATGGGATGACAGCCTG |
| | GATGGCCCCACATTCGGCGGAGGGACCAAGCTGACCGTCCTA |
| | SEQ ID NO: 296 |
| NI-607.820_B7-VL | QSVLTQSPSASGTPGQRVTISCSGSSSNVGSNAVNWYQQLPGTAPKLL |
| | IYSNNRRPSGVPNRFSGSKSGTSASLAISGLQSDDEADYYCAAWDDSL |
| | DGPTFGGGTKLTVL SEQ ID NO: 297 |

Table II depicts the amino acid sequences of the variable regions, i.e. heavy chain and light chain (VH, VL) of anti-SARS-CoV-2 specific human antibodies NI-607.274_B7, NI-607.274_E5, NI-607.275_C5, NI-607.426_D4, NI-607.426_E2, NI-607.426_F11, NI-607.427_C5, NI-607.428_B9, NI-607.429_B9, NI-607.429_E4, NI-607.529_B9, NI-607.529_G4, NI-607.531_C8, NI-607.531D38, NI-607.532_B6, NI-607.532_C11, NI-607.532_C8, NI-607.532_D3, NI-607.532_D4, NI-607.532_D8, NI-607.532_F9, NI-607.649_B11, NI-607.531_E7, NI-607.532_F3, NI-607.649_G7, NI-607.761_B7, NI-607.791_B10, NI-607.531_E3, NI-607.820_B6, and NI-607.820_B7 of the present invention. The sequences between the three CDRs of the VH region (VH-CDR1, VH-CDR2 and VH-CDR3) and the three CDRs of the VL region (VL-CDR1, VL-CDR2 and VL-CDR3) are the framework regions. The CDRs are numbered according to their order in the amino acid sequences and highlighted (bold and underlined amino acids). The Chothia numbering scheme was used (http://www.bioinf.org.uk/abs/; Chothia and Lesk, J. Mol. Biol. 196 (1987), 901-917) as explained in Table III below. Unless otherwise specified, references to the numbering of specific amino acid residue positions in an antibody or SARS-CoV-2-binding fragment, variant, or derivative thereof of the present invention are according to the Chothia numbering system, which however is theoretical and may not equally apply to every antibody of the present invention. For example, depending on the position of the first CDR the following CDRs might be shifted in either direction. Accordingly, in case of any inadvertent errors or inconsistencies regarding indication of CDRs in Table II and/or the sequence listing the person skilled in the art on the basis of the disclosure content of the present application, i.e. the variable heavy (VH) and variable light (VL) chain amino acid sequences of the above mentioned antibodies is well in the position to determine the correct CDR sequences in accordance with Chothia, which shall be used for defining the claimed antibody and SARS-CoV-2-binding fragment thereof. As further explained in the description, within CDRs and/or framework region conservative amino acid substitutions are preferred which take into account the physicochemical properties of the original amino acid either alone or with an adjacent amino acid as illustrated in Mirsky et al., Mol. Biol. Evol. 32 (2014) 806-819 at page 813, FIG. 6 in particular the AB or LG model, for example such that the position of two amino acids is exchanged.

While the invention is illustrated and described by way of reference to the human-derived antibody originally obtained in the experiments performed in accordance with the present invention and described in the Examples it is to be understood that the antibody or antibody fragment of the present invention includes synthetic and biotechnological derivatives of an antibody which means any engineered antibody or antibody-like SARS-CoV-2 binding molecule, synthesized by chemical or recombinant techniques, which retains one or more of the functional properties of the subject antibody, in particular recognizing, binding and potentially neutralizing SARS-CoV-2. Thus, while the present invention may be described for the sake of conciseness by way of reference to an antibody or antibodies, unless stated otherwise synthetic and biotechnological derivatives thereof as well as equivalent SARS-CoV-2 binding molecules are meant and included within the meaning of the term “antibody”. Furthermore, for the sake of clarity, when reference is made to the antibodies of the present invention also the antibodies of the composition of the present invention are meant and vice versa.

Further embodiments of the present invention will be apparent from the description, the Figures and Examples that follow.

### BRIEF DESCRIPTION OF THE DRAWINGS

FIG. 1: Binding specificity and EC₅₀ determination for SARS-CoV-2 S1 RBD domain; see Example 3. Exemplarily, the binding specificities as determined by ELISA of antibodies NI-607.532_C8 and NI-607.649_B22 are shown. (A) Binding specificity of IgG1 antibody NI-607.532_C8 for SARS-CoV-2 S1 RBD domain (•) and to a BSA control (▴). (B) Binding specificity of IgG4 S228 antibody NI-607.532_C8 for SARS-CoV-2 S1 RBD domain (•) and to a BSA control (▴). (C) Binding specificity of IgG1 antibody NI-607.649_B11 for SARS-CoV-2 S1 RBD domain (•) and to a BSA control (▴). (D) Binding specificity of IgG4 S228 antibody NI-607.649_B11 for SARS-CoV-2 S1 RBD domain (•) and to a BSA control (▴). In this experiment, antibody NI-607.532_C8 (IgG1) specifically recognized the SARS-CoV-2 S1 RBD with an EC₅₀ of about 17 pM; antibody NI-607.532_C8 (IgG4 S228P) specifically recognized the SARS-CoV-2 S1 RBD with an EC₅₀ of about 8.5 pM; antibody NI-607.649_B11 (IgG1) specifically recognized the SARS-CoV-2 S1 RBD with an EC₅₀ of about 13 pM; and antibody NI-607.649_B11 (IgG4 S228P) specifically recognized the SARS-CoV-2 S1 RBD with an EC₅₀ of about 8.5 pM.

FIG. 2: Target binding analysis of the antibodies of the present invention; see Example 4. The antibodies of the present invention were tested for binding to SARS-CoV-2 RBD available from SinoBiologics (•) and from Trenzyme (▴), to SARS-CoV-2 Spike protein S1 (♦), to SARS-CoV-2 full Spike protein (S1+S2) (▪), to MERS-CoV RBD (◯), and to SARS-CoV RBD (⋄) using flow cytometry on iQue. Here the results for the exemplarity antibodies NI-607.532_C8 (A) and NI-607.649_B11 (B) are shown. NI-607.532_C8 has a high binding affinity to the RBD and Spike protein of SARS-CoV-2, but a low affinity to the RBD of SARS-CoV and to the RBD of MERS-CoV. NI-607.649_B11 has a high binding affinity to the RBD and Spike protein of SARS-CoV-2 and to the RBD of SARS-CoV, but a low affinity to the RBD of MERS-CoV.

FIG. 3: Neutralization capability of the antibodies of the present invention; see Example 5. The antibodies of the present invention were tested for their neutralization capability, i.e. which were able to disrupt the interaction between the viral RBD protein and the human ACE2 receptor, by a competition ELISA detecting free RBD. Here the results for the exemplarily antibodies NI-607.532_C8 (IgG1 ( [symbol] ); IgG4 S228P ( [symbol] )) and NI-607.649_B11 (IgG1 ( [symbol] ); IgG4 S228P (♂)) with neutralization capability are shown.

FIG. 4: Schematic illustration of antibody binding to the RBD of SARS-CoV-2, thereby preventing interaction of the RBD with the ACE2 receptor. Different neutralizing antibodies are numbered from 1 to 4 and the matrix indicates which antibodies compete with each other (same or similar epitope; dark grey) and which do not compete with each other (different epitope; light grey) for binding to the RBD of SARS-CoV-2; see also Example 6 and Table V.

FIG. 5: Neutralization capability of the antibodies of the present invention as determined by pseudovirus entry assay using pseudovirus expressing the S protein of SARS-CoV-2; see Example 7. In FIG. 5A the results for the exemplarily antibodies NI-607.274_B7, NI-607.426_F1, NI-607.427_C5, and NI-607.429_B9 are shown; in FIG. 5B for antibodies NI-607.429_E4, NI-607.531_C8, NI-607.529_B9, and NI-607.529_G4; in FIG. 5C for antibodies NI-607.532_C11, NI-607.532_D3, NI-607.532_B6, and NI-607.531_D8; in FIG. 5D for antibodies NI-607.532_F3, NI-607.531_E7, NI-607.532_F9, NI-607.649_B11; and in FIG. 5E for antibody NI-607.649_G7 as well as for the anti-SARS-CoV-2 RBD antibody D002 (SinoBiologics; Catalog number: 40150-D002) and for the negative controls (PBS and isotype control).
It has been found that a significant number of the antibodies of the present invention reach the maximum neutralization of 50% or greater. In particular, the IC₅₀ of antibody NI-607.429_B9 as determined by the pseudovirus entry assay is 7.1 pg/mL, the IC₅₀ of antibody NI-607.429_E4 is 35 ng/mL, the IC₅₀ of antibody NI-607.529_B9 is 1.9 pg/mL, the IC₅₀ of antibody NI-607.531_C8 is 86.4 ng/mL (0.572 nM), the IC₅₀ of antibody NI-607.532_B6 is 100 μg/mL, the IC₅₀ of antibody NI-607.532_C11 is 87.2 ng/mL, the IC₅₀ of antibody NI-607.532_D3 is 2.1 μg/mL, the IC₅₀ of antibody NI-607.532_F9 is 363.6 ng/mL, the IC₅₀ of antibody NI-607.649_B11 is 231.8 ng/mL (1.531 nM), the IC50 of antibody NI-607.531_E7 is 3.9 μg/mL, and the IC₅₀ of antibody NI-607.532_F3 is 11.7 μg/mL. Antibodies NI-607.427_C5, NI-607.426_F11, NI-607.274_B7, NI-607.529_G4, NI-607.531_D8, and NI-607.649_G7 showed an IC₅₀>100 μg/mL.

FIG. 6: Neutralization capability of the antibodies of the present invention as determined by cytopathic effect (CPE) inhibition assay; see Example 8. Exemplarily antibodies were tested for their inhibition of viral induced cytotoxicity via infecting the human epithelial colorectal adenocarcinoma cell line Caco-2 with a SARS-CoV-2 isolate. After culturing of 72 hours microscopic pictures were taken to demonstrate the cytopathic effect. Antibodies NI-607.649_B11, NI-607.532_F9, NI-607.532_D3, NI-607.532_C11, NI-607.521_C8, and NI-607.429_E4 show a similar or even better neutralization than the positive control, while neutralization by antibody NI-607.529_G4 is barely detectable.

FIG. 7: Neutralization capability of the antibodies of the present invention as determined by SARS-Cov2-GFP CPE/GFP assay; see Example 9. Vero E6 were cells infected with recombinant SARS-CoV-2-GFP and the infection was scored by high throughput microscopy every four hours. The experiment was carried out in triplicate and mean GFP signal per well are shown in FIGS. 7A, 7C, 7E, and 7G for antibodies NI-607.531_C8, NI-607.649_B11, NI-607.429_E4 and the isotype control, wherein mean values of the confluence of cell monolayer are shown in FIGS. 7B, 7D, 7F, and 7H for antibodies NI-607.531_C8, NI-607.649_B11, NI-607.429_E4 and the isotype control. Antibodies NI-607.531_C8, NI-607.649_B11, and NI-607.429_E4 showed clear reduction in GFP which equals viral gene expression with dilutions 1/3125 to 1/25 (NI-607.531_C8), with dilutions 1/125 and 1/25 (NI-607.649_B11) and with dilutions 1/625 to 1/25 (NI-607.429_E4), respectively, and do not show any signs of cell toxicity, i.e. no enhanced cell toxicity in comparison to the isotype control.

FIG. 8: Prophylactic and therapeutic efficacy of anti-SARS-Cov-2 antibodies of the present invention in the golden Syrian hamster model of SARS-CoV-2 infection. (A) Overview of study design. (B) Impact of NI-607.531_C8 and NI-607.649_B11 on weight loss in prophylaxis. (C) Impact of NI-607.531_C8 and NI-607.649_B11 on weight loss in treatment.

FIG. 9: Direct ELISA (antigens indicated below) against 23 concentrations of recombinant human-derived SARS-CoV-2 antibodies NI-607.531_C8 and NI-607.649_B11 for binding the RBD or full spike protein of SARS-CoV-2.
(A, C) Coated antigens: SARS-CoV-2-S1(RBD)-His 0.32 mg/mL Trenzyme, SARS-CoV-2 (2019-nCoV) Spike S1-His 250 μg/ml Sino 40591-V08H, SARS-CoV-2 Spike Protein (S1+S2 ECD, His-Tag) 250 μg/ml Sino 40589-V08B1, SARS-CoV-2 (2019-nCoV) Spike RBD-His 250 ug/ml Sino 40592-V08B, SARS-CoV-2 (2019-nCoV) Spike RBD(N501Y)-His 250 ug/ml Sino 40592-V08H82, SARS-CoV-2 (2019-nCoV) Spike RBD(Y453F)-His 250 ug/ml Sino 40592-V08H80, SARS-CoV-2 (2019-nCoV) Spike S1(D614G)-His 250 ug/ml Sino 40591-V08H3, SARS-CoV-2 (2019-nCoV) Spike RBD(N439K)-His 250 ug/ml Sino 40592-V08H14.
(B,D) Coated antigens:SARS-CoV-2 (2019-nCoV) Spike RBD-His 250 ug/ml Sino 40592-V08B, SARS-CoV-2 (2019-nCoV) Spike RBD(Y453F)-His 250 ug/ml Sino 40592-V08H80, SARS-CoV-2 (2019-nCoV) Spike S1-His 250 ug/ml Sino 40591-V08H, SARS-CoV-2 (2019-nCoV) Spike S1(K417N, E484K, N501Y, D614G)-His 250 μg/ml Sino 40591-V08H10, SARS-CoV-2 (2019-nCoV) Spike S1(HV69-70 deletion, Y453F, D614G)-His 250 ug/ml Sino 40591-V08H8, SARS-CoV-2 Spike Protein (S1+S2 ECD, His-Tag) 250 ug/ml Sino 40589-V08B1, SARS-CoV-2 full-length Trimeric Spike Recombinant Antigen (B.1.135, South Africa, (Antibody Resistance of SARS-CoV-2 Variants B.1.351 and B.1.1.7 Pengfei Wang, Manoj S. Nair, Lihong Liu, Sho Iketani, Yang Luo, Yicheng Guo, Maple Wang, Jian Yu, Baoshan Zhang, Peter D. Kwong, Barney S. Graham, John R. Mascola, Jennifer Y. Chang, Michael T. Yin, Magdalena Sobieszczyk, Christos A. Kyratsous, Lawrence Shapiro, Zizhang Sheng, Yaoxing Huang, David D. Ho bioRxiv 2021.01.25.428137)) 0.8 mg/ml Bioserv BSV-COV-PR-61, SARS-CoV-2 full-length Trimeric Spike Recombinant Antigen (P.1, Brazil, Increased Resistance of SARS-CoV-2 Variant P.1 to Antibody Neutralization, Pengfei Wang, Ryan G. Casner, Manoj S. Nair, Maple Wang, Jian Yu, Gabriele Cerutti, Lihong Liu, Peter D. Kwong, Yaoxing Huang, Lawrence Shapiro, David D. Ho, bioRxiv 2021.03.01.433466)) 1.12 mg/ml Bioserv BSV-COV-PR-69, SARS-CoV-2 full-length Trimeric Spike Recombinant Antigen (B.1.1.7, UK variant, (Antibody Resistance of SARS-CoV-2 Variants B.1.351 and B.1.1.7 Pengfei Wang, Manoj S. Nair, Lihong Liu, Sho Iketani, Yang Luo, Yicheng Guo, Maple Wang, Jian Yu, Baoshan Zhang, Peter D. Kwong, Barney S. Graham, John R. Mascola, Jennufer Y. Chang, Michael T Yin, Magdalena Sobieszczyk, Christos A. Kyratsous, Lawrence Shapiro, Zizhang Sheng, Yaoxing Huang, David D. Ho bioRxiv 2021.01.25.428137)) 0.92 mg/ml Bioserv BSV-COV-PR-65, Bovine Serum Albumin ≥96% Sigma, Cat #A8022-100G.

FIG. 10: Schematic amino acid representation of spike RBD of SARS-CoV-2, which includes an additional Methionine at the N-terminus for expression purposes (SEQ ID NO: 319). Epitope sites indicated for recombinant human-derived SARS-CoV-2 antibodies NI-607.531_C8 (A) and NI-607.649_B11 (B). Epitope mapping by Hydrogen Deuterium eXchange mass spectrometry (HDX-MS, CovalX): Bars below sequence indicate interaction sides, dark=high, mid grey=medium and light grey=low protection. Epitope mapping by Crosslinking Mass Spectrometry (XL-MS, CovalX): Bars above sequence indicate cross-linked epitope contacts, and vertical lines originating from the bars indicate single amino acid interaction sites. Boxes around individual amino acids represent locations of mutations circulating SARS-CoV-2 variants. Classification is based on Pango lineages (Rambaut, A., Holmes, E. C., O'Toole, A. et al. A dynamic nomenclature proposal for SARS-CoV-2 lineages to assist genomic epidemiology. Nat Microbiol 5, 1403-1407 (2020).), label UK: B.1.1.7, UK variant, (Antibody Resistance of SARS-CoV-2 Variants B.1.351 and B.1.1.7 Pengfei Wang, Manoj S. Nair, Lihong Liu, Sho Iketani, Yang Luo, Yicheng Guo, Maple Wang, Jian Yu, Baoshan Zhang, Peter D. Kwong, Barney S. Graham, John R. Mascola, Jennifer Y. Chang, Michael T. Yin, Magdalena Sobieszczyk, Christos A. Kyratsous, Lawrence Shapiro, Zizhang Sheng, Yaoxing Huang, David D. Ho bioRxiv 2021.01.25.428137), label SA: B.1.135, South Africa, (Antibody Resistance of SARS-CoV-2 Variants B.1.351 and B.1.1.7 Pengfei Wang, Manoj S. Nair, Lihong Liu, Sho Iketani, Yang Luo, Yicheng Guo, Maple Wang, Jian Yu, Baoshan Zhang, Peter D. Kwong, Barney S. Graham, John R. Mascola, Jennifer Y. Chang, Michael T. Yin, Magdalena Sobieszczyk, Christos A. Kyratsous, Lawrence Shapiro, Zizhang Sheng, Yaoxing Huang, David D. Ho bioRxiv 2021.01.25.428137), label BRA: P.1, Brazil, Increased Resistance of SARS-CoV-2 Variant P.1 to Antibody Neutralization, Pengfei Wang, Ryan G. Casner, Manoj S. Nair, Maple Wang, Jian Yu, Gabriele Cerutti, Lihong Liu, Peter D. Kwong, Yaoxing Huang, Lawrence Shapiro, David D. Ho, bioRxiv 2021.03.01.433466), label IN: B.1.617, India, (Convergent evolution of SARS-CoV-2 spike mutations, L452R, E484Q and P681R, in the second wave of COVID-19 in Maharashtra, India, Sarah Cherian, Varsha Potdar, Santosh Jadhav, Pragya Yadav, Nivedita Gupta, Mousmi Das, Soumitra Das, Anurag Agarwal, Sujeet Singh, Priya Abraham, Samiran Panda, Shekhar Mande, Renu Swarup, Balram Bhargava, Rajesh Bhushan, NIC team, INSACOG Consortium, bioRxiv 2021.04.22.440932).

### DETAILED DESCRIPTION OF THE INVENTION

Generally, the present invention relates to human-derived monoclonal anti-SARS-CoV-2 antibodies as well as to compositions and antibody cocktails comprising the antibodies, wherein the antibodies demonstrate the immunological characteristics of any one of the anti-SARS-CoV-2 antibodies illustrated in the Examples further below and which are summarized in Table I. In particular, the antibodies bind with high affinity to the RBD of the S protein of SARS-CoV-2, i.e. with an EC₅₀ of <100 pM, which makes them suitable for both targeting the virus as well as diagnosing viral proteins which are released into body fluids such as blood. Furthermore, the antibodies of the present invention typically do not show any cross-activities with unrelated proteins such as serum albumin, in particular bovine serum albumin, i.e. proteins which are commonly used in the formulation of pharmaceuticals or laboratory use. Accordingly, the antibody of the present invention, also due to the human origin of the antibodies, i.e. maturation of the original antibodies in the human body, can be reasonably expected to be safe as therapeutic agent for the treatment of COVID-19 and other SARS-CoV-2 related diseases and specific as a laboratory reagent for the detection of SARS-CoV-2 without giving false positives. Furthermore, the present invention generally relates to anti-SARS-CoV-2 antibodies and antigen-binding fragments thereof, preferably human-derived monoclonal antibodies as well as to polynucleotides encoding the antibody or antigen binding fragment thereof, wherein the antibody is capable of binding to RBD of the S protein of SARS-CoV-2, and wherein the antibody or antigen-binding fragment thereof is characterized to be of the IgG4 isotype, i.e. the antibody or antigen-binding fragment thereof comprises an IgG4 constant domain, preferably including the S228P mutation. In addition, the antibody preferably binds to a conformational epitope. Furthermore, in one embodiment, the antibody does not bind to the RBD of SARS-CoV while in another embodiment the antibody binds to the RBD of SARS-CoV.

For the avoidance of any doubt it is emphasized that the expressions “in one embodiment” and “in a further embodiment” and the like are meant that any of the embodiments described therein are to be read with a mind to combine each of the features of those embodiments and that the disclosure has to be treated in the same way as if the combination of the features of those embodiments would be spelled out in one embodiment. The same is true for any combination of embodiments and features of the appended claims, which are also intended to be combined with features from corresponding embodiments disclosed in the description, wherein only for the sake of consistency and conciseness the embodiments are characterized by dependencies while in fact each embodiment and combination of features, which could be construed due to the (multiple) dependencies must be seen to be literally disclosed and not considered as a selection among different choices.

Unless otherwise stated, a term as used herein is given the definition as provided in the Oxford Dictionary of Biochemistry and Molecular Biology, Oxford University Press, 1997, revised 2000 and reprinted 2003, ISBN 0 19 850673 2; Second edition published 2006, ISBN 0-19-852917-1 978-0-19852917-0.

Furthermore, regarding anti-SARS-CoV-2 antibodies, their recombinant production in a host cell, purification, modification, formulation in a pharmaceutical composition and therapeutic use as well as terms and feature common in the art can be relied upon by the person skilled in art when carrying out the present invention as claimed; see, e.g., Antibodies A Laboratory Manual 2^{nd} edition, 2014 by Cold Spring Harbor Laboratory Press, Cold Spring Harbor, N.Y., USA, wherein also antibody purification and storage; engineering antibodies, including use of degenerate oligonucleotides, 5′-RACE, phage display, and mutagenesis, immunoblotting protocols and the latest screening and labeling techniques are described.

The terms “anti-SARS-CoV-2 antibody” or “SARS-CoV-2-binding fragment” refers to an antibody or binding fragment directed against the RBD of the S protein of SARS-CoV-2 if not stated otherwise.

The antibodies of the present invention have been originally isolated from human donors after recovery from COVID-19 infection and only those have been considered for further characterization which were capable of specifically binding the RBD of SARS-CoV-2 S with high affinity. In this context, in order to obtain a measure of the binding affinity, the EC₅₀ of the antibodies in the ELISA performed in Example 3 was determined. The term “EC₅₀”, in the context of an in vitro or in vivo assay using an antibody or antigen-binding fragment thereof, refers to the concentration of an antibody or an antigen-binding fragment thereof that induces a response that is 50% of the maximal response, i.e., halfway between the maximal response and the baseline. As mentioned above, only those antibodies having a high affinity to the RBD of SARS-CoV-2 S have been further considered, i.e. those having an EC₅₀ in the picomolar range, preferably having an EC₅₀ of <100 pM as determined in ELISA assays as described in Example 3. Thus, in one embodiment the antibodies of the present invention recognize the RBD of SARS-CoV-2 S with an EC₅₀ of <100, preferably with an EC₅₀ of <90, preferably with an EC₅₀ of <70, preferably with an EC₅₀ of <50 pM, preferably with an EC₅₀ of <40 pM, preferably with an EC₅₀ of <35 pM, more preferably with an EC₅₀ of <30 pM, still more preferably with an EC₅₀ of <20 pM, still more preferably with an EC₅₀ of <15 pM and even more preferably with an EC₅₀ of <12 pM as determined by ELISA assay.

The values as determined for each antibody are listed in Table I and thus, in one embodiment, the EC₅₀ of antibody NI-607.274_B7 as determined by ELISA for the RBD of SARS-CoV-2 S is about 21 pM. In another embodiment, the EC₅₀ of antibody NI-607.274_E5 is about 15 pM. In another embodiment, the EC₅₀ of antibody NI-607.275_C5 is about 20 pM. In another embodiment, the EC₅₀ of antibody NI-607.426_D4 is about 33 pM. In another embodiment, the EC₅₀ of antibody NI-607.426_E2 is about 30 pM. In another embodiment, the EC₅₀ of antibody NI-607.426_F11 is about 17 pM. In another embodiment, the EC₅₀ of antibody NI-607.427_C5 is about 14 pM. In another embodiment, the EC₅₀ of antibody NI-607.428_B9 is about 26 pM. In another embodiment, the EC₅₀ of antibody NI-607.429_B9 is about 14 pM. In another embodiment, the EC₅₀ of antibody NI-607.429_E4 is about 8.6 pM. In another embodiment, the EC₅₀ of antibody NI-607.529_B9 is about 34 pM. In another embodiment, the EC₅₀ of antibody NI-607.529_G4 is about 26 pM. In another embodiment, the EC₅₀ of antibody NI-607.531_C8 is about 11 pM. In another embodiment, the EC₅₀ of antibody NI-607.531_D8 is about 4.5 pM. In another embodiment, the EC₅₀ of antibody NI-607.532_B6 is about 4.5 pM. In another embodiment, the EC₅₀ of antibody NI-607.532_C11 is about 13 pM. In another embodiment, the EC₅₀ of antibody NI-607.532_C8 is about 9.9 pM. In another embodiment, the EC₅₀ of antibody NI-607.532_D3 is about 4.9 pM. In another embodiment, the EC₅₀ of antibody NI-607.532_D4 is about 5.5 pM. In another embodiment, the EC₅₀ of antibody NI-607.532_D8 is about 5.1 pM. In another embodiment, the EC₅₀ of antibody NI-607.532_F9 is about 2.5 pM. In another embodiment, the EC₅₀ of antibody NI-607.649_B11 is about 27 pM. In another embodiment, the EC₅₀ of antibody NI-607.531_E7 is about 27 pM. In another embodiment, the EC₅₀ of antibody NI-607.532_F3 is about 19 pM. In another embodiment, the EC₅₀ of antibody NI-607.649_G7 is about 24 pM. In another embodiment, the EC₅₀ of antibody NI-607.761_B7 is about 1 pM. In another embodiment, the EC₅₀ of antibody NI-607.791_B10 is about 62 pM. In another embodiment, the EC₅₀ of antibody NI-607.531_E3 is about 20 pM. In another embodiment, the EC₅₀ of antibody NI-607.820_B6 is about 29 pM. In another embodiment, the EC₅₀ of antibody NI-607.820_B7 is about 82 pM.

As mentioned, the antibodies do not show any cross-reactivity with BSA which makes them in particular suitable for therapeutic approaches or laboratory use as explained above.

The present invention is illustrated with anti-SARS-CoV-2 antibodies and antigen-binding fragments thereof which are characterized by comprising in their variable region, i.e. binding domain, the variable heavy (VH) and variable light (VL) chain having the amino acid sequences depicted in Table II. The corresponding nucleotide and amino acid sequences are set forth in Table II as well.

As always, the variable domains of each chain contain three hypervariable loops named complementarity determining regions (CDRs, CDR-1, -2, and -3). The CDRs are separated by structurally conserved regions called framework regions (FR-1, -2, -3, and -4) that form a “core” ß-sheet structure displaying these loops on the surface of the variable domain. The length and composition of the CDR sequences are highly variable, especially in the CDR3. The CDRs are approximated to the paratope of the antibody that interacts with the antigen and therefore contains the antigen-binding residues. Accordingly, it is common to define an antibody by its six CDRs. Exemplary sets of CDRs in the above amino acid sequences of the VH and VL chains are depicted Table II. However, as discussed in the following the person skilled in the art is well aware of the fact that in addition or alternatively CDRs may be used, which differ in their amino acid sequence from those set forth in Table II by one, two, three or even more amino acids, especially in case of CDR2 and CDR3. As mentioned in the paragraph below Table II, the person skilled in the art can easily identify the CDRs according to common principles, for example as summarized in www.bioinforg.uk/abs. In this context, while the CDRs of the antibodies listed in Table II are indicated according to Chothia, the person skilled in the art knows that a number of definitions of the CDRs are commonly in use, i.e. the
(i) Kabat definition based on sequence variability;
(ii) Chothia definition based on the location of the structural loop regions;
(iii) AbM definition as a compromise between the two used by Oxford Molecular's AbM antibody modelling software; and
(iv) Contact definition that has been recently introduced by and is based on an analysis of the available complex crystal structures. This definition is likely to be the most useful for performing mutagenesis to modify the affinity of an antibody since these are residues which take part in interactions with the antigen. For lists of CDR residues making contact in each antibody with summary data for each CDR see, e.g., www.bioinf.org.uk/abs which also refers to antibody modelling software such as abYmod available at abymod.abysis.org.

Table III below depicts the relation between the CDR positions defined by the different concepts.

TABLE III
Different concepts of CDR definitions.

| Loop | Kabat | AbM | Chothia¹ | Contact² | IMGT |
|---|---|---|---|---|---|
| L1 | L24--L34 | L24--L34 | L24--L34 | L30--L36 | L27--L32 |
| L2 | L50--L56 | L50--L56 | L50--L56 | L46--L55 | L50--L51 |
| L3 | L89--L97 | L89--L97 | L89--L97 | L89--L96 | L89--L97 |
| H1 | H31--H35B | H26--H35B | H26--H32 . . . 34 | H30--H35B | H26--H35B |
| | (Kabat Numbering)³ | | | | |
| H1 | H31--H35 | H26--H35 | H26--H32 | H30--H35 | H26--H33 |
| | (Chothia Numbering) | | | | |
| H2 | H50--H65 | H50--H58 | H52--H56 | H47--H58 | H51--H56 |
| H3 | H95--H102 | H95--H102 | H95--H102 | H93--H101 | H93--H102 |

¹some of these definitions (particularly for Chothia loops) vary depending on the individual publication examined;
²any of the numbering schemes can be used for these CDR definitions, except the contact definition uses the Chothia or Martin (Enhanced Chothia) definition;
³the end of the Chothia CDR-H1 loop when numbered using the Kabat numbering convention varies between H32 and H34 depending on the length of the loop. (This is because the Kabat numbering scheme places the insertions at H35A and H35B.)

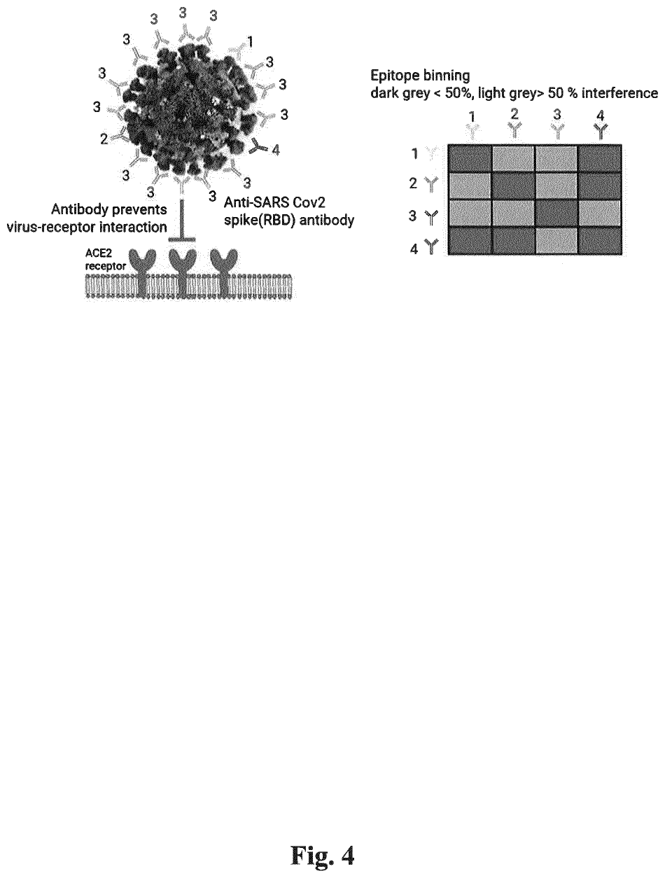

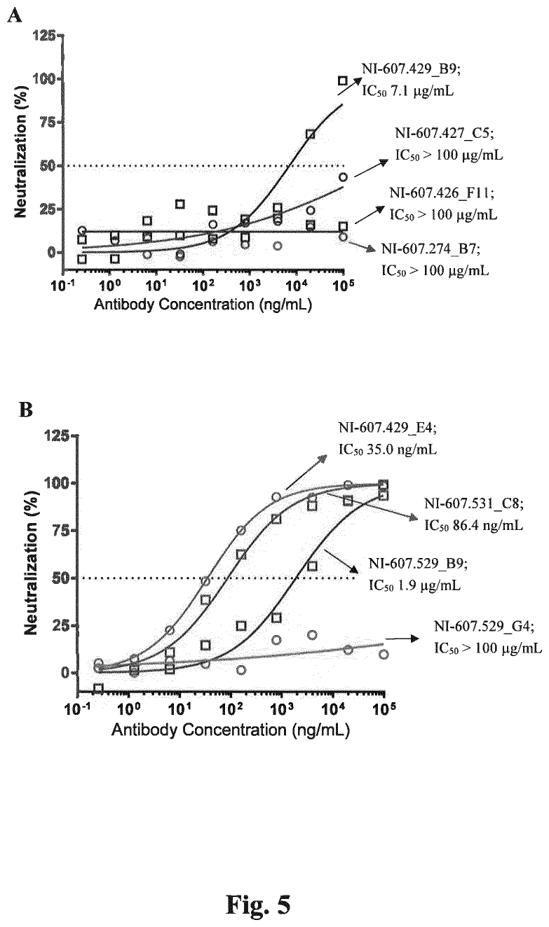

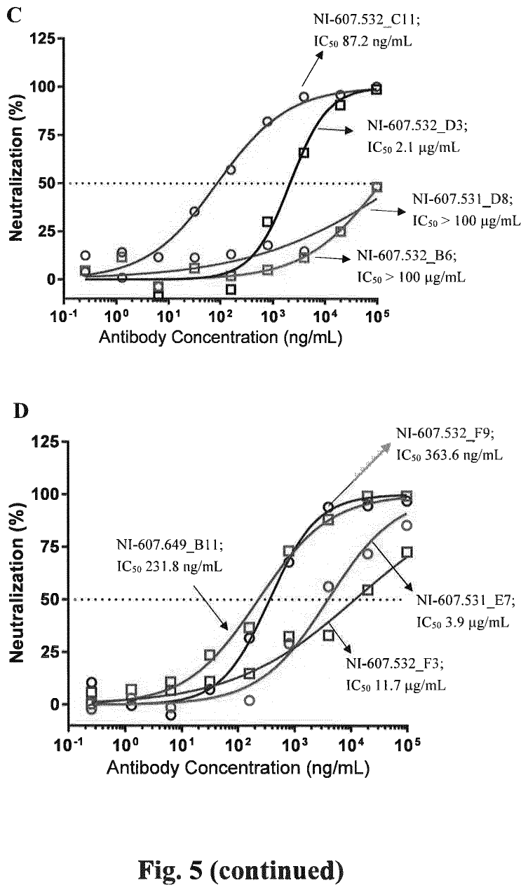

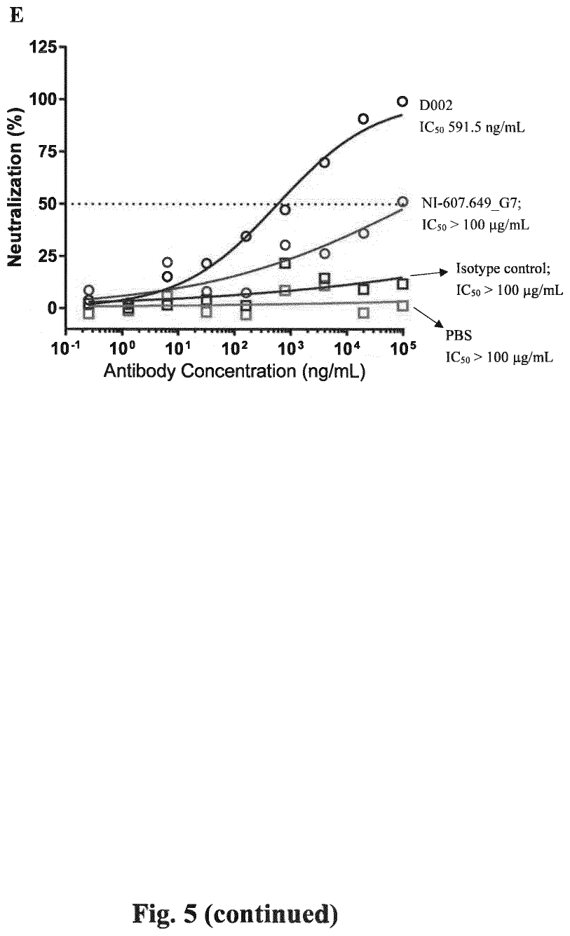

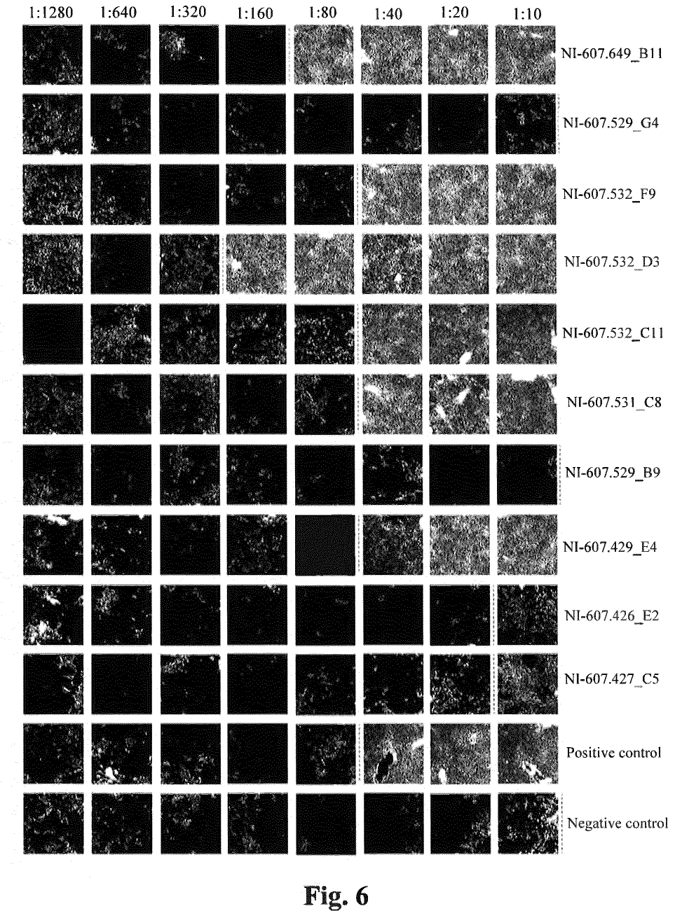

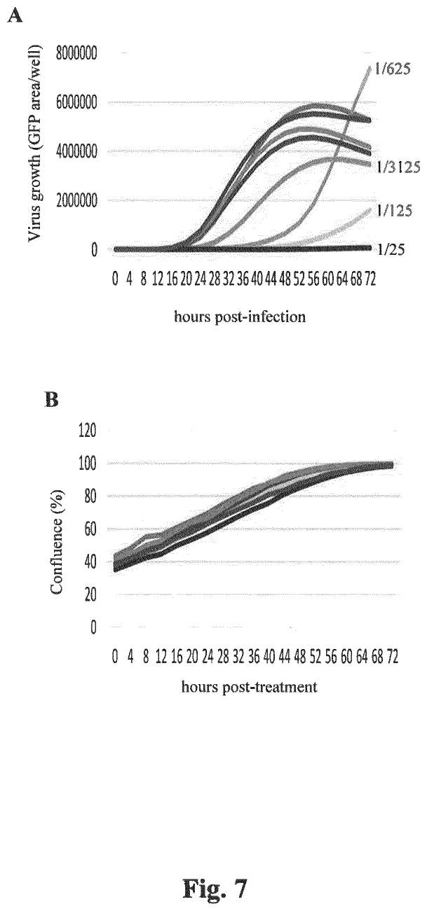

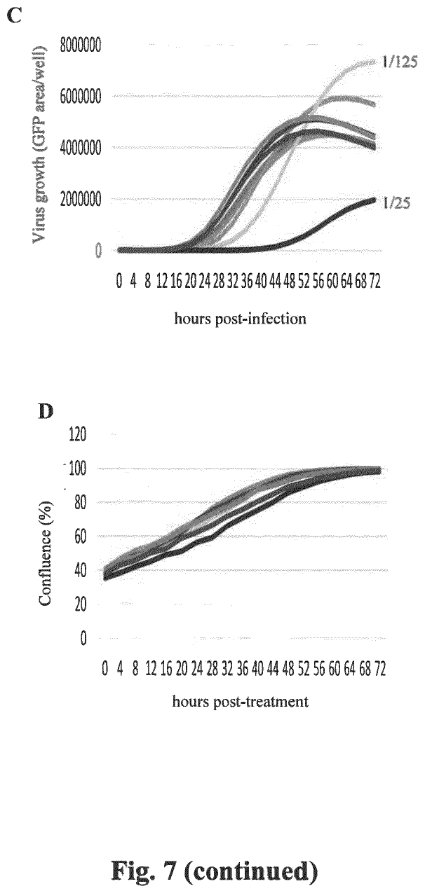

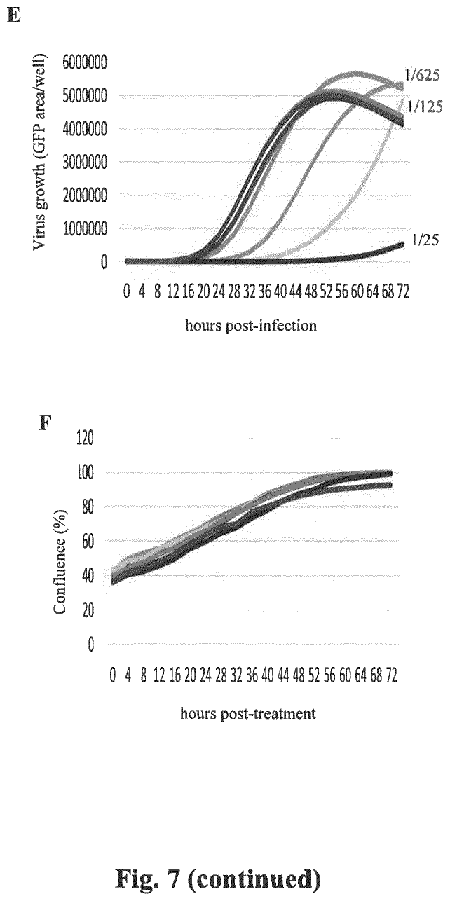

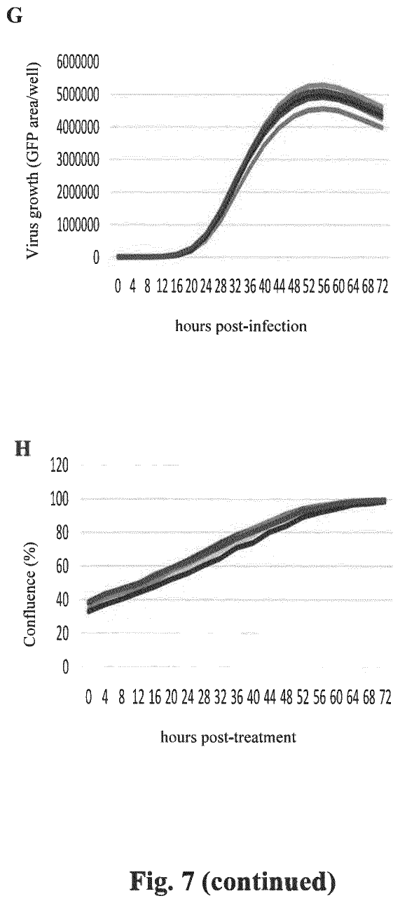

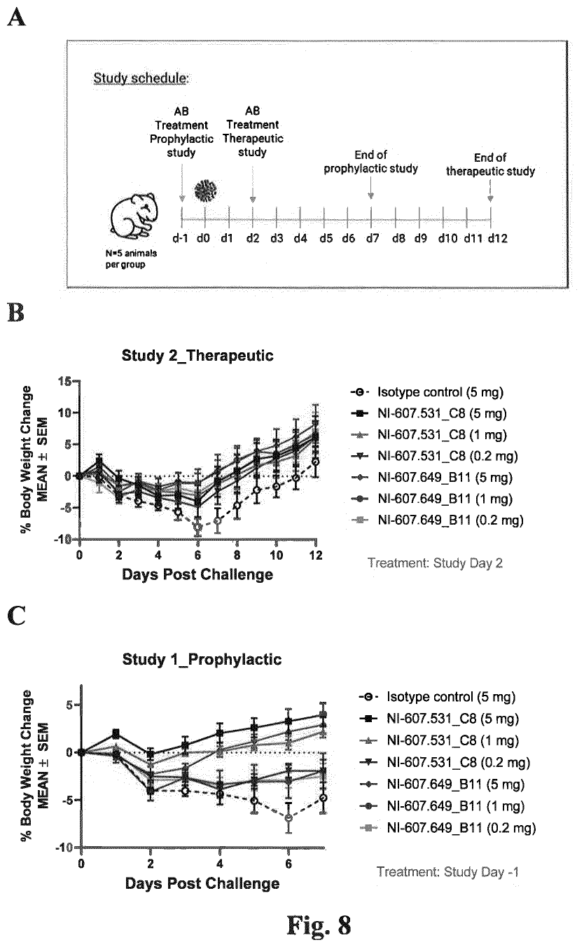

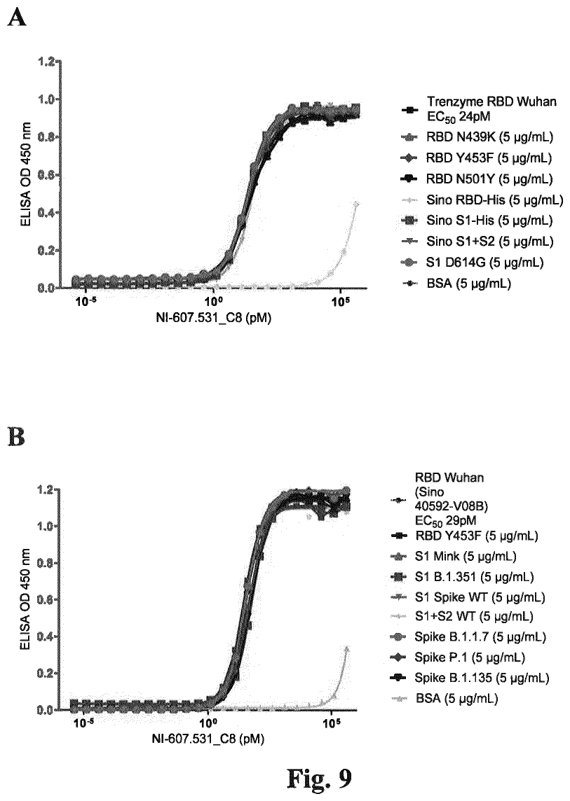

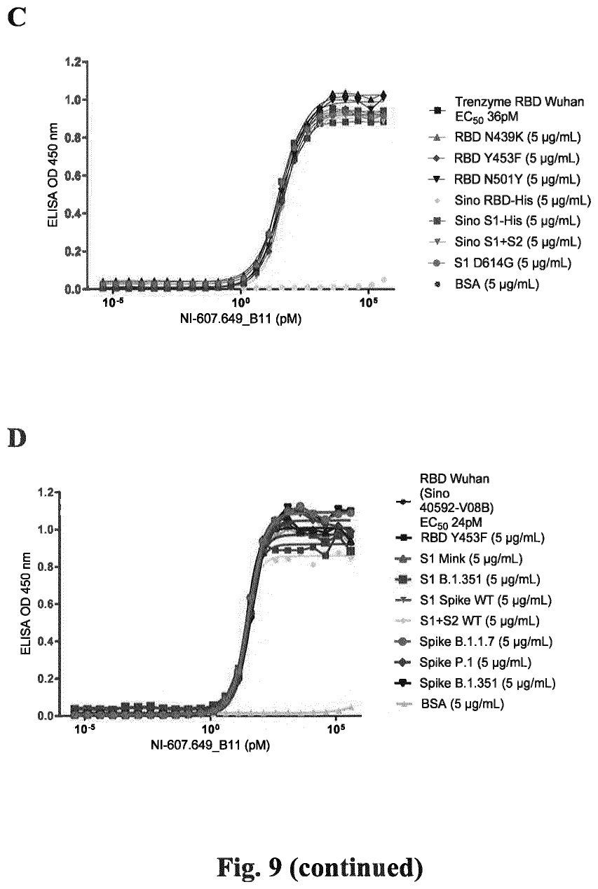

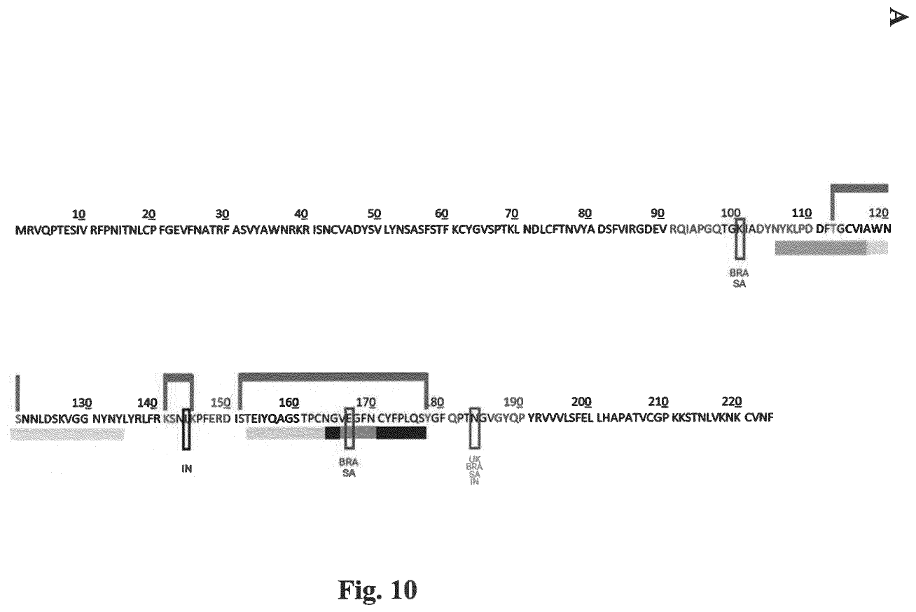

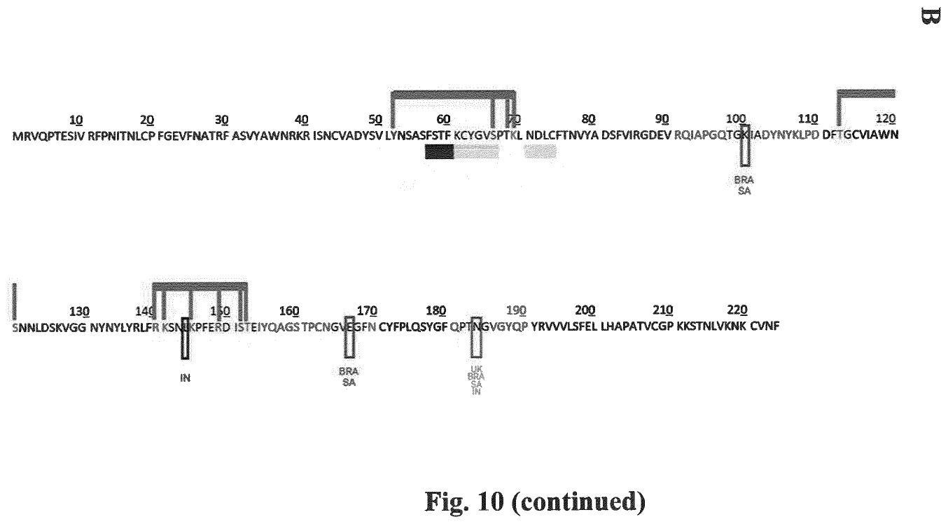

For the mentioned definitions see also Kontermann and Dubel (eds.), Antibody Engineering Vol. 2, DOI 10.1007/978-3-642-01147-4_3, #Springer-Verlag Berlin Heidelberg 2010, in particular Chapter 3, Protein Sequence and Structure Analysis of Antibody Variable Domains at pages 33-51 and Dondelinger et al., Front. Immunol. 9 (2018), 2278 specifically dealing with understanding the significance and implications of antibody numbering and antigen-binding surface/residue definition; see, e.g., Dondelinger et al., FIG. 4 and FIG. 6 illustrating the disparity in the classical CDR definitions according to Kabat supra, Chothia (Chothia and Lesk, J. Mol. Biol. 196 (1987), 901-917), Contact (MacCallum et al, J. Mol. Biol. 262 (1996), 732-745) and IMGT (IMGT®, the international ImMunoGeneTics information System®, www.imgt.org). The AbM definition is a compromise between the two used by Oxford Molecular's AbM antibody modelling software.

This above diagram illustrates the alternative definitions for CDR-H1 (VH-CDR1). The Kabat and Chothia numbering schemes are shown horizontally and the Kabat, Chothia, AbM and Contact definitions of the CDRs are shown with arrows above and below the two numbering schemes.

In one embodiment, the present invention relates to a human-derived recombinant monoclonal anti-SARS-CoV-2 antibody or SARS-CoV-2 binding fragment, synthetic derivative, or biotechnological derivative of antibody NI-607.274_B7, NI-607.274_E5, NI-607.275_C5, NI-607.426_D4, NI-607.426_E2, NI-607.426_F11, NI-607.427_C5, NI-607.428_B9, NI-607.429_B9, NI-607.429_E4, NI-607.529_B9, NI-607.529_G4, NI-607.531_C8, NI-607.531_D8, NI-607.532_B6, NI-607.532_C11, NI-607.532_C8, NI-607.532_D3, NI-607.532_D4, NI-607.532_D8, NI-607.532_F9, NI-607.649_B11, NI-607.531_E7, NI-607.532_F3, NI-607.649_G7, NI-607.761_B7, NI-607.791_B10, NI-607.531_E3, NI-607.820_B6, or NI-607.820_B7 and/or to a composition of said antibodies or fragments/derivatives, wherein the antibody, fragment or derivative thereof comprises a variable heavy (VH) chain comprising VH complementary determining regions (CDRs) 1, 2, and 3, and a variable light (VL) chain comprising VL CDRs 1, 2, and 3 as defined by Chothia, wherein

NI-607.274_B7

VH-CDR1 comprises the amino acid sequence of SEQ ID NO: 3 or a variant thereof, wherein the variant comprises one or two amino acid substitutions,

VH-CDR2 comprises the amino acid sequence of SEQ ID NO: 4 or a variant thereof, wherein the variant comprises one or two amino acid substitutions,

VH-CDR3 comprises the amino acid sequence of SEQ ID NO: 5 or a variant thereof, wherein the variant comprises one or two amino acid substitutions,

VL-CDR1 comprises the amino acid sequence of SEQ ID NO: 8 or a variant thereof, wherein the variant comprises one or two amino acid substitutions,

VL-CDR2 comprises the amino acid sequence of SEQ ID NO: 9 or a variant thereof, wherein the variant comprises one or two amino acid substitutions, and

VL-CDR3 comprises the amino acid sequence of SEQ ID NO: 10 or a variant thereof, wherein the variant comprises one or two amino acid substitutions; or

NI-607.274_E5

VH-CDR1 comprises the amino acid sequence of SEQ ID NO: 13 or a variant thereof, wherein the variant comprises one or two amino acid substitutions,

VH-CDR2 comprises the amino acid sequence of SEQ ID NO: 14 or a variant thereof, wherein the variant comprises one or two amino acid substitutions,

VH-CDR3 comprises the amino acid sequence of SEQ ID NO: 15 or a variant thereof, wherein the variant comprises one or two amino acid substitutions,

VL-CDR1 comprises the amino acid sequence of SEQ ID NO: 18 or a variant thereof, wherein the variant comprises one or two amino acid substitutions,

VL-CDR2 comprises the amino acid sequence of SEQ ID NO: 19 or a variant thereof, wherein the variant comprises one or two amino acid substitutions, and

VL-CDR3 comprises the amino acid sequence of SEQ ID NO: 20 or a variant thereof, wherein the variant comprises one or two amino acid substitutions; or

NI-607.275_C5

VH-CDR1 comprises the amino acid sequence of SEQ ID NO: 23 or a variant thereof, wherein the variant comprises one or two amino acid substitutions,

VH-CDR2 comprises the amino acid sequence of SEQ ID NO: 24 or a variant thereof, wherein the variant comprises one or two amino acid substitutions,

VH-CDR3 comprises the amino acid sequence of SEQ ID NO: 25 or a variant thereof, wherein the variant comprises one or two amino acid substitutions,

VL-CDR1 comprises the amino acid sequence of SEQ ID NO: 28 or a variant thereof, wherein the variant comprises one or two amino acid substitutions,

VL-CDR2 comprises the amino acid sequence of SEQ ID NO: 29 or a variant thereof, wherein the variant comprises one or two amino acid substitutions, and

VL-CDR3 comprises the amino acid sequence of SEQ ID NO: 30 or a variant thereof, wherein the variant comprises one or two amino acid substitutions; or

NI-607.426_D4

VH-CDR1 comprises the amino acid sequence of SEQ ID NO: 33 or a variant thereof, wherein the variant comprises one or two amino acid substitutions,

VH-CDR2 comprises the amino acid sequence of SEQ ID NO: 34 or a variant thereof, wherein the variant comprises one or two amino acid substitutions,

VH-CDR3 comprises the amino acid sequence of SEQ ID NO: 35 or a variant thereof, wherein the variant comprises one or two amino acid substitutions,

VL-CDR1 comprises the amino acid sequence of SEQ ID NO: 38 or a variant thereof, wherein the variant comprises one or two amino acid substitutions,

VL-CDR2 comprises the amino acid sequence of SEQ ID NO: 39 or a variant thereof, wherein the variant comprises one or two amino acid substitutions, and

VL-CDR3 comprises the amino acid sequence of SEQ ID NO: 40 or a variant thereof, wherein the variant comprises one or two amino acid substitutions; or

NI-607.426_E2

VH-CDR1 comprises the amino acid sequence of SEQ ID NO: 43 or a variant thereof, wherein the variant comprises one or two amino acid substitutions,

VH-CDR2 comprises the amino acid sequence of SEQ ID NO: 44 or a variant thereof, wherein the variant comprises one or two amino acid substitutions,

VH-CDR3 comprises the amino acid sequence of SEQ ID NO: 45 or a variant thereof, wherein the variant comprises one or two amino acid substitutions,

VL-CDR1 comprises the amino acid sequence of SEQ ID NO: 48 or a variant thereof, wherein the variant comprises one or two amino acid substitutions,

VL-CDR2 comprises the amino acid sequence of SEQ ID NO: 49 or a variant thereof, wherein the variant comprises one or two amino acid substitutions, and

VL-CDR3 comprises the amino acid sequence of SEQ ID NO: 50 or a variant thereof, wherein the variant comprises one or two amino acid substitutions; or

NI-607.426_F11

VH-CDR1 comprises the amino acid sequence of SEQ ID NO: 53 or a variant thereof, wherein the variant comprises one or two amino acid substitutions,

VH-CDR2 comprises the amino acid sequence of SEQ ID NO: 54 or a variant thereof, wherein the variant comprises one or two amino acid substitutions,

VH-CDR3 comprises the amino acid sequence of SEQ ID NO: 55 or a variant thereof, wherein the variant comprises one or two amino acid substitutions,

VL-CDR1 comprises the amino acid sequence of SEQ ID NO: 58 or a variant thereof, wherein the variant comprises one or two amino acid substitutions,

VL-CDR2 comprises the amino acid sequence of SEQ ID NO: 59 or a variant thereof, wherein the variant comprises one or two amino acid substitutions, and

VL-CDR3 comprises the amino acid sequence of SEQ ID NO: 60 or a variant thereof, wherein the variant comprises one or two amino acid substitutions; or

NI-607.427_C5

VH-CDR1 comprises the amino acid sequence of SEQ ID NO: 63 or a variant thereof, wherein the variant comprises one or two amino acid substitutions,

VH-CDR2 comprises the amino acid sequence of SEQ ID NO: 64 or a variant thereof, wherein the variant comprises one or two amino acid substitutions,

VH-CDR3 comprises the amino acid sequence of SEQ ID NO: 65 or a variant thereof, wherein the variant comprises one or two amino acid substitutions,

VL-CDR1 comprises the amino acid sequence of SEQ ID NO: 68 or a variant thereof, wherein the variant comprises one or two amino acid substitutions,

VL-CDR2 comprises the amino acid sequence of SEQ ID NO: 69 or a variant thereof, wherein the variant comprises one or two amino acid substitutions, and

VL-CDR3 comprises the amino acid sequence of SEQ ID NO: 70 or a variant thereof, wherein the variant comprises one or two amino acid substitutions; or

NI-607.428_B9

VH-CDR1 comprises the amino acid sequence of SEQ ID NO: 73 or a variant thereof, wherein the variant comprises one or two amino acid substitutions,

VH-CDR2 comprises the amino acid sequence of SEQ ID NO: 74 or a variant thereof, wherein the variant comprises one or two amino acid substitutions,

VH-CDR3 comprises the amino acid sequence of SEQ ID NO: 75 or a variant thereof, wherein the variant comprises one or two amino acid substitutions,

VL-CDR1 comprises the amino acid sequence of SEQ ID NO: 78 or a variant thereof, wherein the variant comprises one or two amino acid substitutions,

VL-CDR2 comprises the amino acid sequence of SEQ ID NO: 79 or a variant thereof, wherein the variant comprises one or two amino acid substitutions, and

VL-CDR3 comprises the amino acid sequence of SEQ ID NO: 80 or a variant thereof, wherein the variant comprises one or two amino acid substitutions; or

NI-607.429_B9

VH-CDR1 comprises the amino acid sequence of SEQ ID NO: 83 or a variant thereof, wherein the variant comprises one or two amino acid substitutions,

VH-CDR2 comprises the amino acid sequence of SEQ ID NO: 84 or a variant thereof, wherein the variant comprises one or two amino acid substitutions,

VH-CDR3 comprises the amino acid sequence of SEQ ID NO: 85 or a variant thereof, wherein the variant comprises one or two amino acid substitutions,

VL-CDR1 comprises the amino acid sequence of SEQ ID NO: 88 or a variant thereof, wherein the variant comprises one or two amino acid substitutions,

VL-CDR2 comprises the amino acid sequence of SEQ ID NO: 89 or a variant thereof, wherein the variant comprises one or two amino acid substitutions, and

VL-CDR3 comprises the amino acid sequence of SEQ ID NO: 90 or a variant thereof, wherein the variant comprises one or two amino acid substitutions; or

NI-607.429_E4

VH-CDR1 comprises the amino acid sequence of SEQ ID NO: 93 or a variant thereof, wherein the variant comprises one or two amino acid substitutions,

VH-CDR2 comprises the amino acid sequence of SEQ ID NO: 94 or a variant thereof, wherein the variant comprises one or two amino acid substitutions,

VH-CDR3 comprises the amino acid sequence of SEQ ID NO: 95 or a variant thereof, wherein the variant comprises one or two amino acid substitutions,

VL-CDR1 comprises the amino acid sequence of SEQ ID NO: 98 or a variant thereof, wherein the variant comprises one or two amino acid substitutions,

VL-CDR2 comprises the amino acid sequence of SEQ ID NO: 99 or a variant thereof, wherein the variant comprises one or two amino acid substitutions, and

VL-CDR3 comprises the amino acid sequence of SEQ ID NO: 100 or a variant thereof, wherein the variant comprises one or two amino acid substitutions; or

NI-607.529_B9

VH-CDR1 comprises the amino acid sequence of SEQ ID NO: 103 or a variant thereof, wherein the variant comprises one or two amino acid substitutions,

VH-CDR2 comprises the amino acid sequence of SEQ ID NO: 104 or a variant thereof, wherein the variant comprises one or two amino acid substitutions,

VH-CDR3 comprises the amino acid sequence of SEQ ID NO: 105 or a variant thereof, wherein the variant comprises one or two amino acid substitutions,

VL-CDR1 comprises the amino acid sequence of SEQ ID NO: 108 or a variant thereof, wherein the variant comprises one or two amino acid substitutions,

VL-CDR2 comprises the amino acid sequence of SEQ ID NO: 109 or a variant thereof, wherein the variant comprises one or two amino acid substitutions, and

VL-CDR3 comprises the amino acid sequence of SEQ ID NO: 110 or a variant thereof, wherein the variant comprises one or two amino acid substitutions; or

NI-607.529_G4

VH-CDR1 comprises the amino acid sequence of SEQ ID NO: 113 or a variant thereof, wherein the variant comprises one or two amino acid substitutions,

VH-CDR2 comprises the amino acid sequence of SEQ ID NO: 114 or a variant thereof, wherein the variant comprises one or two amino acid substitutions,

VH-CDR3 comprises the amino acid sequence of SEQ ID NO: 115 or a variant thereof, wherein the variant comprises one or two amino acid substitutions,

VL-CDR1 comprises the amino acid sequence of SEQ ID NO: 118 or a variant thereof, wherein the variant comprises one or two amino acid substitutions,

VL-CDR2 comprises the amino acid sequence of SEQ ID NO: 119 or a variant thereof, wherein the variant comprises one or two amino acid substitutions, and

VL-CDR3 comprises the amino acid sequence of SEQ ID NO: 120 or a variant thereof, wherein the variant comprises one or two amino acid substitutions; or

NI-607.531_C8

VH-CDR1 comprises the amino acid sequence of SEQ ID NO: 123 or a variant thereof, wherein the variant comprises one or two amino acid substitutions,

VH-CDR2 comprises the amino acid sequence of SEQ ID NO: 124 or a variant thereof, wherein the variant comprises one or two amino acid substitutions,

VH-CDR3 comprises the amino acid sequence of SEQ ID NO: 125 or a variant thereof, wherein the variant comprises one or two amino acid substitutions,

VL-CDR1 comprises the amino acid sequence of SEQ ID NO: 128 or a variant thereof, wherein the variant comprises one or two amino acid substitutions,

VL-CDR2 comprises the amino acid sequence of SEQ ID NO: 129 or a variant thereof, wherein the variant comprises one or two amino acid substitutions, and

VL-CDR3 comprises the amino acid sequence of SEQ ID NO: 130 or a variant thereof, wherein the variant comprises one or two amino acid substitutions; or

NI-607.531_D8

VH-CDR1 comprises the amino acid sequence of SEQ ID NO: 133 or a variant thereof, wherein the variant comprises one or two amino acid substitutions,

VH-CDR2 comprises the amino acid sequence of SEQ ID NO: 134 or a variant thereof, wherein the variant comprises one or two amino acid substitutions,

VH-CDR3 comprises the amino acid sequence of SEQ ID NO: 135 or a variant thereof, wherein the variant comprises one or two amino acid substitutions,

VL-CDR1 comprises the amino acid sequence of SEQ ID NO: 138 or a variant thereof, wherein the variant comprises one or two amino acid substitutions,

VL-CDR2 comprises the amino acid sequence of SEQ ID NO: 139 or a variant thereof, wherein the variant comprises one or two amino acid substitutions, and

VL-CDR3 comprises the amino acid sequence of SEQ ID NO: 140 or a variant thereof, wherein the variant comprises one or two amino acid substitutions; or

NI-607.532_B6

VH-CDR1 comprises the amino acid sequence of SEQ ID NO: 143 or a variant thereof, wherein the variant comprises one or two amino acid substitutions,

VH-CDR2 comprises the amino acid sequence of SEQ ID NO: 144 or a variant thereof, wherein the variant comprises one or two amino acid substitutions,

VH-CDR3 comprises the amino acid sequence of SEQ ID NO: 145 or a variant thereof, wherein the variant comprises one or two amino acid substitutions,

VL-CDR1 comprises the amino acid sequence of SEQ ID NO: 148 or a variant thereof, wherein the variant comprises one or two amino acid substitutions,

VL-CDR2 comprises the amino acid sequence of SEQ ID NO: 149 or a variant thereof, wherein the variant comprises one or two amino acid substitutions, and

VL-CDR3 comprises the amino acid sequence of SEQ ID NO: 150 or a variant thereof, wherein the variant comprises one or two amino acid substitutions; or

NI-607.532_C11

VH-CDR1 comprises the amino acid sequence of SEQ ID NO: 153 or a variant thereof, wherein the variant comprises one or two amino acid substitutions,

VH-CDR2 comprises the amino acid sequence of SEQ ID NO: 154 or a variant thereof, wherein the variant comprises one or two amino acid substitutions,

VH-CDR3 comprises the amino acid sequence of SEQ ID NO: 155 or a variant thereof, wherein the variant comprises one or two amino acid substitutions,

VL-CDR1 comprises the amino acid sequence of SEQ ID NO: 158 or a variant thereof, wherein the variant comprises one or two amino acid substitutions,

VL-CDR2 comprises the amino acid sequence of SEQ ID NO: 159 or a variant thereof, wherein the variant comprises one or two amino acid substitutions, and

VL-CDR3 comprises the amino acid sequence of SEQ ID NO: 160 or a variant thereof, wherein the variant comprises one or two amino acid substitutions; or

NI-607.532_C8

VH-CDR1 comprises the amino acid sequence of SEQ ID NO: 163 or a variant thereof, wherein the variant comprises one or two amino acid substitutions,

VH-CDR2 comprises the amino acid sequence of SEQ ID NO: 164 or a variant thereof, wherein the variant comprises one or two amino acid substitutions,

VH-CDR3 comprises the amino acid sequence of SEQ ID NO: 165 or a variant thereof, wherein the variant comprises one or two amino acid substitutions,

VL-CDR1 comprises the amino acid sequence of SEQ ID NO: 168 or a variant thereof, wherein the variant comprises one or two amino acid substitutions,

VL-CDR2 comprises the amino acid sequence of SEQ ID NO: 169 or a variant thereof, wherein the variant comprises one or two amino acid substitutions, and

VL-CDR3 comprises the amino acid sequence of SEQ ID NO: 170 or a variant thereof, wherein the variant comprises one or two amino acid substitutions; or

NI-607.532_D3

VH-CDR1 comprises the amino acid sequence of SEQ ID NO: 173 or a variant thereof, wherein the variant comprises one or two amino acid substitutions,

VH-CDR2 comprises the amino acid sequence of SEQ ID NO: 174 or a variant thereof, wherein the variant comprises one or two amino acid substitutions,

VH-CDR3 comprises the amino acid sequence of SEQ ID NO: 175 or a variant thereof, wherein the variant comprises one or two amino acid substitutions,

VL-CDR1 comprises the amino acid sequence of SEQ ID NO: 178 or a variant thereof, wherein the variant comprises one or two amino acid substitutions,

VL-CDR2 comprises the amino acid sequence of SEQ ID NO: 179 or a variant thereof, wherein the variant comprises one or two amino acid substitutions, and

VL-CDR3 comprises the amino acid sequence of SEQ ID NO: 180 or a variant thereof, wherein the variant comprises one or two amino acid substitutions; or

NI-607.532_D4

VH-CDR1 comprises the amino acid sequence of SEQ ID NO: 183 or a variant thereof, wherein the variant comprises one or two amino acid substitutions,

VH-CDR2 comprises the amino acid sequence of SEQ ID NO: 184 or a variant thereof, wherein the variant comprises one or two amino acid substitutions,

VH-CDR3 comprises the amino acid sequence of SEQ ID NO: 185 or a variant thereof, wherein the variant comprises one or two amino acid substitutions,

VL-CDR1 comprises the amino acid sequence of SEQ ID NO: 188 or a variant thereof, wherein the variant comprises one or two amino acid substitutions,

VL-CDR2 comprises the amino acid sequence of SEQ ID NO: 189 or a variant thereof, wherein the variant comprises one or two amino acid substitutions, and

VL-CDR3 comprises the amino acid sequence of SEQ ID NO: 190 or a variant thereof, wherein the variant comprises one or two amino acid substitutions; or

NI-607.532_D8

VH-CDR1 comprises the amino acid sequence of SEQ ID NO: 193 or a variant thereof, wherein the variant comprises one or two amino acid substitutions,

VH-CDR2 comprises the amino acid sequence of SEQ ID NO: 194 or a variant thereof, wherein the variant comprises one or two amino acid substitutions,

VH-CDR3 comprises the amino acid sequence of SEQ ID NO: 195 or a variant thereof, wherein the variant comprises one or two amino acid substitutions,

VL-CDR1 comprises the amino acid sequence of SEQ ID NO: 198 or a variant thereof, wherein the variant comprises one or two amino acid substitutions,

VL-CDR2 comprises the amino acid sequence of SEQ ID NO: 199 or a variant thereof, wherein the variant comprises one or two amino acid substitutions, and

VL-CDR3 comprises the amino acid sequence of SEQ ID NO: 200 or a variant thereof, wherein the variant comprises one or two amino acid substitutions; or

NI-607.532_F9

VH-CDR1 comprises the amino acid sequence of SEQ ID NO: 203 or a variant thereof, wherein the variant comprises one or two amino acid substitutions,

VH-CDR2 comprises the amino acid sequence of SEQ ID NO: 204 or a variant thereof, wherein the variant comprises one or two amino acid substitutions,

VH-CDR3 comprises the amino acid sequence of SEQ ID NO: 205 or a variant thereof, wherein the variant comprises one or two amino acid substitutions,

VL-CDR1 comprises the amino acid sequence of SEQ ID NO: 208 or a variant thereof, wherein the variant comprises one or two amino acid substitutions,

VL-CDR2 comprises the amino acid sequence of SEQ ID NO: 209 or a variant thereof, wherein the variant comprises one or two amino acid substitutions, and

VL-CDR3 comprises the amino acid sequence of SEQ ID NO: 210 or a variant thereof, wherein the variant comprises one or two amino acid substitutions; or

NI-607.649_B11

VH-CDR1 comprises the amino acid sequence of SEQ ID NO: 213 or a variant thereof, wherein the variant comprises one or two amino acid substitutions,

VH-CDR2 comprises the amino acid sequence of SEQ ID NO: 214 or a variant thereof, wherein the variant comprises one or two amino acid substitutions,

VH-CDR3 comprises the amino acid sequence of SEQ ID NO: 215 or a variant thereof, wherein the variant comprises one or two amino acid substitutions,

VL-CDR1 comprises the amino acid sequence of SEQ ID NO: 218 or a variant thereof, wherein the variant comprises one or two amino acid substitutions,

VL-CDR2 comprises the amino acid sequence of SEQ ID NO: 219 or a variant thereof, wherein the variant comprises one or two amino acid substitutions, and

VL-CDR3 comprises the amino acid sequence of SEQ ID NO: 220 or a variant thereof, wherein the variant comprises one or two amino acid substitutions; or

NI-607.531_E7

VH-CDR1 comprises the amino acid sequence of SEQ ID NO: 223 or a variant thereof, wherein the variant comprises one or two amino acid substitutions,

VH-CDR2 comprises the amino acid sequence of SEQ ID NO: 224 or a variant thereof, wherein the variant comprises one or two amino acid substitutions,

VH-CDR3 comprises the amino acid sequence of SEQ ID NO: 225 or a variant thereof, wherein the variant comprises one or two amino acid substitutions,

VL-CDR1 comprises the amino acid sequence of SEQ ID NO: 228 or a variant thereof, wherein the variant comprises one or two amino acid substitutions,

VL-CDR2 comprises the amino acid sequence of SEQ ID NO: 229 or a variant thereof, wherein the variant comprises one or two amino acid substitutions, and

VL-CDR3 comprises the amino acid sequence of SEQ ID NO: 230 or a variant thereof, wherein the variant comprises one or two amino acid substitutions; or

NI-607.532_F3

VH-CDR1 comprises the amino acid sequence of SEQ ID NO: 233 or a variant thereof, wherein the variant comprises one or two amino acid substitutions,

VH-CDR2 comprises the amino acid sequence of SEQ ID NO: 234 or a variant thereof, wherein the variant comprises one or two amino acid substitutions,

VH-CDR3 comprises the amino acid sequence of SEQ ID NO: 235 or a variant thereof, wherein the variant comprises one or two amino acid substitutions,

VL-CDR1 comprises the amino acid sequence of SEQ ID NO: 238 or a variant thereof, wherein the variant comprises one or two amino acid substitutions,

VL-CDR2 comprises the amino acid sequence of SEQ ID NO: 239 or a variant thereof, wherein the variant comprises one or two amino acid substitutions, and

VL-CDR3 comprises the amino acid sequence of SEQ ID NO: 240 or a variant thereof, wherein the variant comprises one or two amino acid substitutions; or

NI-607.649_G7

VH-CDR1 comprises the amino acid sequence of SEQ ID NO: 243 or a variant thereof, wherein the variant comprises one or two amino acid substitutions,

VH-CDR2 comprises the amino acid sequence of SEQ ID NO: 244 or a variant thereof, wherein the variant comprises one or two amino acid substitutions,

VH-CDR3 comprises the amino acid sequence of SEQ ID NO: 245 or a variant thereof, wherein the variant comprises one or two amino acid substitutions,

VL-CDR1 comprises the amino acid sequence of SEQ ID NO: 248 or a variant thereof, wherein the variant comprises one or two amino acid substitutions,

VL-CDR2 comprises the amino acid sequence of SEQ ID NO: 249 or a variant thereof, wherein the variant comprises one or two amino acid substitutions, and

VL-CDR3 comprises the amino acid sequence of SEQ ID NO: 250 or a variant thereof, wherein the variant comprises one or two amino acid substitutions; or

NI-607.761_B7

VH-CDR1 comprises the amino acid sequence of SEQ ID NO: 253 or a variant thereof, wherein the variant comprises one or two amino acid substitutions,

VH-CDR2 comprises the amino acid sequence of SEQ ID NO: 254 or a variant thereof, wherein the variant comprises one or two amino acid substitutions,

VH-CDR3 comprises the amino acid sequence of SEQ ID NO: 255 or a variant thereof, wherein the variant comprises one or two amino acid substitutions,

VL-CDR1 comprises the amino acid sequence of SEQ ID NO: 258 or a variant thereof, wherein the variant comprises one or two amino acid substitutions,

VL-CDR2 comprises the amino acid sequence of SEQ ID NO: 259 or a variant thereof, wherein the variant comprises one or two amino acid substitutions, and

VL-CDR3 comprises the amino acid sequence of SEQ ID NO: 260 or a variant thereof, wherein the variant comprises one or two amino acid substitutions; or

NI-607.791_B10

VH-CDR1 comprises the amino acid sequence of SEQ ID NO: 263 or a variant thereof, wherein the variant comprises one or two amino acid substitutions,

VH-CDR2 comprises the amino acid sequence of SEQ ID NO: 264 or a variant thereof, wherein the variant comprises one or two amino acid substitutions,

VH-CDR3 comprises the amino acid sequence of SEQ ID NO: 265 or a variant thereof, wherein the variant comprises one or two amino acid substitutions,

VL-CDR1 comprises the amino acid sequence of SEQ ID NO: 268 or a variant thereof, wherein the variant comprises one or two amino acid substitutions,

VL-CDR2 comprises the amino acid sequence of SEQ ID NO: 269 or a variant thereof, wherein the variant comprises one or two amino acid substitutions, and

VL-CDR3 comprises the amino acid sequence of SEQ ID NO: 270 or a variant thereof, wherein the variant comprises one or two amino acid substitutions; or

NI-607.531_E3

VH-CDR1 comprises the amino acid sequence of SEQ ID NO: 273 or a variant thereof, wherein the variant comprises one or two amino acid substitutions,

VH-CDR2 comprises the amino acid sequence of SEQ ID NO: 274 or a variant thereof, wherein the variant comprises one or two amino acid substitutions,

VH-CDR3 comprises the amino acid sequence of SEQ ID NO: 275 or a variant thereof, wherein the variant comprises one or two amino acid substitutions,

VL-CDR1 comprises the amino acid sequence of SEQ ID NO: 278 or a variant thereof, wherein the variant comprises one or two amino acid substitutions,

VL-CDR2 comprises the amino acid sequence of SEQ ID NO: 279 or a variant thereof, wherein the variant comprises one or two amino acid substitutions, and

VL-CDR3 comprises the amino acid sequence of SEQ ID NO: 280 or a variant thereof, wherein the variant comprises one or two amino acid substitutions; or

NI-607.820_B6

VH-CDR1 comprises the amino acid sequence of SEQ ID NO: 283 or a variant thereof, wherein the variant comprises one or two amino acid substitutions,

VH-CDR2 comprises the amino acid sequence of SEQ ID NO: 284 or a variant thereof, wherein the variant comprises one or two amino acid substitutions,

VH-CDR3 comprises the amino acid sequence of SEQ ID NO: 285 or a variant thereof, wherein the variant comprises one or two amino acid substitutions,

VL-CDR1 comprises the amino acid sequence of SEQ ID NO: 288 or a variant thereof, wherein the variant comprises one or two amino acid substitutions,

VL-CDR2 comprises the amino acid sequence of SEQ ID NO: 289 or a variant thereof, wherein the variant comprises one or two amino acid substitutions, and

VL-CDR3 comprises the amino acid sequence of SEQ ID NO: 290 or a variant thereof, wherein the variant comprises one or two amino acid substitutions; or

NI-607.820_B7

VH-CDR1 comprises the amino acid sequence of SEQ ID NO: 293 or a variant thereof, wherein the variant comprises one or two amino acid substitutions,

VH-CDR2 comprises the amino acid sequence of SEQ ID NO: 294 or a variant thereof, wherein the variant comprises one or two amino acid substitutions,

VH-CDR3 comprises the amino acid sequence of SEQ ID NO: 295 or a variant thereof, wherein the variant comprises one or two amino acid substitutions,

VL-CDR1 comprises the amino acid sequence of SEQ ID NO: 298 or a variant thereof, wherein the variant comprises one or two amino acid substitutions,

VL-CDR2 comprises the amino acid sequence of SEQ ID NO: 299 or a variant thereof, wherein the variant comprises one or two amino acid substitutions, and

VL-CDR3 comprises the amino acid sequence of SEQ ID NO: 300 or a variant thereof, wherein the variant comprises one or two amino acid substitutions.

In addition, or alternatively the antibody or antigen-binding fragment thereof of the present invention and the antibodies or antigen-binding fragments thereof of the composition of the present invention, respectively can be characterized in that:

NI-607.274_B7

the VH chain comprises the amino acid sequence depicted in SEQ ID NO: 2 or a variant thereof, wherein the variant comprises one or more amino acid substitutions; and

the VL comprises the amino acid sequence depicted in SEQ ID NO: 7, or a variant thereof, wherein the variant comprises one or more amino acid substitutions; or

NI-607.274_E5

the VH comprises the amino acid sequence depicted in SEQ ID NO: 12 or a variant thereof, wherein the variant comprises one or more amino acid substitutions; and

the VL comprises the amino acid sequence depicted in SEQ ID NO: 17, or a variant thereof, wherein the variant comprises one or more amino acid substitutions; or

NI-607.275_C5

the VH comprises the amino acid sequence depicted in SEQ ID NO: 22 or a variant thereof, wherein the variant comprises one or more amino acid substitutions; and

the VL comprises the amino acid sequence depicted in SEQ ID NO: 27, or a variant thereof, wherein the variant comprises one or more amino acid substitutions;

NI-607.426_D4

the VH chain comprises the amino acid sequence depicted in SEQ ID NO: 32 or a variant thereof, wherein the variant comprises one or more amino acid substitutions; and

the VL comprises the amino acid sequence depicted in SEQ ID NO: 37, or a variant thereof, wherein the variant comprises one or more amino acid substitutions; or

NI-607.426_E2

the VH comprises the amino acid sequence depicted in SEQ ID NO: 42 or a variant thereof, wherein the variant comprises one or more amino acid substitutions; and

the VL comprises the amino acid sequence depicted in SEQ ID NO: 47, or a variant thereof, wherein the variant comprises one or more amino acid substitutions; or

NI-607.426_F11

the VH comprises the amino acid sequence depicted in SEQ ID NO: 52 or a variant thereof, wherein the variant comprises one or more amino acid substitutions; and

the VL comprises the amino acid sequence depicted in SEQ ID NO: 57, or a variant thereof, wherein the variant comprises one or more amino acid substitutions; or

NI-607.427_C5

the VH comprises the amino acid sequence depicted in SEQ ID NO: 62 or a variant thereof, wherein the variant comprises one or more amino acid substitutions; and

the VL comprises the amino acid sequence depicted in SEQ ID NO: 67, or a variant thereof, wherein the variant comprises one or more amino acid substitutions; or

NI-607.428_B9

the VH comprises the amino acid sequence depicted in SEQ ID NO: 72 or a variant thereof, wherein the variant comprises one or more amino acid substitutions; and

the VL comprises the amino acid sequence depicted in SEQ ID NO: 77, or a variant thereof, wherein the variant comprises one or more amino acid substitutions; or

NI-607.429_B9

the VH comprises the amino acid sequence depicted in SEQ ID NO: 82 or a variant thereof, wherein the variant comprises one or more amino acid substitutions; and

the VL comprises the amino acid sequence depicted in SEQ ID NO: 87, or a variant thereof, wherein the variant comprises one or more amino acid substitutions; or

NI-607.429_E4

the VH comprises the amino acid sequence depicted in SEQ ID NO: 92 or a variant thereof, wherein the variant comprises one or more amino acid substitutions; and

the VL comprises the amino acid sequence depicted in SEQ ID NO: 97, or a variant thereof, wherein the variant comprises one or more amino acid substitutions; or

NI-607.529_B9

the VH comprises the amino acid sequence depicted in SEQ ID NO: 102 or a variant thereof, wherein the variant comprises one or more amino acid substitutions; and

the VL comprises the amino acid sequence depicted in SEQ ID NO: 107, or a variant thereof, wherein the variant comprises one or more amino acid substitutions; or

NI-607.529_G4

the VH comprises the amino acid sequence depicted in SEQ ID NO: 112 or a variant thereof, wherein the variant comprises one or more amino acid substitutions; and

the VL comprises the amino acid sequence depicted in SEQ ID NO: 117, or a variant thereof, wherein the variant comprises one or more amino acid substitutions; or

NI-607.531_C8

the VH comprises the amino acid sequence depicted in SEQ ID NO: 122 or a variant thereof, wherein the variant comprises one or more amino acid substitutions; and

the VL comprises the amino acid sequence depicted in SEQ ID NO: 127, or a variant thereof, wherein the variant comprises one or more amino acid substitutions; or

NI-607.531_D8

the VH comprises the amino acid sequence depicted in SEQ ID NO: 132 or a variant thereof, wherein the variant comprises one or more amino acid substitutions; and

the VL comprises the amino acid sequence depicted in SEQ ID NO: 137, or a variant thereof, wherein the variant comprises one or more amino acid substitutions; or

NI-607.532_B6

the VH comprises the amino acid sequence depicted in SEQ ID NO: 142 or a variant thereof, wherein the variant comprises one or more amino acid substitutions; and

the VL comprises the amino acid sequence depicted in SEQ ID NO: 147, or a variant thereof, wherein the variant comprises one or more amino acid substitutions; or

NI-607.532_C11

the VH comprises the amino acid sequence depicted in SEQ ID NO: 152 or a variant thereof, wherein the variant comprises one or more amino acid substitutions; and

the VL comprises the amino acid sequence depicted in SEQ ID NO: 157, or a variant thereof, wherein the variant comprises one or more amino acid substitutions; or

NI-607.532_C8

the VH comprises the amino acid sequence depicted in SEQ ID NO: 162 or a variant thereof, wherein the variant comprises one or more amino acid substitutions; and

the VL comprises the amino acid sequence depicted in SEQ ID NO: 167, or a variant thereof, wherein the variant comprises one or more amino acid substitutions; or

NI-607.532_D3

the VH comprises the amino acid sequence depicted in SEQ ID NO: 172 or a variant thereof, wherein the variant comprises one or more amino acid substitutions; and

the VL comprises the amino acid sequence depicted in SEQ ID NO: 177, or a variant thereof, wherein the variant comprises one or more amino acid substitutions; or

NI-607.532_D4

the VH comprises the amino acid sequence depicted in SEQ ID NO: 182 or a variant thereof, wherein the variant comprises one or more amino acid substitutions; and

the VL comprises the amino acid sequence depicted in SEQ ID NO: 187, or a variant thereof, wherein the variant comprises one or more amino acid substitutions; or

NI-607.532_D8

the VH comprises the amino acid sequence depicted in SEQ ID NO: 192 or a variant thereof, wherein the variant comprises one or more amino acid substitutions; and

the VL comprises the amino acid sequence depicted in SEQ ID NO: 197, or a variant thereof, wherein the variant comprises one or more amino acid substitutions; or

NI-607.532_F9

the VH comprises the amino acid sequence depicted in SEQ ID NO: 202 or a variant thereof, wherein the variant comprises one or more amino acid substitutions; and

the VL comprises the amino acid sequence depicted in SEQ ID NO: 207, or a variant thereof, wherein the variant comprises one or more amino acid substitutions; or

NI-607.649_B11

the VH comprises the amino acid sequence depicted in SEQ ID NO: 212 or a variant thereof, wherein the variant comprises one or more amino acid substitutions; and

the VL comprises the amino acid sequence depicted in SEQ ID NO: 217, or a variant thereof, wherein the variant comprises one or more amino acid substitutions; or

NI-607.531_E7

the VH comprises the amino acid sequence depicted in SEQ ID NO: 222 or a variant thereof, wherein the variant comprises one or more amino acid substitutions; and

the VL comprises the amino acid sequence depicted in SEQ ID NO: 227, or a variant thereof, wherein the variant comprises one or more amino acid substitutions; or

NI-607.532_F3

the VH comprises the amino acid sequence depicted in SEQ ID NO: 232 or a variant thereof, wherein the variant comprises one or more amino acid substitutions; and

the VL comprises the amino acid sequence depicted in SEQ ID NO: 237, or a variant thereof, wherein the variant comprises one or more amino acid substitutions; or

NI-607.649_G7

the VH comprises the amino acid sequence depicted in SEQ ID NO: 242 or a variant thereof, wherein the variant comprises one or more amino acid substitutions; and

the VL comprises the amino acid sequence depicted in SEQ ID NO: 247, or a variant thereof, wherein the variant comprises one or more amino acid substitutions; or

NI-607.761_B7

the VH comprises the amino acid sequence depicted in SEQ ID NO: 252 or a variant thereof, wherein the variant comprises one or more amino acid substitutions; and

the VL comprises the amino acid sequence depicted in SEQ ID NO: 257, or a variant thereof, wherein the variant comprises one or more amino acid substitutions; or

NI-607.791_B10

the VH comprises the amino acid sequence depicted in SEQ ID NO: 262 or a variant thereof, wherein the variant comprises one or more amino acid substitutions; and

the VL comprises the amino acid sequence depicted in SEQ ID NO: 267, or a variant thereof, wherein the variant comprises one or more amino acid substitutions; or

NI-607.531_E3

the VH comprises the amino acid sequence depicted in SEQ ID NO: 272 or a variant thereof, wherein the variant comprises one or more amino acid substitutions; and

the VL comprises the amino acid sequence depicted in SEQ ID NO: 277, or a variant thereof, wherein the variant comprises one or more amino acid substitutions; or

NI-607.820_B6

the VH comprises the amino acid sequence depicted in SEQ ID NO: 282 or a variant thereof, wherein the variant comprises one or more amino acid substitutions; and

the VL comprises the amino acid sequence depicted in SEQ ID NO: 287, or a variant thereof, wherein the variant comprises one or more amino acid substitutions; or

NI-607.820_B7

the VH comprises the amino acid sequence depicted in SEQ ID NO: 292 or a variant thereof, wherein the variant comprises one or more amino acid substitutions; and

the VL comprises the amino acid sequence depicted in SEQ ID NO: 297, or a variant thereof, wherein the variant comprises one or more amino acid substitutions.

In a preferred embodiment, the VH and VL chain amino acid sequences are at least 90% identical to SEQ ID NO: 2 and 7, respectively, to SEQ ID NO: 12 and 17, respectively, to SEQ ID NO: 22 and 27, respectively, to SEQ ID NO: 32 and 37, respectively, to SEQ ID NO: 42 and 47, respectively, to SEQ ID NO: 52 and 57, respectively, to SEQ ID NO: 62 and 67, respectively, to SEQ ID NO: 72 and 77, respectively, to SEQ ID NO: 82 and 87, respectively, to SEQ ID NO: 92 and 97, respectively, to SEQ ID NO: 102 and 107, respectively, to SEQ ID NO: 112 and 117, respectively, to SEQ ID NO: 122 and 127, respectively, to SEQ ID NO: 132 and 137, respectively, to SEQ ID NO: 142 and 147, respectively, to SEQ ID NO: 152 and 157, respectively, to SEQ ID NO: 162 and 167, respectively, to SEQ ID NO: 172 and 177, respectively, to SEQ ID NO: 182 and 187, respectively, to SEQ ID NO: 192 and 197, respectively, to SEQ ID NO: 202 and 207, respectively, to SEQ ID NO: 212 and 217, respectively, to SEQ ID NO: 222 and 227, respectively, to SEQ ID NO: 232 and 237, respectively, to SEQ ID NO: 242 and 247, respectively, to SEQ ID NO: 252 and 257, respectively, to SEQ ID NO: 262 and 267, respectively, to SEQ ID NO: 272 and 277, respectively, to SEQ ID NO: 282 and 287, respectively, or to SEQ ID NO: 292 and 297, respectively.

In these embodiments, preferably one or more of the CDRs according to the Chothia definition are maintained substantially unchanged. Thus, in order to provide anti-SARS-CoV-2 antibodies equivalent to subject antibodies NI-607.274_B7, NI-607.274_E5, NI-607.275_C5, NI-607.426_D4, NI-607.426_E2, NI-607.426_F1, NI-607.427_C5, NI-607.428_B9, NI-607.429_B9, NI-607.429_E4, NI-607.529_B9, NI-607.529_G4, NI-607.531_C8, NI-607.531_D8, NI-607.532_B6, NI-607.532_C11, NI-607.532_C8, NI-607.532_D3, NI-607.532_D4, NI-607.532_D8, NI-607.532_F9, NI-607.649_B11, NI-607.531_E7, NI-607.532_F3, NI-607.649_G7, NI-607.761_B7, NI-607.791_B10, NI-607.531_E3, NI-607.820_B6, and NI-607.820_B7, preferably at least one or two of said one or more, preferably not more than two amino acid substitutions if made in the CDRs as defined according to Kabat are made outside the CDRs as defined by Chothia and/or IMGT and most preferably outside the overlap of the CDRs as defined according to Kabat and Chothia.

For example, regarding amino acid substitutions within the CDRs, variable heavy and light chain and framework amino acid sequences, respectively, preferably conservative amino acid substitutions are performed for example in accordance with the most frequently exchanged amino acids as analyzed and described by Mirsky et al., Mol. Biol. Evol. 32 (2014), 806-819; see FIG. 6 at page 813 of Mirsky et al. In particular, within VH-CDR1, S may be substituted with T; within VH-CDR3, V may be substituted with E, T may be substituted with S and/or M may be substituted with V; within VL-CDR1, R may be substituted with K, R may be substituted with E, and/or T may be substituted; within VL-CDR2, S may be substituted with A and/or A may be substituted with G; and in VL-CDR3, P may be substituted with S. As mentioned, preferably amino acid substitutions are selected which belong to the same category in either or preferably both models LG and AB shown in FIG. 6 of Mirsky et al. (2014), supra, with the LG model being preferred for the tendency to keep amino acid properties, and wherein the amino acid substitutions are selected preferably such that the physiochemical properties of the original amino acid is substantially maintained, i.e. hydrophobic, polar or charged property or for example that in case two or more amino acid substitutions are performed, they compensate each other so as to provide the physicochemical property of the surface all together. In a preferred embodiment, the antibody of the invention comprises a variant of the amino acid sequence of the VH and/or VL region which is at least 90%, 95%, 96%, 97%, 98%, 99% or 100% identical to the VH and VL regions depicted in Table II.

Of course, besides theoretical considerations also experimental approaches exist for identifying CDR variants within a reasonable time and undue burden. For example, Tiller et al., in Front Immunol. 8 (2017), 986 describe facile affinity maturation of antibody variable domains using natural diversity mutagenesis. Indeed, already a few years earlier Rajpal et al., in PNAS 102 (2005), 8466-8471 reported a general method for greatly improving the affinity of antibodies by using combinatorial libraries and illustrated their method with anti-TNF-α antibody D2E7 (HUMIRA©) identifying 38 substitutions in 21 CDR positions that resulted in higher affinity binding to TNF-α. More recently, Cannon et al., in PLOS Computational Biology, https://doi.org/10.1371/joumal.pcbi.1006980 May 1, 2019 described experimentally guided computational antibody affinity maturation with de novo docking, modelling and rational design in silico affinity maturation, together with alanine scanning, that allowed fine-tuning the protein-protein docking model to subsequently enable the identification of two single-point mutations that increase the affinity of a hybridoma-derived antibody, AB1 for its antigen murine CCL20.

Accordingly, though each antibody is unique and may have distinct features, nevertheless once a lead candidate has been provided the person skilled in the art in consideration of the teaching of the present invention as disclosed in the present application, as well as in view of the computational design and experimental approaches developed so far is able to arrive at equivalent anti-SARS-CoV-2 antibodies which keep the desired features of the antibody such as those described for the anti-SARS-CoV-2 antibodies illustrated in the Examples and specifically defined in the claims. In this context, it is well understood that the variant antibody substantially maintains the binding specificity of the parent antibody, for example binding the RBD of SARS-CoV-2 S with an EC50 of <100 pM. Preferably however, the antibody of the present invention comprises in one or both of its immunoglobulin chains one, two or all three CDRs of the variable regions as set forth in Table II. In addition, or alternatively, the above-mentioned framework regions are 80% identical to the framework regions, preferably 85%, 90%, 95%, 96, 97%, 98%, 99% or 100% identical to the framework regions.

For example, antibodies NI-607.426_D4 and NI-607.428_B9 have the same VH chain amino acid sequence except two amino acids in the framework regions 1 and 3 which differ from each other. In particular, the VH sequence of antibody NI-607.426_D4 has a glutamate (E) at positon 1 and a methionine at position 93 of SEQ ID NO: 32, whereas antibody NI-607.428_B9 has a glutamine (Q) at position 1 and a valine (V) at positon 93 of SEQ ID NO: 72. However, as can be derived from Table I, their binding affinities and neutralization capabilities are substantially the same. Thus, a few amino acid substitutions in the VH and/or VL chain, in particular in the framework regions do not substantially change the binding characteristics of the antibodies of the present invention.

Furthermore, antibodies NI-607.532_D3 and NI-607.532_D4 have the same VL chain amino acid sequence, but a different VH chain amino acid sequence as depicted in SEQ ID NOs: 172, 177, 182, and 187. However, both antibodies show substantially the same binding characteristic, i.e., their EC₅₀ values for binding to the RBD of SARS-CoV-2, SASR-CoV and MERS-CoV are substantially the same as shown in Table I. Thus, differences in the VH chain amino acid sequence do not seem to influence the binding of an antibody, if the VL chain amino acid sequence remains same.

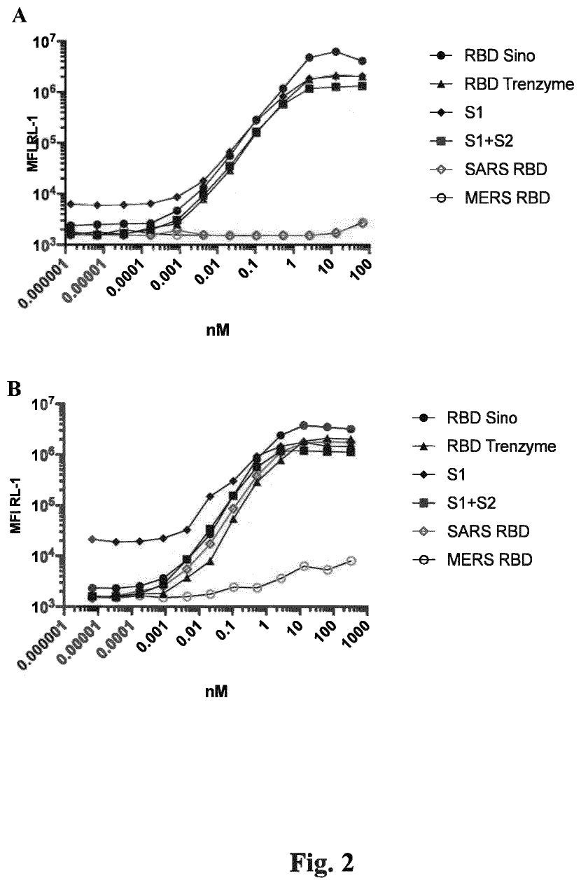

As mentioned above, SARS-CoV-2 belongs to the broad family of viruses known as coronaviruses. It is the seventh known coronavirus to infect people, after 229E, NL63, OC43, HKU1, MERS-CoV, and the original SARS-CoV. In the past, human monoclonal antibodies to the S glycoprotein have been developed and shown to mediate immunity to betacoronaviruses including MERS-CoV. However, in the present case, it is desired to identify antibodies which bind to the RBD of SARS-CoV-2, but not to the RBD of MERS-CoV. Thus, a corresponding iQue analysis has been performed identifying the binding affinity to the RBD of MERS-CoV which revealed only a moderate binding of antibodies to the RBD of MERS-CoV; see Example 4 and FIG. 2. In particular, the EC₅₀ value for binding to MERS-CoV is between 200 and 1000 nM,

Accordingly, in one embodiment the antibody of the present invention and the antibodies of the composition of the present invention, respectively, or at least one antibody of the composition do not substantially bind to the corresponding RBD of MERS-CoV.

In a preferred embodiment the antibody of the present invention and the antibodies of the composition of the present invention, respectively, or at least one antibody of the composition binds to the corresponding RBD of MERS-CoV with an EC₅₀ between 200 and 1000 nM, preferably between 300 and 1000 nM, more preferably between 400 and 1000 nM, more preferably between 500 and 1000 nM, more preferably between 600 and 1000 nM, more preferably between 700 and 1000 nM, more preferably between 800 and 1000 nM, more preferably between 900 and 1000 nM and more preferably with an EC₅₀ of about 1000 nM.

The composition may comprise two, three, four, five, six, seven, eight, nine, ten, eleven, twelve, 13, 14, 15, 16, 17, 18, 19, 20, 21, 22, 23, 24, 25, 26, 27, 28, 29, or 30 antibodies that do not substantially bind to the corresponding RBD of MERS-CoV.

As can be derived from Table I, antibodies have been identified that show a similar binding affinity to the RBD of SARS-CoV and to the RBD of SARS-CoV-2. This is exemplarily shown in FIG. 2B. For example, antibody NI-607.427_C5 has been identified to bind to the RBD of SARS-CoV-2 with an EC₅₀ of 3.2 nM and to the RBD of SARS-CoV with an EC₅₀ of 3 nM as determined via iQue. Antibody NI-607.529_G4 binds to the RBD of SARS-CoV-2 with an EC₅₀ of 1.3 nM and to the RBD of SARS-CoV with an EC₅₀ of 10 nM. Antibody NI-607.531_E3 binds to the RBD of SARS-CoV-2 with an EC₅₀ of 1.4 nM and to the RBD of SARS-CoV with an EC₅₀ of 2.6 nM. Antibody NI-607.532_B6 binds to the RBD of SARS-CoV-2 with an EC₅₀ of 1.1 nM and to the RBD of SARS-CoV with an EC₅₀ of 4.2 nM. Antibody NI-607.532_D8 binds to the RBD of SARS-CoV-2 with an EC₅₀ of 14.4 nM and to the RBD of SARS-CoV with an EC₅₀ of 32 nM. Antibody NI-607.532_F3 binds to the RBD of SARS-CoV-2 with an EC₅₀ of 1.2 nM and to the RBD of SARS-CoV with an EC₅₀ of 1.3 nM. Antibody NI-607.649_B11 binds to the RBD of SARS-CoV-2 with an EC₅₀ of 1.4 nM and to the RBD of SARS-CoV with an EC₅₀ of 1.7 nM. Antibody NI-607.649_G7 binds to the RBD of SARS-CoV-2 with an EC₅₀ of 5.7 nM and to the RBD of SARS-CoV with an EC₅₀ of 22.2 nM.

Such antibodies may be used in treatment approaches not only for COVID-19, but also for SARS and other potential future diseases related to (novel) coronaviruses as outlined above.

Furthermore, antibodies have been identified that bind with high affinity to both the RBD of SARS-CoV-2 and SARS-CoV, but that nevertheless bind to the RBD of SARS-CoV with an EC₅₀ which is at least one order of magnitude higher than its EC₅₀ for binding to the RBD of SARS-CoV-2. In other words, antibodies have been identified that bind with high affinity to both the RBD of SARS-CoV-2 and SARS-CoV, but that nevertheless bind to the RBD of SARS-CoV with an EC₅₀ which is two- to three-fold higher than its EC₅₀ for binding to the RBD of SARS-CoV-2.

For example, antibody NI-607.529_B9 binds with an EC₅₀ of 1 nM to the RBD of SARS-CoV-2 and with an EC₅₀ of 20 nM to the RBD of SARS-CoV as determined via iQue and antibody NI-607.820_B6 binds with an EC₅₀ of 2.4 nM to the RBD of SARS-CoV-2 and with an EC₅₀ of 60.2 nM to the RBD of SARS-CoV as determined via iQue. Accordingly, due to its high binding affinity to the RBD of both viruses these antibodies are also particularly useful for the above-mentioned treatment approaches.

Thus, in one embodiment the antibody of the present invention and the antibodies of the composition of the present invention, respectively. or at least one antibody of the composition binds to the RBD of SARS-CoV with an EC₅₀ which is in the same order of magnitude as the EC₅₀ for its binding to the RBD of SARS-CoV-2 as determined by iQue analysis. In another embodiment, the antibody of the present invention and the antibodies of the composition of the present invention, respectively or at least one antibody of the composition bind with high affinity to the RBD of SARS-CoV-2 and SARS-CoV in the low nanomolar range, preferably between 0.5 nM and 80 nM, preferably between 0.5 nM and 40 nM or between 1 nM and 70 nM, more preferably between 1 nM and 20 nM or between 2 nM and 61 nM as determined by iQue, optionally wherein the antibody binds to the RBD of SARS-CoV with an EC₅₀ which is two- to three-fold higher than its EC₅₀ for binding to the RBD of SARS-CoV-2.

The composition may comprise two, three, four, five, six, seven, eight, nine, ten, eleven, twelve, 13, 14, 15, 16, 17, 18, 19, 20, 21, 22, 23, 24, 25, 26, 27, 28, 29, or 30 antibodies that bind to the RBD of SARS-CoV with an EC₅₀ which is in the same order of magnitude as the EC₅₀ for its binding to the RBD of SARS-CoV-2 and/or that bind with high affinity to the RBD of SARS-CoV-2 and SARS-CoV in the low nanomolar range.

Both SARS-CoV-2 and SARS-CoV belong to the sarbecovirus subgenus and their S glycoproteins share about 80% amino acid sequence identity. Thus, it is challenging to identify antibodies that bind to the RBD of SARS-CoV-2 S, but not to the RBD of SARS-CoV S. However, as can be further derived from Table I and shown in FIG. 2A, antibodies have been identified that bind with high affinity to the RBD of SARS-CoV-2 S but that do not, or at least to a lower amount bind the RBD of SARS-CoV S. For example, antibody NI-607.274_B7 has been identified to bind to the RBD of SARS-CoV-2 with an EC₅₀ of 0.67 nM and to the RBD of SARS-CoV with an EC₅₀ of 900 nM as determined via iQue. Antibody NI-607.274_E5 binds to the RBD of SARS-CoV-2 with an EC₅₀ of 0.39 nM and to the RBD of SARS-CoV with an EC₅₀ of 900 nM. Antibody NI-607.275_C5 binds to the RBD of SARS-CoV-2 with an EC₅₀ of 1.5 nM and to the RBD of SARS-CoV with an EC₅₀ of 800 nM. Antibody NI-607.426_E2 binds to the RBD of SARS-CoV-2 with an EC₅₀ of 2.06 nM and to the RBD of SARS-CoV with an EC₅₀ of 600 nM. Antibody NI-607.426_D4 binds to the RBD of SARS-CoV-2 with an EC₅₀ of 0.76 nM and to the RBD of SARS-CoV with an EC₅₀ of 1000 nM. Antibody NI-607.426_F11 binds to the RBD of SARS-CoV-2 with an EC₅₀ of 0.89 nM and to the RBD of SARS-CoV with an EC₅₀ of 1000 nM. Antibody NI-607.428_B9 binds to the RBD of SARS-CoV-2 with an EC₅₀ of 1.3 nM and to the RBD of SARS-CoV with an EC₅₀ of 800 nM. Antibody NI-607.429_B9 binds to the RBD of SARS-CoV-2 with an EC₅₀ of 1.87 nM and to the RBD of SARS-CoV with an EC₅₀ of 600 nM. Antibody NI-607.531_C8 binds to the RBD of SARS-CoV-2 with an EC₅₀ of 1.2 nM and to the RBD of SARS-CoV with an EC₅₀ of 1000 nM.

Antibody NI-607.531_D8 binds to the RBD of SARS-CoV-2 with an EC₅₀ of 1.4 nM and to the RBD of SARS-CoV with an EC₅₀ of 1000 nM. Antibody NI-607.531_E7 binds to the RBD of SARS-CoV-2 with an EC₅₀ of 1.3 nM and to the RBD of SARS-CoV with an EC₅₀ of 600 nM. Antibody NI-607.532_C11 binds to the RBD of SARS-CoV-2 with an EC₅₀ of 2.4 nM and to the RBD of SARS-CoV with an EC₅₀ of 1000 nM. Antibody NI-607.532_D3 binds to the RBD of SARS-CoV-2 with an EC₅₀ of 1 nM and to the RBD of SARS-CoV with an EC₅₀ of 1000 nM. Antibody NI-607.532_D4 binds to the RBD of SARS-CoV-2 with an EC₅₀ of 1 nM and to the RBD of SARS-CoV with an EC₅₀ of 1000 nM. Antibody NI-607.532_F9 binds to the RBD of SARS-CoV-2 with an EC₅₀ of 3.3 nM and to the RBD of SARS-CoV with an EC₅₀ of 1000 nM. Antibody NI-607.529_B9 binds to the RBD of SARS-CoV-2 with an EC₅₀ of 1 nM and to the RBD of SARS-CoV with an EC₅₀ of 20 nM. Antibody NI-607.791_B10 binds to the RBD of SARS-CoV-2 with an EC₅₀ of 13.7 nM and to the RBD of SARS-CoV with an EC₅₀ of 400 nM. Antibody NI-607.820_B6 binds to the RBD of SARS-CoV-2 with an EC₅₀ of 2.4 nM and to the RBD of SARS-CoV with an EC₅₀ of 60.2 nM. Antibody NI-607.820_B7 binds to the RBD of SARS-CoV-2 with an EC₅₀ of 9 nM and to the RBD of SARS-CoV with an EC₅₀ of 800 nM.

Such antibodies might not only be useful in therapy of COVID-19 but have high potential for diagnostics since the antibodies are more specific for SARS-CoV-2 than for the closely related SARS-CoV.

Thus, in one embodiment, the antibody of the present invention and the antibodies of the composition of the present invention, respectively, or at least one antibody of the composition bind to the RBD of SARS-CoV with an EC₅₀ which is at least one or two order of magnitudes higher, i.e. 10 times, preferably 15 times, more preferably 20 times higher or 100 times, preferably 200 times, more preferably 300 times, more preferably 400 times, more preferably 500 times, more preferably 600 times, more preferably 700 times and even more preferably 800 times higher than the EC₅₀ for its binding to the RBD of SARS-CoV-2 or wherein the antibody does not substantially bind to the RBD of SARS-CoV.

The composition may comprise two, three, four, five, six, seven, eight, nine, ten, eleven, twelve, 13, 14, 15, 16, 17, 18, 19, 20, 21, 22, 23, 24, 25, 26, 27, 28, 29, or 30 antibodies that bind to the RBD of SARS-CoV with an EC₅₀ which is at least one or two order of magnitudes higher than the EC₅₀ for its binding to the RBD of SARS-CoV-2 or wherein the antibody does not substantially bind to the RBD of SARS-CoV.

SARS-CoV-2 has been shown to enter the target cells through an endosomal pathway, wherein the S protein binds to cellular receptor angiotensin-converting enzyme 2 (ACE2). Following entry of the virus into the host cell, the viral RNA is unveiled in the cytoplasm. Afterwards, the RNA is translated into proteins by the cell's machinery and proteins and RNA are assembled into new virion in the Golgi. The virions are then released from the infected cell through exocytosis leading to propagation of the virus. Previous studies revealed that a large number of antibodies showed neutralization activity by targeting the RBD of either SARS-CoV or Middle East respiratory syndrome coronavirus (MERS-CoV) presumably by disrupting the virus-receptor engagement. Therefore, in accordance with the present invention preferably antibodies have been identified binding epitopes which overlap with ACE2-binding sites in SARS-CoV-2 RBD, thereby interfering with the virus/receptor interactions by both steric hindrance and direct interface-residue competition; see FIG. 4. Besides preventing virus uptake into the cell through ACE2 mediated endocytosis, antibody bound to the virus particles may serve as flag for immune effector cells and include phagocytosis or may otherwise interfere with the life cycle and spreading of the virus. Therefore, antibodies that are capable of inhibiting binding the SARS-CoV-2 RBD to ACE2 are called neutralizing antibodies. However, as mentioned hereinbefore, also antibodies of the present invention, which specifically bind SARS-CoV-2 with a low EC₅₀ but do not block RBD-ACE-2 binding may contribute to tackle the viral infection, for example by suppressing the systemic spread of the virus.

In experiments performed within the scope of the present invention, a competition ELISA was used to identify RBD-binding antibodies with virus neutralization potential. In particular, it was determined whether the tested antibodies disrupt the interaction between the viral RBD protein and the human ACE2 receptor, which mediates viral entry into host cells; see Example.

In this context, some of the tested antibodies indeed showed the desired activity as indicated in Table I, wherein the inhibition potency of the antibodies is expressed as IC₅₀. The half maximal inhibitory concentration (“IC₅₀”) is a measure of the potency of a substance in inhibiting a specific biological or biochemical function. IC₅₀ is a quantitative measure that indicates how much of a particular inhibitory substance (e.g., antibody) is needed to inhibit, in vitro, a given biological process or biological component by 50%.

In one embodiment, the antibody of the present invention and the antibodies of the composition of the present invention, respectively or at least one antibody of the composition is capable of inhibiting binding of the SARS-CoV-2 RBD to ACE2 at an IC₅₀ of <8000 pM, preferably at an IC₅₀ of <7000 pM, more preferably at an IC₅₀ of <6000 pM, more preferably at an IC₅₀ of <5000 pM, more preferably at an IC₅₀ of <4000 pM, more preferably at an IC₅₀ of <3000 pM, more preferably at an IC₅₀ of <2000 pM, more preferably at an IC₅₀ of <1500 pM, more preferably at an IC₅₀ of <1000 pM, more preferably at an IC₅₀ of <950 pM, more preferably at an IC₅₀ of <900 pM, more preferably at an IC₅₀ of <850 pM, more preferably at an IC₅₀ of <800 pM, more preferably at an IC₅₀ of <750 pM, more preferably at an IC₅₀ of <700 pM, more preferably at an IC₅₀ of <650 pM, more preferably at an IC₅₀ of <600 pM, more preferably at an IC₅₀ of <550 pM, more preferably at an IC₅₀ of <500 pM, more preferably at an IC₅₀ of <450 pM, more preferably at an IC50 of <400 pM, more preferably at an IC50 of <350 pM, more preferably at an IC₅₀ of <300 pM, more preferably at an IC₅₀ of <250 pM, more preferably at an IC₅₀ of <200 pM, more preferably at an IC₅₀ of <175 pM, even more preferably at an IC₅₀ of <120 pM. The composition may comprise two, three, four, five, six, seven, eight, nine, ten, eleven, twelve, 13, 14, 15, 16, 17, 18, 19, 20, 21, 22, 23, 24, 25, 26, 27, 28, 29, or 30 antibodies that are capable of inhibiting binding of the RBD to ACE2 at an IC₅₀ as mentioned above.

In a preferred embodiment, the antibody of the present invention and the antibodies of the composition of the present invention, respectively or at least one antibody of the composition is capable of inhibiting binding of the SARS-CoV-2 RBD to ACE2 at an IC₅₀ of <3000 pM, preferably <200 pM.

The values for each antibody are listed in Table I and thus, in one embodiment, the IC₅₀ of antibody NI-607.274_B7 as determined by competitive ELISA for the RBD of SARS-CoV-2 S is about 7800 pM. In another embodiment, the IC₅₀ of antibody NI-607.274_E5 is about 12600 pM. In another embodiment, the IC₅₀ of antibody NI-607.275_C5 is about 36833 pM. In another embodiment, the IC₅₀ of antibody NI-607.426_D4 is about 24353 pM. In another embodiment, the IC₅₀ of antibody NI-607.426_E2 is about 10693 pM. In another embodiment, the IC₅₀ of antibody NI-607.426_F11 is about 1142 pM. In another embodiment, the IC₅₀ of antibody NI-607.427_C5 is about 2877 pM. In another embodiment, the IC₅₀ of antibody NI-607.428_B9 is about 15787 pM. In another embodiment, the IC₅₀ of antibody NI-607.429_B9 is about 74 pM. In another embodiment, the IC₅₀ of antibody NI-607.429_E4 is about 275 pM. In another embodiment, the IC₅₀ of antibody NI-607.529_B9 is about 116 pM. In another embodiment, the IC₅₀ of antibody NI-607.531_C8 is about 153 pM. In another embodiment, the IC₅₀ of antibody NI-607.532_C11 is about 826 pM. In another embodiment, the IC₅₀ of antibody NI-607.532_D3 is about 917 pM. In another embodiment, the IC₅₀ of antibody NI-607.532_F9 is about 775 pM. In another embodiment, the IC₅₀ of antibody NI-607.649_B11 is about 80 pM. In another embodiment, the IC₅₀ of antibody NI-607.531_E7 is about 3666 pM. In another embodiment, the IC₅₀ of antibody NI-607.532_F3 is about 353 pM. In another embodiment, the IC₅₀ of antibody NI-607.761_B7 is about 56000 pM.

To further verify the neutralization capability of the antibodies, a pseudovirus entry assay has been performed as described in Example 7 and exemplary antibodies have been tested, in particular those that have been shown to inhibit the interaction between ACE2 and the RBD of the S protein of SARS-CoV-2.

The results of the assays are shown in FIG. 5 including the IC₅₀ values of the tested antibodies, and it has been found that a significant number of the antibodies reached the maximum neutralization of 50% or greater. In particular, the IC₅₀ of antibody NI-607.429_B9 as determined by the pseudovirus entry assay is 7.1 μg/mL (FIG. 5A), the IC₅₀ of antibody NI-607.429_E4 is 35 ng/mL (FIG. 5B), the IC₅₀ of antibody NI-607.529_B9 is 1.9 μg/mL (FIG. 5B), the IC₅₀ of antibody NI-607.531_C8 is 86.4 ng/mL (0.572 nM) (FIG. 5B), the IC₅₀ of antibody NI-607.532_B6 is 100 μg/mL (FIG. 5C), the IC₅₀ of antibody NI-607.532_C11 is 87.2 ng/mL (FIG. 5C), the IC₅₀ of antibody NI-607.532_D3 is 2.1 μg/mL (FIG. 5C), the IC₅₀ of antibody NI-607.532_F9 is 363.6 ng/mL (FIG. 5D), the IC₅₀ of antibody NI-607.649_B11 is 231.8 ng/mL (1.531 nM) (FIG. 5D), the IC₅₀ of antibody NI-607.531_E7 is 3.9 μg/mL (FIG. 5D), and the IC₅₀ of antibody NI-607.532_F3 is 11.7 μg/mL (FIG. 5D). The other antibodies, i.e., NI-607.427_C5 (FIG. 5A), NI-607.426_F11 (FIG. 5A), NI-607.274_B7 (FIG. 5A), NI-607.529_G4 (FIG. 5B), NI-607.531_D8 (FIG. 5C), and NI-607.649_G7 (FIG. 5E) showed an IC₅₀>100 μg/mL.

In FIG. 5E a positive control, i.e., antibody D002 of SinoBiologics as mentioned above as well as two negative controls, i.e., one isotype control and PBS are shown, wherein the positive control shows an IC₅₀ of 591.5 ng/mL. Thus, in accordance with the present invention antibodies have been identified, i.e., antibodies NI-607.429_E4, NI-607.531_C8, NI-607.532_C11, NI-607.532_F9, and NI-607.649_B11 that show a greater neutralizing effect than the commercially available positive control which has a high affinity towards the RBD of SARS-CoV-2 and serves as a benchmark for high affinity anti-SARS-CoV-2 antibodies.

Thus, in one embodiment, the antibody of the present invention and the antibodies of the composition of the present invention, respectively, or at least one antibody of the composition is capable of neutralizing SARS-CoV-2 and the pseudovirus illustrated in Example 7, respectively, at an IC₅₀ of <100 μg/mL, preferably at an IC₅₀ of <15 μg/mL, preferably at an IC₅₀ of <12 μg/mL, preferably at an IC₅₀ of <4 μg/mL, preferably at an IC₅₀ of <2 μg/mL, preferably at an IC₅₀ of <1 μg/mL, preferably at an IC₅₀ of <600 ng/mL, preferably at an IC₅₀ of <400 ng/mL, preferably at an IC₅₀ of <300 ng/mL, preferably at an IC₅₀ of <100 ng/mL, preferably at an IC₅₀ of <50 ng/mL. In a further preferred embodiment, the IC₅₀ is between 100 ng/mL and 70 ng/mL, preferably between 80 ng/mL and 90 ng/mL and most preferably of about 86 ng/mL. In another preferred embodiment the IC₅₀ is between 250 ng/mL and 200 ng/mL, preferably between 240 ng/mL and 210 ng/mL and most preferably of about 230 ng/mL.

The antibodies were also tested for their capability to inhibit the infection of fully replication competent SARS-Cov-2 virus. In this context, a cytopathic effect (CPE) inhibition assay was performed as described in Example 8. The results of the assay are shown in the microscopic pictures of FIG. 6. As can be derived from FIG. 6, antibodies NI-607.649_B11, NI-607.532_F9, NI-607.532_D3, NI-607.532_C11, NI-607.521_C8, and NI-607.429_E4 show a similar or even better neutralization effect than the positive control, while antibody neutralization by antibody NI-607.529_G4 is barely detectable. These results are consistent with the virus neutralization assay as described in Example 7.

As illustrated in Example 8 and FIG. 6, antibodies NI-607.427_C5, NI-607.429_B9, and NI-607.529_B9 neutralize SARS-CoV-2 at a dilution of 1:10, i.e. at a concentration of 15 μg/mL (100 nM), antibodies NI-607.531_C8 and NI-607.532_F9 neutralize SARS-CoV-2 already at a dilution of 1:40, i.e. at a concentration of 3.75 μg/mL (25 nM), antibodies NI-607.429_E4, NI-607.532_C11, and NI-607.649_B11 neutralize SARS-CoV-2 already at a dilution of 1:80, i.e. at a concentration of 1.875 μg/mL (12.5 nM), and antibody NI-607.532_D3 neutralizes SARS-CoV-2 already at a dilution of 1:160, i.e. at a concentration of 0.9375 μg/mL (6.25 nM).

In one embodiment, the antibody of the present invention and the antibodies of the composition of the present invention, respectively, or at least one antibody of the composition is capable of neutralizing SARS-CoV-2 at a concentration of ≤30 μg/mL (200 nM), preferably of ≤15 μg/mL/100 nM, preferably of ≤3.75 μg/mL/25 nM or of ≤1.875 μg/mL/12.5 nM or of ≤0.9375 μg/mL/6.25 nM, or at a concentration between 3.75 μg/mL (25 nM) and 1.875 μg/mL (12.5 nM).

The antibodies of the present invention were tested for their capability to inhibit the infection of cells with fully replication competent SARS-CoV-2-GFP viruses via monitoring GFP expression as described in Example 9. The results of exemplary antibodies are illustrated in FIG. 7. In contrast to the isotype control (see FIG. 7G), antibody NI-607.531_C8 showed clear reduction in GFP which equals viral gene expression and growth with dilutions 1/3125, 1/625, 1/125 and 1/25, i.e. at concentrations of 0.32 μg/mL (2.1 nM), 1.6 μg/mL (10.7 nM), 8 μg/mL (53.3 nM), and 40 μg/mL (266.6 nM). Antibody NI-607.649_B11 showed clear reduction in GFP with dilutions 1/125 and 1/25, i.e., at concentrations of 8 μg/mL (53.3 nM), and 40 μg/mL (266.6 nM). Antibody NI-607.429_E4 showed clear reduction in GFP with dilutions 1/625, 1/125 and 1/25, i.e., at concentrations of 1.6 μg/mL (10.7 nM), 8 μg/mL (53.3 nM), and 40 μg/mL (266.6 nM). Antibodies NI-607.429_B9 and NI-607.529_B9 showed clear reduction in GFP with dilution 1/25, i.e., at concentrations of 40 μg/mL (266.6 nM). Antibody NI-607.532_C11 showed clear reduction in GFP with dilution 1/125 and 1/25, i.e., at concentrations of 8 μg/mL (53.3 nM), and 40 μg/mL (266.6 nM). Antibodies NI-607.532_D3 and NI-607.532_F9 showed clear reduction in GFP with dilutions 1/625, 1/125 and 1/25, i.e., at concentrations of 1.6 μg/mL (10.7 nM), 8 μg/mL (53.3 nM), and 40 μg/mL (266.6 nM). Antibodies NI-607.427_C5 and NI-607.532_F3 showed no clear reduction in GFP with all dilutions tested.

The results are quite remarkable since Vero E6 cells were incubated with the antibodies only for one hour before the supernatant including the antibody was removed and replaced with fresh medium without antibody. Thus, the antibodies show a virus neutralizing effect already at a short time exposure to the virus.

Moreover, the cells were infected with the virus at a multiplicity of infection (MOI) of 1, a concentration which is usually used for the generation of persistently infected cells and one to two magnitudes higher than commonly applied in such assay; see, e.g., Thao, T. T. N. et al. Rapid reconstruction of SARS-CoV-2 using a synthetic genomics platform. Nature https://doi.org/10.1038/s41586-020-2294-9 (2020).

Nevertheless, even under these conditions some antibodies of the present invention, i.e., NI-607.531_C8 and NI-607.532_C11 are capable of completely inhibiting viral growth for 72 hours.

Thus, antibodies of the present invention would be particularly suitable to treat and/or prevent virus infection or spread by short time exposure early in infection and for example to infection points such as goblet and ciliated cells in the nose, which have been identified as likely initial infection points for the novel coronavirus that causes COVID-19 (see, e.g., Waradon Sungnak et al. (2020): “Single-Cell Transcriptomics Data Survey Reveals SARS-CoV-2 Entry Factors Highly Expressed in Nasal Epithelial Cells Together with Innate Immune Genes”. Nature Medicine. DOI: 10.1038/s41591-020-0868-6) and/or were the viral load is high such as present in the throat during early mild or prodromal stages (see, e.g., Wölfel R et al. Virological assessment of hospitalized patients with COVID-2019. Nature 2020 Apr. 1; doi.org/10.1038/s41586-020-2196-x).

For example, if a subject is notified to have just been in contact with COVID-19 Sars-CoV-2, e.g. by a coronavirus Tracking App one or more antibodies and composition of the present invention, respectively, may be immediately applied, for example by way of a spray, nebulizer, liquid drops, powder and the like adapted for nasal application or to the throat; see also infra compositions and administration routes in accordance with the present invention.

In one embodiment, the antibody of the present invention and the antibodies of the composition of the present invention, respectively, or at least one antibody of the composition is capable of neutralizing SARS-CoV-2 (SARS-CoV-2-GFP) at a concentration of ≤80 μg/mL/533.2 nM, preferably of ≤40 μg/mL/266.6 nM, preferably of ≤8 μg/mL/53.2 nM or of ≤1.6 μg/mL/10.7 nM, or of ≤0.32 μg/mL/2.1 nM, or at a concentration between 40 μg/mL/533.2 nM and 0.32 μg/mL/2.1 nM, or at a concentration between 40 μg/mL/266.6 nM and 8 μg/mL/53.2 nM.

Furthermore, the experiments performed in accordance with the present invention illustrated in Example 9 confirmed that the subject antibodies do not show any signs of cell toxicity, i.e., no enhanced cell toxicity in comparison to the isotype control. Thus, due to lack of toxicity even at high concentration the antibodies of the present invention can be used as full IgG, for example in first aid treatment to prevent infection and spread of the virus at an early stage of infection as mentioned above, where high dose may be required, rather than as antibody coded polynucleotide, for example RNA, see also infra.

In summary, several antibodies showed clear dose dependent reduction of viral driven GFP expression in comparison to the isotype control. At the same time the antibodies did not show any signs of cell toxicity.

Furthermore, in vivo studies can be performed in an ACE2 mouse model as described in Wu et al., Science 10.1126/science.abc2241 (2020) and/or in the rhesus macaque SARS-CoV-2 infection model as described in Shi et al., Nature https://doi.org/10.1038/s41586-020-2381-y (2020).

Since the antibodies of the present invention have been screened for binding to the RBD of SARS-CoV-2 they might bind same or similar epitopes, for example overlapping epitopes. On the other hand, the antibodies might bind different epitopes. The latter ones might be especially useful in therapeutic approaches when administered simultaneously in co-treatment approaches. In the context of the present application, “co-treatment” with two or more compounds is defined as administration of the two or more compounds to the patient within a specific time, usually about 24 h, including separate administration of two medicaments each containing one of the compounds as well as simultaneous administration whether or not the two compounds are combined in one formulation or whether they are in two separate formulations.

Accordingly, a cross-competition assay as described in Example 6 has been performed, in which the competitive binding of antibody pairs to the SARS-CoV-2-S1 (RBD) peptide was characterized. Whether the antibody pairs compete with each other can be derived from the matrix (Table V) shown in Example 6, wherein those antibodies that compete with each other share the same or close epitope and those that do not compete with each others have different epitopes, for example epitopes with no or only few overlapping amino acids. The principle is also shown in FIG. 4.

For the sake of conciseness, not all combinations are spelled out here, but can be of course derived from said matrix. For example, antibodies NI-607.531_C8 and NI-607.529_B9 and NI-607.531_C8 and NI-607.649_B11 do not share the same discontinuous epitope and do not compete with each other, respectively. In contrast, for example antibody NI-607.429_E4 shares the same or close epitope with antibody NI-607.531_C8 and NI-607.649_B11 and thus competes with those antibodies. Accordingly, antibody NI-607.429_E4 may be expected to also share one or more the biological properties of antibody NI-607.531_C8 and NI-607.649_11.

In one aspect, the antibodies and antigen-binding fragments thereof of the present invention bind or are capable of binding one or more or all of the circulating SARS-CoV-2 variants. Current circulating variants are listed by Centers for Disease Control and Prevention (CDC) on www.cdc.gov. The database is continuously updated. In one embodiment, the antibodies or the antigen-binding fragments of the present invention bind to or are capable of binding one or more or all of the following SARS-CoV-2 variants: S1 Mink, B1.351, B1.1.7, P1, B.1.135, RBD N439K, RBD Y453F, RBD N501Y, S1 D614G. It is apparent that presence of mutations occurring in afore-mentioned variants does not affect the binding efficacy of the antibodies to the corresponding variants of SARS-CoV-2. The binding of the antibodies of the present invention can be determined by direct ELISA (see, Example 11, FIG. 9).

In one aspect, the antibodies or the antigen-binding fragments thereof of the present invention bind to a discontinuous epitope of the RBD of the SARS-CoV-2. In one embodiment, the antibodies or the antigen-binding fragments thereof of the present invention specifically bind to a discontinuous epitope of the RBD of the SARS-CoV-2. The RBD of SARS-CoV-2 comprises and amino acid sequence SEQ ID NO: 301.

In one embodiment the epitope is determined by Cross-linking Mass Spectrometry (XL-MS) (see, Example 12; FIG. 10 for illustration purposes only). In one embodiment, a discontinuous epitope comprises a first, a second, and/or a third epitope. In one embodiment, the antibody, or antigen-binding fragment thereof binds to one or more of the first, the second and third epitopes. In one embodiment the first epitope comprises amino acid residues 112 to 120 (SEQ ID NO: 302) of SEQ ID NO: 301, the second epitope comprises amino acid residues 140 to 144 (SEQ ID NO: 303) of SEQ ID NO: 301 and the third epitope comprises amino acid residues 151 to 177 (SEQ ID NO: 304) of SEQ ID NO: 301. In one embodiment the first epitope comprises amino acid residues 51 to 68 (SEQ ID NO: 312) of SEQ ID NO: 301, the second epitope comprises amino acid residues 112 to 120 (SEQ ID NO: 302) of SEQ ID NO: 301 and the third epitope comprises amino acid residues 139 to 152 (SEQ ID NO: 313) of SEQ ID NO: 301. In one embodiment, the first, second and/or third epitope may vary by one or two amino acids. In one embodiment, the interaction site between the antibody of antigen-binding fragment thereof of the present invention includes the following amino acids on SARS-CoV-2 spike protein RBD (numbering based on SEQ IDF NO: 301): 112T, 120S, 140K, 141S, 144K, 151S, 177Y, 51Y, 65S, 67T, 68K, 139R, 148R, 152T.

In one embodiment, the interaction site(s) between NI-607.531_C8 and SARS-CoV-2-S includes the following amino acids on SARS-CoV-2 spike protein RBD (numbering based on SEQ ID NO: 301): 112T, 120S, 140K, 141S, 144K, 151S, 177Y. In one embodiment, stretch regions of interaction between NI-607.531_C8 and SARS-CoV-2-S correspond to amino acids 112-120 (SEQ ID NO: 302), 140-144 (SEQ ID NO: 303) and 151-177 (SEQ ID NO: 304) of SARS-CoV-2-RBD (SEQ ID NO: 301), (see, FIG. 10,A for illustration).

In one embodiment, the interaction site(s) between NI-607.649_B11 and SARS-CoV-2-S includes the following amino acids on SARS-CoV-2 spike protein RBD (numbering based on SEQ ID NO: 301): 51Y, 65S, 67T, 68K, 112T, 120S, 139R, 140K, 144K, 148R, 151S, 152T. In one embodiment, stretch regions of interaction between NI-607.649_B11 and SARS-CoV-2-S correspond to 51-68 (SEQ ID NO: 312), 112-120 (SEQ ID NO: 302) and 139-152 (SEQ ID NO:313) of SARS-CoV-2-S (see, FIG. 10,B for illustration).

In one embodiment the epitope is determined by hydrogen/deuterium exchange mass spectrometry (HDX-MS) (see, Example 12, FIG. 10 for illustration purposes only). In one embodiment a discontinuous epitope comprises a first and/or a second epitope. In one embodiment, the first epitope comprises amino acid residues 105 to 135 (SEQ ID NO: 305) of SEQ ID NO: 301. In one embodiment, the first epitope comprises amino acid residues 105 to 134 (SEQ ID NO: 306), 106 to 135 (SEQ ID NO: 307) or 106 to 134 (SEQ ID NO: 308) of SEQ ID NO: 301. In one embodiment, the second epitope comprises amino acid residues 153 to 178 (SEQ ID NO: 309) of SEQ ID NO: 301. In one embodiment, the second epitope comprises amino acid residues 155 to 178 (SEQ ID NO: 310) of SEQ ID NO: 301 or amino acid residues 164 to 176 (SEQ ID NO: 311). In one embodiment, the first epitope comprises amino acid residues 55 to 65 (SEQ ID NO: 314) of SEQ ID NO: 301. In one embodiment, the first epitope comprises amino acid residues 56 to 65 (SEQ ID NO: 315) of SEQ ID NO: 301. In one embodiment, the second epitope comprises amino acid residues 69 to 73 (SEQ ID NO: 316) of SEQ ID NO: 301. In one embodiment, the second epitope comprises amino acid residues 70 to 73 (SEQ ID NO: 317) of SEQ ID NO 301.

It is prudent to expect that the antibodies or the antigen-binding fragments thereof of the invention bind to the B.1.617. Locations of mutations in the RBD of SARS-CoV-2 characterizing B.1.617 are depicted in FIG. 10 (A,B) as “IN”. The mutations are located outside the cross-linking sites, as determined by the HDX-MS in case of both NI-607.531_C8 and NI-607.649_B11, therefore the efficacy of the antibody's binding to the B.1.617 variant may not be affected.

In one embodiment, the antibody of the present invention and the antibodies of the composition of the present invention, respectively, comprise or consist of at least one antibody of the above mentioned aspect, i.e., an antibody having one more of the biological properties of antibody NI-607.531_C8 and NI-607.649_B11, respectively, as illustrated in the Examples and competing with either antibody for binding SARS-CoV-2 RBD or at least sharing one epitope of the same discontinuous epitope of either antibody and/or the interacting amino acid residues common to both antibodies as shown in FIG. 10.

In one embodiment, at least two of the antibodies of the composition of the present invention and of the antibodies of the present invention, respectively compete with each other, or three, four, five, six, seven, eight, nine, ten, eleven, twelve, 13, 14, 15, 16, 17, 18, 19, 20, 21, 22, 23, 24, 25, 26, 27, 28, 29, or all of said antibodies.

In one embodiment, at least two of the antibodies of the composition of the present invention and of the antibodies of the present invention, respectively do not compete with each other, or three, four, five, six, seven, eight, nine, ten, eleven, twelve, 13, 14, 15, 16, 17, 18, 19, 20, 21, 22, 23, 24, 25, 26, 27, 28, 29, or all of said antibodies.

In one embodiment, at least one antibody of the composition of the present invention and one antibody of the present invention, respectively, or two, three, four, five, six, seven, eight, nine, ten, eleven, twelve, 13, 14, 15, 16, 17, 18, 19, 20, 21, 22, 23, 24, 25, 26, 27, 28, or 29 of the antibodies compete with antibody NI-607.529_B9 for binding to the RBD of SARS-CoV-2. In a preferred embodiment, antibody NI-607.426_F11, NI-607.427_C5, and/or NI-607.429_B9 compete with antibody NI-607.529_B9 for binding to the RBD of SARS-CoV-2.

In another embodiment, at least one antibody of the composition of the present invention and one antibody of the present invention, respectively, or two, three, four, five, six, seven, eight, nine, ten, eleven, twelve, 13, 14, 15, 16, 17, 18, 19, 20, 21, 22, 23, 24, 25, 26, 27, 28, or 29 of the antibodies of the composition compete with antibody NI-607.531_C8 for binding to the RBD of SARS-CoV-2. In a preferred embodiment, antibodies NI-607.532_F9, NI-607.532_C11, NI-607.532_D3, and NI-607.429_E4 compete with antibody NI-607.531_C8 for binding to the RBD of SARS-CoV-2.

In another embodiment, at least one antibody of the composition of the present invention and one antibody of the present invention, respectively, or two, three, four, five, six, seven, eight, nine, ten, eleven, twelve, 13, 14, 15, 16, 17, 18, 19, 20, 21, 22, 23, 24, 25, 26, 27, 28, or 29 of the antibodies compete with antibody NI-607.649_B11 for binding to the RBD of SARS-CoV-2. For example, antibody NI-607.532_C11, NI-607.532_F9, NI-607.426_F11, NI-607.427_C5, NI-607.429_E4 and/or NI-607.429_B9 compete with antibody NI-607. 649_B11 for binding to the RBD of SARS-CoV-2.

In one embodiment, at least one antibody of the composition of the present invention and one antibody of the present invention, respectively, or two, three, four, five, six, seven, eight, nine, ten, eleven, twelve, 13, 14, 15, 16, 17, 18, 19, 20, 21, 22, 23, 24, 25, 26, 27, 28, or 29 of the antibodies of the composition do not compete with antibody NI-607.529_B9 for binding to the RBD of SARS-CoV-2. For example, antibodies NI-607.531_C8, NI-607.532_C11, NI-607.532_D3, NI-607.532_F9, and NI-607.429_E4 do not compete with antibody NI-607.529_B9 for binding to the RBD of SARS-CoV-2.

In another embodiment, at least one antibody of the composition of the present invention and one antibody of the present invention, respectively, or two, three, four, five, six, seven, eight, nine, ten, eleven, twelve, 13, 14, 15, 16, 17, 18, 19, 20, 21, 22, 23, 24, 25, 26, 27, 28, or 29 of the antibodies of the composition do not compete with antibody NI-607.531_C8 for binding to the RBD of SARS-CoV-2. For example, antibodies NI-607.426_F11, NI-607.427_C5, NI-607.429_B9, NI-607.529_B9, and NI-607.649_B11 do not compete with antibody NI-607.531_C8 for binding to the RBD of SARS-CoV-2.

In another embodiment, at least one antibody of the composition of the present invention and one antibody of the present invention, respectively, or two, three, four, five, six, seven, eight, nine, ten, eleven, twelve, 13, 14, 15, 16, 17, 18, 19, 20, 21, 22, 23, 24, 25, 26, 27, 28, or 29 of the antibodies of the composition do not compete with antibody NI-607.649_B11 for binding to the RBD of SARS-CoV-2. For example, antibodies NI-607.531_C8, and NI-607.532_D3 do not compete with antibody NI-607.531_C8 for binding to the RBD of SARS-CoV-2.

These finding might be important for preparing an antibody cocktail comprising at least two antibodies of the present invention. In particular, it has been shown by Pinot et al. (2020), supra, that an antibody cocktail may enhance SARS-CoV-2 neutralization activity. It could be shown that the combination of a weakly neutralizing antibody with an antibody with high neutralization potency lead to an even enhanced neutralization potency, compared to single antibodies. Accordingly, it is prudent to expect that this synergistic effect will be achieved with antibodies of the present invention. It is particular preferred that the antibody cocktail of the present invention comprises antibodies which do not compete with each other for binding to the RBD of SARS-CoV-2, i.e. that they bind to different antigenic binding sites. Thus, the antibody combinations mentioned above could be used in the cocktail of the present invention; see also in Pinot et al. (2020), supra, where up to three antibodies are combined. Of course, two or more of the above-mentioned embodiments can be advantageously combined so as to arrive at a multi-specific antibody cocktail.

In general the cocktail of the present invention may comprise any one of the above-defined antibodies or fragments thereof and any possible combination of said antibodies or fragments. Thus, in one embodiment of the present invention, the cocktail comprises two, three, four, five, six, seven, eight, nine, ten, eleven, twelve, 13, 14, 15, 16, 17, 18, 19, 20, 21, 22, 23, 24, 25, 26, 27, 28, 29, or 30 antibodies of the present invention in any combination. Furthermore, the antibody cocktail may comprise one or more of the anti-SARS-CoV-2 antibodies described so far, for example any one of those disclosed in Pinot et al. (2020), supra, Wu et al. (2020), supra, and/or Shi et al. (2020), supra.

In one embodiment, the antibody cocktail comprises at least antibody NI-607.531_C8, NI-607.529_B9, NI-607.649_B11 or NI-607.429_E4, but preferably at least antibody NI-607.531_C8. In another embodiment, the cocktail comprises at least antibodies NI-607.531_C8 and NI-607.529_B9. In another embodiment, the cocktail comprises at least antibodies NI-607.531_C8 and NI-607.649_B11. In another embodiment, the cocktail comprises at least antibodies NI-607.531_C8, NI-607.529_B9 and NI-607.649_B11.

In a further additional or alternative embodiment of the present invention the anti-SARS-CoV-2 antibody, antigen-binding fragment, synthetic or biotechnological variant thereof can be optimized to have appropriate binding affinity to the target and stability properties. Therefore, at least one amino acid in the CDR or variable region, which is prone to modifications selected from the group consisting of glycosylation, oxidation, deamination, peptide bond cleavage, iso-aspartate formation and/or unpaired cysteine is substituted by a mutated amino acid that lacks such alteration or wherein at least one carbohydrate moiety is deleted or added chemically or enzymatically to the antibody, see, e.g. Liu et al., J. Pharm. Sci. 97 (2008), 2426-2447; Beck et al., Nat. Rev. Immunol. 10 (2010), 345-352; Haberger et al., MAbs. 6 (2014), 327-339.

An immunoglobulin or its encoding cDNA may be further modified. Thus, in a further embodiment, the method of the present invention comprises any one of the step(s) of producing a chimeric antibody, murinized antibody, single-chain antibody, Fab-fragment, bi-specific antibody, fusion antibody, labeled antibody or an analog of any one of those. Corresponding methods are known to the person skilled in the art and are described, e.g., in Harlow and Lane “Antibodies, A Laboratory Manual”, CSH Press, Cold Spring Harbor (1988) First edition; Second edition by Edward A. Greenfield, Dana-Farber Cancer Institute© 2014, ISBN 978-1-936113-81-1. For example, Fab and F(ab′)2 fragments may be produced recombinantly or by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Such fragments are sufficient for use, for example, in immunodiagnostic procedures involving coupling the immunospecific portions of immunoglobulins to detecting reagents such as radioisotopes.

In one embodiment, the antibody of the present invention and at least one or more (as defined above) of the antibodies of the composition of the present invention, respectively, may thus be provided in a format selected from the group consisting of a single chain Fv fragment (scFv), an F(ab′) fragment, an F(ab) fragment, and an F(ab′)2 fragment, an Fd, an Fv, a single-chain antibody, and a disulfide-linked Fv (sdFv) and/or wherein the antibody is a chimeric, for example murine-human, a murinized, a bispecific antibody or an IgG.

The five primary classes of immunoglobulins are IgG, IgM, IgA, IgD and IgE. These are distinguished by the type of heavy chain found in the molecule. IgG molecules have heavy chains known as gamma-chains; IgMs have mu-chains; IgAs have alpha-chains; IgEs have epsilon-chains; and IgDs have delta-chains; see for review, e.g., Schroeder et al., Structure and function of immunoglobulins. J. Allergy Clin. Immunol. 125 (2010), S41-S52. In principle, the antibodies of the present invention may be of any kind of class and antibody fragment as long as the binding specificity towards SARS-CoV-2 as indicated in Table I and illustrated in the appended Examples for the corresponding reference antibody remains unaffected in kind. However, preferably complete IgG antibodies are used, wherein the antibody comprises a constant domain. The constant domain may be native, i.e., originally cloned together with the variable domain or heterologous, for example, a murine constant in case animal studies are envisaged. Preferably, the constant domain is of human origin with a different IgG subtype, e.g. IgG1 versus IgG4 or a different allotype and allele, respectively, compared to the constant domain of the antibody as naturally occurred in human. The definition of “allotypes” requires that antibody reagents are available to determine the allotypes serologically. If the determination is only done at the sequence level, the polymorphisms have to be described as “alleles”. This does not hinder to establish a correspondence with allotypes if the correspondence allele-allotype has been experimentally proven, or if the individual sequence is identical to a sequence for which it has been demonstrated.

In one embodiment of the present invention, the constant domain is heterologous to at least one of the CDRs and the VH and VL chains, respectively, e.g. an immunoglobulin heavy chain constant domain and/or immunoglobulin light chain constant domain, preferably of the IgG type. In addition, or alternatively, the heterologous part of the antibody may be a mammalian secretory signal peptide. Put in other words, in one embodiment the anti-SARS-CoV-2 antibody and SARS-CoV-2 binding fragment, synthetic derivative, and biotechnological derivative thereof of the present invention is a (i) fusion protein comprising a polypeptide sequence which is heterologous to the VH region and/or VL region, or at least one CDR; and/or (ii) a non-natural variant of a polypeptide derived from an immunoglobulin, said non-natural variant comprising a heavy chain constant region that comprises one or more amino acid deletions, substitutions, and/or additions relative to a wild type polypeptide.

As mentioned, five immunoglobulin isotypes exist, of which immunoglobulin G (IgG) is most abundant in human serum. Notably, as indicated in Table I one patient (identifier 531 and 532) with a very mild disease course (only headaches and loss of smell) had a significant amount of IgA antibodies that scored positive in the screen, thus tempting to assume that IgA contributes to protection against SARS-CoV-2. Indeed, for influenza Gould et al. in Front. Microbiol. 8 (2017):900. doi: 10.3389/fmicb.2017.00900 report that nasal IgA provides protection against human influenza challenge in volunteers with low serum influenza antibody titer. Therefore, in one embodiment the antibody of the present invention is or is originally derived from IgA. Indeed, IgA-based monoclonal antibodies are set to emerge as new and potent options in the therapeutic arena including passive immunization with monomeric IgA for viral infections; see for review, e.g., Sousa-Pereira and Woof, IgA: Structure, Function, and Developability. Antibodies 2019, 8(4), 57; https://doi.org/10.3390/antib8040057 and Breedveld and Egmond, IgA and FcαRI: Pathological Roles and Therapeutic Opportunities. Front. Immunol. 10 (2019):553. doi: 10.3389/fimmu.2019.00553. For example, generation of an IgA monoclonal anti-influenza antibody provided protection against sublethal H5N1 infection after a single dose through intranasal administration; see Ye et. al., Clin. Vaccine Immunol. 17 (2010), 1363-1370. In this context, Seibert et al. reported that IgG antibodies may prevent pathogenesis associated with influenza virus infection but do not protect from virus infection by airborne transmission, while IgA antibodies are more important for preventing transmission of influenza viruses; see Seibert et al., J. Virology 87 (2013), 7793-7804. In view of the preliminary data obtained in accordance with the present invention it is prudent to expect that IgA antibodies can similarly contribute to control infection by SARS-CoV-2.

Therefore, in one aspect the present invention generally relates to antibodies and antigen-binding fragments thereof which are capable of binding to the RBD of the S protein of SARS-CoV-2 preferably with an EC₅₀ of <100 pM, and which are IgA, either naturally human-derived or genetically engineered, preferably wherein the antibody is an antibody of the present invention. In this aspect, the antibody is preferably intended for intranasal administration; see also infra in context with the administration regime of the pharmaceutical compositions of the present invention via pulmonary or mucosal routes.

As mentioned, IgA were found as the predominant class of anti-SARS-CoV-2 antibodies in one patient who had mild symptoms of the disease, which—without intending to be bound by theory—could lead to the conclusion that IgA antibodies play an important role in dampening mucosal infections. However, as also known IgA can also have detrimental effects in inflammatory or autoimmune diseases. Since the course of COVID-19 disease is most severe in patients with pre-existing conditions such side effects should be avoided. Therefore, in one embodiment the antibody of the present invention derived from an IgA is switched to an IgG. Thus, preferably, the immunoglobulin heavy and/or light chain constant domain present in the antibody of the present invention is of the IgG type. The four subclasses, IgG1, IgG2, IgG3, and IgG4, which are highly conserved, differ in their constant region, particularly in their hinges and upper CH2 domains. These regions are involved in binding to both IgG-Fc receptors (FcgR) and C1q. As a result, the different subclasses have different effector functions, both in terms of triggering FcgR-expressing cells, resulting in phagocytosis or antibody-dependent cell-mediated cytotoxicity, and activating complement. The Fc regions also contain a binding epitope for the neonatal Fc receptor (FcRn), responsible for the extended half-life, placental transport, and bidirectional transport of IgG through mucosal surfaces. However, FcRn is also expressed in myeloid cells, where it participates in both phagocytosis and antigen presentation together with classical FcgR and complement. How these properties, IgG-polymorphisms and post-translational modification of the antibodies in the form of glycosylation, affect IgG-function is described in Vidarsson et al., (2014) IgG subclasses and allotypes: from structure to effector function. Front. Immunol. 5:520. doi:10.3389/fimmu.2014.00520 and de Taeye et al., Antibodies 2019, 8, 30; doi:10.3390/antib8020030. Preferably, the immunoglobulin heavy and/or light chain constant domain present in the antibody of the present invention is of the IgG type.

Accordingly, in certain embodiments of the present invention a specific IgG type is preferred, for example the IgG4 or IgG1 isotype and/or the constant region of the antibody, or antigen-binding fragment, variant, or derivative thereof has been altered so as to provide desired biochemical characteristics.

In particular, in one embodiment the Fc portion of the antibody may be mutated to alter, i.e., to decrease or increase immune effector function or to increase its half-life using techniques known in the art. Thus, in one embodiment the Fc portion of the antibody is mutated to decrease immune effector function and in another embodiment the Fc portion of the antibody is mutated to increase immune effector function. In another embodiment, the antibody is mutated to increase its half-life.

For example, it may be that constant region modifications consistent with the instant invention moderate complement binding and thus reduce the serum half-life and nonspecific association of a conjugated cytotoxin. Other modifications of the constant region may be used to modify disulfide linkages or oligosaccharide moieties that allow for enhanced tissue antigen interaction due to increased antigen specificity or antibody flexibility. Furthermore, mutations in the Fc region can be made that lead to enhanced antibody dependent cell-mediated cytotoxicity (ADCC) or antibody-dependent cellular phagocytosis (ADCP) via increasing FcγRIIIa binding and/or decreasing FcγRIIIb binding and via increasing FcγRIIa binding and/or FcγRIIIa binding, respectively. For example, the GASDALIE Fc mutant (G236A/S239D/A330L/I332E) exhibits a higher affinity for FcγRIIIa. Another possibility is the enhancement of complement-dependent cytotoxicity (CDC) via increasing C1q binding and/or hexamerization.

In other embodiments, certain antibodies for use in the diagnostic and treatment methods described herein have a constant region, e.g., an IgG heavy chain constant region, which is altered to eliminate glycosylation, referred to elsewhere herein as aglycosylated or “agly” antibodies. Such “agly” antibodies may be prepared enzymatically as well as by engineering the consensus glycosylation site(s) in the constant region. It is believed that “agly” antibodies have a reduced effector function and thus an improved safety and stability profile in vivo. Methods of producing aglycosylated antibodies, having desired effector function are found for example in international application WO 2005/018572, which is incorporated by reference in its entirety. A further approach to reduce the effector function of antibodies is the reduction of FcγR and C1q binding by mutations in the Fc region.

A summary is for example given in the review of Wang et al., Protein Cell 9 (2018), 63-73, wherein Table 1 provides examples of modifications to modulate antibody effector function and the half-life of an antibody and which mutations described therein are herein incorporated by reference. The resulting physiological profile, bioavailability and other biochemical effects of the modifications, such as SARS-CoV-2 RBD binding and neutralization, biodistribution and serum half-life, may easily be measured and quantified using well know immunological techniques without undue experimentation.

As mentioned, in some instances inflammatory responses should be avoided for which reason effector functions of the constant domain of the antibody may be attenuated or eliminated altogether. For example, recombinant human IgG antibodies (hIgGs) completely devoid of binding to Fcγ receptors (FcγRs) and complement protein C1q, and thus with abolished immune effector functions, are of use for various therapeutic applications. It was found that the combination of Leu234Ala and Leu235Ala (commonly called LALA mutations) or the SPLE mutation eliminated FcγRIIa binding and were shown to eliminate detectable binding to FcγRI, IIa, and IIIa for both IgG1 and IgG4 and that the LALA-PG mutation was an improvement over LALA mutations alone in that they nullified Fc function in mouse and human IgG; for corresponding review see, e.g., Saunders (2019) Conceptual Approaches to Modulating Antibody Effector Functions and Circulation Half-Life. Front. Immunol. 10:1296.doi: 10.3389/fimmu.2019.01296 and Schlothauer et al., Protein Engineering, Design and Selection 29 (2016), 457-466. The introduction of the LALA mutation in the Fc region of an anti-SARS-CoV-2 antibody was already shown to be effective; see Shi et al., (2020), supra.

Another early approach to reduce effector function is to mutate the glycosylation site at N297 with mutations such as N297A, N297Q, and N297G. The half-life of an antibody can be increased via introducing the following mutations M252Y/S254T/T256E or M428L/N434S; see Wang et al. 2018.

Thus, in one embodiment, at least one or more (as defined above) of the antibodies of the compositions of the present invention and the antibody of the present invention, respectively is an IgG, or a recombinant IgG antibody or antibody fragment comprising an Fc portion mutated to eliminate or enhance FcR interactions, such as a LALA, N297, GASD/ALIE, or is glycan modified to eliminate or enhance FcR interactions, such as enzymatic or chemical addition or removal of glycans, or genetic modification of a glycosylation pattern, or comprises an Fc portion mutated to alter FcRn interactions to increase in vivo half-life and in vivo protection, such as a YTE or LS mutation.

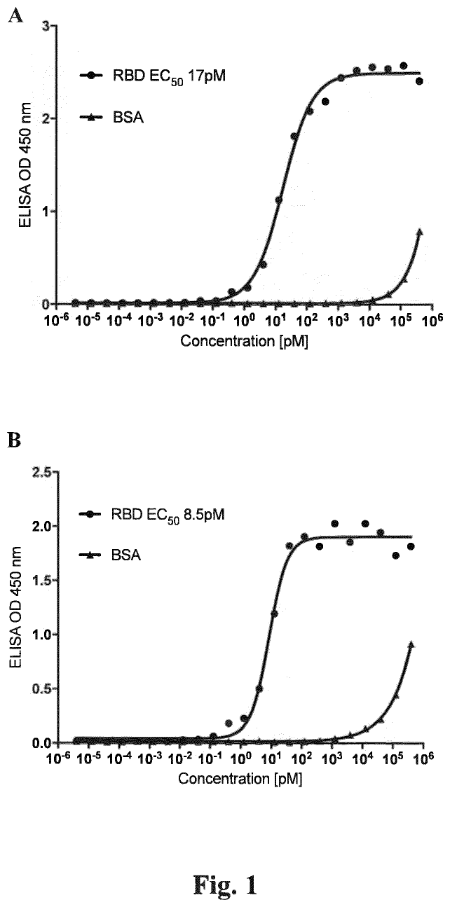

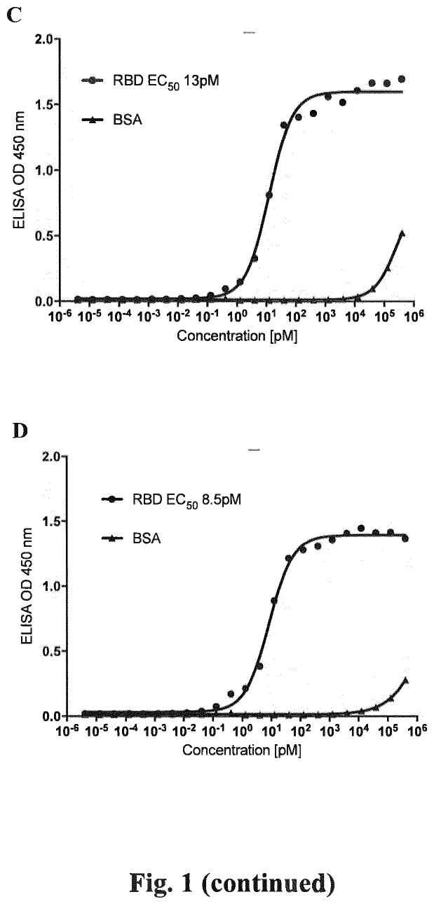

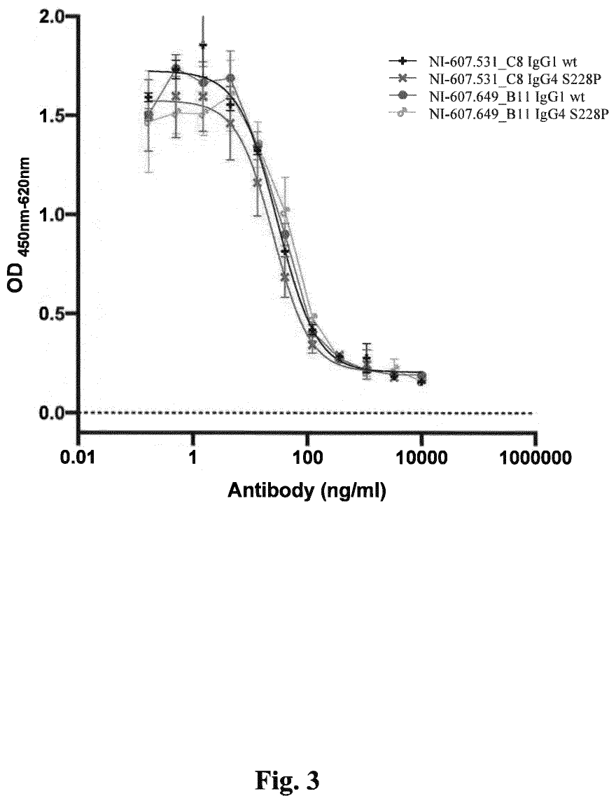

Given the potential risk of antibody-dependent enhancement (ADE) effect as observed in SARS-CoV infection, a reduced effector function is desirable. Thus, in a preferred embodiment, the antibody is of the IgG4 class or isotype. Human immunoglobulin G isotype 4 (IgG4) antibodies are potential candidates for antibody therapy when reduced immune effector functions are desirable. IgG4 antibodies are dynamic molecules able to undergo a process known as Fab arm exchange (FAE). This results in functionally monovalent, bispecific antibodies (bsAbs) with unknown specificity and hence, potentially, reduced therapeutic efficacy. In a particular preferred embodiment the antibody of the present invention and at least one of the antibodies of the composition of the present invention, respectively is of the IgG4 class or isotype including the S228P mutation. The S228P mutation prevents in vivo and in vitro IgG4 Fab-arm exchange as demonstrated using a combination of novel quantitative immunoassays and physiological matrix preparation; see Silva et al., J. Biol. Chem. 290 (2015), 5462-5469. Antibodies which are switched from IgG1 to IgG4 and in particular to IgG4 including the S228P mutation usually retain their binding specificities. This has been exemplarily shown based on antibodies NI-607.531_C8 and NI-607.649_B11, wherein the IgG1 antibodies showed a similar EC₅₀ value for binding to SARS-CoV-2 and a similar neutralization capability (IC₅₀) than their corresponding IgG4 S228P mutants as depicted in FIG. 1(A) to (D) and FIG. 3 and described in Examples 3 and 5, respectively.

Indeed, as demonstrated in Example 10 and shown in FIG. 8, anti-SARS-CoV-2 antibodies which are of the IgG4 type are efficacious in the prophylactic and therapeutic treatment of SARS-Cov-2 infection in a recognized animal model of COVID-19. Therefore, the present invention also generally relates to anti-SARS-CoV-2 antibodies of the IgG4 type, which naturally have reduced FcγR and C1q interactions and immune effector functions as compared with, e.g., hIgG1. In this context, all embodiments disclosed herein for the subject antibodies disclosed herein by way of the amino acid and nucleotide sequences of their VH and VL chain, and their CDRs, respectively, are generally applicable to anti-SARS-CoV-2 antibodies of the IgG4 type and their use in accordance with the present invention, for example embodiments for the polynucleotide encoding the antibody, vectors, host cells, recombinant expression of the antibody, labeling or otherwise modifications, formulation in a pharmaceutical composition and diagnostic use.

In another preferred embodiment, the antibody is of the IgG1 class or isotype preferably, wherein the antibody is an IgG1 variant comprising the amino acid substitutions L234A, L235A (LALA) and even more preferred the amino acid substitutions L234A, L235A, P329G (LALA-PG). For example, in order to avoid recruitment of immune cells through Fcγ-receptors and enable a short systemic half-life in circulation, FcγR binding can be abolished by introduction of P329G LALA mutations, see Schlothauer et al. (2016), supra, while FcRn binding and recycling can be abolished by introduction of Triple A (I253A, H310A, H435A) mutations; see, e.g., Regula et al., EMBO Mol. Med., 8 (2016), 1265-1288. Interestingly, the introduction of Triple A mutations also reduced viscosity, an important feature for nasal administration since higher viscosity may increase bioavailability from nasal formulations designed for systemic delivery of the antibody; see, e.g., for review Erdö et al., Brain Research Bulletin 143 (2018), 155-170.

The present invention also relates to one or more polynucleotide(s) encoding the antibody or antigen-binding fragment thereof of the present invention and at least one antibody of the composition of the present invention, respectively or an immunoglobulin VH and VL thereof, preferably wherein the polynucleotide(s) is (are) cDNA. In addition, the term polynucleotide comprehends the term nucleic acid which denotes any single- or double-stranded polynucleotide which are either deoxyribonucleic acids (DNA) or ribonucleic acids (RNA), thus including mRNA and modifications thereof.

In a preferred embodiment of the present invention, the polynucleotide comprises, consists essentially of, or consists of a nucleic acid having a polynucleotide sequence encoding the VH or VL chain of an anti-SARS-CoV-2 antibody as depicted in Table II. In this respect, the person skilled in the art will readily appreciate that the polynucleotides encoding the light and/or heavy chain may be encoded by one or more polynucleotides. In one embodiment therefore, the polynucleotide comprises, consists essentially of, or consists of a nucleic acid having a polynucleotide sequence of the VH and the VL chain of an anti-SARS-CoV-2 antibody as depicted in Table II.

In one embodiment of the present invention, the polynucleotide(s) are linked to a heterologous nucleic acid, for example expression control sequences such as a promoter, transcription and/or translation enhancer sequences, internal ribosome binding sites, nucleic acids encoding a peptide leader sequence for recombinant expression in a host and the like. Accordingly, the present invention relates to a polynucleotide encoding a human-derived recombinant anti-SARS-CoV-2 antibody or SARS-CoV-2 binding fragment, synthetic derivative, or biotechnological derivative thereof having any one, preferably at least two and most preferably all functional features, i.e. binding and optionally neutralizing properties as indicated for the reference antibody in Table I and illustrated in the Examples, wherein the respective reference antibody is indicated in parenthesis [NI-607.XXX.YY] and the polynucleotide encodes
(i) [NI-607.274_B7] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 3 or a variant thereof, wherein the variant comprises one or two amino acid substitutions,
   (b) VH-CDR2 comprises the amino acid sequence of SEQ ID NO: 4 or a variant thereof, wherein the variant comprises one or two amino acid substitutions,
   (c) VH-CDR3 comprises the amino acid sequence of SEQ ID NO: 5 or a variant thereof, wherein the variant comprises one or two amino acid substitutions,
   (d) VL-CDR1 comprises the amino acid sequence of SEQ ID NO: 8 or a variant thereof, wherein the variant comprises one or two amino acid substitutions,
   (e) VL-CDR2 comprises the amino acid sequence of SEQ ID NO: 9 or a variant thereof, wherein the variant comprises one or two amino acid substitutions, and
   (f) VL-CDR3 comprises the amino acid sequence of SEQ ID NO: 10 or a variant thereof, wherein the variant comprises one or two amino acid substitutions; and/or
(ii) [NI-607.274_B7] a VH chain and/or a VL chain, wherein
   (a) the VH chain comprises the amino acid sequence depicted in SEQ ID NO: 2 or a variant thereof, wherein the variant comprises one or more amino acid substitutions; or
   (b) the VL comprises the amino acid sequence depicted in SEQ ID NO: 7, or a variant thereof, wherein the variant comprises one or more amino acid substitutions;
   preferably wherein the VH and VL chain amino acid sequence is at least 90% identical to SEQ ID NO: 2 and 7, respectively; or
(iii) [NI-607.274_E5] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 13 or a variant thereof, wherein the variant comprises one or two amino acid substitutions,
   (b) VH-CDR2 comprises the amino acid sequence of SEQ ID NO: 14 or a variant thereof, wherein the variant comprises one or two amino acid substitutions,
   (c) VH-CDR3 comprises the amino acid sequence of SEQ ID NO: 15 or a variant thereof, wherein the variant comprises one or two amino acid substitutions,
   (d) VL-CDR1 comprises the amino acid sequence of SEQ ID NO: 18 or a variant thereof, wherein the variant comprises one or two amino acid substitutions,
   (e) VL-CDR2 comprises the amino acid sequence of SEQ ID NO: 19 or a variant thereof, wherein the variant comprises one or two amino acid substitutions, and
   (f) VL-CDR3 comprises the amino acid sequence of SEQ ID NO: 20 or a variant thereof, wherein the variant comprises one or two amino acid substitutions; and/or
(iv) [NI-607.274_E5] a VH chain and/or a VL chain, wherein
   (a) the VH chain comprises the amino acid sequence depicted in SEQ ID NO: 12 or a variant thereof, wherein the variant comprises one or more amino acid substitutions; or
   (b) the VL comprises the amino acid sequence depicted in SEQ ID NO: 17, or a variant thereof, wherein the variant comprises one or more amino acid substitutions;
   preferably wherein the VH and VL chain amino acid sequence is at least 90% identical to SEQ ID NO: 12 and 17, respectively; or
(v) [NI-607.275_C5] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 23 or a variant thereof, wherein the variant comprises one or two amino acid substitutions,
   (b) VH-CDR2 comprises the amino acid sequence of SEQ ID NO: 24 or a variant thereof, wherein the variant comprises one or two amino acid substitutions,
   (c) VH-CDR3 comprises the amino acid sequence of SEQ ID NO: 25 or a variant thereof, wherein the variant comprises one or two amino acid substitutions,
   (d) VL-CDR1 comprises the amino acid sequence of SEQ ID NO: 28 or a variant thereof, wherein the variant comprises one or two amino acid substitutions,
   (e) VL-CDR2 comprises the amino acid sequence of SEQ ID NO: 29 or a variant thereof, wherein the variant comprises one or two amino acid substitutions, and
   (f) VL-CDR3 comprises the amino acid sequence of SEQ ID NO: 30 or a variant thereof, wherein the variant comprises one or two amino acid substitutions; and/or
(vi) [NI-607.275_C5] a VH chain and/or a VL chain, wherein
   (a) the VH chain comprises the amino acid sequence depicted in SEQ ID NO: 22 or a variant thereof, wherein the variant comprises one or more amino acid substitutions; or
   (b) the VL comprises the amino acid sequence depicted in SEQ ID NO: 27, or a variant thereof, wherein the variant comprises one or more amino acid substitutions;
   preferably wherein the VH and VL chain amino acid sequence is at least 90% identical to SEQ ID NO: 22 and 27, respectively; or
(vii) [NI-607.426_D4] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 33 or a variant thereof, wherein the variant comprises one or two amino acid substitutions,
   (b) VH-CDR2 comprises the amino acid sequence of SEQ ID NO: 34 or a variant thereof, wherein the variant comprises one or two amino acid substitutions,
   (c) VH-CDR3 comprises the amino acid sequence of SEQ ID NO: 35 or a variant thereof, wherein the variant comprises one or two amino acid substitutions,
   (d) VL-CDR1 comprises the amino acid sequence of SEQ ID NO: 38 or a variant thereof, wherein the variant comprises one or two amino acid substitutions,
   (e) VL-CDR2 comprises the amino acid sequence of SEQ ID NO: 39 or a variant thereof, wherein the variant comprises one or two amino acid substitutions, and
   (f) VL-CDR3 comprises the amino acid sequence of SEQ ID NO: 40 or a variant thereof, wherein the variant comprises one or two amino acid substitutions; and/or
(viii) [NI-607.426_D4] a VH chain and/or a VL chain, wherein
   (a) the VH chain comprises the amino acid sequence depicted in SEQ ID NO: 32 or a variant thereof, wherein the variant comprises one or more amino acid substitutions; or
   (b) the VL comprises the amino acid sequence depicted in SEQ ID NO: 37, or a variant thereof, wherein the variant comprises one or more amino acid substitutions;
   preferably wherein the VH and VL chain amino acid sequence is at least 90% identical to SEQ ID NO: 32 and 37, respectively; or
(ix) [NI-607.426_E2] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 43 or a variant thereof, wherein the variant comprises one or two amino acid substitutions,
   (b) VH-CDR2 comprises the amino acid sequence of SEQ ID NO: 44 or a variant thereof, wherein the variant comprises one or two amino acid substitutions,
   (c) VH-CDR3 comprises the amino acid sequence of SEQ ID NO: 45 or a variant thereof, wherein the variant comprises one or two amino acid substitutions,
   (d) VL-CDR1 comprises the amino acid sequence of SEQ ID NO: 48 or a variant thereof, wherein the variant comprises one or two amino acid substitutions,
   (e) VL-CDR2 comprises the amino acid sequence of SEQ ID NO: 49 or a variant thereof, wherein the variant comprises one or two amino acid substitutions, and
   (f) VL-CDR3 comprises the amino acid sequence of SEQ ID NO: 50 or a variant thereof, wherein the variant comprises one or two amino acid substitutions; and/or
(x) [NI-607.426_E2] a VH chain and/or a VL chain, wherein
   (a) the VH chain comprises the amino acid sequence depicted in SEQ ID NO: 42 or a variant thereof, wherein the variant comprises one or more amino acid substitutions; or
   (b) the VL comprises the amino acid sequence depicted in SEQ ID NO: 47, or a variant thereof, wherein the variant comprises one or more amino acid substitutions;
   preferably wherein the VH and VL chain amino acid sequence is at least 90% identical to SEQ ID NO: 42 and 47, respectively; or
(xi) [NI-607.426_F11] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 53 or a variant thereof, wherein the variant comprises one or two amino acid substitutions,
   (b) VH-CDR2 comprises the amino acid sequence of SEQ ID NO: 54 or a variant thereof, wherein the variant comprises one or two amino acid substitutions,
   (c) VH-CDR3 comprises the amino acid sequence of SEQ ID NO: 55 or a variant thereof, wherein the variant comprises one or two amino acid substitutions,
   (d) VL-CDR1 comprises the amino acid sequence of SEQ ID NO: 58 or a variant thereof, wherein the variant comprises one or two amino acid substitutions,
   (e) VL-CDR2 comprises the amino acid sequence of SEQ ID NO: 59 or a variant thereof, wherein the variant comprises one or two amino acid substitutions, and
   (f) VL-CDR3 comprises the amino acid sequence of SEQ ID NO: 60 or a variant thereof, wherein the variant comprises one or two amino acid substitutions; and/or
(xii) [NI-607.426_F11] a VH chain and/or a VL chain, wherein
   (a) the VH chain comprises the amino acid sequence depicted in SEQ ID NO: 52 or a variant thereof, wherein the variant comprises one or more amino acid substitutions; or
   (b) the VL comprises the amino acid sequence depicted in SEQ ID NO: 57, or a variant thereof, wherein the variant comprises one or more amino acid substitutions;
   preferably wherein the VH and VL chain amino acid sequence is at least 90% identical to SEQ ID NO: 52 and 57, respectively; or
(xiii) [NI-607.427_C5] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 63 or a variant thereof, wherein the variant comprises one or two amino acid substitutions,
   (b) VH-CDR2 comprises the amino acid sequence of SEQ ID NO: 64 or a variant thereof, wherein the variant comprises one or two amino acid substitutions,
   (c) VH-CDR3 comprises the amino acid sequence of SEQ ID NO: 65 or a variant thereof, wherein the variant comprises one or two amino acid substitutions,
   (d) VL-CDR1 comprises the amino acid sequence of SEQ ID NO: 68 or a variant thereof, wherein the variant comprises one or two amino acid substitutions,
   (e) VL-CDR2 comprises the amino acid sequence of SEQ ID NO: 69 or a variant thereof, wherein the variant comprises one or two amino acid substitutions, and
   (f) VL-CDR3 comprises the amino acid sequence of SEQ ID NO: 70 or a variant thereof, wherein the variant comprises one or two amino acid substitutions; and/or
(xiv) [NI-607.427_C5] a VH chain and/or a VL chain, wherein
   (a) the VH chain comprises the amino acid sequence depicted in SEQ ID NO: 62 or a variant thereof, wherein the variant comprises one or more amino acid substitutions; or
   (b) the VL comprises the amino acid sequence depicted in SEQ ID NO: 67, or a variant thereof, wherein the variant comprises one or more amino acid substitutions;
   preferably wherein the VH and VL chain amino acid sequence is at least 90% identical to SEQ ID NO: 62 and 67, respectively; or
(xv) [NI-607.428_B9] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 73 or a variant thereof, wherein the variant comprises one or two amino acid substitutions,
   (b) VH-CDR2 comprises the amino acid sequence of SEQ ID NO: 74 or a variant thereof, wherein the variant comprises one or two amino acid substitutions,
   (c) VH-CDR3 comprises the amino acid sequence of SEQ ID NO: 75 or a variant thereof, wherein the variant comprises one or two amino acid substitutions,
   (d) VL-CDR1 comprises the amino acid sequence of SEQ ID NO: 78 or a variant thereof, wherein the variant comprises one or two amino acid substitutions,
   (e) VL-CDR2 comprises the amino acid sequence of SEQ ID NO: 79 or a variant thereof, wherein the variant comprises one or two amino acid substitutions, and
   (f) VL-CDR3 comprises the amino acid sequence of SEQ ID NO: 80 or a variant thereof, wherein the variant comprises one or two amino acid substitutions; and/or
(xvi) [NI-607.428_B9] a VH chain and/or a VL chain, wherein
   (a) the VH chain comprises the amino acid sequence depicted in SEQ ID NO: 72 or a variant thereof, wherein the variant comprises one or more amino acid substitutions; or
   (b) the VL comprises the amino acid sequence depicted in SEQ ID NO: 77, or a variant thereof, wherein the variant comprises one or more amino acid substitutions;
   preferably wherein the VH and VL chain amino acid sequence is at least 90% identical to SEQ ID NO: 72 and 77, respectively; or
(xvii) [NI-607.429_B9] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 83 or a variant thereof, wherein the variant comprises one or two amino acid substitutions,
   (b) VH-CDR2 comprises the amino acid sequence of SEQ ID NO: 84 or a variant thereof, wherein the variant comprises one or two amino acid substitutions,
   (c) VH-CDR3 comprises the amino acid sequence of SEQ ID NO: 85 or a variant thereof, wherein the variant comprises one or two amino acid substitutions,
   (d) VL-CDR1 comprises the amino acid sequence of SEQ ID NO: 88 or a variant thereof, wherein the variant comprises one or two amino acid substitutions,
   (e) VL-CDR2 comprises the amino acid sequence of SEQ ID NO: 89 or a variant thereof, wherein the variant comprises one or two amino acid substitutions, and
   (f) VL-CDR3 comprises the amino acid sequence of SEQ ID NO: 90 or a variant thereof, wherein the variant comprises one or two amino acid substitutions; and/or
(xviii) [NI-607.429_B9] a VH chain and/or a VL chain, wherein
   (a) the VH chain comprises the amino acid sequence depicted in SEQ ID NO: 82 or a variant thereof, wherein the variant comprises one or more amino acid substitutions; or
   (b) the VL comprises the amino acid sequence depicted in SEQ ID NO: 87, or a variant thereof, wherein the variant comprises one or more amino acid substitutions;
   preferably wherein the VH and VL chain amino acid sequence is at least 90% identical to SEQ ID NO: 82 and 87, respectively; or
(xix) [NI-607.429_E4] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 93 or a variant thereof, wherein the variant comprises one or two amino acid substitutions,
   (b) VH-CDR2 comprises the amino acid sequence of SEQ ID NO: 94 or a variant thereof, wherein the variant comprises one or two amino acid substitutions,
   (c) VH-CDR3 comprises the amino acid sequence of SEQ ID NO: 95 or a variant thereof, wherein the variant comprises one or two amino acid substitutions,
   (d) VL-CDR1 comprises the amino acid sequence of SEQ ID NO: 98 or a variant thereof, wherein the variant comprises one or two amino acid substitutions,
   (e) VL-CDR2 comprises the amino acid sequence of SEQ ID NO: 99 or a variant thereof, wherein the variant comprises one or two amino acid substitutions, and
   (f) VL-CDR3 comprises the amino acid sequence of SEQ ID NO: 100 or a variant thereof, wherein the variant comprises one or two amino acid substitutions; and/or
(xx) [NI-607.429_E4] a VH chain and/or a VL chain, wherein
   (a) the VH chain comprises the amino acid sequence depicted in SEQ ID NO: 92 or a variant thereof, wherein the variant comprises one or more amino acid substitutions; or
   (b) the VL comprises the amino acid sequence depicted in SEQ ID NO: 97, or a variant thereof, wherein the variant comprises one or more amino acid substitutions;
   preferably wherein the VH and VL chain amino acid sequence is at least 90% identical to SEQ ID NO: 92 and 97, respectively; or
(xxi) [NI-607.529_B9] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 103 or a variant thereof, wherein the variant comprises one or two amino acid substitutions,
   (b) VH-CDR2 comprises the amino acid sequence of SEQ ID NO: 104 or a variant thereof, wherein the variant comprises one or two amino acid substitutions,
   (c) VH-CDR3 comprises the amino acid sequence of SEQ ID NO: 105 or a variant thereof, wherein the variant comprises one or two amino acid substitutions,
   (d) VL-CDR1 comprises the amino acid sequence of SEQ ID NO: 108 or a variant thereof, wherein the variant comprises one or two amino acid substitutions,
   (e) VL-CDR2 comprises the amino acid sequence of SEQ ID NO: 109 or a variant thereof, wherein the variant comprises one or two amino acid substitutions, and
   (f) VL-CDR3 comprises the amino acid sequence of SEQ ID NO: 110 or a variant thereof, wherein the variant comprises one or two amino acid substitutions; and/or
(xxii) [NI-607.529_B9] a VH chain and/or a VL chain, wherein
   (a) the VH chain comprises the amino acid sequence depicted in SEQ ID NO: 102 or a variant thereof, wherein the variant comprises one or more amino acid substitutions; or
   (b) the VL comprises the amino acid sequence depicted in SEQ ID NO: 107, or a variant thereof, wherein the variant comprises one or more amino acid substitutions;
   preferably wherein the VH and VL chain amino acid sequence is at least 90% identical to SEQ ID NO: 102 and 107, respectively; or
(xxiii) [NI-607.529_G4] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 113 or a variant thereof, wherein the variant comprises one or two amino acid substitutions,
   (b) VH-CDR2 comprises the amino acid sequence of SEQ ID NO: 114 or a variant thereof, wherein the variant comprises one or two amino acid substitutions,
   (c) VH-CDR3 comprises the amino acid sequence of SEQ ID NO: 115 or a variant thereof, wherein the variant comprises one or two amino acid substitutions,
   (d) VL-CDR1 comprises the amino acid sequence of SEQ ID NO: 118 or a variant thereof, wherein the variant comprises one or two amino acid substitutions,
   (e) VL-CDR2 comprises the amino acid sequence of SEQ ID NO: 119 or a variant thereof, wherein the variant comprises one or two amino acid substitutions, and
   (f) VL-CDR3 comprises the amino acid sequence of SEQ ID NO: 120 or a variant thereof, wherein the variant comprises one or two amino acid substitutions; and/or
(xxiv) [NI-607.529_G4] a VH chain and/or a VL chain, wherein
   (a) the VH chain comprises the amino acid sequence depicted in SEQ ID NO: 112 or a variant thereof, wherein the variant comprises one or more amino acid substitutions; or
   (b) the VL comprises the amino acid sequence depicted in SEQ ID NO: 117, or a variant thereof, wherein the variant comprises one or more amino acid substitutions;
   preferably wherein the VH and VL chain amino acid sequence is at least 90% identical to SEQ ID NO: 112 and 117, respectively; or
(xxv) [NI-607.531_C8] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 123 or a variant thereof, wherein the variant comprises one or two amino acid substitutions,
   (b) VH-CDR2 comprises the amino acid sequence of SEQ ID NO: 124 or a variant thereof, wherein the variant comprises one or two amino acid substitutions,
   (c) VH-CDR3 comprises the amino acid sequence of SEQ ID NO: 125 or a variant thereof, wherein the variant comprises one or two amino acid substitutions,
   (d) VL-CDR1 comprises the amino acid sequence of SEQ ID NO: 128 or a variant thereof, wherein the variant comprises one or two amino acid substitutions,
   (e) VL-CDR2 comprises the amino acid sequence of SEQ ID NO: 129 or a variant thereof, wherein the variant comprises one or two amino acid substitutions, and
   (f) VL-CDR3 comprises the amino acid sequence of SEQ ID NO: 130 or a variant thereof, wherein the variant comprises one or two amino acid substitutions; and/or
(xxvi) [NI-607.531_C8] a VH chain and/or a VL chain, wherein
   (a) the VH chain comprises the amino acid sequence depicted in SEQ ID NO: 122 or a variant thereof, wherein the variant comprises one or more amino acid substitutions; or
   (b) the VL comprises the amino acid sequence depicted in SEQ ID NO: 127, or a variant thereof, wherein the variant comprises one or more amino acid substitutions;
   preferably wherein the VH and VL chain amino acid sequence is at least 90% identical to SEQ ID NO: 122 and 127, respectively; or
(xxvii) [NI-607.531_D8] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 133 or a variant thereof, wherein the variant comprises one or two amino acid substitutions,
   (b) VH-CDR2 comprises the amino acid sequence of SEQ ID NO: 134 or a variant thereof, wherein the variant comprises one or two amino acid substitutions,
   (c) VH-CDR3 comprises the amino acid sequence of SEQ ID NO: 135 or a variant thereof, wherein the variant comprises one or two amino acid substitutions,
   (d) VL-CDR1 comprises the amino acid sequence of SEQ ID NO: 138 or a variant thereof, wherein the variant comprises one or two amino acid substitutions,
   (e) VL-CDR2 comprises the amino acid sequence of SEQ ID NO: 139 or a variant thereof, wherein the variant comprises one or two amino acid substitutions, and
   (f) VL-CDR3 comprises the amino acid sequence of SEQ ID NO: 140 or a variant thereof, wherein the variant comprises one or two amino acid substitutions; and/or
(xxviii) [NI-607.531_D8] a VH chain and/or a VL chain, wherein
   (a) the VH chain comprises the amino acid sequence depicted in SEQ ID NO: 132 or a variant thereof, wherein the variant comprises one or more amino acid substitutions; or
   (b) the VL comprises the amino acid sequence depicted in SEQ ID NO: 137, or a variant thereof, wherein the variant comprises one or more amino acid substitutions;
   preferably wherein the VH and VL chain amino acid sequence is at least 90% identical to SEQ ID NO: 132 and 137, respectively; or
(xxix) [NI-607.532_B6] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 143 or a variant thereof, wherein the variant comprises one or two amino acid substitutions,
   (b) VH-CDR2 comprises the amino acid sequence of SEQ ID NO: 144 or a variant thereof, wherein the variant comprises one or two amino acid substitutions,
   (c) VH-CDR3 comprises the amino acid sequence of SEQ ID NO: 145 or a variant thereof, wherein the variant comprises one or two amino acid substitutions,
   (d) VL-CDR1 comprises the amino acid sequence of SEQ ID NO: 148 or a variant thereof, wherein the variant comprises one or two amino acid substitutions,
   (e) VL-CDR2 comprises the amino acid sequence of SEQ ID NO: 149 or a variant thereof, wherein the variant comprises one or two amino acid substitutions, and
   (f) VL-CDR3 comprises the amino acid sequence of SEQ ID NO: 150 or a variant thereof, wherein the variant comprises one or two amino acid substitutions; and/or
(xxx) [NI-607.532_B6] a VH chain and/or a VL chain, wherein
   (a) the VH chain comprises the amino acid sequence depicted in SEQ ID NO: 142 or a variant thereof, wherein the variant comprises one or more amino acid substitutions; or
   (b) the VL comprises the amino acid sequence depicted in SEQ ID NO: 147, or a variant thereof, wherein the variant comprises one or more amino acid substitutions;
   preferably wherein the VH and VL chain amino acid sequence is at least 90% identical to SEQ ID NO: 142 and 147, respectively; or
(xxxi) [NI-607.532_C11] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 153 or a variant thereof, wherein the variant comprises one or two amino acid substitutions,
   (b) VH-CDR2 comprises the amino acid sequence of SEQ ID NO: 154 or a variant thereof, wherein the variant comprises one or two amino acid substitutions,
   (c) VH-CDR3 comprises the amino acid sequence of SEQ ID NO: 155 or a variant thereof, wherein the variant comprises one or two amino acid substitutions,
   (d) VL-CDR1 comprises the amino acid sequence of SEQ ID NO: 158 or a variant thereof, wherein the variant comprises one or two amino acid substitutions,
   (e) VL-CDR2 comprises the amino acid sequence of SEQ ID NO: 159 or a variant thereof, wherein the variant comprises one or two amino acid substitutions, and
   (f) VL-CDR3 comprises the amino acid sequence of SEQ ID NO: 160 or a variant thereof, wherein the variant comprises one or two amino acid substitutions; and/or
(xxxii) [NI-607.532_C11] a VH chain and/or a VL chain, wherein
   (a) the VH chain comprises the amino acid sequence depicted in SEQ ID NO: 152 or a variant thereof, wherein the variant comprises one or more amino acid substitutions; or
   (b) the VL comprises the amino acid sequence depicted in SEQ ID NO: 157, or a variant thereof, wherein the variant comprises one or more amino acid substitutions;
   preferably wherein the VH and VL chain amino acid sequence is at least 90% identical to SEQ ID NO: 152 and 157, respectively; or
(xxxiii) [NI-607.532_C8] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 163 or a variant thereof, wherein the variant comprises one or two amino acid substitutions,
   (b) VH-CDR2 comprises the amino acid sequence of SEQ ID NO: 164 or a variant thereof, wherein the variant comprises one or two amino acid substitutions,
   (c) VH-CDR3 comprises the amino acid sequence of SEQ ID NO: 165 or a variant thereof, wherein the variant comprises one or two amino acid substitutions,
   (d) VL-CDR1 comprises the amino acid sequence of SEQ ID NO: 168 or a variant thereof, wherein the variant comprises one or two amino acid substitutions,
   (e) VL-CDR2 comprises the amino acid sequence of SEQ ID NO: 169 or a variant thereof, wherein the variant comprises one or two amino acid substitutions, and
   (f) VL-CDR3 comprises the amino acid sequence of SEQ ID NO: 170 or a variant thereof, wherein the variant comprises one or two amino acid substitutions; and/or
(xxxiv) [NI-607.532_C8] a VH chain and/or a VL chain, wherein
   (a) the VH chain comprises the amino acid sequence depicted in SEQ ID NO: 162 or a variant thereof, wherein the variant comprises one or more amino acid substitutions; or
   (b) the VL comprises the amino acid sequence depicted in SEQ ID NO: 167, or a variant thereof, wherein the variant comprises one or more amino acid substitutions;
   preferably wherein the VH and VL chain amino acid sequence is at least 90% identical to SEQ ID NO: 162 and 167, respectively; or
(xxxv) [NI-607.532_D3] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 173 or a variant thereof, wherein the variant comprises one or two amino acid substitutions,
   (b) VH-CDR2 comprises the amino acid sequence of SEQ ID NO: 174 or a variant thereof, wherein the variant comprises one or two amino acid substitutions,
   (c) VH-CDR3 comprises the amino acid sequence of SEQ ID NO: 175 or a variant thereof, wherein the variant comprises one or two amino acid substitutions,
   (d) VL-CDR1 comprises the amino acid sequence of SEQ ID NO: 178 or a variant thereof, wherein the variant comprises one or two amino acid substitutions,
   (e) VL-CDR2 comprises the amino acid sequence of SEQ ID NO: 179 or a variant thereof, wherein the variant comprises one or two amino acid substitutions, and
   (f) VL-CDR3 comprises the amino acid sequence of SEQ ID NO: 180 or a variant thereof, wherein the variant comprises one or two amino acid substitutions; and/or
(xxxvi) [NI-607.532_D3] a VH chain and/or a VL chain, wherein
   (a) the VH chain comprises the amino acid sequence depicted in SEQ ID NO: 172 or a variant thereof, wherein the variant comprises one or more amino acid substitutions; or
   (b) the VL comprises the amino acid sequence depicted in SEQ ID NO: 177, or a variant thereof, wherein the variant comprises one or more amino acid substitutions;
   preferably wherein the VH and VL chain amino acid sequence is at least 90% identical to SEQ ID NO: 172 and 177, respectively; or
(xxxvii) [NI-607.532_D4] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 183 or a variant thereof, wherein the variant comprises one or two amino acid substitutions,
   (b) VH-CDR2 comprises the amino acid sequence of SEQ ID NO: 184 or a variant thereof, wherein the variant comprises one or two amino acid substitutions,
   (c) VH-CDR3 comprises the amino acid sequence of SEQ ID NO: 185 or a variant thereof, wherein the variant comprises one or two amino acid substitutions,
   (d) VL-CDR1 comprises the amino acid sequence of SEQ ID NO: 188 or a variant thereof, wherein the variant comprises one or two amino acid substitutions,
   (e) VL-CDR2 comprises the amino acid sequence of SEQ ID NO: 189 or a variant thereof, wherein the variant comprises one or two amino acid substitutions, and
   (f) VL-CDR3 comprises the amino acid sequence of SEQ ID NO: 190 or a variant thereof, wherein the variant comprises one or two amino acid substitutions; and/or
(xxxviii) [NI-607.532_D4] a VH chain and/or a VL chain, wherein
   (a) the VH chain comprises the amino acid sequence depicted in SEQ ID NO: 182 or a variant thereof, wherein the variant comprises one or more amino acid substitutions; or
   (b) the VL comprises the amino acid sequence depicted in SEQ ID NO: 187, or a variant thereof, wherein the variant comprises one or more amino acid substitutions;
   preferably wherein the VH and VL chain amino acid sequence is at least 90% identical to SEQ ID NO: 182 and 187, respectively; or
(xxxix) [NI-607.532_D8] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 193 or a variant thereof, wherein the variant comprises one or two amino acid substitutions,
   (b) VH-CDR2 comprises the amino acid sequence of SEQ ID NO: 194 or a variant thereof, wherein the variant comprises one or two amino acid substitutions,
   (c) VH-CDR3 comprises the amino acid sequence of SEQ ID NO: 195 or a variant thereof, wherein the variant comprises one or two amino acid substitutions,
   (d) VL-CDR1 comprises the amino acid sequence of SEQ ID NO: 198 or a variant thereof, wherein the variant comprises one or two amino acid substitutions,
   (e) VL-CDR2 comprises the amino acid sequence of SEQ ID NO: 199 or a variant thereof, wherein the variant comprises one or two amino acid substitutions, and
   (f) VL-CDR3 comprises the amino acid sequence of SEQ ID NO: 200 or a variant thereof, wherein the variant comprises one or two amino acid substitutions; and/or
(xxxx) [NI-607.532_D8] a VH chain and/or a VL chain, wherein
   (a) the VH chain comprises the amino acid sequence depicted in SEQ ID NO: 192 or a variant thereof, wherein the variant comprises one or more amino acid substitutions; or
   (b) the VL comprises the amino acid sequence depicted in SEQ ID NO: 197, or a variant thereof, wherein the variant comprises one or more amino acid substitutions;
   preferably wherein the VH and VL chain amino acid sequence is at least 90% identical to SEQ ID NO: 192 and 197, respectively; or
(xli) [NI-607.532_F9] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 203 or a variant thereof, wherein the variant comprises one or two amino acid substitutions,
   (b) VH-CDR2 comprises the amino acid sequence of SEQ ID NO: 204 or a variant thereof, wherein the variant comprises one or two amino acid substitutions,
   (c) VH-CDR3 comprises the amino acid sequence of SEQ ID NO: 205 or a variant thereof, wherein the variant comprises one or two amino acid substitutions,
   (d) VL-CDR1 comprises the amino acid sequence of SEQ ID NO: 208 or a variant thereof, wherein the variant comprises one or two amino acid substitutions,
   (e) VL-CDR2 comprises the amino acid sequence of SEQ ID NO: 209 or a variant thereof, wherein the variant comprises one or two amino acid substitutions, and
   (f) VL-CDR3 comprises the amino acid sequence of SEQ ID NO: 210 or a variant thereof, wherein the variant comprises one or two amino acid substitutions; and/or
(xlii) [NI-607.532_F9] a VH chain and/or a VL chain, wherein
   (a) the VH chain comprises the amino acid sequence depicted in SEQ ID NO: 202 or a variant thereof, wherein the variant comprises one or more amino acid substitutions; or
   (b) the VL comprises the amino acid sequence depicted in SEQ ID NO: 207, or a variant thereof, wherein the variant comprises one or more amino acid substitutions;
   preferably wherein the VH and VL chain amino acid sequence is at least 90% identical to SEQ ID NO: 202 and 207, respectively; or
(xliii) [NI-607.649_B11] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 213 or a variant thereof, wherein the variant comprises one or two amino acid substitutions,
   (b) VH-CDR2 comprises the amino acid sequence of SEQ ID NO: 214 or a variant thereof, wherein the variant comprises one or two amino acid substitutions,
   (c) VH-CDR3 comprises the amino acid sequence of SEQ ID NO: 215 or a variant thereof, wherein the variant comprises one or two amino acid substitutions,
   (d) VL-CDR1 comprises the amino acid sequence of SEQ ID NO: 218 or a variant thereof, wherein the variant comprises one or two amino acid substitutions,
   (e) VL-CDR2 comprises the amino acid sequence of SEQ ID NO: 219 or a variant thereof, wherein the variant comprises one or two amino acid substitutions, and
   (f) VL-CDR3 comprises the amino acid sequence of SEQ ID NO: 220 or a variant thereof, wherein the variant comprises one or two amino acid substitutions; and/or
(xliv) [NI-607.649_B11] a VH chain and/or a VL chain, wherein
   (a) the VH chain comprises the amino acid sequence depicted in SEQ ID NO: 212 or a variant thereof, wherein the variant comprises one or more amino acid substitutions; or
   (b) the VL comprises the amino acid sequence depicted in SEQ ID NO: 217, or a variant thereof, wherein the variant comprises one or more amino acid substitutions;
   preferably wherein the VH and VL chain amino acid sequence is at least 90% identical to SEQ ID NO: 212 and 217, respectively; or
(xlv) [NI-607.531_E7] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 223 or a variant thereof, wherein the variant comprises one or two amino acid substitutions,
   (b) VH-CDR2 comprises the amino acid sequence of SEQ ID NO: 224 or a variant thereof, wherein the variant comprises one or two amino acid substitutions,
   (c) VH-CDR3 comprises the amino acid sequence of SEQ ID NO: 225 or a variant thereof, wherein the variant comprises one or two amino acid substitutions,
   (d) VL-CDR1 comprises the amino acid sequence of SEQ ID NO: 228 or a variant thereof, wherein the variant comprises one or two amino acid substitutions,
   (e) VL-CDR2 comprises the amino acid sequence of SEQ ID NO: 229 or a variant thereof, wherein the variant comprises one or two amino acid substitutions, and
   (f) VL-CDR3 comprises the amino acid sequence of SEQ ID NO: 230 or a variant thereof, wherein the variant comprises one or two amino acid substitutions; and/or
(xlvi) [NI-607.531_E7] a VH chain and/or a VL chain, wherein
   (a) the VH chain comprises the amino acid sequence depicted in SEQ ID NO: 222 or a variant thereof, wherein the variant comprises one or more amino acid substitutions; or
   (b) the VL comprises the amino acid sequence depicted in SEQ ID NO: 227, or a variant thereof, wherein the variant comprises one or more amino acid substitutions;
   preferably wherein the VH and VL chain amino acid sequence is at least 90% identical to SEQ ID NO: 222 and 227, respectively; or
(xlvii) [NI-607.532_F3] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 233 or a variant thereof, wherein the variant comprises one or two amino acid substitutions,
   (b) VH-CDR2 comprises the amino acid sequence of SEQ ID NO: 234 or a variant thereof, wherein the variant comprises one or two amino acid substitutions,
   (c) VH-CDR3 comprises the amino acid sequence of SEQ ID NO: 235 or a variant thereof, wherein the variant comprises one or two amino acid substitutions,
   (d) VL-CDR1 comprises the amino acid sequence of SEQ ID NO: 238 or a variant thereof, wherein the variant comprises one or two amino acid substitutions,
   (e) VL-CDR2 comprises the amino acid sequence of SEQ ID NO: 239 or a variant thereof, wherein the variant comprises one or two amino acid substitutions, and
   (f) VL-CDR3 comprises the amino acid sequence of SEQ ID NO: 240 or a variant thereof, wherein the variant comprises one or two amino acid substitutions; and/or
(xlviii) [NI-607.532_F3] a VH chain and/or a VL chain, wherein
   (a) the VH chain comprises the amino acid sequence depicted in SEQ ID NO: 232 or a variant thereof, wherein the variant comprises one or more amino acid substitutions; or
   (b) the VL comprises the amino acid sequence depicted in SEQ ID NO: 237, or a variant thereof, wherein the variant comprises one or more amino acid substitutions;
   preferably wherein the VH and VL chain amino acid sequence is at least 90% identical to SEQ ID NO: 232 and 237, respectively; or
(xlix) [NI-607.649_G7] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 243 or a variant thereof, wherein the variant comprises one or two amino acid substitutions,
   (b) VH-CDR2 comprises the amino acid sequence of SEQ ID NO: 244 or a variant thereof, wherein the variant comprises one or two amino acid substitutions,
   (c) VH-CDR3 comprises the amino acid sequence of SEQ ID NO: 245 or a variant thereof, wherein the variant comprises one or two amino acid substitutions,
   (d) VL-CDR1 comprises the amino acid sequence of SEQ ID NO: 248 or a variant thereof, wherein the variant comprises one or two amino acid substitutions,
   (e) VL-CDR2 comprises the amino acid sequence of SEQ ID NO: 249 or a variant thereof, wherein the variant comprises one or two amino acid substitutions, and
   (f) VL-CDR3 comprises the amino acid sequence of SEQ ID NO: 250 or a variant thereof, wherein the variant comprises one or two amino acid substitutions; and/or
(l) [NI-607.649_G7] a VH chain and/or a VL chain, wherein
   (a) the VH chain comprises the amino acid sequence depicted in SEQ ID NO: 242 or a variant thereof, wherein the variant comprises one or more amino acid substitutions; or
   (b) the VL comprises the amino acid sequence depicted in SEQ ID NO: 247, or a variant thereof, wherein the variant comprises one or more amino acid substitutions;
   preferably wherein the VH and VL chain amino acid sequence is at least 90% identical to SEQ ID NO: 242 and 247, respectively; or
(li) [NI-607.761_B7] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 253 or a variant thereof, wherein the variant comprises one or two amino acid substitutions,
   (b) VH-CDR2 comprises the amino acid sequence of SEQ ID NO: 254 or a variant thereof, wherein the variant comprises one or two amino acid substitutions,
   (c) VH-CDR3 comprises the amino acid sequence of SEQ ID NO: 255 or a variant thereof, wherein the variant comprises one or two amino acid substitutions,
   (d) VL-CDR1 comprises the amino acid sequence of SEQ ID NO: 258 or a variant thereof, wherein the variant comprises one or two amino acid substitutions,
   (e) VL-CDR2 comprises the amino acid sequence of SEQ ID NO: 259 or a variant thereof, wherein the variant comprises one or two amino acid substitutions, and
   (f) VL-CDR3 comprises the amino acid sequence of SEQ ID NO: 260 or a variant thereof, wherein the variant comprises one or two amino acid substitutions; and/or
(lii) [NI-607.761_B7] a VH chain and/or a VL chain, wherein
   (a) the VH chain comprises the amino acid sequence depicted in SEQ ID NO: 252 or a variant thereof, wherein the variant comprises one or more amino acid substitutions; or
   (b) the VL comprises the amino acid sequence depicted in SEQ ID NO: 257, or a variant thereof, wherein the variant comprises one or more amino acid substitutions;
   preferably wherein the VH and VL chain amino acid sequence is at least 90% identical to SEQ ID NO: 252 and 257, respectively; or
(liii) [NI-607.791_B10] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 263 or a variant thereof, wherein the variant comprises one or two amino acid substitutions,
   (b) VH-CDR2 comprises the amino acid sequence of SEQ ID NO: 264 or a variant thereof, wherein the variant comprises one or two amino acid substitutions,
   (c) VH-CDR3 comprises the amino acid sequence of SEQ ID NO: 265 or a variant thereof, wherein the variant comprises one or two amino acid substitutions,
   (d) VL-CDR1 comprises the amino acid sequence of SEQ ID NO: 268 or a variant thereof, wherein the variant comprises one or two amino acid substitutions,
   (e) VL-CDR2 comprises the amino acid sequence of SEQ ID NO: 269 or a variant thereof, wherein the variant comprises one or two amino acid substitutions, and
   (f) VL-CDR3 comprises the amino acid sequence of SEQ ID NO: 270 or a variant thereof, wherein the variant comprises one or two amino acid substitutions; and/or
(liv) [NI-607.791_B10] a VH chain and/or a VL chain, wherein
   (a) the VH chain comprises the amino acid sequence depicted in SEQ ID NO: 262 or a variant thereof, wherein the variant comprises one or more amino acid substitutions; or
   (b) the VL comprises the amino acid sequence depicted in SEQ ID NO: 267, or a variant thereof, wherein the variant comprises one or more amino acid substitutions;
   preferably wherein the VH and VL chain amino acid sequence is at least 90% identical to SEQ ID NO: 262 and 267, respectively; or
(lv) [NI-607.531_E3] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 273 or a variant thereof, wherein the variant comprises one or two amino acid substitutions,
   (b) VH-CDR2 comprises the amino acid sequence of SEQ ID NO: 274 or a variant thereof, wherein the variant comprises one or two amino acid substitutions,
   (c) VH-CDR3 comprises the amino acid sequence of SEQ ID NO: 275 or a variant thereof, wherein the variant comprises one or two amino acid substitutions,
   (d) VL-CDR1 comprises the amino acid sequence of SEQ ID NO: 278 or a variant thereof, wherein the variant comprises one or two amino acid substitutions,
   (e) VL-CDR2 comprises the amino acid sequence of SEQ ID NO: 279 or a variant thereof, wherein the variant comprises one or two amino acid substitutions, and
   (f) VL-CDR3 comprises the amino acid sequence of SEQ ID NO: 280 or a variant thereof, wherein the variant comprises one or two amino acid substitutions; and/or
(lvi) [NI-607.531_E3] a VH chain and/or a VL chain, wherein
   (a) the VH chain comprises the amino acid sequence depicted in SEQ ID NO: 272 or a variant thereof, wherein the variant comprises one or more amino acid substitutions; or
   (b) the VL comprises the amino acid sequence depicted in SEQ ID NO: 277, or a variant thereof, wherein the variant comprises one or more amino acid substitutions;
   preferably wherein the VH and VL chain amino acid sequence is at least 90% identical to SEQ ID NO: 272 and 277, respectively; or
(lvii) [NI-607.820_B6] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 283 or a variant thereof, wherein the variant comprises one or two amino acid substitutions,
   (b) VH-CDR2 comprises the amino acid sequence of SEQ ID NO: 284 or a variant thereof, wherein the variant comprises one or two amino acid substitutions,
   (c) VH-CDR3 comprises the amino acid sequence of SEQ ID NO: 285 or a variant thereof, wherein the variant comprises one or two amino acid substitutions,
   (d) VL-CDR1 comprises the amino acid sequence of SEQ ID NO: 288 or a variant thereof, wherein the variant comprises one or two amino acid substitutions,
   (e) VL-CDR2 comprises the amino acid sequence of SEQ ID NO: 289 or a variant thereof, wherein the variant comprises one or two amino acid substitutions, and
   (f) VL-CDR3 comprises the amino acid sequence of SEQ ID NO: 290 or a variant thereof, wherein the variant comprises one or two amino acid substitutions; and/or
(lviii) [NI-607.820_B6] a VH chain and/or a VL chain, wherein
   (a) the VH chain comprises the amino acid sequence depicted in SEQ ID NO: 282 or a variant thereof, wherein the variant comprises one or more amino acid substitutions; or
   (b) the VL comprises the amino acid sequence depicted in SEQ ID NO: 287, or a variant thereof, wherein the variant comprises one or more amino acid substitutions;
   preferably wherein the VH and VL chain amino acid sequence is at least 90% identical to SEQ ID NO: 282 and 287, respectively; or
(lix) [NI-607.820_B7] a VH chain comprising CDRs 1, 2, and 3, and/or a VL chain comprising VL CDRs 1, 2, and 3 as defined by Chothia, wherein
   (a) VH-CDR1 comprises the amino acid sequence of SEQ ID NO: 293 or a variant thereof, wherein the variant comprises one or two amino acid substitutions,
   (b) VH-CDR2 comprises the amino acid sequence of SEQ ID NO: 294 or a variant thereof, wherein the variant comprises one or two amino acid substitutions,
   (c) VH-CDR3 comprises the amino acid sequence of SEQ ID NO: 295 or a variant thereof, wherein the variant comprises one or two amino acid substitutions,
   (d) VL-CDR1 comprises the amino acid sequence of SEQ ID NO: 298 or a variant thereof, wherein the variant comprises one or two amino acid substitutions,
   (e) VL-CDR2 comprises the amino acid sequence of SEQ ID NO: 299 or a variant thereof, wherein the variant comprises one or two amino acid substitutions, and
   (f) VL-CDR3 comprises the amino acid sequence of SEQ ID NO: 300 or a variant thereof, wherein the variant comprises one or two amino acid substitutions; and/or
(lx) [NI-607.820_B7] a VH chain and/or a VL chain, wherein
   (a) the VH chain comprises the amino acid sequence depicted in SEQ ID NO: 292 or a variant thereof, wherein the variant comprises one or more amino acid substitutions; or
   (b) the VL comprises the amino acid sequence depicted in SEQ ID NO: 297, or a variant thereof, wherein the variant comprises one or more amino acid substitutions;
   preferably wherein the VH and VL chain amino acid sequence is at least 90% identical to SEQ ID NO: 292 and 297, respectively.

In addition, the present invention relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 3, 4, and 5, respectively, and wherein the VH when paired with a light chain variable region (VL) comprising the amino acid sequence set forth in SEQ ID NO: 7 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 8, 9, and 10, respectively, and wherein the VL when paired with a VH comprising the amino acid sequence set forth in SEQ ID NO: 2 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 3, 4, and 5, respectively; and
   (ii) an immunoglobulin light chain or a fragment thereof comprising a VL comprising CDRs 1, 2, and 3 with the amino acid sequences set forth in SEQ ID NOs: 8, 9, and 10, respectively;
(d) a polynucleotide encoding an immunoglobulin heavy chain or a fragment thereof comprising a VH comprising the amino acid sequence set forth in SEQ ID NO: 2, wherein the VH when paired with a VL comprising the amino acid sequence set forth in SEQ ID NO: 7 binds to SARS-CoV-2;
(e) a polynucleotide encoding an immunoglobulin light chain or a fragment thereof comprising a VL comprising the amino acid sequence set forth in SEQ ID NO: 7, wherein the VL when paired with a VH comprising the amino acid sequence set forth in SEQ ID NO: 2 binds to SARS-CoV-2;
(f) a polynucleotide encoding an immunoglobulin heavy chain or a fragment thereof comprising a VH comprising the amino acid sequence set forth in SEQ ID NO: 2 and an immunoglobulin light chain or a fragment thereof comprising a VL comprising the amino acid sequence set forth in SEQ ID NO: 7;
(g) a polynucleotide as in any one of (a)-(f), wherein a CDR comprises one or more, preferably no more than two amino acid substitution and/or the variable region sequence is at least 90% identical to SEQ ID NO: 2 or SEQ ID NO: 7.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 13, 14, and 15, respectively, and wherein the VH when paired with a light chain variable region (VL) comprising the amino acid sequence set forth in SEQ ID NO: 17 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 18, 19, and 20, respectively, and wherein the VL when paired with a VH comprising the amino acid sequence set forth in SEQ ID NO: 12 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 13, 14, and 15, respectively; and
   (ii) an immunoglobulin light chain or a fragment thereof comprising a VL comprising CDRs 1, 2, and 3 with the amino acid sequences set forth in SEQ ID NOs: 18, 19, and 20, respectively;
(d) a polynucleotide encoding an immunoglobulin heavy chain or a fragment thereof comprising a VH comprising the amino acid sequence set forth in SEQ ID NO: 12, wherein the VH when paired with a VL comprising the amino acid sequence set forth in SEQ ID NO: 17 binds to SARS-CoV-2;
(e) a polynucleotide encoding an immunoglobulin light chain or a fragment thereof comprising a VL comprising the amino acid sequence set forth in SEQ ID NO: 17, wherein the VL when paired with a VH comprising the amino acid sequence set forth in SEQ ID NO: 12 binds to SARS-CoV-2;
(f) a polynucleotide encoding an immunoglobulin heavy chain or a fragment thereof comprising a VH comprising the amino acid sequence set forth in SEQ ID NO: 12 and an immunoglobulin light chain or a fragment thereof comprising a VL comprising the amino acid sequence set forth in SEQ ID NO: 17;
(g) a polynucleotide as in any one of (a)-(f), wherein a CDR comprises one or more, preferably no more than two amino acid substitution and/or the variable region sequence is at least 90% identical to SEQ ID NO: 12 or SEQ ID NO: 17.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 23, 24, and 25, respectively, and wherein the VH when paired with a light chain variable region (VL) comprising the amino acid sequence set forth in SEQ ID NO: 27 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 28, 29, and 30, respectively, and wherein the VL when paired with a VH comprising the amino acid sequence set forth in SEQ ID NO: 22 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 23, 24, and 25, respectively; and
   (ii) an immunoglobulin light chain or a fragment thereof comprising a VL comprising CDRs 1, 2, and 3 with the amino acid sequences set forth in SEQ ID NOs: 28, 29, and 30, respectively;
(d) a polynucleotide encoding an immunoglobulin heavy chain or a fragment thereof comprising a VH comprising the amino acid sequence set forth in SEQ ID NO: 22, wherein the VH when paired with a VL comprising the amino acid sequence set forth in SEQ ID NO: 27 binds to SARS-CoV-2;
(e) a polynucleotide encoding an immunoglobulin light chain or a fragment thereof comprising a VL comprising the amino acid sequence set forth in SEQ ID NO: 27, wherein the VL when paired with a VH comprising the amino acid sequence set forth in SEQ ID NO: 22 binds to SARS-CoV-2;
(f) a polynucleotide encoding an immunoglobulin heavy chain or a fragment thereof comprising a VH comprising the amino acid sequence set forth in SEQ ID NO: 22 and an immunoglobulin light chain or a fragment thereof comprising a VL comprising the amino acid sequence set forth in SEQ ID NO: 27;
(g) a polynucleotide as in any one of (a)-(f), wherein a CDR comprises one or more, preferably no more than two amino acid substitution and/or the variable region sequence is at least 90% identical to SEQ ID NO: 22 or SEQ ID NO: 27.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 33, 34, and 35, respectively, and wherein the VH when paired with a light chain variable region (VL) comprising the amino acid sequence set forth in SEQ ID NO: 37 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 38, 39, and 40, respectively, and wherein the VL when paired with a VH comprising the amino acid sequence set forth in SEQ ID NO: 32 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 33, 34, and 35, respectively; and
   (ii) an immunoglobulin light chain or a fragment thereof comprising a VL comprising CDRs 1, 2, and 3 with the amino acid sequences set forth in SEQ ID NOs: 38, 39, and 40, respectively;
(d) a polynucleotide encoding an immunoglobulin heavy chain or a fragment thereof comprising a VH comprising the amino acid sequence set forth in SEQ ID NO: 32, wherein the VH when paired with a VL comprising the amino acid sequence set forth in SEQ ID NO: 37 binds to SARS-CoV-2;
(e) a polynucleotide encoding an immunoglobulin light chain or a fragment thereof comprising a VL comprising the amino acid sequence set forth in SEQ ID NO: 37, wherein the VL when paired with a VH comprising the amino acid sequence set forth in SEQ ID NO: 32 binds to SARS-CoV-2;
(f) a polynucleotide encoding an immunoglobulin heavy chain or a fragment thereof comprising a VH comprising the amino acid sequence set forth in SEQ ID NO: 32 and an immunoglobulin light chain or a fragment thereof comprising a VL comprising the amino acid sequence set forth in SEQ ID NO: 37;
(g) a polynucleotide as in any one of (a)-(f), wherein a CDR comprises one or more, preferably no more than two amino acid substitution and/or the variable region sequence is at least 90% identical to SEQ ID NO: 32 or SEQ ID NO: 37.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 43, 44, and 45, respectively, and wherein the VH when paired with a light chain variable region (VL) comprising the amino acid sequence set forth in SEQ ID NO: 47 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 48, 49, and 50, respectively, and wherein the VL when paired with a VH comprising the amino acid sequence set forth in SEQ ID NO: 42 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 43, 44, and 45, respectively; and
   (ii) an immunoglobulin light chain or a fragment thereof comprising a VL comprising CDRs 1, 2, and 3 with the amino acid sequences set forth in SEQ ID NOs: 48, 49, and 50, respectively;
(d) a polynucleotide encoding an immunoglobulin heavy chain or a fragment thereof comprising a VH comprising the amino acid sequence set forth in SEQ ID NO: 42, wherein the VH when paired with a VL comprising the amino acid sequence set forth in SEQ ID NO: 47 binds to SARS-CoV-2;
(e) a polynucleotide encoding an immunoglobulin light chain or a fragment thereof comprising a VL comprising the amino acid sequence set forth in SEQ ID NO: 47, wherein the VL when paired with a VH comprising the amino acid sequence set forth in SEQ ID NO: 42 binds to SARS-CoV-2;
(f) a polynucleotide encoding an immunoglobulin heavy chain or a fragment thereof comprising a VH comprising the amino acid sequence set forth in SEQ ID NO: 42 and an immunoglobulin light chain or a fragment thereof comprising a VL comprising the amino acid sequence set forth in SEQ ID NO: 47;
(g) a polynucleotide as in any one of (a)-(f), wherein a CDR comprises one or more, preferably no more than two amino acid substitution and/or the variable region sequence is at least 90% identical to SEQ ID NO: 42 or SEQ ID NO: 47.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 53, 54, and 55, respectively, and wherein the VH when paired with a light chain variable region (VL) comprising the amino acid sequence set forth in SEQ ID NO: 57 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 58, 59, and 60, respectively, and wherein the VL when paired with a VH comprising the amino acid sequence set forth in SEQ ID NO: 52 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 53, 54, and 55, respectively; and
   (ii) an immunoglobulin light chain or a fragment thereof comprising a VL comprising CDRs 1, 2, and 3 with the amino acid sequences set forth in SEQ ID NOs: 58, 59, and 60, respectively;
(d) a polynucleotide encoding an immunoglobulin heavy chain or a fragment thereof comprising a VH comprising the amino acid sequence set forth in SEQ ID NO: 52, wherein the VH when paired with a VL comprising the amino acid sequence set forth in SEQ ID NO: 57 binds to SARS-CoV-2;
(e) a polynucleotide encoding an immunoglobulin light chain or a fragment thereof comprising a VL comprising the amino acid sequence set forth in SEQ ID NO: 57, wherein the VL when paired with a VH comprising the amino acid sequence set forth in SEQ ID NO: 52 binds to SARS-CoV-2;
(f) a polynucleotide encoding an immunoglobulin heavy chain or a fragment thereof comprising a VH comprising the amino acid sequence set forth in SEQ ID NO: 52 and an immunoglobulin light chain or a fragment thereof comprising a VL comprising the amino acid sequence set forth in SEQ ID NO: 57;
(g) a polynucleotide as in any one of (a)-(f), wherein a CDR comprises one or more, preferably no more than two amino acid substitution and/or the variable region sequence is at least 90% identical to SEQ ID NO: 52 or SEQ ID NO: 57.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 63, 64, and 65, respectively, and wherein the VH when paired with a light chain variable region (VL) comprising the amino acid sequence set forth in SEQ ID NO: 67 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 68, 69, and 70, respectively, and wherein the VL when paired with a VH comprising the amino acid sequence set forth in SEQ ID NO: 62 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 63, 64, and 65, respectively; and
   (ii) an immunoglobulin light chain or a fragment thereof comprising a VL comprising CDRs 1, 2, and 3 with the amino acid sequences set forth in SEQ ID NOs: 68, 69, and 70, respectively;
(d) a polynucleotide encoding an immunoglobulin heavy chain or a fragment thereof comprising a VH comprising the amino acid sequence set forth in SEQ ID NO: 62, wherein the VH when paired with a VL comprising the amino acid sequence set forth in SEQ ID NO: 67 binds to SARS-CoV-2;
(e) a polynucleotide encoding an immunoglobulin light chain or a fragment thereof comprising a VL comprising the amino acid sequence set forth in SEQ ID NO: 67, wherein the VL when paired with a VH comprising the amino acid sequence set forth in SEQ ID NO: 62 binds to SARS-CoV-2;
(f) a polynucleotide encoding an immunoglobulin heavy chain or a fragment thereof comprising a VH comprising the amino acid sequence set forth in SEQ ID NO: 62 and an immunoglobulin light chain or a fragment thereof comprising a VL comprising the amino acid sequence set forth in SEQ ID NO: 67;
(g) a polynucleotide as in any one of (a)-(f), wherein a CDR comprises one or more, preferably no more than two amino acid substitution and/or the variable region sequence is at least 90% identical to SEQ ID NO: 62 or SEQ ID NO: 67.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 73, 74, and 75, respectively, and wherein the VH when paired with a light chain variable region (VL) comprising the amino acid sequence set forth in SEQ ID NO: 77 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 78, 79, and 80, respectively, and wherein the VL when paired with a VH comprising the amino acid sequence set forth in SEQ ID NO: 72 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 73, 74, and 75, respectively; and
   (ii) an immunoglobulin light chain or a fragment thereof comprising a VL comprising CDRs 1, 2, and 3 with the amino acid sequences set forth in SEQ ID NOs: 78, 79, and 80, respectively;
(d) a polynucleotide encoding an immunoglobulin heavy chain or a fragment thereof comprising a VH comprising the amino acid sequence set forth in SEQ ID NO: 72, wherein the VH when paired with a VL comprising the amino acid sequence set forth in SEQ ID NO: 77 binds to SARS-CoV-2;
(e) a polynucleotide encoding an immunoglobulin light chain or a fragment thereof comprising a VL comprising the amino acid sequence set forth in SEQ ID NO: 77, wherein the VL when paired with a VH comprising the amino acid sequence set forth in SEQ ID NO: 72 binds to SARS-CoV-2;
(f) a polynucleotide encoding an immunoglobulin heavy chain or a fragment thereof comprising a VH comprising the amino acid sequence set forth in SEQ ID NO: 72 and an immunoglobulin light chain or a fragment thereof comprising a VL comprising the amino acid sequence set forth in SEQ ID NO: 77;
(g) a polynucleotide as in any one of (a)-(f), wherein a CDR comprises one or more, preferably no more than two amino acid substitution and/or the variable region sequence is at least 90% identical to SEQ ID NO: 72 or SEQ ID NO: 77.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 83, 84, and 85, respectively, and wherein the VH when paired with a light chain variable region (VL) comprising the amino acid sequence set forth in SEQ ID NO: 87 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 88, 89, and 90, respectively, and wherein the VL when paired with a VH comprising the amino acid sequence set forth in SEQ ID NO: 82 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 83, 84, and 85, respectively; and
   (ii) an immunoglobulin light chain or a fragment thereof comprising a VL comprising CDRs 1, 2, and 3 with the amino acid sequences set forth in SEQ ID NOs: 88, 89, and 90, respectively;
(d) a polynucleotide encoding an immunoglobulin heavy chain or a fragment thereof comprising a VH comprising the amino acid sequence set forth in SEQ ID NO: 82, wherein the VH when paired with a VL comprising the amino acid sequence set forth in SEQ ID NO: 87 binds to SARS-CoV-2;
(e) a polynucleotide encoding an immunoglobulin light chain or a fragment thereof comprising a VL comprising the amino acid sequence set forth in SEQ ID NO: 87, wherein the VL when paired with a VH comprising the amino acid sequence set forth in SEQ ID NO: 82 binds to SARS-CoV-2;
(f) a polynucleotide encoding an immunoglobulin heavy chain or a fragment thereof comprising a VH comprising the amino acid sequence set forth in SEQ ID NO: 82 and an immunoglobulin light chain or a fragment thereof comprising a VL comprising the amino acid sequence set forth in SEQ ID NO: 87;
(g) a polynucleotide as in any one of (a)-(f), wherein a CDR comprises one or more, preferably no more than two amino acid substitution and/or the variable region sequence is at least 90% identical to SEQ ID NO: 82 or SEQ ID NO: 87.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 93, 94, and 95, respectively, and wherein the VH when paired with a light chain variable region (VL) comprising the amino acid sequence set forth in SEQ ID NO: 97 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 98, 99, and 100, respectively, and wherein the VL when paired with a VH comprising the amino acid sequence set forth in SEQ ID NO: 92 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 93, 94, and 95, respectively; and
   (ii) an immunoglobulin light chain or a fragment thereof comprising a VL comprising CDRs 1, 2, and 3 with the amino acid sequences set forth in SEQ ID NOs: 98, 99, and 100, respectively;
(d) a polynucleotide encoding an immunoglobulin heavy chain or a fragment thereof comprising a VH comprising the amino acid sequence set forth in SEQ ID NO: 92, wherein the VH when paired with a VL comprising the amino acid sequence set forth in SEQ ID NO: 97 binds to SARS-CoV-2;
(e) a polynucleotide encoding an immunoglobulin light chain or a fragment thereof comprising a VL comprising the amino acid sequence set forth in SEQ ID NO: 97, wherein the VL when paired with a VH comprising the amino acid sequence set forth in SEQ ID NO: 92 binds to SARS-CoV-2;
(f) a polynucleotide encoding an immunoglobulin heavy chain or a fragment thereof comprising a VH comprising the amino acid sequence set forth in SEQ ID NO: 92 and an immunoglobulin light chain or a fragment thereof comprising a VL comprising the amino acid sequence set forth in SEQ ID NO: 97;
(g) a polynucleotide as in any one of (a)-(f), wherein a CDR comprises one or more, preferably no more than two amino acid substitution and/or the variable region sequence is at least 90% identical to SEQ ID NO: 92 or SEQ ID NO: 97.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 103, 104, and 105, respectively, and wherein the VH when paired with a light chain variable region (VL) comprising the amino acid sequence set forth in SEQ ID NO: 107 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 108, 109, and 110, respectively, and wherein the VL when paired with a VH comprising the amino acid sequence set forth in SEQ ID NO: 102 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 103, 104, and 105, respectively; and
   (ii) an immunoglobulin light chain or a fragment thereof comprising a VL comprising CDRs 1, 2, and 3 with the amino acid sequences set forth in SEQ ID NOs: 108, 109, and 110, respectively;
(d) a polynucleotide encoding an immunoglobulin heavy chain or a fragment thereof comprising a VH comprising the amino acid sequence set forth in SEQ ID NO: 102, wherein the VH when paired with a VL comprising the amino acid sequence set forth in SEQ ID NO: 107 binds to SARS-CoV-2;
(e) a polynucleotide encoding an immunoglobulin light chain or a fragment thereof comprising a VL comprising the amino acid sequence set forth in SEQ ID NO: 107, wherein the VL when paired with a VH comprising the amino acid sequence set forth in SEQ ID NO: 102 binds to SARS-CoV-2;
(f) a polynucleotide encoding an immunoglobulin heavy chain or a fragment thereof comprising a VH comprising the amino acid sequence set forth in SEQ ID NO: 102 and an immunoglobulin light chain or a fragment thereof comprising a VL comprising the amino acid sequence set forth in SEQ ID NO: 107;
(g) a polynucleotide as in any one of (a)-(f), wherein a CDR comprises one or more, preferably no more than two amino acid substitution and/or the variable region sequence is at least 90% identical to SEQ ID NO: 102 or SEQ ID NO: 107.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 113, 114, and 115, respectively, and wherein the VH when paired with a light chain variable region (VL) comprising the amino acid sequence set forth in SEQ ID NO: 117 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 118, 119, and 120, respectively, and wherein the VL when paired with a VH comprising the amino acid sequence set forth in SEQ ID NO: 112 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 113, 114, and 115, respectively; and
   (ii) an immunoglobulin light chain or a fragment thereof comprising a VL comprising CDRs 1, 2, and 3 with the amino acid sequences set forth in SEQ ID NOs: 118, 119, and 120, respectively;
(d) a polynucleotide encoding an immunoglobulin heavy chain or a fragment thereof comprising a VH comprising the amino acid sequence set forth in SEQ ID NO: 112, wherein the VH when paired with a VL comprising the amino acid sequence set forth in SEQ ID NO: 117 binds to SARS-CoV-2;
(e) a polynucleotide encoding an immunoglobulin light chain or a fragment thereof comprising a VL comprising the amino acid sequence set forth in SEQ ID NO: 117, wherein the VL when paired with a VH comprising the amino acid sequence set forth in SEQ ID NO: 112 binds to SARS-CoV-2;
(f) a polynucleotide encoding an immunoglobulin heavy chain or a fragment thereof comprising a VH comprising the amino acid sequence set forth in SEQ ID NO: 112 and an immunoglobulin light chain or a fragment thereof comprising a VL comprising the amino acid sequence set forth in SEQ ID NO: 117;
(g) a polynucleotide as in any one of (a)-(f), wherein a CDR comprises one or more, preferably no more than two amino acid substitution and/or the variable region sequence is at least 90% identical to SEQ ID NO: 112 or SEQ ID NO: 117.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 123, 124, and 125, respectively, and wherein the VH when paired with a light chain variable region (VL) comprising the amino acid sequence set forth in SEQ ID NO: 127 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 128, 129, and 130, respectively, and wherein the VL when paired with a VH comprising the amino acid sequence set forth in SEQ ID NO: 122 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 123, 124, and 125, respectively; and
   (ii) an immunoglobulin light chain or a fragment thereof comprising a VL comprising CDRs 1, 2, and 3 with the amino acid sequences set forth in SEQ ID NOs: 128, 129, and 130, respectively;
(d) a polynucleotide encoding an immunoglobulin heavy chain or a fragment thereof comprising a VH comprising the amino acid sequence set forth in SEQ ID NO: 122, wherein the VH when paired with a VL comprising the amino acid sequence set forth in SEQ ID NO: 127 binds to SARS-CoV-2;
(e) a polynucleotide encoding an immunoglobulin light chain or a fragment thereof comprising a VL comprising the amino acid sequence set forth in SEQ ID NO: 127, wherein the VL when paired with a VH comprising the amino acid sequence set forth in SEQ ID NO: 122 binds to SARS-CoV-2;
(f) a polynucleotide encoding an immunoglobulin heavy chain or a fragment thereof comprising a VH comprising the amino acid sequence set forth in SEQ ID NO: 122 and an immunoglobulin light chain or a fragment thereof comprising a VL comprising the amino acid sequence set forth in SEQ ID NO: 127;
(g) a polynucleotide as in any one of (a)-(f), wherein a CDR comprises one or more, preferably no more than two amino acid substitution and/or the variable region sequence is at least 90% identical to SEQ ID NO: 122 or SEQ ID NO: 127.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 133, 134, and 135, respectively, and wherein the VH when paired with a light chain variable region (VL) comprising the amino acid sequence set forth in SEQ ID NO: 137 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 138, 139, and 140, respectively, and wherein the VL when paired with a VH comprising the amino acid sequence set forth in SEQ ID NO: 132 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 133, 134, and 135, respectively; and
   (ii) an immunoglobulin light chain or a fragment thereof comprising a VL comprising CDRs 1, 2, and 3 with the amino acid sequences set forth in SEQ ID NOs: 138, 139, and 140, respectively;
(d) a polynucleotide encoding an immunoglobulin heavy chain or a fragment thereof comprising a VH comprising the amino acid sequence set forth in SEQ ID NO: 132, wherein the VH when paired with a VL comprising the amino acid sequence set forth in SEQ ID NO: 137 binds to SARS-CoV-2;
(e) a polynucleotide encoding an immunoglobulin light chain or a fragment thereof comprising a VL comprising the amino acid sequence set forth in SEQ ID NO: 137, wherein the VL when paired with a VH comprising the amino acid sequence set forth in SEQ ID NO: 132 binds to SARS-CoV-2;
(f) a polynucleotide encoding an immunoglobulin heavy chain or a fragment thereof comprising a VH comprising the amino acid sequence set forth in SEQ ID NO: 132 and an immunoglobulin light chain or a fragment thereof comprising a VL comprising the amino acid sequence set forth in SEQ ID NO: 137;
(g) a polynucleotide as in any one of (a)-(f), wherein a CDR comprises one or more, preferably no more than two amino acid substitution and/or the variable region sequence is at least 90% identical to SEQ ID NO: 132 or SEQ ID NO: 137.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 143, 144, and 145, respectively, and wherein the VH when paired with a light chain variable region (VL) comprising the amino acid sequence set forth in SEQ ID NO: 147 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 148, 149, and 150, respectively, and wherein the VL when paired with a VH comprising the amino acid sequence set forth in SEQ ID NO: 142 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 143, 144 and 145, respectively; and
   (ii) an immunoglobulin light chain or a fragment thereof comprising a VL comprising CDRs 1, 2, and 3 with the amino acid sequences set forth in SEQ ID NOs: 148, 149, and 150, respectively;
(d) a polynucleotide encoding an immunoglobulin heavy chain or a fragment thereof comprising a VH comprising the amino acid sequence set forth in SEQ ID NO: 142, wherein the VH when paired with a VL comprising the amino acid sequence set forth in SEQ ID NO: 147 binds to SARS-CoV-2;
(e) a polynucleotide encoding an immunoglobulin light chain or a fragment thereof comprising a VL comprising the amino acid sequence set forth in SEQ ID NO: 147, wherein the VL when paired with a VH comprising the amino acid sequence set forth in SEQ ID NO: 142 binds to SARS-CoV-2;
(f) a polynucleotide encoding an immunoglobulin heavy chain or a fragment thereof comprising a VH comprising the amino acid sequence set forth in SEQ ID NO: 142 and an immunoglobulin light chain or a fragment thereof comprising a VL comprising the amino acid sequence set forth in SEQ ID NO: 147;
(g) a polynucleotide as in any one of (a)-(f), wherein a CDR comprises one or more, preferably no more than two amino acid substitution and/or the variable region sequence is at least 90% identical to SEQ ID NO: 142 or SEQ ID NO: 147.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 153, 154, and 155, respectively, and wherein the VH when paired with a light chain variable region (VL) comprising the amino acid sequence set forth in SEQ ID NO: 157 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 158, 159, and 160, respectively, and wherein the VL when paired with a VH comprising the amino acid sequence set forth in SEQ ID NO: 152 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 153, 154, and 155, respectively; and
   (ii) an immunoglobulin light chain or a fragment thereof comprising a VL comprising CDRs 1, 2, and 3 with the amino acid sequences set forth in SEQ ID NOs: 158, 159, and 160, respectively;
(d) a polynucleotide encoding an immunoglobulin heavy chain or a fragment thereof comprising a VH comprising the amino acid sequence set forth in SEQ ID NO: 152, wherein the VH when paired with a VL comprising the amino acid sequence set forth in SEQ ID NO: 157 binds to SARS-CoV-2;
(e) a polynucleotide encoding an immunoglobulin light chain or a fragment thereof comprising a VL comprising the amino acid sequence set forth in SEQ ID NO: 157, wherein the VL when paired with a VH comprising the amino acid sequence set forth in SEQ ID NO: 152 binds to SARS-CoV-2;
(f) a polynucleotide encoding an immunoglobulin heavy chain or a fragment thereof comprising a VH comprising the amino acid sequence set forth in SEQ ID NO: 152 and an immunoglobulin light chain or a fragment thereof comprising a VL comprising the amino acid sequence set forth in SEQ ID NO: 157;
(g) a polynucleotide as in any one of (a)-(f), wherein a CDR comprises one or more, preferably no more than two amino acid substitution and/or the variable region sequence is at least 90% identical to SEQ ID NO: 152 or SEQ ID NO: 157.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 163, 164, and 165, respectively, and wherein the VH when paired with a light chain variable region (VL) comprising the amino acid sequence set forth in SEQ ID NO: 167 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 168, 169, and 170, respectively, and wherein the VL when paired with a VH comprising the amino acid sequence set forth in SEQ ID NO: 162 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 163, 164, and 165, respectively; and
   (ii) an immunoglobulin light chain or a fragment thereof comprising a VL comprising CDRs 1, 2, and 3 with the amino acid sequences set forth in SEQ ID NOs: 168, 169, and 170, respectively;
(d) a polynucleotide encoding an immunoglobulin heavy chain or a fragment thereof comprising a VH comprising the amino acid sequence set forth in SEQ ID NO: 162, wherein the VH when paired with a VL comprising the amino acid sequence set forth in SEQ ID NO: 167 binds to SARS-CoV-2;
(e) a polynucleotide encoding an immunoglobulin light chain or a fragment thereof comprising a VL comprising the amino acid sequence set forth in SEQ ID NO: 167, wherein the VL when paired with a VH comprising the amino acid sequence set forth in SEQ ID NO: 162 binds to SARS-CoV-2;
(f) a polynucleotide encoding an immunoglobulin heavy chain or a fragment thereof comprising a VH comprising the amino acid sequence set forth in SEQ ID NO: 162 and an immunoglobulin light chain or a fragment thereof comprising a VL comprising the amino acid sequence set forth in SEQ ID NO: 167;
(g) a polynucleotide as in any one of (a)-(f), wherein a CDR comprises one or more, preferably no more than two amino acid substitution and/or the variable region sequence is at least 90% identical to SEQ ID NO: 162 or SEQ ID NO: 167.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 173, 174, and 175, respectively, and wherein the VH when paired with a light chain variable region (VL) comprising the amino acid sequence set forth in SEQ ID NO: 177 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 178, 179, and 180, respectively, and wherein the VL when paired with a VH comprising the amino acid sequence set forth in SEQ ID NO: 172 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 173, 174, and 175, respectively; and
   (ii) an immunoglobulin light chain or a fragment thereof comprising a VL comprising CDRs 1, 2, and 3 with the amino acid sequences set forth in SEQ ID NOs: 178, 179, and 180, respectively;
(d) a polynucleotide encoding an immunoglobulin heavy chain or a fragment thereof comprising a VH comprising the amino acid sequence set forth in SEQ ID NO: 172, wherein the VH when paired with a VL comprising the amino acid sequence set forth in SEQ ID NO: 177 binds to SARS-CoV-2;
(e) a polynucleotide encoding an immunoglobulin light chain or a fragment thereof comprising a VL comprising the amino acid sequence set forth in SEQ ID NO: 177, wherein the VL when paired with a VH comprising the amino acid sequence set forth in SEQ ID NO: 172 binds to SARS-CoV-2;
(f) a polynucleotide encoding an immunoglobulin heavy chain or a fragment thereof comprising a VH comprising the amino acid sequence set forth in SEQ ID NO: 172 and an immunoglobulin light chain or a fragment thereof comprising a VL comprising the amino acid sequence set forth in SEQ ID NO: 177;
(g) a polynucleotide as in any one of (a)-(f), wherein a CDR comprises one or more, preferably no more than two amino acid substitution and/or the variable region sequence is at least 90% identical to SEQ ID NO: 172 or SEQ ID NO: 177.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 183, 184, and 185, respectively, and wherein the VH when paired with a light chain variable region (VL) comprising the amino acid sequence set forth in SEQ ID NO: 187 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 188, 189, and 190, respectively, and wherein the VL when paired with a VH comprising the amino acid sequence set forth in SEQ ID NO: 182 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 183, 184, and 185, respectively; and
   (ii) an immunoglobulin light chain or a fragment thereof comprising a VL comprising CDRs 1, 2, and 3 with the amino acid sequences set forth in SEQ ID NOs: 188, 189, and 190, respectively;
(d) a polynucleotide encoding an immunoglobulin heavy chain or a fragment thereof comprising a VH comprising the amino acid sequence set forth in SEQ ID NO: 182, wherein the VH when paired with a VL comprising the amino acid sequence set forth in SEQ ID NO: 187 binds to SARS-CoV-2;
(e) a polynucleotide encoding an immunoglobulin light chain or a fragment thereof comprising a VL comprising the amino acid sequence set forth in SEQ ID NO: 187, wherein the VL when paired with a VH comprising the amino acid sequence set forth in SEQ ID NO: 182 binds to SARS-CoV-2;
(f) a polynucleotide encoding an immunoglobulin heavy chain or a fragment thereof comprising a VH comprising the amino acid sequence set forth in SEQ ID NO: 182 and an immunoglobulin light chain or a fragment thereof comprising a VL comprising the amino acid sequence set forth in SEQ ID NO: 187;
(g) a polynucleotide as in any one of (a)-(f), wherein a CDR comprises one or more, preferably no more than two amino acid substitution and/or the variable region sequence is at least 90% identical to SEQ ID NO: 182 or SEQ ID NO: 187.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 193, 194, and 195, respectively, and wherein the VH when paired with a light chain variable region (VL) comprising the amino acid sequence set forth in SEQ ID NO: 197 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 198, 199, and 200, respectively, and wherein the VL when paired with a VH comprising the amino acid sequence set forth in SEQ ID NO: 192 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 193, 194, and 195, respectively; and
   (ii) an immunoglobulin light chain or a fragment thereof comprising a VL comprising CDRs 1, 2, and 3 with the amino acid sequences set forth in SEQ ID NOs: 198, 199, and 200, respectively;
(d) a polynucleotide encoding an immunoglobulin heavy chain or a fragment thereof comprising a VH comprising the amino acid sequence set forth in SEQ ID NO: 192, wherein the VH when paired with a VL comprising the amino acid sequence set forth in SEQ ID NO: 197 binds to SARS-CoV-2;
(e) a polynucleotide encoding an immunoglobulin light chain or a fragment thereof comprising a VL comprising the amino acid sequence set forth in SEQ ID NO: 197, wherein the VL when paired with a VH comprising the amino acid sequence set forth in SEQ ID NO: 192 binds to SARS-CoV-2;
(f) a polynucleotide encoding an immunoglobulin heavy chain or a fragment thereof comprising a VH comprising the amino acid sequence set forth in SEQ ID NO: 192 and an immunoglobulin light chain or a fragment thereof comprising a VL comprising the amino acid sequence set forth in SEQ ID NO: 197;
(g) a polynucleotide as in any one of (a)-(f), wherein a CDR comprises one or more, preferably no more than two amino acid substitution and/or the variable region sequence is at least 90% identical to SEQ ID NO: 192 or SEQ ID NO: 197.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 203, 204, and 205, respectively, and wherein the VH when paired with a light chain variable region (VL) comprising the amino acid sequence set forth in SEQ ID NO: 207 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 208, 209, and 210, respectively, and wherein the VL when paired with a VH comprising the amino acid sequence set forth in SEQ ID NO: 202 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 203, 204, and 205, respectively; and
   (ii) an immunoglobulin light chain or a fragment thereof comprising a VL comprising CDRs 1, 2, and 3 with the amino acid sequences set forth in SEQ ID NOs: 208, 209, and 210, respectively;
(d) a polynucleotide encoding an immunoglobulin heavy chain or a fragment thereof comprising a VH comprising the amino acid sequence set forth in SEQ ID NO: 202, wherein the VH when paired with a VL comprising the amino acid sequence set forth in SEQ ID NO: 207 binds to SARS-CoV-2;
(e) a polynucleotide encoding an immunoglobulin light chain or a fragment thereof comprising a VL comprising the amino acid sequence set forth in SEQ ID NO: 207, wherein the VL when paired with a VH comprising the amino acid sequence set forth in SEQ ID NO: 202 binds to SARS-CoV-2;
(f) a polynucleotide encoding an immunoglobulin heavy chain or a fragment thereof comprising a VH comprising the amino acid sequence set forth in SEQ ID NO: 202 and an immunoglobulin light chain or a fragment thereof comprising a VL comprising the amino acid sequence set forth in SEQ ID NO: 207;
(g) a polynucleotide as in any one of (a)-(f), wherein a CDR comprises one or more, preferably no more than two amino acid substitution and/or the variable region sequence is at least 90% identical to SEQ ID NO: 202 or SEQ ID NO: 207.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 213, 214, and 215, respectively, and wherein the VH when paired with a light chain variable region (VL) comprising the amino acid sequence set forth in SEQ ID NO: 217 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 218, 219, and 220, respectively, and wherein the VL when paired with a VH comprising the amino acid sequence set forth in SEQ ID NO: 212 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 213, 214, and 215, respectively; and
   (ii) an immunoglobulin light chain or a fragment thereof comprising a VL comprising CDRs 1, 2, and 3 with the amino acid sequences set forth in SEQ ID NOs: 218, 219, and 220, respectively;
(d) a polynucleotide encoding an immunoglobulin heavy chain or a fragment thereof comprising a VH comprising the amino acid sequence set forth in SEQ ID NO: 212, wherein the VH when paired with a VL comprising the amino acid sequence set forth in SEQ ID NO: 217 binds to SARS-CoV-2;
(e) a polynucleotide encoding an immunoglobulin light chain or a fragment thereof comprising a VL comprising the amino acid sequence set forth in SEQ ID NO: 217, wherein the VL when paired with a VH comprising the amino acid sequence set forth in SEQ ID NO: 212 binds to SARS-CoV-2;
(f) a polynucleotide encoding an immunoglobulin heavy chain or a fragment thereof comprising a VH comprising the amino acid sequence set forth in SEQ ID NO: 212 and an immunoglobulin light chain or a fragment thereof comprising a VL comprising the amino acid sequence set forth in SEQ ID NO: 217;
(g) a polynucleotide as in any one of (a)-(f), wherein a CDR comprises one or more, preferably no more than two amino acid substitution and/or the variable region sequence is at least 90% identical to SEQ ID NO: 212 or SEQ ID NO: 217.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 223, 224, and 225, respectively, and wherein the VH when paired with a light chain variable region (VL) comprising the amino acid sequence set forth in SEQ ID NO: 227 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 228, 229, and 230, respectively, and wherein the VL when paired with a VH comprising the amino acid sequence set forth in SEQ ID NO: 222 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 223, 224, and 225, respectively; and
   (ii) an immunoglobulin light chain or a fragment thereof comprising a VL comprising CDRs 1, 2, and 3 with the amino acid sequences set forth in SEQ ID NOs: 228, 229, and 230, respectively;
(d) a polynucleotide encoding an immunoglobulin heavy chain or a fragment thereof comprising a VH comprising the amino acid sequence set forth in SEQ ID NO: 222, wherein the VH when paired with a VL comprising the amino acid sequence set forth in SEQ ID NO: 227 binds to SARS-CoV-2;
(e) a polynucleotide encoding an immunoglobulin light chain or a fragment thereof comprising a VL comprising the amino acid sequence set forth in SEQ ID NO: 227, wherein the VL when paired with a VH comprising the amino acid sequence set forth in SEQ ID NO: 222 binds to SARS-CoV-2;
(f) a polynucleotide encoding an immunoglobulin heavy chain or a fragment thereof comprising a VH comprising the amino acid sequence set forth in SEQ ID NO: 222 and an immunoglobulin light chain or a fragment thereof comprising a VL comprising the amino acid sequence set forth in SEQ ID NO: 227;
(g) a polynucleotide as in any one of (a)-(f), wherein a CDR comprises one or more, preferably no more than two amino acid substitution and/or the variable region sequence is at least 90% identical to SEQ ID NO: 222 or SEQ ID NO: 227.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 233, 234, and 235, respectively, and wherein the VH when paired with a light chain variable region (VL) comprising the amino acid sequence set forth in SEQ ID NO: 237 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 238, 239, and 240, respectively, and wherein the VL when paired with a VH comprising the amino acid sequence set forth in SEQ ID NO: 232 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 233, 234, and 235, respectively; and
   (ii) an immunoglobulin light chain or a fragment thereof comprising a VL comprising CDRs 1, 2, and 3 with the amino acid sequences set forth in SEQ ID NOs: 238, 239, and 240, respectively;
(d) a polynucleotide encoding an immunoglobulin heavy chain or a fragment thereof comprising a VH comprising the amino acid sequence set forth in SEQ ID NO: 232, wherein the VH when paired with a VL comprising the amino acid sequence set forth in SEQ ID NO: 237 binds to SARS-CoV-2;
(e) a polynucleotide encoding an immunoglobulin light chain or a fragment thereof comprising a VL comprising the amino acid sequence set forth in SEQ ID NO: 237, wherein the VL when paired with a VH comprising the amino acid sequence set forth in SEQ ID NO: 232 binds to SARS-CoV-2;
(f) a polynucleotide encoding an immunoglobulin heavy chain or a fragment thereof comprising a VH comprising the amino acid sequence set forth in SEQ ID NO: 232 and an immunoglobulin light chain or a fragment thereof comprising a VL comprising the amino acid sequence set forth in SEQ ID NO: 237;
(g) a polynucleotide as in any one of (a)-(f), wherein a CDR comprises one or more, preferably no more than two amino acid substitution and/or the variable region sequence is at least 90% identical to SEQ ID NO: 232 or SEQ ID NO: 237.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 243, 244, and 245, respectively, and wherein the VH when paired with a light chain variable region (VL) comprising the amino acid sequence set forth in SEQ ID NO: 247 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 248, 249, and 250, respectively, and wherein the VL when paired with a VH comprising the amino acid sequence set forth in SEQ ID NO: 242 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 243, 244, and 245, respectively; and
   (ii) an immunoglobulin light chain or a fragment thereof comprising a VL comprising CDRs 1, 2, and 3 with the amino acid sequences set forth in SEQ ID NOs: 248, 249, and 250, respectively;
(d) a polynucleotide encoding an immunoglobulin heavy chain or a fragment thereof comprising a VH comprising the amino acid sequence set forth in SEQ ID NO: 242, wherein the VH when paired with a VL comprising the amino acid sequence set forth in SEQ ID NO: 247 binds to SARS-CoV-2;
(e) a polynucleotide encoding an immunoglobulin light chain or a fragment thereof comprising a VL comprising the amino acid sequence set forth in SEQ ID NO: 247, wherein the VL when paired with a VH comprising the amino acid sequence set forth in SEQ ID NO: 242 binds to SARS-CoV-2;
(f) a polynucleotide encoding an immunoglobulin heavy chain or a fragment thereof comprising a VH comprising the amino acid sequence set forth in SEQ ID NO: 242 and an immunoglobulin light chain or a fragment thereof comprising a VL comprising the amino acid sequence set forth in SEQ ID NO: 247;
(g) a polynucleotide as in any one of (a)-(f), wherein a CDR comprises one or more, preferably no more than two amino acid substitution and/or the variable region sequence is at least 90% identical to SEQ ID NO: 242 or SEQ ID NO: 247.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 253, 254, and 255, respectively, and wherein the VH when paired with a light chain variable region (VL) comprising the amino acid sequence set forth in SEQ ID NO: 257 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 258, 259, and 260, respectively, and wherein the VL when paired with a VH comprising the amino acid sequence set forth in SEQ ID NO: 252 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 253, 254, and 255, respectively; and
   (ii) an immunoglobulin light chain or a fragment thereof comprising a VL comprising CDRs 1, 2, and 3 with the amino acid sequences set forth in SEQ ID NOs: 258, 259, and 260, respectively;
(d) a polynucleotide encoding an immunoglobulin heavy chain or a fragment thereof comprising a VH comprising the amino acid sequence set forth in SEQ ID NO: 252, wherein the VH when paired with a VL comprising the amino acid sequence set forth in SEQ ID NO: 257 binds to SARS-CoV-2;
(e) a polynucleotide encoding an immunoglobulin light chain or a fragment thereof comprising a VL comprising the amino acid sequence set forth in SEQ ID NO: 257, wherein the VL when paired with a VH comprising the amino acid sequence set forth in SEQ ID NO: 252 binds to SARS-CoV-2;
(f) a polynucleotide encoding an immunoglobulin heavy chain or a fragment thereof comprising a VH comprising the amino acid sequence set forth in SEQ ID NO: 252 and an immunoglobulin light chain or a fragment thereof comprising a VL comprising the amino acid sequence set forth in SEQ ID NO: 257;
(g) a polynucleotide as in any one of (a)-(f), wherein a CDR comprises one or more, preferably no more than two amino acid substitution and/or the variable region sequence is at least 90% identical to SEQ ID NO: 252 or SEQ ID NO: 257.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 263, 264, and 265, respectively, and wherein the VH when paired with a light chain variable region (VL) comprising the amino acid sequence set forth in SEQ ID NO: 267 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 268, 269, and 270, respectively, and wherein the VL when paired with a VH comprising the amino acid sequence set forth in SEQ ID NO: 262 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 263, 264, and 265, respectively; and
   (ii) an immunoglobulin light chain or a fragment thereof comprising a VL comprising CDRs 1, 2, and 3 with the amino acid sequences set forth in SEQ ID NOs: 268, 269, and 270, respectively;
(d) a polynucleotide encoding an immunoglobulin heavy chain or a fragment thereof comprising a VH comprising the amino acid sequence set forth in SEQ ID NO: 262, wherein the VH when paired with a VL comprising the amino acid sequence set forth in SEQ ID NO: 267 binds to SARS-CoV-2;
(e) a polynucleotide encoding an immunoglobulin light chain or a fragment thereof comprising a VL comprising the amino acid sequence set forth in SEQ ID NO: 267, wherein the VL when paired with a VH comprising the amino acid sequence set forth in SEQ ID NO: 262 binds to SARS-CoV-2;
(f) a polynucleotide encoding an immunoglobulin heavy chain or a fragment thereof comprising a VH comprising the amino acid sequence set forth in SEQ ID NO: 262 and an immunoglobulin light chain or a fragment thereof comprising a VL comprising the amino acid sequence set forth in SEQ ID NO: 267;
(g) a polynucleotide as in any one of (a)-(f), wherein a CDR comprises one or more, preferably no more than two amino acid substitution and/or the variable region sequence is at least 90% identical to SEQ ID NO: 262 or SEQ ID NO: 267.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 273, 274, and 275, respectively, and wherein the VH when paired with a light chain variable region (VL) comprising the amino acid sequence set forth in SEQ ID NO: 277 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 278, 279, and 280, respectively, and wherein the VL when paired with a VH comprising the amino acid sequence set forth in SEQ ID NO: 272 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 273, 274, and 275, respectively; and
   (ii) an immunoglobulin light chain or a fragment thereof comprising a VL comprising CDRs 1, 2, and 3 with the amino acid sequences set forth in SEQ ID NOs: 278, 279, and 280, respectively;
(d) a polynucleotide encoding an immunoglobulin heavy chain or a fragment thereof comprising a VH comprising the amino acid sequence set forth in SEQ ID NO: 272, wherein the VH when paired with a VL comprising the amino acid sequence set forth in SEQ ID NO: 277 binds to SARS-CoV-2;
(e) a polynucleotide encoding an immunoglobulin light chain or a fragment thereof comprising a VL comprising the amino acid sequence set forth in SEQ ID NO: 277, wherein the VL when paired with a VH comprising the amino acid sequence set forth in SEQ ID NO: 272 binds to SARS-CoV-2;
(f) a polynucleotide encoding an immunoglobulin heavy chain or a fragment thereof comprising a VH comprising the amino acid sequence set forth in SEQ ID NO: 272 and an immunoglobulin light chain or a fragment thereof comprising a VL comprising the amino acid sequence set forth in SEQ ID NO: 277;
(g) a polynucleotide as in any one of (a)-(f), wherein a CDR comprises one or more, preferably no more than two amino acid substitution and/or the variable region sequence is at least 90% identical to SEQ ID NO: 272 or SEQ ID NO: 277.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 283, 284, and 285, respectively, and wherein the VH when paired with a light chain variable region (VL) comprising the amino acid sequence set forth in SEQ ID NO: 287 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 288, 289, and 290, respectively, and wherein the VL when paired with a VH comprising the amino acid sequence set forth in SEQ ID NO: 282 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 283, 284, and 285, respectively; and
   (ii) an immunoglobulin light chain or a fragment thereof comprising a VL comprising CDRs 1, 2, and 3 with the amino acid sequences set forth in SEQ ID NOs: 288, 289, and 290, respectively;
(d) a polynucleotide encoding an immunoglobulin heavy chain or a fragment thereof comprising a VH comprising the amino acid sequence set forth in SEQ ID NO: 282, wherein the VH when paired with a VL comprising the amino acid sequence set forth in SEQ ID NO: 287 binds to SARS-CoV-2;
(e) a polynucleotide encoding an immunoglobulin light chain or a fragment thereof comprising a VL comprising the amino acid sequence set forth in SEQ ID NO: 287, wherein the VL when paired with a VH comprising the amino acid sequence set forth in SEQ ID NO: 282 binds to SARS-CoV-2;
(f) a polynucleotide encoding an immunoglobulin heavy chain or a fragment thereof comprising a VH comprising the amino acid sequence set forth in SEQ ID NO: 282 and an immunoglobulin light chain or a fragment thereof comprising a VL comprising the amino acid sequence set forth in SEQ ID NO: 287;
(g) a polynucleotide as in any one of (a)-(f), wherein a CDR comprises one or more, preferably no more than two amino acid substitution and/or the variable region sequence is at least 90% identical to SEQ ID NO: 282 or SEQ ID NO: 287.

The present invention further relates to a polynucleotide linked to a heterologous nucleic acid, wherein the polynucleotide is selected from the group consisting of:
(a) a polynucleotide encoding an immunoglobulin heavy chain or a fragment thereof comprising a heavy chain variable region (VH) comprising CDRs 1, 2, and 3 with the amino acid sequences set forth in SEQ ID NOs: 293, 294, and 295, respectively, and wherein the VH when paired with a light chain variable region (VL) comprising the amino acid sequence set forth in SEQ ID NO: 297 binds to SARS-CoV-2;
(b) a polynucleotide encoding an immunoglobulin light chain or a fragment thereof comprising a VL comprising CDRs 1, 2, and 3 with the amino acid sequences set forth in SEQ ID NOs: 298, 299, and 300, respectively, and wherein the VL when paired with a VH comprising the amino acid sequence set forth in SEQ ID NO: 292 binds to SARS-CoV-2;
(c) a polynucleotide encoding
   (i) an immunoglobulin heavy chain or a fragment thereof comprising a VH comprising CDRs 1, 2, and 3 with the amino acid sequences set forth in SEQ ID NOs: 293, 294, and 295, respectively; and
   (ii) an immunoglobulin light chain or a fragment thereof comprising a VL comprising CDRs 1, 2, and 3 with the amino acid sequences set forth in SEQ ID NOs: 298, 299, and 300, respectively;
(d) a polynucleotide encoding an immunoglobulin heavy chain or a fragment thereof comprising a VH comprising the amino acid sequence set forth in SEQ ID NO: 292, wherein the VH when paired with a VL comprising the amino acid sequence set forth in SEQ ID NO: 297 binds to SARS-CoV-2;
(e) a polynucleotide encoding an immunoglobulin light chain or a fragment thereof comprising a VL comprising the amino acid sequence set forth in SEQ ID NO: 297, wherein the VL when paired with a VH comprising the amino acid sequence set forth in SEQ ID NO: 292 binds to SARS-CoV-2;
(f) a polynucleotide encoding an immunoglobulin heavy chain or a fragment thereof comprising a VH comprising the amino acid sequence set forth in SEQ ID NO: 292 and an immunoglobulin light chain or a fragment thereof comprising a VL comprising the amino acid sequence set forth in SEQ ID NO: 297;
(g) a polynucleotide as in any one of (a)-(f), wherein a CDR comprises one or more, preferably no more than two amino acid substitution and/or the variable region sequence is at least 90% identical to SEQ ID NO: 292 or SEQ ID NO: 297.

Furthermore, the present invention relates to a vector and vectors comprising one or more of the above-described polynucleotides, preferably wherein the vector is an expression vector and the one or more polynucleotide(s) are operably linked to expression control sequences.

The polynucleotides may be produced and, if desired manipulated using methods well known in the art for the manipulation of nucleotide sequences, e.g., recombinant DNA techniques, site directed mutagenesis, PCR, etc. (see, for example, the techniques described in Molecular Cloning: A Laboratory Manual (Fourth Edition): Three-volume set; Green and Sambrook (2012) ISBN 10: 1936113422/ISBN 13: 9781936113422 Cold Spring Harbor Laboratory Press; update (2014) ISBN 978-1-936113-42-2 and Ausubel et al., eds., Current Protocols in Molecular Biology, John Wiley & Sons, NY (1998) and updates, which are both incorporated by reference herein in their entireties), to generate antibodies having a different amino acid sequence, for example to create amino acid substitutions, deletions, and/or insertions.

As mentioned above, the polynucleotide(s) of the present invention include RNA and may be used for translation in cells for therapeutics. Thus, the polynucleotide(s), in particular RNA(s) of the present invention can be used for generating the antibodies of the present invention in target cells. Various approaches for the production of suitable RNA are known to the person skilled in the art and are commercially available, e.g., kits for in vitro transcription, capping of RNA and for making poly(A)-tailed mRNA for translation in cells. In WO 2008/083949 A2 antibody-coding non-modified and modified RNA for expression of the corresponding antibody are described, pharmaceutical compositions comprising such RNA for the treatment of virus diseases as well as transcription methods and methods for expressing the antibody. In WO 2009/127230 A1 modified (m)RNA suitable for suppressing and/or avoiding an innate immunostimulatory response is described. Furthermore, a technology used by CELLSCRIPT™ has been developed, wherein the RNA contains pseudouridine (Ψ) and/or 5-methylcytidine (m5C) in place of the corresponding U or C canonical nucleosides. Such RNA has been shown to be less immunogenic and is translated into protein at much higher levels than the corresponding mRNA that does not contain modified nucleosides. The corresponding technology is described e.g. in Karikó et al., Immunity 23 (2005), 165-175, Karikó et al., Molecular Therapy 16 (2008), 1833-1840 and Anderson et al., Nucleic Acids Res 38 (2010), 5884-5892. Furthermore, EP 1 604 688 A1 describes stabilized and translation optimized mRNA having an enhanced G/C-content and optimized codon usage. Further approaches for the modification of RNA are described for example in Kormann et al., Nature Biotechnology 29 (2011), 154-157 and WO 2007/024708 A2.

Thus, in one embodiment the polynucleotide(s) of the present invention is/are RNA which can mRNA or derived thereof either unmodified or modified as described above and suitable for translation into the corresponding antibody.

Once a polynucleotide encoding an antibody molecule or a heavy or light chain of an antibody, or portion thereof (preferably containing the heavy or light chain variable domain), the vector for the production of the antibody molecule may be produced by recombinant DNA technology using techniques well known in the art. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operable linked to a promoter. Such vectors may include the nucleotide sequence encoding the constant region of the antibody molecule (see, e.g., international applications WO 86/05807 and WO 89/01036; and U.S. Pat. No. 5,122,464) and the variable domain of the antibody may be cloned into such a vector for expression of the entire heavy or light chain.

The term “vector” or “expression vector” is used herein to mean vectors used in accordance with the present invention as a vehicle for introducing into and expressing a desired gene in a host cell. In general, vectors compatible with the instant invention will comprise a selection marker, appropriate restriction sites to facilitate cloning of the desired gene and the ability to enter and/or replicate in eukaryotic or prokaryotic cells. The marker may provide for prototrophy to an auxotrophic host, biocide resistance (e.g., antibiotics), or resistance to heavy metals such as copper. The selectable marker gene can either be directly linked to the DNA sequences to be expressed, or introduced into the same cell by co-transformation. Additional elements may also be needed for optimal synthesis of mRNA. These elements may include signal sequences, splice signals, as well as transcriptional promoters, enhancers, and termination signals. For the expression of double-chained antibodies, a single vector or vectors encoding both the heavy and light chains may be co-expressed in the host cell for expression of the entire immunoglobulin molecule, as detailed below. A review on vector-related stratagems for enhanced monoclonal antibody production in mammalian cells is provided for example in Gupta et al., Biotechnology Advances 37 (2019), https://doi.org/10.1016/j.biotechadv.2019.107415.

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain is advantageously placed before the heavy chain to avoid an excess of toxic free heavy chain; see Proudfoot, Nature 322 (1986), 52; Kohler, Proc. Natl. Acad. Sci. USA 77 (1980), 2197. The coding sequences for the heavy and light chains may comprise cDNA or genomic DNA. The expression vector(s) is (are) transferred to a host cell by conventional techniques and the transfected cells are then cultured by conventional techniques to produce an antibody for use in the methods described herein. Accordingly, the present invention also relates to host cells comprising one or more polynucleotides or a vector or vectors of the present invention.

As used herein, “host cells” refers to cells which harbor vectors constructed using recombinant DNA techniques and encoding at least one heterologous gene. In descriptions of processes for isolation of antibodies from recombinant hosts, the terms “cell” and “cell culture” are used interchangeably to denote the source of antibody unless it is clearly specified otherwise. In other words, recovery of polypeptide from the “cells” may mean either from spun down whole cells, or from the cell culture containing both the medium and the suspended cells.

Antibodies used for laboratory research/diagnosis may be expressed in any suitable host, e.g. in mammalian cells, bacterial cells, yeasts, plant cells or insect cells. However, currently almost all therapeutic antibodies are still produced in mammalian cell lines in order to reduce the risk of immunogenicity due to altered, non-human glycosylation patterns. However, recent developments of glycosylation-engineered yeast, insect cell lines, and transgenic plants are promising to obtain antibodies with “human-like” post-translational modifications. Furthermore, smaller antibody fragments including bispecific antibodies without any glycosylation are successfully produced in bacteria and have advanced to clinical testing. The first therapeutic antibody products from a non-mammalian source can be expected in coming next years. A review on current antibody production systems that can be applied for preparing the human-derived recombinant anti-SARS-CoV-2 antibody or SARS-CoV-2-binding fragment, synthetic derivative, or biotechnological derivative thereof of the present invention including their usability for different applications is given in Frenzel et al., Front Immunol. 4 (2013), 217, published online on Jul. 29, 2013 doi: 10.3389/fimmu.2013.00217 and transient expression of human antibodies in mammalian cells is described by Vazquez-Lombardi et al., Nature protocols 13 (2018), 99-117; and Hunter et al., Optimization of protein expression in mammalian cells. Current Protocols in Protein Science 95 (2019), e77. doi: 10.1002/cpps.77. Once an antibody molecule of the invention has been recombinantly expressed, the whole antibodies, their dimers, individual light and heavy chains, or other immunoglobulin forms of the present invention can be purified according to standard procedures of the art, including for example, by chromatography (e.g., ion exchange, affinity, particularly by affinity for the specific antigen after Protein A, and sizing column chromatography), centrifugation, differential solubility, e.g. ammonium sulfate precipitation, or by any other standard technique for the purification of proteins; see, e.g., Scopes, “Protein Purification”, Springer Verlag, N.Y. (1982) and Antibodies A Laboratory Manual 2nd edition, 2014 by Cold Spring Harbor Laboratory Press, Cold Spring Harbor, N.Y., USA. Thus, the present invention also relates to a method for preparing an anti-SARS-CoV-2 antibody and/or fragments thereof or immunoglobulin chain(s) thereof, said method comprising:
(a) culturing the host cell as defined hereinabove, which cell comprised the polynucleotide(s) or vector(s) as defined hereinbefore under conditions allowing for expression of the anti-SARS-CoV-2 antibody, SARS-CoV-2 binding fragment or immunoglobulin chain(s) thereof, and
(b) isolating the anti-SARS-CoV-2 antibody, SARS-CoV-2 binding fragment or immunoglobulin chain(s) thereof from the culture.

Alternatively, the anti-SARS-CoV-2 antibody and/or fragments thereof or immunoglobulin chain(s) thereof can be produced by expressing a polynucleotide encoding the antibody or antigen-binding fragment thereof of the antibody or antigen-binding fragment thereof as defined hereinbefore within a cell or cell-free expression system. Cell-free expression systems are known to the person skilled in the art and can be employed within the scope of the present invention. For example, successful synthesis of different antibody formats, including single-chain variable fragments (scFvs), Fab fragments, as well as complete IgGs, has already been shown in E. coli, Sf21, reticulocyte, wheat germ, and CHO cell-free systems; see review of Dondapati et al., BioDrugs 34 (2020), 327-348 as well as the references cited therein. Furthermore, the present invention also relates to the anti-SARS-CoV-2 antibody, SARS-CoV-2-binding fragment and immunoglobulin chain(s) thereof encoded by a polynucleotide as defined hereinabove and/or obtainable by the method for their recombinant production mentioned above.

The present invention also relates to a method of diagnosing an infection with SARS-CoV-2, i.e. COVID-19 or another SARS-CoV-2 induced disease in a subject, the method comprising determining the presence of SARS-CoV-2 or SARS-CoV-2 protein fragments, in particular the S protein in a sample of a subject to be diagnosed via contacting the sample with the composition of the present invention or with at least one antibody of the composition of present invention and with the antibody of the present invention, respectively under conditions enabling the formation of antibody-antigen complexes. The level of such complexes is then determined by methods known in the art, wherein a level significantly higher than that formed in a control sample indicates the disease in the tested individual. Thus, the present invention relates to an in vitro immunoassay comprising the antibody or antigen-binding fragment thereof of the invention.

In case, SARS-CoV-2 or the corresponding virus proteins are present in the sample, the antibody or antibodies bind to those proteins and can be detected afterwards. Preferably those antibodies are used for diagnostic approaches which specifically bind to SARS-CoV-2, but which do not substantially bind to SARS-CoV and MERS-CoV, i.e. which bind to the RBD of SARS-CoV with an EC₅₀ which is at least one or two order of magnitudes higher, i.e. 10 times, preferably 15 times, more preferably 20 times higher or 100 times, preferably 200 times, more preferably 300 times, more preferably 400 times, more preferably 500 times, more preferably 600 times, more preferably 700 times and even more preferably 800 times higher than the EC₅₀ for its binding to the RBD of SARS-CoV-2 or which do not substantially bind to the RBD of SARS-CoV. For example, the following antibodies are preferably used: NI-607.274_B7, NI-607.274_E5, NI-607.275_C5, NI-607.426_E2, NI-607.426_D4, NI-607.426_F11, NI-607.428_B9. NI-607.429_B9. NI-607.5291B9. NI-607.531_C8. NI-607.5311D8, NI-607.531_E7, NI-607.532_C11, NI-607.532_D3, NI-607.532_D4, NI-607.532_F9, NI-607.791_B10, NI-607.820_B6 and/or NI-607.820_B7.

The sample to be analyzed may be any body fluid suspected to contain SARS-CoV-2, for example a blood sample, a plasma sample, a serum sample, a lymph sample or any other body fluid sample, such as a saliva, CSF or a urine sample. In a preferred embodiment, the sample to be analyzed is respiratory sample material.

The subject to be diagnosed may be asymptomatic or preclinical for the disease.

The level of SARS-CoV-2 may be assessed by any suitable method known in the art comprising, e.g., Western blot, immunoprecipitation, enzyme-linked immunosorbent assay (ELISA), radioimmunoassay (RIA), or fluorescent activated cell sorting (FACS).

Furthermore, the anti-SARS-CoV-2 antibody or SARS-CoV-2-binding fragment thereof can be used for in vivo imaging of SARS-CoV-2. Thus, in one embodiment, said in vivo imaging of SARS-CoV-2 comprises positron emission tomography (PET), single photon emission tomography (SPECT), near infrared (NIR) optical imaging or magnetic resonance imaging (MRI). A review of the basic features of radionuclide imaging and the characteristics of ideal tracer molecules, and how antibodies can be evaluated for their suitability as virus-specific imaging probes is provided by, e.g., Bray et al., Antiviral Research 88 (2010), 129-142.

In certain embodiments, the antibody polypeptide comprises an amino acid sequence or one or more moieties not normally associated with an antibody. Thus, the present invention further encompasses antibodies, or antigen-binding fragments, variants, or derivatives thereof of the invention conjugated to a diagnostic or therapeutic agent. The antibodies can be used diagnostically to, for example, demonstrate presence of an SARS-CoV-2 infection, to monitor the progression of infection with SARS-CoV-2, or the response to a treatment of COVID-19 disease in a subject e.g., determine the efficacy of a given treatment and/or prevention regimen. Detection can be facilitated by coupling the antibody, or antigen-binding fragment, variant, or derivative thereof to a detectable substance. For example, the antibody or SARS-CoV-2-binding fragment thereof such a single-chain Fv antibody fragment of the invention may comprise a flexible linker sequence, or may be modified to add a functional moiety or detectable label (e.g., PEG, a drug, a toxin, or a label such as a fluorescent, (chemo/bio)luminescent, radioactive, enzyme, nuclear magnetic, heavy metal, a tag, a flag and the like); see, e.g., Antibodies A Laboratory Manual 2nd edition, 2014 by Cold Spring Harbor Laboratory Press, Cold Spring Harbor, N.Y., USA for general techniques; Dean and Palmer, Nat. Chem. Biol. 10 (2014), 512-523, for advances in fluorescence labeling strategies for dynamic cellular imaging; and Falck and Müller, Antibodies 7 (2018), 4; doi:10.3390/antib7010004 for enzyme-based labeling strategies for antibody-drug conjugates and antibody mimetics.

Furthermore, the anti-SARS-CoV-2 antibody or SARS-CoV-2-binding fragments of the present invention can comprise a brain targeting entity and/or is contained in or conjugated to a vehicle such as an exosome or nanoparticle for delivery to the brain, for example for preventing clotting but also in context with amyloidosis.

In the vast majority of cases, COVID-19 is a respiratory infection that causes fever, aches, tiredness, sore throat, cough and, in more severe cases, shortness of breath and respiratory distress. However, it has already been shown that COVID-19 can also infect cells outside of the respiratory tract and cause a wide range of symptoms from gastrointestinal disease (diarrhoea and nausea) to heart damage and blood clotting disorders. Recently, it has been observed that blood clots are a frequent complication of COVID-19 and studies from the Netherlands and France suggest that clots arise in 20-30% of critically ill COVID-19 patients. However, it is not clear why this clotting occurs and one hypothesis is that SARS-CoV-2 is directly attacking the endothelial cells that line the blood vessels. Endothelial cells harbor the same ACE2 receptor that the virus uses to enter lung cells. And there is evidence that endothelial cells can become infected: researchers from the University Hospital Zurich in Switzerland and Brigham and Women's Hospital in Boston, Mass., observed SARS-CoV-2 in endothelial cells inside kidney tissue. Viral infection can damage these cells, prompting them to churn out proteins that trigger the process of blood clotting. Blood thinners don't reliably prevent clotting in people with COVID-19, and young people are dying of strokes caused by the blockages in the brain; see Willyard, Nature 581 (2020), 250 “Coronavirus blood-clot mystery intensifies”. Several recent studies have further identified the presence of neurological symptoms in COVID-19 cases and SARS-CoV-2 may cause neurological disorders by directly infecting the brain. Cells in the human brain also express the ACE2 protein on their surface. Furthermore, the infection of endothelial cells may allow the virus to pass from the respiratory tract to the blood and then across the blood-brain barrier into the brain. Once in the brain, replication of the virus may cause neurological disorders.

Furthermore, in the context of Alzheimer's disease research, it is hypnotized that virus infections might trigger amyloidosis via upregulation of many genes involved in amyloidosis.

As it is well known in the art, the blood-brain barrier (BBB) restricts drug efficacy for central nervous system (CNS) diseases. For example, monoclonal antibodies do not cross the BBB efficiently, reaching a maximum of 0.11% at 1 hour after injection (Banks et al. (2002), Peptides 23, 2223-2226). The BBB is a specialized structural, physiological and biochemical barrier and serves as the first interface between the changeable environment of blood and the extracellular fluid in the CNS. The BBB regulates the homeostasis of the nervous system by strictly controlling the movement of small molecules or macromolecules from the blood to the brain. It only permits selective transport of molecules that are essential for brain function. In detail, more than 98% of small molecule drugs and almost 100% of large molecule drugs are precluded from drug delivery to brain (Redzic (2011) Fluids Barriers CNS 8, 3; Pardridge (2005) NeuroRx, 2, 3-14). Thus, the polypeptide and the antibody, antigen-binding fragment thereof, variant or derivative thereof, respectively may be modified in order to be able to penetrate the BBB.

For example, said antibodies and binding fragments can be fused to cell-penetrating peptides (CPPs), which qualify as brain targeting entity and which are usually short cationic and/or amphipathic peptides that have the ability to transport the associated molecular cargo (e.g., peptides, proteins, antibodies, etc.) across cellular membranes. However, also anionic CCPs have been reported. Examples are given in Sharma et al. (2016) Int. J. Mol. Sci. 17, 806 and instructions how to fuse an antibody with a CPP are for example provided in Gaston et al. (2019) Sci. Rep. 9, 18688 doi:10.1038/s41598-019-55091-0. Furthermore, polyamine modification has been shown to dramatically increase the penetration of i.a. antibodies across the BBB (Poduslo and Curran (1996) J. Neurochem. 66, 1599-1609). The most investigated method to deliver macromolecules into the brain is via receptor-mediated transcytosis (RMT) and the main RMT receptors that have been studied are the transferrin receptor (TfR) and insulin receptor (IR). Thus, RMT receptors are also brain targeting entities. For example, bispecific antibodies have emerged as promising scaffolds to deliver therapeutic antibodies to the brain via engineering the antibody to incorporate one arm with specificity against a BBB RMT receptor, which drives their transmission across the BBB, and the other arm against a CNS therapeutic agent. Essentially, bispecific antibodies can be generated by fusion of antibody fragments such as Fabs, scFv or single domain antibodies into the N- or C-terminal of a convention IgG molecule or by heterodimerization strategies such as the “knobs-into-holes” technology developed by Genentech; see for details Neves et al. (2016) Trends Biotech. 34, 36-48.

Thus, the anti-SARS-CoV-2 antibody of the present invention can be a bispecific antibody binding to SARS-CoV-2 and to a BBB RMT receptor. Alternatively, since as mentioned and demonstrated in the Examples highly potent antibodies have been identified which bind to different locations on the SARS-CoV-2 RBD, bispecific antibodies can be generated which combine the two specificities, for example of any two antibodies which are shown in Example 6 to not compete with each other for binding the SARS-CoV-2 RBD peptide, e.g. antibody NI-607.529_B9 and NI-607.531_C8 or antibody NI-607.649_B11 and NI-607.531_C8. The generation of different therapeutic bispecific antibody formats including asymmetric heterodimeric monovalent 1+1 bispecific antibodies and asymmetric heterodimeric bispecific antibodies with 2+1 valency in combination with approaches enabling Fc-hetermodimerization like knob-into-hole technology as well as the generation of tetravalent symmetric bispecific antibodies with 2+2 valency, also known as Tandem-Fab based IgG antibodies using CrossMab technology is described; see, e.g., Klein et al., Methods 154 (2019), 21-31 and other publications in this Volume 154. Accordingly, rather than a cocktail of monospecific antibodies, alternatively or in addition bispecific antibodies may be used, wherein at least one binding domain is derived from an antibody of the present invention.

In another approach, lipid nanoparticles/nanoexosomes can be used, for example to deliver the antibodies or binding fragments of the present invention across the BBB. For example, dually decorated nanoliposomes with an anti-SARS-CoV-2 monoclonal antibody and an anti-RMT antibody, e.g. anti-TfR monoclonal antibody using biotin streptavidin conjugation can be used for improved delivery across the blood brain barrier. This principle is outlined in Markoutsa et al. (2012) Eur. J. Pharm. Biopharm. 81, 49-56) with an anti-AP antibody instead of an anti-SARS-CoV-2 antibody.

Furthermore, as summarized in Tosi et al. (2013) (Curr. Med. Chem. 20, 2212-25), biodegradable nanoparticles formulated from poly(D,L-lactide-co-glycolide) (PLGA) have been extensively investigated for sustained and targeted delivery of different agents, including antibodies across the BBB. Thus, the antibodies and binding fragments of the present invention are conjugated to nanoparticles and nanoexosomes, respectively.

Accordingly, in one embodiment, the anti-SARS-CoV-2 antibody or SARS-CoV-2-binding fragment thereof of the present invention is capable to penetrate the BBB.

An antibody polypeptide of the invention may comprise, consist essentially of, or consist of a fusion protein. Fusion proteins are chimeric molecules which comprise, for example, an immunoglobulin SARS-CoV-2-binding domain with at least one target binding site, and at least one heterologous portion, i.e. a portion with which it is not naturally linked in nature. The amino acid sequences may normally exist in separate proteins that are brought together in the fusion polypeptide or they may normally exist in the same protein but are placed in a new arrangement in the fusion polypeptide. Fusion proteins may be created, for example, by chemical synthesis, or by creating and translating a polynucleotide in which the peptide regions are encoded in the desired relationship.

The term “heterologous” as applied to a polynucleotide or a polypeptide, means that the polynucleotide or polypeptide is derived from a distinct entity from that of the rest of the entity to which it is being compared. For instance, as used herein, a “heterologous polypeptide” to be fused to an antibody, or an antigen-binding fragment, variant, or analog thereof is derived from a non-immunoglobulin polypeptide of the same species, or an immunoglobulin or non-immunoglobulin polypeptide of a different species.

The human-derived recombinant anti-SARS-CoV-2 antibody or SARS-CoV-2-binding fragment, synthetic derivative, or biotechnological derivative thereof, optionally as fusion protein and/or labeled as described hereinbefore is then provided for various applications in accordance with standard techniques known in the art; see, e.g., Antibodies A Laboratory Manual 2nd edition, 2014 by Cold Spring Harbor Laboratory Press, Cold Spring Harbor, N.Y., USA. Current advancements in therapeutic antibody design, manufacture, and formulation are described in Sifniotis et al., Antibodies 2019, 8(2), 36; https://doi.org/10.3390/antib8020036, wherein also developments in computational approaches for the strategic design of antibodies with modulated functions are discussed.

The present invention relates to compositions comprising the afore-mentioned SARS-CoV-2-antibody or SARS-CoV-2-binding fragment, variant or biotechnological derivative thereof, or the polynucleotide(s), vector(s) or cell of the invention as defined hereinbefore. In one embodiment, the composition of the present invention is a pharmaceutical composition and further comprises a pharmaceutically acceptable carrier. In one embodiment, the pharmaceutical composition comprises one or more of those antibodies that bind with the same order of magnitude to the RBD of SARS-CoV-2 and to the RBD of SARS-CoV which include, but are not limited to antibody NI-607.427_C5, NI-607.529_G4, NI-607.531_E3, NI-607.532_B6, NI-607.532_D8, NI-607.532_F3, NI-607.649_B11, and NI-607.649_G7 or the corresponding polynucleotide(s), vector(s) or cell.

In another embodiment, the composition comprises one or more of those antibodies that bind with high affinity to the RBD of SARS-CoV-2 and SARS-CoV in the low nanomolar range, preferably between 0.5 nM and 80 nM, preferably between 0.5 nM and 40 nM or between 1 nM and 70 nM, more preferably between 1 nM and 20 nM or between 2 nM and 61 nM as determined by iQue, optionally wherein the antibody binds to the RBD of SARS-CoV with an EC₅₀ which is two- to three-fold higher than its EC₅₀ for binding to the RBD of SARS-CoV-2 polynucleotide(s), vector(s) or cell. Such antibodies include, but are not limited to antibody NI-607.529_B9 and antibody NI-607.820_B6.

In another embodiment, the compositions comprises one or more of those antibodies that bind to the RBD of SARS-CoV with an EC₅₀ which is at least one or two order of magnitudes higher than the EC₅₀ for its binding to the RBD of SARS-CoV-2, i.e. 10 times, preferably 15 times, more preferably 20 times higher or 100 times, preferably 200 times, more preferably 300 times, more preferably 400 times, more preferably 500 times, more preferably 600 times, more preferably 700 times and even more preferably 800 times higher than the EC₅₀ for its binding to the RBD of SARS-CoV-2 or wherein the antibody does not substantially bind to the RBD of SARS-CoV polynucleotide(s), vector(s) or cell. Those antibodies include, but are not limited to antibody NI-607.274_B7, NI-607.274_E5, NI-607.275_C5, NI-607.426_E2, NI-607.426_D4, NI-607.426_F11, NI-607.428_B9, NI-607.429_B9, NI-607.529_B9, NI-607.531_C8, NI-607.531_D8, NI-607.531_E7, NI-607.532_C11, NI-607.532_D3, NI-607.532_D4, NI-607.532_F9, NI-607.791_B10, NI-607.820_B6 and NI-607.820_B7.

However, the composition may comprise any of the above defined antibodies and any possible combination of the antibodies polynucleotide(s), vector(s) or cell.

The present invention also provides the pharmaceutical and diagnostic composition, respectively, in form of a pack or kit comprising one or more containers filled with one or more of the above described ingredients, e.g., anti-SARS-CoV-2 antibody, SARS-CoV-2-binding fragment, biotechnological derivative or variant thereof, polynucleotide, preferably RNA, vector or cell of the present invention. Associated with such container(s) can be a notice in the form prescribed by a governmental agency regulating the manufacture, use or sale of pharmaceuticals or biological products, which notice reflects approval by the agency of manufacture, use or sale for human administration. In addition, or alternatively the kit comprises reagents and/or instructions for use in appropriate immuno-based diagnostic assays. The composition, e.g. kit of the present invention is of course particularly suitable for the risk assessment, diagnosis, prevention and treatment of a disease or disorder which is accompanied with the presence of SARS-CoV-2, and in particular applicable for the treatment of disorders generally associated with SARS-CoV-2 as discussed herein above.

The pharmaceutical compositions of the present invention can be formulated according to methods well known in the art; see for example, Remington: The Science and Practice of Pharmacy (2000) by the University of Sciences in Philadelphia, ISBN 0-683-306472. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well-known conventional methods. For example, in international applications WO 2020/089342 A1, WO 2019/207060 A1 and WO 2018/232355 A1 lipid-based formulations and polymer-based formulations, respectively for efficient administration of RNA to a subject are described. The pharmaceutical compositions can be administered to the subject at a suitable dose. Administration of the suitable compositions may be effected by different ways, e.g., by intravenous, intraperitoneal, subcutaneous, intramuscular, intranasal, aerosol, topical or intradermal administration, spinal or brain delivery or inhalation (nasal or oral route). As just recently reported, tracking the ease with which SARS-CoV-2 infects various cell types in the respiratory tract a gradient of infectivity was found that decreases from the upper to the lower respiratory tract: the most easily infected cells are in the nasal cavity, and the least easily infected deep in the lungs; see Hou et al. Cell http://doi.org/dw2j; 2020. That gradient mapped neatly onto the distribution of cells that express ACE2, a protein that SARS-CoV-2 uses to enter cells. Furthermore, in the course of studies with influenza virus improved therapeutic protection was observed in animals treated with antibodies locally (intranasal or aerosol) compared with those treated systemically (intravenous or intraperitoneal); see Leyva-Grado et al. Antimicrob Agents Chemother 59 (2015), 4162-4172 and Tiwari et al., Nature Communications 9 (2018), 3999 developed a modular, synthetic mRNA-based approach to express neutralizing antibodies directly in the lung via aerosol, to prevent infections with respiratory syncytial virus. Furthermore, Johler et al., PLoS ONE 10 (2015), e0137504 describe that aerosolisation of cationic in vitro transcribed mRNA complexes constitute a potentially powerful means to transfect cells in the lung.

Therefore, in a preferred embodiment, the antibody or antigen-binding fragment thereof of the present invention, preferably as IgG4 or the corresponding polynucleotide, preferably mRNA as well as composition or cocktail thereof is designed for local administration, preferably nasal or aerosol administration. Topical application of nebulized human IgG, IgA and IgAM exemplified with the lungs of rats and non-human primates is described in Vonarburg et al. Respiratory Research 20 (2019):99. https://doi.org/10.1186/s12931-019-1057-3. Carriers for the targeted delivery of aerosolized macromolecules for pulmonary pathologies are reviewed for example in Osman et al., Expert Opinion on Drug Delivery 15 (2018), 821-834.

The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently.

As mentioned above, SARS-CoV-2 does not only affect the respiratory tract, but also various organs in the human body, for example kidneys, liver, heart, brain, pancreas, adrenal glands, or lymphatic system. Secher et al., Front. Immunol. 10 (2019), 2760 reported that inhalation comprising the intranasal and oral respiratory routes, targets drugs into the respiratory tract and is used for locally—and systemically—acting drugs as it allows a straight delivery to the diseased organ and a portal to the blood circulation, considering the extensive alveolus-capillary interface.

Hence, the present invention also relates to a method of treating a disease or disorder associated with SARS-CoV-2 including COVID-19 and infection with SARS-CoV-2, which method comprises administering to a subject in need thereof a therapeutically effective amount of any one of the afore-described human-derived antibodies or corresponding polynucleotides of the instant invention. Preferably, the antibody or composition comprising one or more of the same or corresponding polynucleotide(s) is designed for topical mucosal and/or pulmonary delivery, preferably as aerosol; optionally together with a system for aerosol drug delivery such as nebulizer, for example the mesh nebulizer as described by Pritchard et al., Therapeutic Delivery 9 (2018), 121-136, the Idehaler® mesh nebulizer, a vibrating mesh nebulizers (eFlow® rapid Nebuliser System—PARI) or the Spray nozzle unit by MedSpray, a metered dose inhaler (MDI), dry powder inhalers (DPI), soft mist inhalers, or intratracheal nebulizing catheters. Aerosols might be administered with various delivery devices, for example with those as mentioned above, during mechanical ventilations (see Dhand, Respir. Care 62 (2017), 1343-1367) which might be particularly important for COVID-19 patients in an advanced disease state which require mechanical ventilation.

Several documents are cited throughout the text of this specification. The contents of all cited references (including literature references, issued patents, published patent applications as cited throughout this application including the background section and manufacturer's specifications, instructions, etc.) are hereby expressly incorporated by reference; however, there is no admission that any document cited is indeed prior art as to the present invention.

A more complete understanding can be obtained by reference to the following specific Examples which are provided herein for purposes of illustration only and are not intended to limit the scope of the invention.

### EXAMPLES

### Example 1: Isolation and Identification of Anti-SARS-CoV-2 Antibodies

Initially, 28 clinically interesting donors were recruited, i.e., male and female donors between 18 and 60 years old which have successfully recovered from COVID-19. Peripheral blood was drawn from these donors under appropriate informed consent. Plasma cells (PCs) and memory B cell cultures were isolated from freshly isolated PBMCs (Peripheral Blood Mononuclear Cells) and memory B cells were differentiated. Afterwards, the PCs and differentiated memory B cells were assessed for target specificity utilizing the Reverse Translational Medicine™ (RTM™) technology, a proprietary technology platform by Neurimmune AG originally described in the international application WO 2008/081008A1 but modified, further refined and specifically adapted to the RBD target, and in addition implementing a method similar as described in Zost et al., bioRxiv (2020), doi:10.1101/2020.05.12.091462. In particular, human-derived antibodies targeting the RBD of SARS-CoV-2 S glycoprotein have been cloned and identified visualized in FIG. S2b of Zost et al., bioRxiv (2020), doi:10.1101/2020.05.12.091462, wherein human B cells producing anti-SARS-CoV-2 antibodies are bound by RBD-coated beads and wherein the anti-SARS-CoV-2 antibodies are detected by secondary fluorescent antibodies. This approach has been further modified and refined in accordance with the RTM™ technology proprietary to Neurimmune AG.

High-throughput analysis was also performed to characterize the subclass of the native antibody; see Table IV.

TABLE IV
Characterization of the subclass of the
antibodies of the present invention.

| Antibody | Subclass |
|---|---|
| NI-607.274_B7 | IgG |
| NI-607.274_E5 | IgG |
| NI-607.275_C5 | IgG |
| NI-607.426_D4 | IgG |
| NI-607.426_E2 | IgG |
| NI-607.426_F11 | IgG |
| NI-607.427_C5 | IgG |
| NI-607.428_B9 | IgG |
| NI-607.429_B9 | IgG |
| NI-607.429_E4 | IgG |
| NI-607.529_B9 | IgG |
| NI-607.529_G4 | IgG |
| NI-607.531_C8 | IgG |
| NI-607.531_D8 | IgG |
| NI-607.532_B6 | IgG |
| NI-607.532_C11 | IgA |
| NI-607.532_C8 | IgA |
| NI-607.532_D3 | IgA |
| NI-607.532_D4 | IgA |
| NI-607.532_D8 | IgA |
| NI-607.532_F9 | IgG* |
| NI-607.649_B11 | IgA* |
| NI-607.531_E7 | IgA* |
| NI-607.532_F3 | IgA |
| NI-607.649_G7 | IgG |
| NI-607.761_B7 | IgG |
| NI-607.791_B10 | IgG |
| NI-607.531_E3 | IgA* |
| NI-607.820_B6 | IgG |
| NI-607.820_B7 | IgG |

*= presumably.

### Example 2: Determination of Antibody Sequence and Recombinant Expression

The amino acid sequences of the variable regions of the anti-SARS-CoV-2 antibodies were determined on the basis of their mRNA and cDNA sequences, respectively, obtained from human memory B cells and PCs; see above and Table II. Recombinant expression of complete human IgG1 antibodies with a human or mouse constant domain was performed substantially as described in the Examples of WO 2008/081008A1, e.g., as described in the Methods section at page 99 and 100. Similarly, IgG4 and IgG1 variant L234A, L235A, P329G (LALA-PG) are produced. The framework and complementarity determining regions (CDRs) were determined by comparison with reference antibody sequences following analysis principles as outlined in Dondelinger et al., Front. Immunol. 9 (2018), 1-15. Annotation and numbering of sequences was performed following the guidelines in the Chothia numbering scheme (Chothia et al., Nature 342 (1989), 877-883).

### Example 3: Binding Characteristics as Determined by ELISA

To determine the binding specificity and the half maximal effective concentration (EC₅₀) of recombinant human-derived SARS-CoV-2 antibodies NI-607.274_B7, NI-607.274_E5, NI-607.275_C5, NI-607.426_D4, NI-607.426_E2, NI-607.426_F11, NI-607.427_C5, NI-607.428_B9, NI-607.429_B9, NI-607.429_E4, NI-607.529_B9, NI-607.529_G4, NI-607.531_C8, NI-607.531_D8, NI-607.532_B6, NI-607.532_C11, NI-607.532_C8, NI-607.532_D3, NI-607.532_D4, NI-607.532_D8, NI-607.532_F9, NI-607.649_B11, NI-607.531_E7, NI-607.532_F3, NI-607.649_G7, NI-607.761_B7, NI-607.791_B10, NI-607.531_E3, NI-607.820_B6, and NI-607.820_B7 for binding the RBD of SARS-CoV-2 S an ELISA EC₅₀ analysis was performed.

In brief, direct ELISA was performed using 96-well microplates (Corning Incorporated, Corning, USA) coated with either SARS-CoV-2-S1 (RBD)-His protein (Trenzyme, Germany) or with BSA (Sigma-Aldrich, Buchs, Switzerland) at a concentration of 5 μg/ml in PBS for 2 h at room temperature with gentle shaking on the orbital shaker. Afterwards, plates were washed twice with 150 μl PBS-Tween20 (PBS-T). Non-specific binding sites were blocked for 1 h at room temperature with gentle shaking with 5% (w/v) BSA in PBS-T. Afterwards, plates were washed twice with 150 μl PBS-T. Antibodies NI-607.274_B7, NI-607.274_E5, NI-607.275_C5, NI-607.426_D4, NI-607.426_E2, NI-607.426_F11, NI-607.427_C5, NI-607.428_B9, NI-607.429_B9, NI-607.429_E4, NI-607.529_B9, NI-607.529_G4, NI-607.531_C8. NI-607.531_D8. NI-607.532_B6. NI-607.532_C11, NI-607.532_C8, NI-607.532_D3, NI-607.532_D4, NI-607.532_D8, NI-607.532_F9, NI-607.649_B11, NI-607.531_E7, NI-607.532_F3, NI-607.649_G7, NI-607.761_B7, NI-607.791_B10, NI-607.531_E3, NI-607.820_B6, and NI-607.820_B7 were diluted in PBS (23 serial dilutions from 400 nM stock) and incubated for 2 h at room temperature with gentle shaking on the orbital shaker, followed by two washing steps with PBS-T and incubation with a donkey anti-human IgG Fcγ-specific antibody conjugated with HRP (Jackson ImmunoResearch Laboratories, Inc., West Grove, USA) for 1 h at room temperature with gentle shaking. After four washing steps, binding was determined by measurement of HRP activity in a standard colorimetric assay. EC₅₀ values were estimated by non-linear regression using GraphPad Prism software (San Diego, USA).

The binding specificity and EC₅₀ of human-derived SARS-CoV-2-specific antibodies were determined by ELISA and are listed in Table I. Antibody NI-607.274_B7 specifically recognizes the RBD of SARS-CoV-2 S with an EC₅₀ of 21 pM. Antibody NI-607.274_E5 specifically recognizes the RBD of SARS-CoV-2 S with an EC₅₀ of 15 pM. Antibody NI-607.275_C5 specifically recognizes the RBD of SARS-CoV-2 S with an EC₅₀ of 20 pM. Antibody NI-607.426_D4 specifically recognizes the RBD of SARS-CoV-2 S with an EC₅₀ of 33 pM. Antibody NI-607.426_E2 specifically recognizes the RBD of SARS-CoV-2 S with an EC₅₀ of 30 pM. Antibody NI-607.426_F11 specifically recognizes the RBD of SARS-CoV-2 S with an EC₅₀ of 17 pM. Antibody NI-607.427_C5 specifically recognizes the RBD of SARS-CoV-2 S with an EC₅₀ of 14 pM. Antibody NI-607.428_B9 specifically recognizes the RBD of SARS-CoV-2 S with an EC₅₀ of 26 pM. Antibody NI-607.429_B9 specifically recognizes the RBD of SARS-CoV-2 S with an EC₅₀ of 14 pM. Antibody NI-607.429_E4 specifically recognizes the RBD of SARS-CoV-2 S with an EC₅₀ of 8.6 pM. Antibody NI-607.529_B9 specifically recognizes the RBD of SARS-CoV-2 S with an EC₅₀ of 34 pM. Antibody NI-607.529_G4 specifically recognizes the RBD of SARS-CoV-2 S with an EC₅₀ of 26 pM. Antibody NI-607.531_C8 specifically recognizes the RBD of SARS-CoV-2 S with an EC₅₀ of 11 pM. Antibodies NI-607.531_D8 and NI-607.532_B6 specifically recognize the RBD of SARS-CoV-2 S with an EC₅₀ of 4.5 pM. Antibody NI-607.532_C11 specifically recognizes the RBD of SARS-CoV-2 S with an EC₅₀ of 13 pM. Antibody NI-607.532_C8 specifically recognizes the RBD of SARS-CoV-2 S with an EC₅₀ of 9.9 to 17 pM. Antibody NI-607.532_D3 specifically recognizes the RBD of SARS-CoV-2 S with an EC₅₀ of 4.9 pM. Antibody NI-607.532_D4C5 specifically recognizes the RBD of SARS-CoV-2 S with an EC₅₀ of 5.5 pM. Antibody NI-607.532_D8 specifically recognizes the RBD of SARS-CoV-2 S with an EC₅₀ of 5.1 pM. Antibody NI-607.532_F9 specifically recognizes the RBD of SARS-CoV-2 S with an EC₅₀ of 2.5 pM. Antibody NI-607.649_B11 specifically recognizes the RBD of SARS-CoV-2 S with an EC₅₀ of 13 to 27 pM. Antibody NI-607.531_E7 specifically recognizes the RBD of SARS-CoV-2 S with an EC₅₀ of 27 pM. Antibody NI-607.532_F3 specifically recognizes the RBD of SARS-CoV-2 S with an EC₅₀ of 19 pM. Antibody NI-607.761_B7 specifically recognize the RBD of SARS-CoV-2 S with an EC₅₀ of 1 pM. Antibody NI-607.649_G7 specifically recognizes the RBD of SARS-CoV-2 S with an EC₅₀ of 24 pM. Antibody NI-607.791_B10 specifically recognizes the RBD of SARS-CoV-2 S with an EC₅₀ of 62 pM. Antibody NI-607.531_E3 specifically recognizes the RBD of SARS-CoV-2 S with an EC₅₀ of 20 pM. Antibody NI-607.820_B6 specifically recognizes the RBD of SARS-CoV-2 S with an EC₅₀ of 29 pM. Antibody NI-607.820_B7 specifically recognizes the RBD of SARS-CoV-2 S with an EC₅₀ of 82 pM.

Furthermore, the EC₅₀ values of constant domain switched antibodies have been analyzed. In particular, exemplarily results are shown in FIG. 1 for the IgG1 antibody NI-607.531_C8 (A) in comparison to the corresponding IgG4 S228P antibody (B) and for the IgG1 antibody NI-607.649_B11 (C) in comparison to the corresponding IgG4 S228P antibody (D). The results show that the EC₅₀ values do not substantially change and are still in the same order of magnitude. In particular, both IgG4 S228P antibodies showed an EC₅₀ of about 8.5 pM which is similar to the EC₅₀ values of their corresponding IgG1 antibodies, i.e. about 17 pM and 21 pM, respectively.

In conclusion, high-throughput immune repertoire analyses of donors successfully recovered from COVID-19 lead to the successful cloning and recombinant production of human monoclonal antibodies targeting SARS-CoV-2 with high affinity.

### Example 4: Binding Characteristics as Determined by iQue

The antibodies of the present invention were tested for target binding using flow cytometry on iQue Screener PLUS (IntelliCyt). To get an estimation of the affinity, the antibodies were serially diluted for the assay. Polystyrene particles were conjugated with the following targets by passive adsorption to validate target binding: SARS-CoV-2 Spike S1 RBD protein (His tag) (Trendzyme), 2019-nCov Spike protein (RBD His tag) (SinoBiologics), 2019-nCov Spike protein (S1 mFC tag), recombinant SARS-CoV Spike protein (RBD, His tag), MERS-CoV Spike protein fragment (RBD, aa 367-606, His tag), and 2019-nCoV Spike protein (Si+S2 ECD, His tag) (SinoBiologics), respectively. Specificity of the binding interaction was tested by including beads conjugated with a non-related protein.

Bead staining was performed as follows: 0.5% bead dilution of the to be tested 5% bead sample (120 μl 5% beads+1080 μl PBS) was prepared and 5 μL of 0.5% beads were added to the respective well of a v-bottom plate. Titrations started at 50 μg/ml. The titration of the control antibody D002 (monoclonal mouse SARS-CoV Spike Antibody (clone D002)—huFc part, SinoBiologics), of a control antibody directed to a non-related protein and an isotype control antibody started at 10 μg/mL.

1° antibody Staining

1.1×1° antibody concentration was prepared to stain beads with 5 μg/mL of the respective control antibody. 1:5 dilution series of antibodies was prepared, i.e. 100 μL antibody were mixed with 400 μL FACS-T buffer (PBS+2% FBS superior+1 mM EDTA+0.02% Tween20). 50 μL/well 1.1×1° antibody concentration was added to the respective beads and incubated for 45 min at room temperature. Afterwards, 50 μl FACS-T buffer were added to all wells and centrifugation was performed for 5 min at 450×g. The plate was flicked to remove supernatant and carefully tapped once on a clean tissue to prevent spill-over of residual drops to other wells. The beads were washed twice with 100 μl FACS-T buffer by repeating the last two steps (centrifugation and plate flicking), directly followed by incubation with the second antibody (Goat Anti-Human IgG (H+L) AF647 AffiniPure F(ab′)₂ Fragment—Jackson ImmunoResearch).

2^{nd} Antibody Staining

1:1000 second antibody mix was prepared in FACS-T buffer and beads were incubated with 50 μl of the antibody mix for 30 min at room temperature in the dark. Afterwards, 50 μl FACS-T buffer were added to all wells and centrifugation was performed for 5 min at 450×g. The plate was flicked to remove supernatant and carefully tapped once on a clean tissue to prevent spill-over of residual drops to other wells. The beads were washed twice with 100 μl FACS-T buffer by repeating the last two steps (centrifugation and plate flicking), followed by resuspension of the beads in 10 μl FACS-T buffer and direct continuation with iQue acquisition according to the user manual.

In particular, the following antibodies were tested: NI-607.274_B7, NI-607.274_E5, NI-607.275_C5, NI-607.426_D4, NI-607.426_E2, NI-607.426_F11, NI-607.427_C5, NI-607.428_B9, NI-607.429_B9, NI-607.429_E4, NI-607.529_B9, NI-607.529_G4, NI-607.531_C8, NI-607.531_D8, NI-607.532_B6, NI-607.532_C11, NI-607.532_C8, NI-607.532_D3, NI-607.532_D4, NI-607.532_D8, NI-607.532_F9, NI-607.649_B11, NI-607.531_E7, NI-607.532_F3, NI-607.649_G7, NI-607.761_B7, NI-607.791_B10, NI-607.531_E3, NI-607.820_B6, and NI-607.820_B7.

EC₅₀ values were estimated by non-linear regression using GraphPad Prism software (San Diego, USA).

As can be derived from Table I, all tested antibodies show a good EC₅₀ as also determined by ELISA; see Example 3. Furthermore, there are two groups of antibodies: those that specifically bind the RBD of SARS-CoV-2, but do not bind the RBD of SARS-CoV and those who bind both the RBD of SARS-CoV-2 and of SARS-CoV with high affinity; see also FIG. 2 for illustration. Furthermore, moderate to low binding to the RBD of MERS-CoV was observed for the tested antibodies, i.e. between 200 and 1000 nM EC₅₀ and no or only weak cross-reactivity to the non-related protein control was observed (data not shown).

### Example 5: Assessment of the Neutralization Capabilities of NI-607 Antibodies Via Competition ELISA

A competition ELISA was performed to screen for neutralizing anti-SARS-CoV-2 antibodies which disrupt the interaction between the viral RBD protein and the human ACE2 receptor, which mediates viral entry into host cells.

Evaluation of the neutralizing potential of anti-SARS-CoV-2 antibodies was based on a competition ELISA determining the IC₅₀ value. In brief, the competition ELISA was performed using 96-well half-area microplates (Corning Incorporated, Corning, USA) coated with ACE2 (Trenzyme, Germany) at a concentration of 2 μg/ml in PBS overnight at 4° C. Afterwards, plates were washed four times with 300 μl PBS+0.05% Tween 20 (PBS-T). Non-specific binding sites were blocked for 90 min at room temperature with 1% (w/v) BSA in PBS-T. While blocking, the anti-RBD antibody as positive control (40150-D002, SinoBiologics) and the antibodies to be tested were diluted to suitable concentrations and incubated for 1 h at room temperature in a non-binding plate. After blocking, the plates were washed four times with 300 μl PBS-T. Antibodies NI-607.274_B7, NI-607.274_E5, NI-607.275_C5, NI-607.426_D4, NI-607.426_E2, NI-607.426_F11, NI-607.427_C5, NI-607.428_B9, NI-607.429_B9, NI-607.429_E4, NI-607.529_B9, NI-607.529_G4, NI-607.531_C8, NI-607.531_D8, NI-607.532_B6, NI-607.532_C11, NI-607.532_C8, NI-607.532_D3, NI-607.532_D4, NI-607.532_D8, NI-607.532_F9, NI-607.649_B11, NI-607.531_E7, NI-607.532_F3, NI-607.649_G7, NI-607.761_B7, NI-607.791_B10, NI-607.531_E3, NI-607.820_B6, and NI-607.820_B7 as well as the anti-RBD antibody were added to the ACE2 coated plates and incubated for 1 h at room temperature with gentle shaking (450 rpm) on the orbital shaker, followed by four washing steps with PBS-T and incubation with a donkey anti-human IgG Fcγ-specific antibody conjugated with HRP (Jackson ImmunoResearch Laboratories, Inc., West Grove, USA) for 1 hour at room temperature with gentle shaking. After four washing steps, binding was determined by measurement of HRP activity in a standard colorimetric assay. IC₅₀ values were estimated by non-linear regression using GraphPad Prism software (San Diego, USA).

As can be derived from Table I, about half of the tested antibodies show IC₅₀ values in the picomolar range which are regarded as having the highest neutralization potency and can be expected to be most suitable for therapeutic approaches.

Furthermore, the IC₅₀ values of constant domain switched antibodies have been analyzed. In particular, IgG1 antibodies have been compared to IgG4 S228P antibodies and exemplarily results are shown in FIG. 3 for the antibodies NI-607.531_C8 and NI-607.649_B11. The results show that the IC₅₀ values between the two variants do not substantially differ and are in the same order of magnitude. In particular, the IgG4 S228P antibody NI-607.531_C8 showed an IC₅₀ of about 191 ng/mL (1.3 nM) and the corresponding IgG1 antibody an IC₅₀ of about 132 ng/mL (0.89 nM). The IgG4 antibody NI-607.649_B11 showed an IC₅₀ of about 47 ng/mL (0.31 nM) and the corresponding IgG1 antibody an IC₅₀ of about 52 ng/mL (0.35 nM). The IgG4 S228P antibody NI-607.427_C5 showed an IC₅₀ of about 2791 ng/mL (18.6 nM) and the corresponding IgG1 antibody an IC₅₀ of about 2152 ng/mL (14.3 nM). The IgG4 antibody NI-607.429_E4 showed an IC₅₀ of about 699 ng/mL (4.65 nM) and the corresponding IgG1 antibody an IC₅₀ of about 345 ng/mL (2.3 nM). The IgG4 S228P antibody NI-607.532_C11 showed an IC₅₀ of about 2317 ng/mL (15.4 nM) and the corresponding IgG1 antibody an IC₅₀ of about 1117 ng/mL (7.44 nM). The IgG4 antibody NI-607.532_D3 showed an IC₅₀ of about 457 ng/mL (3 nM) and the corresponding IgG1 antibody an IC₅₀ of about 457 ng/mL (3 nM).

The IgG4 S228P antibody NI-607.532_F9 showed an IC₅₀ of about 1092 ng/mL (7.3 nM) and the corresponding IgG1 antibody an IC₅₀ of about 668 ng/mL (4.4 nM).

### Example 6: Determination of Competing Antibodies

A cross competition assay was performed in which the competitive binding of antibody pairs to the SARS-CoV-2-S1 RBD peptide was characterized. For this approach, a classical sandwich format was used, involving immobilizing the first antibody onto the biosensor (AHC), followed by incubation with the antigen, and then by the second sandwiching antibody. Experiments conducted following application note from the device manufacturer (https://www.fortebio.com/sites/default/files/en/assets/app-note/cross-competition-or-epitope-binning-assays-on-octet-htx-system.pdf); see also FIG. 4 for illustration.

Initially, 1× kinetics buffer (KB) was prepared by diluting the 10× kinetics buffer (Fortebio, Calif., USA) which contains 0.1% BSA, 0.02% Tween20, and 0.05% sodium azide in PBS. This 1×KB was used as diluent for ligand and analyte as well as neutralization buffer. In the following, the sensors were hydrated in 1×KB for 10 min and a 10 mM glycine buffer in ddH₂O used as regeneration buffer was prepared. The assay was performed with antibodies at a concentration of 100 nM and the isotype control was used to block the AHC sensors. In particular, antibodies NI-607.429_E4, NI-607.427_C5, NI-607.429_B9, 607.426_F11, NI-607.531_C8, NI-607.532_C11, NI-607.532_D3, NI-607.532_F9, NI-607.529_B9, NI-607.649_B11, NI-607.532_F3, and NI-607.531_E7 were tested.

The data were analyzed using the Octet Data Analysis Software Version 8.2—Process epitope binning data and based on the observed nm shift, an epitope matrix was generated (see Table 5), wherein green (here shown in bold) indicates that the epitope is not shared and wherein red (here shown in underlined) represents a shared epitope. An arbitrary threshold of 50% signal increase is necessary to get a bold font (epitope not shared), underline font=same epitope.

TABLE V
Epitope matrix

| | | | | | | | | | | | |
|---|---|---|---|---|---|---|---|---|---|---|---|
| 429 E4 | 427 C5 | 426 F11 | 531 C8 | 532 C11 | 532 D3 | 532 F9 | 529 B9 | 649 B11 | | | |
| 427 C5 | 429 E4 | 429 B9 | 426 F11 | 531 C8 | 532 C11 | 532 D3 | 532 F9 | 529 B9 | 649 B11 | | |
| 429 B9 | 429 E4 | 427 C5 | 426 F11 | 531 C8 | 532 C11 | 532 D3 | 532 F9 | 529 B9 | 649 B11 | | |
| 426 F11 | 429 E4 | 429 B9 | 531 C8 | 532 C11 | 532 D3 | 532 F9 | 529 B9 | | | | |
| 531 C8 | 532 F9 | 532 C11 | 532 D3 | 426 F11 | 427 C5 | 429 E4 | 429 B9 | 529 B9 | 649 B11 | 532 F3 | F31 E7 |
| 532 C11 | 532 F9 | 532 D3 | 531 C8 | 426 F11 | 427 C5 | 429 E4 | 429 B9 | 529 B9 | 649 B11 | 532 F3 | F31 E7 |
| 532 D3 | 532 F9 | 532 C11 | 531 C8 | 426 F11 | 427 C5 | 429 E4 | 429 B9 | 529 B9 | 649 B11 | 532 F3 | F31 E7 |
| 532 F9 | 532 C11 | 532 D3 | 531 C8 | 426 F11 | 427 C5 | 429 E4 | 429 B9 | 529 B9 | 649 B11 | 532 F3 | F31 E7 |
| 529 B9 | 531 C8 | 532 C11 | 532 D3 | 532 F9 | 426 F11 | 427 C5 | 429 E4 | 429 B9 | | | |
| 649 B11 | 531 C8 | 532 C11 | 532 D3 | 532 F9 | 426 F11 | 427 C5 | 429 E4 | 429 B9 | | | |
| 532 F3 | 531 C8 | 532 C11 | 532 D3 | | | | | | | | |
| 531 E7 | 531 C8 | 532 C11 | 532 D3 | 532 F9 | | | | | | | |

### Example 7: NI-607 Antibodies are Capable of Interfering with SARS-CoV-2 Spike (S) Protein Mediated Entry of Pseudovirus into the Cell

Pseudovirus antiviral assays were performed with vesicular stomatitis virus (VSV) pseudoviruses expressing the SARS-CoV-2 Spike (S) protein. Such virus (Spike (SARS-CoV-2) Pseudotyped Lentivirus (Luciferase Reporter)) can for example be obtained from BPS Bioscience, Inc (San Diego, Calif., USA) (Catalog #79942). Infections were monitored using a luciferase assay. Data points were performed in duplicates at seven concentrations. Assays were performed with ten test-items. Tests included controls with vehicle alone and an inhibitor blocking entry of SARS-CoV-2 pseudoviruses assessed at one concentration in duplicates. Detailed description of the assay as performed in the course of the present invention can be found in Nie et al., Emerging Microbes & Infections 9 (2020), 680-686 (doi: 10.1080/22221751.2020.1743767).

The neutralization assays were performed with HEK 293T-ACE2, a human embryonic kidney cell line overexpressing ACE-2, the receptor of SARS-CoV-2 virus. Thirty thousand 293T-ACE2 cells were seeded the day before in white plates in the presence of hygromycin (100 μg/mL). The day of the neutralization assay, 25 μL of media containing pseudovirus was mixed with 25 μL of serial dilutions of the test-item in a different plate, and then incubated for 1 h at 37° C. Pseudovirus and test-item dilutions were performed in DMEM media containing 5% heat-inactivated fetal bovine serum (DMEM5). After the 60-minute incubation, the test-item/pseudovirus mixture was added to the 293T-ACE2 cells. Infection was allowed for twenty-four hours. Firefly luciferase activity was monitored at 24 h using the Britelite reporter gene assay (Perkin Elmer).

The results of the assays are shown in FIG. 5 including the IC₅₀ values of the tested antibodies, and it has been found that eleven antibodies reached the maximum neutralization of 50% or greater. In particular, the IC₅₀ of antibody NI-607.429_B9 as determined by the pseudovirus entry assay is 7.1 μg/mL (FIG. 5A), the IC₅₀ of antibody NI-607.429_E4 is 35 ng/mL (FIG. 5B), the IC₅₀ of antibody NI-607.529_B9 is 1.9 μg/mL (FIG. 5B), the IC₅₀ of antibody NI-607.531_C8 is 86.4 ng/mL (0.572 nM) (FIG. 5B), the IC₅₀ of antibody NI-607.532_B6 is 100 μg/mL (FIG. 5C), the IC₅₀ of antibody NI-607.532_C11 is 87.2 ng/mL (FIG. 5C), the IC₅₀ of antibody NI-607.532_D3 is 2.1 μg/mL (FIG. 5C), the IC₅₀ of antibody NI-607.532_F9 is 363.6 ng/mL (FIG. 5D), the IC₅₀ of antibody NI-607.649_B11 is 231.8 ng/mL (1.531 nM) (FIG. 5D), the IC₅₀ of antibody NI-607.531_E7 is 3.9 μg/mL (FIG. 5D), and the IC₅₀ of antibody NI-607.532_F3 is 11.7 μg/mL (FIG. 5D).

The other antibodies, i.e., NI-607.427_C5 (FIG. 5A), NI-607.426_F11 (FIG. 5A), NI-607.274_B7 (FIG. 5A), NI-607.529_G4 (FIG. 5B), NI-607.531_D8 (FIG. 5C), and NI-607.649_G7 (FIG. 5E) showed an IC₅₀>100 μg/mL.

In FIG. 5E a positive control, i.e., antibody D002 (SinoBiologics; Catalog number: 40150-D002) as well as two negative controls (PBS and isotype control) are shown, wherein the positive control reaches the maximum neutralization of 50% and shows an IC₅₀ of 591.5 ng/mL.

Thus, antibodies have been identified that show a greater neutralizing effect than the commercially available positive control which shows a high affinity towards the RBD of SARS-CoV-2.

### Example 8: NI-607 Antibodies Inhibit Viral Induced Cytotoxicity in Human Cell Line Caco-2

Antibodies were tested for their inhibition of viral induced cytotoxicity using the human epithelial colorectal adenocarcinoma cell line Caco-2 and a SARS-CoV-2 isolate. Caco-2 cells were seeded at 5×10⁴ cells per 96 well to reach confluent monolayers. Antibodies were diluted in culture medium and incubated for 1 hour at 37° C. with SARS-Cov-2 virus (Frankfurt isolate, 100TCid50=MOI 0.01, Bojkova et al., Nature (2020), Proteomics of SARS-CoV-2-infected host cells reveals therapy targets, doi:10.1038/s41586-020-2332-7). Virus antibody mixture was added to confluent cells and incubated for up to 72 hours. CPE was scored by phase contrast microscopy and subsequent image analysis (the scoring was performed visually). Patient serum diluted at 1/160 was used as positive control and an isotype control as negative control.

The detailed description of the assay as performed in the course of the present invention can be found in Ellinger et al., Identification of inhibitors of SARS-CoV-2 in-vitro cellular toxicity in human (Caco-2) cells using a large scale drug repurposing collection (2020), 1-19. doi:10.21203/RS.3.RS-23951/V1.

The following antibody concentrations have been tested:

| Concentration [nM] | Concentration[μg/ml] | Dilution |
|---|---|---|
| 1000 | 150 | 0 |
| 100 | 15 | 10 |
| 50 | 7.5 | 20 |
| 25 | 3.75 | 40 |
| 12.5 | 1.875 | 80 |
| 6.25 | 0.9375 | 160 |
| 3.125 | 0.46875 | 320 |
| 1.5625 | 0.234375 | 640 |
| 0.78125 | 0.1171875 | 1280 |

As illustrated in FIG. 6, antibodies NI-607.649_B11, NI-607.532_F9, NI-607.532_D3, NI-607.532_C11, NI-607.521_C8, and NI-607.429_E4 show a similar or even better neutralizing efficacy than the positive control and also antibodies NI-607.529_B9, NI-607.429_B9, and NI-607.427_C5 show neutralization capability, while antibody NI-607.529_G4 does hardly show neutralization of the virus. These results are consistent with the virus neutralization assay as described in Example 7.

### Example 9: NI-607 Antibodies Inhibit Fully Replication Competent SARS-CoV-2 Virus

The antibodies of the present invention were tested if they could inhibit the infection of cells with fully replication competent SARS-CoV-2 viruses. SARS-CoV-2-GFP viruses as described in Thao et al., (Nature (2020), doi: 10.1038/s41586-020-2294-9) were pre-incubated with respective antibodies for 30 min at room temperature in medium and then added to Vero E6 cells at a high MOI of 1. After 1 hour of incubation the supernatant was removed and replaced with fresh medium without antibodies and the cells were further incubated at 37° C. for up to 72 hrs. GFP expression was monitored by high throughput microscopy every four hours for up to 72 hrs and quantified.

The following antibody concentrations have been tested:

| Concentration [nM] | Concentration[μg/ml] | Dilution |
|---|---|---|
| 6666 | 1000 | 0 |
| 266.64 | 40 | 25 |
| 53.328 | 8 | 125 |
| 10.6656 | 1.6 | 625 |
| 2.13312 | 0.32 | 3125 |
| 0.426624 | 0.064 | 15625 |
| 0.0853248 | 0.0128 | 78125 |
| 0.01706496 | 0.00256 | 390625 |
| 0.003412992 | 0.000512 | 1953125 |

As illustrated in FIGS. 7A, 7C, and 7E antibodies NI-607.531_C8, NI-607.649_B11 and NI-607.429_E4 showed clear reduction in GFP which equals viral gene expression and viral growth with dilutions 1/3125 to 1/25 (NI-607.531_C8), with dilutions 1/125 and 1/25 (NI-607.649_B11) and with dilutions 1/625 to 1/25 (NI-607.429_E4), respectively. In contrast, the isotype control showed no reduction in viral replication at all tested dilutions (see FIG. 7G).

Furthermore, antibodies NI-607.429_B9 and NI-607.529_B9 showed clear reduction in GFP with dilution 1/25, antibody NI-607.532_C11 showed clear reduction in GFP with dilution 1/125 and 1/25, antibodies NI-607.532_D3 and NI-607.532_F9 showed clear reduction in GFP with dilutions 1/625 to 1/25 (data not shown).

As illustrated in FIGS. 7B, 7D, 7F, and 7H, antibodies NI-607.531_C8, NI-607.649_B11 and NI-607.429_E4 do not show any signs of cell toxicity in comparison to the isotype control. Antibodies NI-607.427_C5, NI-607.429_B9, NI-607.529_B9, NI-607.532_C11, NI-607.532_D3, NI-607.532_F9, and NI-607.532_F3 have been tested as well and also those show no signs of cell toxicity (data not shown).

Thus, in summary, several antibodies show clear dose dependent reduction of viral driven GFP expression in comparison to the isotype control. At the same time the antibodies do not show any signs of cell toxicity.

### Example 10: Single Human Monoclonal Antibody Prevents and Treats SARS-CoV-2 Infection in COVID-19 Animal Model

Two studies were conducted following methods described in Baum et al. “REGN-COV2 antibody cocktail prevents and treats SARS-CoV-2 infection in rhesus macaques and hamsters”. Preprint at bioRxiv https://doi.org/10.1101/2020.08.02.233320 (2020). Challenge route intranasal 2.3×10e4 PFU SARS-Cov2, age of animals 6-8 weeks, gender mixed female and male. Weight was measured each day. Study 1 (prophylactic study) was measuring the response with antibody application at day −1 and virus inoculation at day 0. Animal weights were recorded for further seven days. In study 2 (therapeutic study) virus was inoculated at day 0, antibody was administered at day +2 and animal weights were followed up to day +12. Both studies were conducted in comparison to a non-related IgG4 S228P isotype control at 5 mg/animal. For both studies two anti SARS-CoV2 antibodies were evaluated (NI-607.531_C8 and NI-607.649_B11) at 5, 1 and 0.2 mg per animal with 5 animals per dose group. The results are shown in FIG. 5.

In view of previous studies in the golden Syrian hamster model and the predictability of the doses used in the model for use in humans, it appears as if that for achieving serum concentrations of the antibody as observed in the hamster model similar doses can be used in humans; see Roberts et. al. (2006), supra, and Morrey et al., Antimicrob. Agents Chemother. 51 (2007), 2396-2402. Accordingly, considering the doses as applied to the animals the following doses per kilogram (mg/kg) can be calculated and expected to be efficacious in human patients to the same or similar extent as observed in the hamster model.

| Challenge route | Challenge dose | Age | Gender |
|---|---|---|---|
| Intranasal | 2.3 × 10e4 PFU | 6-8 weeks | Male/female |

| Group | | Dose/kg | | Dose/animal | |
|---|---|---|---|---|---|
| Antibody 1 | 47B11 | 42 | mg/kg | 5 | mg |
| Antibody 2 | NI-607.531_C8 | 42 | mg/kg | 5 | mg |
| Antibody 2 | NI-607.531_C8 | 8.3 | mg/kg | 1 | mg |
| Antibody 2 | NI-607.531_C8 | 1.6 | mg/kg | 0.2 | mg |
| Antibody 3 | NI-607.649_B11 | 42 | mg/kg | 5 | mg |
| Antibody 3 | NI-607.649_B11 | 8.3 | mg/kg | 1 | mg |
| Antibody 3 | NI-607.649_B11 | 1.6 | mg/kg | 0.2 | mg |

Accordingly, the experiments performed in accordance with the present invention confirm that human-derived anti-SARS-Cov-2 antibodies are provided that are suitable for use in the prophylactic and/or therapeutic treatment of COVID-19 disease, in particular wherein the treatment is characterized by parenteral injection or infusion of the antibody to a subject in need thereof. As used in this study, for the prophylactic treatment the antibody is preferably administered about one day before the expected exposure of the subject to SARS-CoV-2 and the therapeutic treatment is performed by administering the antibody about two days after infection with SARS-CoV-2. Preferred administration routes in these settings are intravenous (IV) infusion or subcutaneous (SC) injection. As summarized in the table above, suitable doses comprise single doses of 1 to 2 mg/kg, 8 to 10 mg/kg, 40 to 45 mg/kg or 50 mg/kg, preferably 8 to 10 mg/kg or 40 to 45 mg/kg or any dose in between. In the prophylactic treatment, the antibody may be administered before the subject is at risk to be exposed to SARS-CoV-2, for example when the subject is working or living in environment of potential SARS-CoV-2 infection such as a Hospital, clinic, nursing home, retirement home, school, or COVID-19 hot spot.

### Example 11: Binding Characteristics Against Circulating SARS Cov2 Mutants as Determined by ELISA

To determine the binding specificity and the half maximal effective concentration (EC50) of recombinant human-derived SARS-CoV-2 antibodies NI-607.531_C8 and NI-607.649_B11 for binding the RBD or full spike protein of SARS-CoV-2 S an ELISA EC50 analysis was performed.

In brief, direct ELISA was performed using 96-well microplates (Corning Incorporated, Corning, USA) coated with either: (FIG. 9A,C) Coated antigens: SARS-CoV-2-S1(RBD)-His 0.32 mg/mL Trenzyme, SARS-CoV-2 (2019-nCoV) Spike S1-His 250 ug/ml Sino 40591-V08H, SARS-CoV-2 Spike Protein (S1+S2 ECD, His-Tag) 250 ug/ml Sino 40589-V08B1, SARS-CoV-2 (2019-nCoV) Spike RBD-His 250 ug/ml Sino 40592-V08B, SARS-CoV-2 (2019-nCoV) Spike RBD(N501Y)-His 250 ug/ml Sino 40592-V08H82, SARS-CoV-2 (2019-nCoV) Spike RBD(Y453F)-His 250 ug/ml Sino 40592-V08H80, SARS-CoV-2 (2019-nCoV) Spike S1(D614G)-His 250 ug/ml Sino 40591-V08H3, SARS-CoV-2 (2019-nCoV) Spike RBD(N439K)-His 250 ug/ml Sino 40592-V08H14 (FIG. 9. B,D) Coated antigens:SARS-CoV-2 (2019-nCoV) Spike RBD-His 250 ug/ml Sino 40592-V08B, SARS-CoV-2 (2019-nCoV) Spike RBD(Y453F)-His 250 ug/ml Sino 40592-V08H80, SARS-CoV-2 (2019-nCoV) Spike S1-His 250 ug/ml Sino 40591-V08H, SARS-CoV-2 (2019-nCoV) Spike S1(K417N, E484K, N501Y, D614G)-His 250 ug/ml Sino 40591-V08H10, SARS-CoV-2 (2019-nCoV) Spike S1(HV69-70 deletion, Y453F, D614G)-His 250 ug/ml Sino 40591-V08H8, SARS-CoV-2 Spike Protein (S1+S2 ECD, His-Tag) 250 ug/ml Sino 40589-V08B1, SARS-CoV-2 full-length Trimeric Spike Recombinant Antigen (B.1.135, South Africa, (Antibody Resistance of SARS-CoV-2 Variants B.1.351 and B.1.1.7 Pengfei Wang, Manoj S. Nair, Lihong Liu, Sho Iketani, Yang Luo, Yicheng Guo, Maple Wang, Jian Yu, Baoshan Zhang, Peter D. Kwong, Barney S. Graham, John R. Mascola, Jennifer Y. Chang, Michael T. Yin, Magdalena Sobieszczyk, Christos A. Kyratsous, Lawrence Shapiro, Zizhang Sheng, Yaoxing Huang, David D. Ho bioRxiv 2021.01.25.428137) 0.8 mg/ml Bioserv BSV-COV-PR-61, SARS-CoV-2 full-length Trimeric Spike Recombinant Antigen (P.1, Brazil, Increased Resistance of SARS-CoV-2 Variant P.1 to Antibody Neutralization, Pengfei Wang, Ryan G. Casner, Manoj S. Nair, Maple Wang, Jian Yu, Gabriele Cerutti, Lihong Liu, Peter D. Kwong, Yaoxing Huang, Lawrence Shapiro, David D. Ho, bioRxiv 2021.03.01.433466)) 1.12 mg/ml Bioserv BSV-COV-PR-69, SARS-CoV-2 full-length Trimeric Spike Recombinant Antigen (B.1.1.7, UK variant, (Antibody Resistance of SARS-CoV-2 Variants B.1.351 and B.1.1.7 Pengfei Wang, Manoj S. Nair, Lihong Liu, Sho Iketani, Yang Luo, Yicheng Guo, Maple Wang, Jian Yu, Baoshan Zhang, Peter D. Kwong, Barney S. Graham, John R. Mascola, Jennifer Y. Chang, Michael T. Yin, Magdalena Sobieszczyk, Christos A. Kyratsous, Lawrence Shapiro, Zizhang Sheng, Yaoxing Huang, David D. Ho bioRxiv 2021.01.25.428137)) 0.92 mg/ml Bioserv BSV-COV-PR-65, Bovine Serum Albumin ≥96% Sigma, Cat #A8022-100G at a concentration of 5 μg/ml in PBS for 2 h at room temperature with gentle shaking on the orbital shaker. Afterwards, plates were washed twice with 150 μl PBS-Tween20 (PBS-T). Non-specific binding sites were blocked for 1 h at room temperature with gentle shaking with 5% (w/v) BSA in PBS-T. Afterwards, plates were washed twice with 150 μl PBS-T. Antibodies NI-607.531_C8 and NI-607.649_B11 were diluted in PBS (23 serial dilutions from 400 nM stock) and incubated for 2 h at room temperature with gentle shaking on the orbital shaker, followed by two washing steps with PBS-T and incubation with a donkey anti-human IgG Fcg-specific antibody conjugated with HRP (Jackson ImmunoResearch Laboratories, Inc., West Grove, USA) for 1 h at room temperature with gentle shaking. After four washing steps, binding was determined by measurement of HRP activity in a standard colorimetric assay. EC50 values were estimated by non-linear regression using GraphPad Prism software (San Diego, USA). The binding specificity and EC50 of human-derived SARS-CoV-2-specific antibodies were determined by ELISA. Antibody NI-607.531_C8 specifically recognizes the RBD of full spike protein of SARS-CoV-2 wt and mutant strains with an EC50 of 25+−5 pM. Antibody NI-607.649_B11 specifically recognizes the RBD of full spike protein of SARS-CoV-2 wt and mutant strains with an EC50 of 31+−7 pM. The results show that the EC50 values do not substantially change and are still in the same order of magnitude. In conclusion, high-throughput immune repertoire analyses of donors successfully recovered from COVID-19 lead to the successful cloning and recombinant production of human monoclonal antibodies targeting SARS-CoV-2 with high affinity and preserving efficacy to circulated mutants.

### Example 12: Epitope Mapping

1. Cross-Linking Mass Spectrometry

Discontinuous epitope mapping was performed using a combination of crosslinking and deuterium exchange mass spectrometry following standard methods.

Cross-linking mass spectrometry has been described previously (see, for example international applications WO2006/116893A1; WO2010/136539A1; Slavin et al., 2021 https://doi.org/10.1101/2021.02.04.429751. For example, epitope mapping of neutralizing nanobodies (Nbs) to SARS-CoV-2 spike protein RBD employing an integrative approach by using size exclusion chromatography (SEC), cross-linking and mass spectrometry, and structural modeling has been described; see, e.g., Xiang et al., Science 370 (2020), 1479-1484 and references cited therein.

High-Mass MALDI mass spectrometry was performed using an Autoflex II MALDI ToFToF mass spectrometer (Bruker).

In order to determine the epitope of NI-607.531_C8/SARS-CoV-2-S and NI-607.649_B11/SARS-CoV-2-S complexes with high resolution, the protein complex was incubated with cross-linkers and subjected to multi-enzymatic cleavage (Bich, C et al. Anal. Chem., 2010, 82 (1), pp 172-179),). After enrichment of the cross-linked peptides, the samples were analyzed by high resolution mass spectrometry (nLC-LTQ-Orbitrap mass spectrometry). The cross-linked peptides were analyzed using XQuest and Stavrox software.

After Trypsin, Chymotrypsin, ASP-N, Elastase and Thermolysin proteolysis of the protein complex SARS-CoV-2-S/NI-607.531_C8 or SARS-CoV-2-S/NI-607.649_B11 with deuterated d0d12, the nLC-orbitrap MS/MS analysis detected 11 cross-linked peptides between SARS-CoV-2 spike protein and the antibody NI-607.531_C8 and 14 cross-linked peptides between SARS-CoV-2-S and the antibody NI-607.649_B11.

The analysis indicates that the interaction between NI-607.531_C8 and SARS-CoV-2-S includes the following amino acids on SARS-CoV-2 spike protein RBD (numbering based on SEQ ID NO: 301): 112T, 120S, 140K, 141S, 144K, 1511S, 177Y. Stretch regions of interaction between NI-607.531_C8 and SARS-CoV-2-S correspond to amino acids 112-120 (SEQ ID NO: 302), 140-144 (SEQ ID NO: 303) and 151-177 (SEQ ID NO: 304) of SARS-CoV-2-RBD (SEQ ID NO: 301), (see, FIG. 10,A for illustration).

Furthermore, the analysis indicates that the interaction between NI-607.649_B11 and SARS-CoV-2-S includes the following amino acids on SARS-CoV-2 spike protein RBD (numbering based on SEQ ID NO: 301): 51Y, 65S, 67T, 68K, 112T, 120S, 139R, 140K, 144K, 148R, 151S, 152T. Stretch regions of interaction between NI-607.649_B11 and SARS-CoV-2-S correspond to 51-68 (SEQ ID NO: 312), 112-120 (SEQ ID NO: 302) and 139-152 (SEQ ID NO:313) of SARS-CoV-2-S (see, FIG. 10,13 for illustration).

2. Hydrogen Deuterium Exchange Mass Spectrometry (HDX-MS)

Epitope mapping with HDX-MS is also well known in the art. For example, the combination approach using amide hydrogen/deuterium exchange coupled with proteolysis and mass spectrometry (HDX-MS) and computational docking has been described to be applied to investigate antigen-antibody interactions, wherein the identified epitopes are in good agreement with those identified using high-resolution X-ray crystallography; see, e.g., Pandit et al., J. Mol. Recognit. 25 (2012), 114-124. The general principle of the technology for example illustrated in FIG. 3 of Pradzinska et al., Amino Acids 48 (2016), 2809-2820 at page 2813.

In addition, the practical utility of hydrogen/deuterium exchange mass spectrometry (HDX-MS) in epitope mapping studies of a cohort of four monoclonal antibodies targeting Major histocompatibility complex class I chain-related A and B (MICA/B) that act as ligands to natural killer cell receptors, NKG2D, expressed on immune cells, followed by electron-transfer dissociation allowing high-resolution refinement of binding epitopes has been described in Huang et al., Analytical and Bioanalytical Chemistry 412 (2020), 1693-1700. Likewise, Huang et al., MABS 10 (2018), 95-103 describe the use of hydrogen/deuterium exchange mass spectrometry (HDX-MS) to obtain molecular-level details of anti-TL1A monoclonal antibody 1 (mAb1) binding epitope on TL1A, wherein HDX coupled with electron-transfer dissociation MS provided residue-level epitope information.

For mapping of a discontinuous epitope by hydrogen-deuterium exchange mass spectrometry (HDX-MS) and/or fast photochemical oxidation of proteins (FPOP) epitope mapping illustrated for a monoclonal antibody that specifically binds to human CD27 (hCD27) reference is made to international application WO2019/019195452 A1.

Binding epitope characterization of SARS-CoV-2 antibodies using hydrogen-deuterium exchange (HDX) followed by mass spectrometry (MS) to obtain epitope information for antibodies and to gain finer epitope sequence detail for several antibodies has also been described; see., e.g., Jones et al., Sci. Transl. Med. 13, eabf1906 (2021), 1-17 and in particular for nanobodies (Nbs), originated from an immunized alpaca which bind with high affinities to the glycosylated SARS-CoV-2 Spike receptor domain (RBD), Wagner et al., EMBO Rep. 22 (2021): e52325. doi: 10.15252/embr.202052325, the disclosure content of each document being incorporated by reference, in particular the material and method sections relating to HDX-MS.

For the characterization of SARS-CoV-2-S1(RBD)-His/NI-607.531_C8-IgG4-S228P and SARS-CoV-2-S1(RBD)-His/NI-607.649_B11-IgG4-S228P complexes, the measurements were performed using an Autoflex II MALDI ToF ToF mass spectrometer (Bruker).

For the HDX-MS analysis, the measurements were performed using a LEAP HDX sample handling robot (LEAP instrument) in line with an LTQ XL Orbitrap mass spectrometer (Thermo Scientific). The LEAP HDX sample handling robot allow to perform all the necessary pipetting, quenching and proteolysis step before mass spectrometric analysis of hydrogen/deuterium exchange.

For the H/D experiments on SARS-CoV-2-S1(RBD)-His was mixed with either NI-607.531_C8-IgG4-S228P or NI-607.649_B11-IgG4-S228P77 and 81 peptides, respectively, were identified both in control and exchange experiments after the incubation times 15 s, 50 s, 150 s, 500 s, 1500 s and 5000 s. For each incubation time, the % of incorporation of deuterium was determined based on average values of triplicate experiments. The determination of the deuterium incorporation in each pepsin peptides of SARS-CoV-2-S1(RBD)-His mixed with NI-607.531_C8-IgG4-S228P or NI-607.649_B11-IgG4-S228P for each incubation time allows to obtain deuterium exchange heat map. By comparing the heat maps obtained in different conditions for the same protein, an HDX heat map can be obtained, which corresponds to the comparison between the heat map obtained for SARS-CoV-2-S1(RBD)-His and SARS-CoV-2-S1(RBD)-His mixed with NI-607.531_C8-IgG4-S228P or NI-607.649_B11-IgG4-S228P. As illustrated in FIG. 10, HDX-MS data analysis shows that (A) amino acids 105 to 135 (SEQ ID NO: 305), 105 to 134 (SEQ ID NO: 306), 106 to 135 (SEQ ID NO: 307), 106 to 134 (SEQ ID NO: 308), 153 to 178 (SEQ ID NO: 309), 155 to 178 (SEQ ID NO: 310), 164 to 176 (SEQ ID NO: 311) of the RBD of SARS-CoV-2 (SEQ-ID NO 301) showed significant protection upon binding NI-607.531_C8-IgG4-S228P and (B) amino acids 55 to 65 (SEQ ID NO: 314), 56 to 65 (SEQ ID NO: 315), 69 to 73 (SEQ ID NO: 316), 70 to 73 (SEQ ID NO: 317) of RBD of SARS-CoV-2 (SEQ-ID NO 301) showed significant protection upon NI-607.649_B11 binding.

1. A recombinant human-derived monoclonal antibody or antigen-binding fragment thereof, wherein the antibody is capable of binding to the receptor-binding domain (RBD) of the spike (S) protein of the Severe Acute Respiratory Syndrome Coronavirus-2 (SARS-CoV-2).

2. The antibody or antigen-binding fragment thereof of claim 1, comprising an IgG4 constant domain.

3. The antibody or antigen-binding fragment thereof of claim 2, wherein the IgG4 constant domain includes the S228P mutation.

4. The antibody or antigen-binding fragment thereof of any one of claim 1 to 3, wherein the binding to RBD is with an EC50 of <100 pM.

5. The antibody or antigen-binding fragment thereof of any one of claims 1 to 4, wherein the antibody or antigen-binding fragment thereof binds to a conformational epitope.

6. The antibody or antigen-binding fragment thereof of any one of claims 1 to 5, wherein the antibody or antigen-binding fragment thereof binds to the RBD of SARS-CoV with an EC50 which is at least one or two orders of magnitude higher than the EC50 for its binding to the RBD of SARS-CoV-2, or wherein the antibody or antigen-binding fragment thereof does not substantially bind to the RBD of SARS-CoV.

7. The antibody or antigen-binding fragment thereof of any one of claims 1 to 5, wherein the antibody binds to the RBD of SARS-CoV.

8. The antibody or antigen-binding fragment thereof of any one of claims 1 to 7, wherein the antibody or antigen-binding fragment thereof binds to the RBD of SARS-CoV with an EC50 which is in the same order of magnitude as the EC50 for its binding to the RBD of SARS-CoV-2.

9. The antibody or antigen-binding fragment thereof of any one of claims 1 to 8, wherein the antibody or antigen-binding fragment thereof is capable of binding to one or more SARS-CoV-2 variants selected from the group consisting of S1 Mink, B1.351, B1.1.7, P1, B.1.135, RBD N439K, RBD Y453F, RBD N501Y, S1 D614G.

10. The antibody or antigen-binding fragment thereof of claim 9, wherein the antibody or antigen-binding fragment thereof is capable of binding to all said SARS-Cov-2 variants.

11. The antibody or antigen binding fragment thereof of any one of claims 1 to 10 characterized by a variable region comprising:
(i) a variable heavy (VH) chain comprising VH complementary determining regions (CDRs) 1, 2, and 3, and a variable light (VL) chain comprising VL CDRs 1, 2, and 3, selected from the group consisting of:
(a) [NI-607.531_C8], wherein VH-CDR1 comprises the amino acid sequence of SEQ ID NO: 123 or a variant thereof, wherein the variant comprises one or two amino acid substitutions,
VH-CDR2 comprises the amino acid sequence of SEQ ID NO: 124 or a variant thereof, wherein the variant comprises one or two amino acid substitutions,
VH-CDR3 comprises the amino acid sequence of SEQ ID NO: 125 or a variant thereof, wherein the variant comprises one or two amino acid substitutions,
VL-CDR1 comprises the amino acid sequence of SEQ ID NO: 128 or a variant thereof, wherein the variant comprises one or two amino acid substitutions,
VL-CDR2 comprises the amino acid sequence of SEQ ID NO: 129 or a variant thereof, wherein the variant comprises one or two amino acid substitutions, and
VL-CDR3 comprises the amino acid sequence of SEQ ID NO: 130 or a variant thereof, wherein the variant comprises one or two amino acid substitutions;
(b) [NI-607.649_B11], wherein VH-CDR1 comprises the amino acid sequence of SEQ ID NO: 213 or a variant thereof, wherein the variant comprises one or two amino acid substitutions,
VH-CDR2 comprises the amino acid sequence of SEQ ID NO: 214 or a variant thereof, wherein the variant comprises one or two amino acid substitutions,
VH-CDR3 comprises the amino acid sequence of SEQ ID NO: 215 or a variant thereof, wherein the variant comprises one or two amino acid substitutions,
VL-CDR1 comprises the amino acid sequence of SEQ ID NO: 218 or a variant thereof, wherein the variant comprises one or two amino acid substitutions,
VL-CDR2 comprises the amino acid sequence of SEQ ID NO: 219 or a variant thereof, wherein the variant comprises one or two amino acid substitutions, and
VL-CDR3 comprises the amino acid sequence of SEQ ID NO: 220 or a variant thereof, wherein the variant comprises one or two amino acid substitutions;
(c) [NI-607.275_C5], wherein VH-CDR1 comprises the amino acid sequence of SEQ ID NO: 23 or a variant thereof, wherein the variant comprises one or two amino acid substitutions,
VH-CDR2 comprises the amino acid sequence of SEQ ID NO: 24 or a variant thereof, wherein the variant comprises one or two amino acid substitutions,
VH-CDR3 comprises the amino acid sequence of SEQ ID NO: 25 or a variant thereof, wherein the variant comprises one or two amino acid substitutions,
VL-CDR1 comprises the amino acid sequence of SEQ ID NO: 28 or a variant thereof, wherein the variant comprises one or two amino acid substitutions,
VL-CDR2 comprises the amino acid sequence of SEQ ID NO: 29 or a variant thereof, wherein the variant comprises one or two amino acid substitutions, and
VL-CDR3 comprises the amino acid sequence of SEQ ID NO: 30 or a variant thereof, wherein the variant comprises one or two amino acid substitutions;
(d) [NI-607.426_D4], wherein VH-CDR1 comprises the amino acid sequence of SEQ ID NO: 33 or a variant thereof, wherein the variant comprises one or two amino acid substitutions,
VH-CDR2 comprises the amino acid sequence of SEQ ID NO: 34 or a variant thereof, wherein the variant comprises one or two amino acid substitutions,
VH-CDR3 comprises the amino acid sequence of SEQ ID NO: 35 or a variant thereof, wherein the variant comprises one or two amino acid substitutions,
VL-CDR1 comprises the amino acid sequence of SEQ ID NO: 38 or a variant thereof, wherein the variant comprises one or two amino acid substitutions,
VL-CDR2 comprises the amino acid sequence of SEQ ID NO: 39 or a variant thereof, wherein the variant comprises one or two amino acid substitutions, and
VL-CDR3 comprises the amino acid sequence of SEQ ID NO: 40 or a variant thereof, wherein the variant comprises one or two amino acid substitutions;
(e) [NI-607.426_E2], wherein VH-CDR1 comprises the amino acid sequence of SEQ ID NO: 43 or a variant thereof, wherein the variant comprises one or two amino acid substitutions,
VH-CDR2 comprises the amino acid sequence of SEQ ID NO: 44 or a variant thereof, wherein the variant comprises one or two amino acid substitutions,
VH-CDR3 comprises the amino acid sequence of SEQ ID NO: 45 or a variant thereof, wherein the variant comprises one or two amino acid substitutions,
VL-CDR1 comprises the amino acid sequence of SEQ ID NO: 48 or a variant thereof, wherein the variant comprises one or two amino acid substitutions,
VL-CDR2 comprises the amino acid sequence of SEQ ID NO: 49 or a variant thereof, wherein the variant comprises one or two amino acid substitutions, and
VL-CDR3 comprises the amino acid sequence of SEQ ID NO: 50 or a variant thereof, wherein the variant comprises one or two amino acid substitutions; or
(f) [NI-607.426_F11], wherein VH-CDR1 comprises the amino acid sequence of SEQ ID NO: 53 or a variant thereof, wherein the variant comprises one or two amino acid substitutions,
VH-CDR2 comprises the amino acid sequence of SEQ ID NO: 54 or a variant thereof, wherein the variant comprises one or two amino acid substitutions,
VH-CDR3 comprises the amino acid sequence of SEQ ID NO: 55 or a variant thereof, wherein the variant comprises one or two amino acid substitutions,
VL-CDR1 comprises the amino acid sequence of SEQ ID NO: 58 or a variant thereof, wherein the variant comprises one or two amino acid substitutions,
VL-CDR2 comprises the amino acid sequence of SEQ ID NO: 59 or a variant thereof, wherein the variant comprises one or two amino acid substitutions, and
VL-CDR3 comprises the amino acid sequence of SEQ ID NO: 60 or a variant thereof, wherein the variant comprises one or two amino acid substitutions;
(g) [NI-607.427_C5], wherein VH-CDR1 comprises the amino acid sequence of SEQ ID NO: 63 or a variant thereof, wherein the variant comprises one or two amino acid substitutions,
VH-CDR2 comprises the amino acid sequence of SEQ ID NO: 64 or a variant thereof, wherein the variant comprises one or two amino acid substitutions,
VH-CDR3 comprises the amino acid sequence of SEQ ID NO: 65 or a variant thereof, wherein the variant comprises one or two amino acid substitutions,
VL-CDR1 comprises the amino acid sequence of SEQ ID NO: 68 or a variant thereof, wherein the variant comprises one or two amino acid substitutions,
VL-CDR2 comprises the amino acid sequence of SEQ ID NO: 69 or a variant thereof, wherein the variant comprises one or two amino acid substitutions, and
VL-CDR3 comprises the amino acid sequence of SEQ ID NO: 70 or a variant thereof, wherein the variant comprises one or two amino acid substitutions;
(h) [NI-607.428_B9], wherein VH-CDR1 comprises the amino acid sequence of SEQ ID NO: 73 or a variant thereof, wherein the variant comprises one or two amino acid substitutions,
VH-CDR2 comprises the amino acid sequence of SEQ ID NO: 74 or a variant thereof, wherein the variant comprises one or two amino acid substitutions,
VH-CDR3 comprises the amino acid sequence of SEQ ID NO: 75 or a variant thereof, wherein the variant comprises one or two amino acid substitutions,
VL-CDR1 comprises the amino acid sequence of SEQ ID NO: 78 or a variant thereof, wherein the variant comprises one or two amino acid substitutions,
VL-CDR2 comprises the amino acid sequence of SEQ ID NO: 79 or a variant thereof, wherein the variant comprises one or two amino acid substitutions, and
VL-CDR3 comprises the amino acid sequence of SEQ ID NO: 80 or a variant thereof, wherein the variant comprises one or two amino acid substitutions;
(i) [NI-607.429_B9], wherein VH-CDR1 comprises the amino acid sequence of SEQ ID NO: 83 or a variant thereof, wherein the variant comprises one or two amino acid substitutions,
VH-CDR2 comprises the amino acid sequence of SEQ ID NO: 84 or a variant thereof, wherein the variant comprises one or two amino acid substitutions,
VH-CDR3 comprises the amino acid sequence of SEQ ID NO: 85 or a variant thereof, wherein the variant comprises one or two amino acid substitutions,
VL-CDR1 comprises the amino acid sequence of SEQ ID NO: 88 or a variant thereof, wherein the variant comprises one or two amino acid substitutions,
VL-CDR2 comprises the amino acid sequence of SEQ ID NO: 89 or a variant thereof, wherein the variant comprises one or two amino acid substitutions, and
VL-CDR3 comprises the amino acid sequence of SEQ ID NO: 90 or a variant thereof, wherein the variant comprises one or two amino acid substitutions;
(j) [NI-607.429_E4], wherein VH-CDR1 comprises the amino acid sequence of SEQ ID NO: 93 or a variant thereof, wherein the variant comprises one or two amino acid substitutions,
VH-CDR2 comprises the amino acid sequence of SEQ ID NO: 94 or a variant thereof, wherein the variant comprises one or two amino acid substitutions,
VH-CDR3 comprises the amino acid sequence of SEQ ID NO: 95 or a variant thereof, wherein the variant comprises one or two amino acid substitutions,
VL-CDR1 comprises the amino acid sequence of SEQ ID NO: 98 or a variant thereof, wherein the variant comprises one or two amino acid substitutions,
VL-CDR2 comprises the amino acid sequence of SEQ ID NO: 99 or a variant thereof, wherein the variant comprises one or two amino acid substitutions, and
VL-CDR3 comprises the amino acid sequence of SEQ ID NO: 100 or a variant thereof, wherein the variant comprises one or two amino acid substitutions;
(k) [NI-607.529_B9], wherein VH-CDR1 comprises the amino acid sequence of SEQ ID NO: 103 or a variant thereof, wherein the variant comprises one or two amino acid substitutions,
VH-CDR2 comprises the amino acid sequence of SEQ ID NO: 104 or a variant thereof, wherein the variant comprises one or two amino acid substitutions,
VH-CDR3 comprises the amino acid sequence of SEQ ID NO: 105 or a variant thereof, wherein the variant comprises one or two amino acid substitutions,
VL-CDR1 comprises the amino acid sequence of SEQ ID NO: 108 or a variant thereof, wherein the variant comprises one or two amino acid substitutions,
VL-CDR2 comprises the amino acid sequence of SEQ ID NO: 109 or a variant thereof, wherein the variant comprises one or two amino acid substitutions, and
VL-CDR3 comprises the amino acid sequence of SEQ ID NO: 110 or a variant thereof, wherein the variant comprises one or two amino acid substitutions;
(l) [NI-607.529_G4], wherein VH-CDR1 comprises the amino acid sequence of SEQ ID NO: 113 or a variant thereof, wherein the variant comprises one or two amino acid substitutions,
VH-CDR2 comprises the amino acid sequence of SEQ ID NO: 114 or a variant thereof, wherein the variant comprises one or two amino acid substitutions,
VH-CDR3 comprises the amino acid sequence of SEQ ID NO: 115 or a variant thereof, wherein the variant comprises one or two amino acid substitutions,
VL-CDR1 comprises the amino acid sequence of SEQ ID NO: 118 or a variant thereof, wherein the variant comprises one or two amino acid substitutions,
VL-CDR2 comprises the amino acid sequence of SEQ ID NO: 119 or a variant thereof, wherein the variant comprises one or two amino acid substitutions, and
VL-CDR3 comprises the amino acid sequence of SEQ ID NO: 120 or a variant thereof, wherein the variant comprises one or two amino acid substitutions;
(m) [NI-607.274_B7], wherein VH-CDR1 comprises the amino acid sequence of SEQ ID NO: 3 or a variant thereof, wherein the variant comprises one or two amino acid substitutions,
VH-CDR2 comprises the amino acid sequence of SEQ ID NO: 4 or a variant thereof, wherein the variant comprises one or two amino acid substitutions,
VH-CDR3 comprises the amino acid sequence of SEQ ID NO: 5 or a variant thereof, wherein the variant comprises one or two amino acid substitutions,
VL-CDR1 comprises the amino acid sequence of SEQ ID NO: 8 or a variant thereof, wherein the variant comprises one or two amino acid substitutions,
VL-CDR2 comprises the amino acid sequence of SEQ ID NO: 9 or a variant thereof, wherein the variant comprises one or two amino acid substitutions, and
VL-CDR3 comprises the amino acid sequence of SEQ ID NO: 10 or a variant thereof, wherein the variant comprises one or two amino acid substitutions;
(n) [NI-607.531_D8], wherein VH-CDR1 comprises the amino acid sequence of SEQ ID NO: 133 or a variant thereof, wherein the variant comprises one or two amino acid substitutions,
VH-CDR2 comprises the amino acid sequence of SEQ ID NO: 134 or a variant thereof, wherein the variant comprises one or two amino acid substitutions,
VH-CDR3 comprises the amino acid sequence of SEQ ID NO: 135 or a variant thereof, wherein the variant comprises one or two amino acid substitutions,
VL-CDR1 comprises the amino acid sequence of SEQ ID NO: 138 or a variant thereof, wherein the variant comprises one or two amino acid substitutions,
VL-CDR2 comprises the amino acid sequence of SEQ ID NO: 139 or a variant thereof, wherein the variant comprises one or two amino acid substitutions, and
VL-CDR3 comprises the amino acid sequence of SEQ ID NO: 140 or a variant thereof, wherein the variant comprises one or two amino acid substitutions;
(o) [NI-607.532_B6], wherein VH-CDR1 comprises the amino acid sequence of SEQ ID NO: 143 or a variant thereof, wherein the variant comprises one or two amino acid substitutions,
VH-CDR2 comprises the amino acid sequence of SEQ ID NO: 144 or a variant thereof, wherein the variant comprises one or two amino acid substitutions,
VH-CDR3 comprises the amino acid sequence of SEQ ID NO: 145 or a variant thereof, wherein the variant comprises one or two amino acid substitutions,
VL-CDR1 comprises the amino acid sequence of SEQ ID NO: 148 or a variant thereof, wherein the variant comprises one or two amino acid substitutions,
VL-CDR2 comprises the amino acid sequence of SEQ ID NO: 149 or a variant thereof, wherein the variant comprises one or two amino acid substitutions, and
VL-CDR3 comprises the amino acid sequence of SEQ ID NO: 150 or a variant thereof, wherein the variant comprises one or two amino acid substitutions;
(p) [NI-607.532_C11], wherein VH-CDR1 comprises the amino acid sequence of SEQ ID NO: 153 or a variant thereof, wherein the variant comprises one or two amino acid substitutions,
VH-CDR2 comprises the amino acid sequence of SEQ ID NO: 154 or a variant thereof, wherein the variant comprises one or two amino acid substitutions,
VH-CDR3 comprises the amino acid sequence of SEQ ID NO: 155 or a variant thereof, wherein the variant comprises one or two amino acid substitutions,
VL-CDR1 comprises the amino acid sequence of SEQ ID NO: 158 or a variant thereof, wherein the variant comprises one or two amino acid substitutions,
VL-CDR2 comprises the amino acid sequence of SEQ ID NO: 159 or a variant thereof, wherein the variant comprises one or two amino acid substitutions, and
VL-CDR3 comprises the amino acid sequence of SEQ ID NO: 160 or a variant thereof, wherein the variant comprises one or two amino acid substitutions;
(q) [NI-607.532_C8], wherein VH-CDR1 comprises the amino acid sequence of SEQ ID NO: 163 or a variant thereof, wherein the variant comprises one or two amino acid substitutions,
VH-CDR2 comprises the amino acid sequence of SEQ ID NO: 164 or a variant thereof, wherein the variant comprises one or two amino acid substitutions,
VH-CDR3 comprises the amino acid sequence of SEQ ID NO: 165 or a variant thereof, wherein the variant comprises one or two amino acid substitutions,
VL-CDR1 comprises the amino acid sequence of SEQ ID NO: 168 or a variant thereof, wherein the variant comprises one or two amino acid substitutions,
VL-CDR2 comprises the amino acid sequence of SEQ ID NO: 169 or a variant thereof, wherein the variant comprises one or two amino acid substitutions, and
VL-CDR3 comprises the amino acid sequence of SEQ ID NO: 170 or a variant thereof, wherein the variant comprises one or two amino acid substitutions;
(r) [NI-607.532_D3], wherein VH-CDR1 comprises the amino acid sequence of SEQ ID NO: 173 or a variant thereof, wherein the variant comprises one or two amino acid substitutions,
VH-CDR2 comprises the amino acid sequence of SEQ ID NO: 174 or a variant thereof, wherein the variant comprises one or two amino acid substitutions,
VH-CDR3 comprises the amino acid sequence of SEQ ID NO: 175 or a variant thereof, wherein the variant comprises one or two amino acid substitutions,
VL-CDR1 comprises the amino acid sequence of SEQ ID NO: 178 or a variant thereof, wherein the variant comprises one or two amino acid substitutions,
VL-CDR2 comprises the amino acid sequence of SEQ ID NO: 179 or a variant thereof, wherein the variant comprises one or two amino acid substitutions, and
VL-CDR3 comprises the amino acid sequence of SEQ ID NO: 180 or a variant thereof, wherein the variant comprises one or two amino acid substitutions;
(s) [NI-607.532_D4], wherein VH-CDR1 comprises the amino acid sequence of SEQ ID NO: 183 or a variant thereof, wherein the variant comprises one or two amino acid substitutions,
VH-CDR2 comprises the amino acid sequence of SEQ ID NO: 184 or a variant thereof, wherein the variant comprises one or two amino acid substitutions,
VH-CDR3 comprises the amino acid sequence of SEQ ID NO: 185 or a variant thereof, wherein the variant comprises one or two amino acid substitutions,
VL-CDR1 comprises the amino acid sequence of SEQ ID NO: 188 or a variant thereof, wherein the variant comprises one or two amino acid substitutions,
VL-CDR2 comprises the amino acid sequence of SEQ ID NO: 189 or a variant thereof, wherein the variant comprises one or two amino acid substitutions, and
VL-CDR3 comprises the amino acid sequence of SEQ ID NO: 190 or a variant thereof, wherein the variant comprises one or two amino acid substitutions;
(t) [NI-607.532_D8], wherein VH-CDR1 comprises the amino acid sequence of SEQ ID NO: 193 or a variant thereof, wherein the variant comprises one or two amino acid substitutions,
VH-CDR2 comprises the amino acid sequence of SEQ ID NO: 194 or a variant thereof, wherein the variant comprises one or two amino acid substitutions,
VH-CDR3 comprises the amino acid sequence of SEQ ID NO: 195 or a variant thereof, wherein the variant comprises one or two amino acid substitutions,
VL-CDR1 comprises the amino acid sequence of SEQ ID NO: 198 or a variant thereof, wherein the variant comprises one or two amino acid substitutions,
VL-CDR2 comprises the amino acid sequence of SEQ ID NO: 199 or a variant thereof, wherein the variant comprises one or two amino acid substitutions, and
VL-CDR3 comprises the amino acid sequence of SEQ ID NO: 200 or a variant thereof, wherein the variant comprises one or two amino acid substitutions;
(u) [NI-607.532_F9], wherein VH-CDR1 comprises the amino acid sequence of SEQ ID NO: 203 or a variant thereof, wherein the variant comprises one or two amino acid substitutions,
VH-CDR2 comprises the amino acid sequence of SEQ ID NO: 204 or a variant thereof, wherein the variant comprises one or two amino acid substitutions,
VH-CDR3 comprises the amino acid sequence of SEQ ID NO: 205 or a variant thereof, wherein the variant comprises one or two amino acid substitutions,
VL-CDR1 comprises the amino acid sequence of SEQ ID NO: 208 or a variant thereof, wherein the variant comprises one or two amino acid substitutions,
VL-CDR2 comprises the amino acid sequence of SEQ ID NO: 209 or a variant thereof, wherein the variant comprises one or two amino acid substitutions, and
VL-CDR3 comprises the amino acid sequence of SEQ ID NO: 210 or a variant thereof, wherein the variant comprises one or two amino acid substitutions;
(v) [NI-607.274_E5], wherein VH-CDR1 comprises the amino acid sequence of SEQ ID NO: 13 or a variant thereof, wherein the variant comprises one or two amino acid substitutions,
VH-CDR2 comprises the amino acid sequence of SEQ ID NO: 14 or a variant thereof, wherein the variant comprises one or two amino acid substitutions,
VH-CDR3 comprises the amino acid sequence of SEQ ID NO: 15 or a variant thereof, wherein the variant comprises one or two amino acid substitutions,
VL-CDR1 comprises the amino acid sequence of SEQ ID NO: 18 or a variant thereof, wherein the variant comprises one or two amino acid substitutions,
VL-CDR2 comprises the amino acid sequence of SEQ ID NO: 19 or a variant thereof, wherein the variant comprises one or two amino acid substitutions, and
VL-CDR3 comprises the amino acid sequence of SEQ ID NO: 20 or a variant thereof, wherein the variant comprises one or two amino acid substitutions;
(w) [NI-607.531_E7], wherein VH-CDR1 comprises the amino acid sequence of SEQ ID NO: 223 or a variant thereof, wherein the variant comprises one or two amino acid substitutions,
VH-CDR2 comprises the amino acid sequence of SEQ ID NO: 224 or a variant thereof, wherein the variant comprises one or two amino acid substitutions,
VH-CDR3 comprises the amino acid sequence of SEQ ID NO: 225 or a variant thereof, wherein the variant comprises one or two amino acid substitutions,
VL-CDR1 comprises the amino acid sequence of SEQ ID NO: 228 or a variant thereof, wherein the variant comprises one or two amino acid substitutions,
VL-CDR2 comprises the amino acid sequence of SEQ ID NO: 229 or a variant thereof, wherein the variant comprises one or two amino acid substitutions, and
VL-CDR3 comprises the amino acid sequence of SEQ ID NO: 230 or a variant thereof, wherein the variant comprises one or two amino acid substitutions;
(x) [NI-607.532_F3], wherein VH-CDR1 comprises the amino acid sequence of SEQ ID NO: 233 or a variant thereof, wherein the variant comprises one or two amino acid substitutions,
VH-CDR2 comprises the amino acid sequence of SEQ ID NO: 234 or a variant thereof, wherein the variant comprises one or two amino acid substitutions,
VH-CDR3 comprises the amino acid sequence of SEQ ID NO: 235 or a variant thereof, wherein the variant comprises one or two amino acid substitutions,
VL-CDR1 comprises the amino acid sequence of SEQ ID NO: 238 or a variant thereof, wherein the variant comprises one or two amino acid substitutions,
VL-CDR2 comprises the amino acid sequence of SEQ ID NO: 239 or a variant thereof, wherein the variant comprises one or two amino acid substitutions, and
VL-CDR3 comprises the amino acid sequence of SEQ ID NO: 240 or a variant thereof, wherein the variant comprises one or two amino acid substitutions;
(y) [NI-607.649_G7], wherein VH-CDR1 comprises the amino acid sequence of SEQ ID NO: 243 or a variant thereof, wherein the variant comprises one or two amino acid substitutions,
VH-CDR2 comprises the amino acid sequence of SEQ ID NO: 244 or a variant thereof, wherein the variant comprises one or two amino acid substitutions,
VH-CDR3 comprises the amino acid sequence of SEQ ID NO: 245 or a variant thereof, wherein the variant comprises one or two amino acid substitutions,
VL-CDR1 comprises the amino acid sequence of SEQ ID NO: 248 or a variant thereof, wherein the variant comprises one or two amino acid substitutions,
VL-CDR2 comprises the amino acid sequence of SEQ ID NO: 249 or a variant thereof, wherein the variant comprises one or two amino acid substitutions, and
VL-CDR3 comprises the amino acid sequence of SEQ ID NO: 250 or a variant thereof, wherein the variant comprises one or two amino acid substitutions;
(z) [NI-607.761_B7], wherein VH-CDR1 comprises the amino acid sequence of SEQ ID NO: 253 or a variant thereof, wherein the variant comprises one or two amino acid substitutions,
VH-CDR2 comprises the amino acid sequence of SEQ ID NO: 254 or a variant thereof, wherein the variant comprises one or two amino acid substitutions,
VH-CDR3 comprises the amino acid sequence of SEQ ID NO: 255 or a variant thereof, wherein the variant comprises one or two amino acid substitutions,
VL-CDR1 comprises the amino acid sequence of SEQ ID NO: 258 or a variant thereof, wherein the variant comprises one or two amino acid substitutions,
VL-CDR2 comprises the amino acid sequence of SEQ ID NO: 259 or a variant thereof, wherein the variant comprises one or two amino acid substitutions, and
VL-CDR3 comprises the amino acid sequence of SEQ ID NO: 260 or a variant thereof, wherein the variant comprises one or two amino acid substitutions;
(za) [NI-607.791_B10], wherein VH-CDR1 comprises the amino acid sequence of SEQ ID NO: 263 or a variant thereof, wherein the variant comprises one or two amino acid substitutions,
VH-CDR2 comprises the amino acid sequence of SEQ ID NO: 264 or a variant thereof, wherein the variant comprises one or two amino acid substitutions,
VH-CDR3 comprises the amino acid sequence of SEQ ID NO: 265 or a variant thereof, wherein the variant comprises one or two amino acid substitutions,
VL-CDR1 comprises the amino acid sequence of SEQ ID NO: 268 or a variant thereof, wherein the variant comprises one or two amino acid substitutions,
VL-CDR2 comprises the amino acid sequence of SEQ ID NO: 269 or a variant thereof, wherein the variant comprises one or two amino acid substitutions, and
VL-CDR3 comprises the amino acid sequence of SEQ ID NO: 270 or a variant thereof, wherein the variant comprises one or two amino acid substitutions;
(zb) [NI-607.531_E3], wherein VH-CDR1 comprises the amino acid sequence of SEQ ID NO: 273 or a variant thereof, wherein the variant comprises one or two amino acid substitutions,
VH-CDR2 comprises the amino acid sequence of SEQ ID NO: 274 or a variant thereof, wherein the variant comprises one or two amino acid substitutions,
VH-CDR3 comprises the amino acid sequence of SEQ ID NO: 275 or a variant thereof, wherein the variant comprises one or two amino acid substitutions,
VL-CDR1 comprises the amino acid sequence of SEQ ID NO: 278 or a variant thereof, wherein the variant comprises one or two amino acid substitutions,
VL-CDR2 comprises the amino acid sequence of SEQ ID NO: 279 or a variant thereof, wherein the variant comprises one or two amino acid substitutions, and
VL-CDR3 comprises the amino acid sequence of SEQ ID NO: 280 or a variant thereof, wherein the variant comprises one or two amino acid substitutions;
(zc) [NI-607.820_B6], wherein VH-CDR1 comprises the amino acid sequence of SEQ ID NO: 283 or a variant thereof, wherein the variant comprises one or two amino acid substitutions,
VH-CDR2 comprises the amino acid sequence of SEQ ID NO: 284 or a variant thereof, wherein the variant comprises one or two amino acid substitutions,
VH-CDR3 comprises the amino acid sequence of SEQ ID NO: 285 or a variant thereof, wherein the variant comprises one or two amino acid substitutions,
VL-CDR1 comprises the amino acid sequence of SEQ ID NO: 288 or a variant thereof, wherein the variant comprises one or two amino acid substitutions,
VL-CDR2 comprises the amino acid sequence of SEQ ID NO: 289 or a variant thereof, wherein the variant comprises one or two amino acid substitutions, and
VL-CDR3 comprises the amino acid sequence of SEQ ID NO: 290 or a variant thereof, wherein the variant comprises one or two amino acid substitutions; and
(zd) [NI-607.820_B7], wherein VH-CDR1 comprises the amino acid sequence of SEQ ID NO: 293 or a variant thereof, wherein the variant comprises one or two amino acid substitutions,
VH-CDR2 comprises the amino acid sequence of SEQ ID NO: 294 or a variant thereof, wherein the variant comprises one or two amino acid substitutions,
VH-CDR3 comprises the amino acid sequence of SEQ ID NO: 295 or a variant thereof, wherein the variant comprises one or two amino acid substitutions,
VL-CDR1 comprises the amino acid sequence of SEQ ID NO: 298 or a variant thereof, wherein the variant comprises one or two amino acid substitutions,
VL-CDR2 comprises the amino acid sequence of SEQ ID NO: 299 or a variant thereof, wherein the variant comprises one or two amino acid substitutions, and
VL-CDR3 comprises the amino acid sequence of SEQ ID NO: 300 or a variant thereof, wherein the variant comprises one or two amino acid substitutions; and/or
(ii) a VH and/or VL chain comprising an amino acid sequence selected from the group consisting of:
(a) [NI-607.531_C8] the amino acid sequence of SEQ ID NO: 122, or SEQ ID NO: 127, or an amino acid sequence being at least 90% identical to the amino acid sequence of SEQ ID NO: 122 and SEQ ID NO: 127;
(b) [NI-607.649_B11] the amino acid sequence of SEQ ID NO: 212, or SEQ ID NO: 217, or an amino acid sequence being at least 90% identical to the amino acid sequence of SEQ ID NO: 212 and SEQ ID NO: 217;
(c) [NI-607.275_C5] the amino acid sequence of SEQ ID NO: 22, or SEQ ID NO: 27, or an amino acid sequence being at least 90% identical to the amino acid sequence of SEQ ID NO: 22 and SEQ ID NO: 27;
(d) [NI-607.426_D4] the amino acid sequence of SEQ ID NO: 32, or SEQ ID NO: 37, or an amino acid sequence being at least 90% identical to the amino acid sequence of SEQ ID NO: 32 and SEQ ID NO: 37;
(e) [NI-607.426_E2] the amino acid sequence of SEQ ID NO: 42, or SEQ ID NO: 47, or an amino acid sequence being at least 90% identical to the amino acid sequence of SEQ ID NO: 42 and SEQ ID NO: 47;
(f) [NI-607.426_F11] the amino acid sequence of SEQ ID NO: 52, or SEQ ID NO: 57, or an amino acid sequence being at least 90% identical to the amino acid sequence of SEQ ID NO: 52 and SEQ ID NO: 57;
(g) [NI-607.427_C5] the amino acid sequence of SEQ ID NO: 62, or SEQ ID NO: 67, or an amino acid sequence being at least 90% identical to the amino acid sequence of SEQ ID NO: 62 and SEQ ID NO: 67;
(h) [NI-607.428_B9] the amino acid sequence of SEQ ID NO: 72, or SEQ ID NO: 77, or an amino acid sequence being at least 90% identical to the amino acid sequence of SEQ ID NO: 72 and SEQ ID NO: 77;
(i) [NI-607.429_B9] the amino acid sequence of SEQ ID NO: 82, or SEQ ID NO: 87, or an amino acid sequence being at least 90% identical to the amino acid sequence of SEQ ID NO: 82 and SEQ ID NO: 87;
(j) [NI-607.429_E4] the amino acid sequence of SEQ ID NO: 92, or SEQ ID NO: 97, or an amino acid sequence being at least 90% identical to the amino acid sequence of SEQ ID NO: 92 and SEQ ID NO: 97;
(k) [NI-607.529_B9] the amino acid sequence of SEQ ID NO: 102, or SEQ ID NO: 107, or an amino acid sequence being at least 90% identical to the amino acid sequence of SEQ ID NO: 102 and SEQ ID NO: 107;
(l) [NI-607.529_G4] the amino acid sequence of SEQ ID NO: 112, or SEQ ID NO: 117, or an amino acid sequence being at least 90% identical to the amino acid sequence of SEQ ID NO: 112 and SEQ ID NO: 117;
(m) [NI-607.274_B7] the amino acid sequence of SEQ ID NO: 2, or SEQ ID NO: 7, or an amino acid sequence being at least 90% identical to the amino acid sequence of SEQ ID NO: 2 and SEQ ID NO: 7;
(n) [NI-607.531_D8] the amino acid sequence of SEQ ID NO: 132, or SEQ ID NO: 137, or an amino acid sequence being at least 90% identical to the amino acid sequence of SEQ ID NO: 132 and SEQ ID NO: 137;
(o) [NI-607.532_B6] the amino acid sequence of SEQ ID NO: 142, or SEQ ID NO: 147, or an amino acid sequence being at least 90% identical to the amino acid sequence of SEQ ID NO: 142 and SEQ ID NO: 147;
(p) [NI-607.532_C11] the amino acid sequence of SEQ ID NO: 152, or SEQ ID NO: 157, or an amino acid sequence being at least 90% identical to the amino acid sequence of SEQ ID NO: 152 and SEQ ID NO: 157;
(q) [NI-607.532_C8] the amino acid sequence of SEQ ID NO: 162, or SEQ ID NO: 167, or an amino acid sequence being at least 90% identical to the amino acid sequence of SEQ ID NO: 162 and SEQ ID NO: 167;
(r) [NI-607.532_D3] the amino acid sequence of SEQ ID NO: 172, or SEQ ID NO: 177, or an amino acid sequence being at least 90% identical to the amino acid sequence of SEQ ID NO: 172 and SEQ ID NO: 177;
(s) [NI-607.532_D4] the amino acid sequence of SEQ ID NO: 182, or SEQ ID NO: 187, or an amino acid sequence being at least 90% identical to the amino acid sequence of SEQ ID NO: 182 and SEQ ID NO: 187;
(t) [NI-607.532_D8] the amino acid sequence of SEQ ID NO: 192, or SEQ ID NO: 197, or an amino acid sequence being at least 90% identical to the amino acid sequence of SEQ ID NO: 192 and SEQ ID NO: 197;
(u) [NI-607.532_F9] the amino acid sequence of SEQ ID NO: 202, or SEQ ID NO: 207, or an amino acid sequence being at least 90% identical to the amino acid sequence of SEQ ID NO: 202 and SEQ ID NO: 207;
(v) [NI-607.274_E5] the amino acid sequence of SEQ ID NO: 12, or SEQ ID NO: 17, or an amino acid sequence being at least 90% identical to the amino acid sequence of SEQ ID NO: 12 and SEQ ID NO: 17;
(w) [NI-607.531_E7] the amino acid sequence of SEQ ID NO: 222, or SEQ ID NO: 227, or an amino acid sequence being at least 90% identical to the amino acid sequence of SEQ ID NO: 222 and SEQ ID NO: 227;
(x) [NI-607.532_F3] the amino acid sequence of SEQ ID NO: 232, or SEQ ID NO: 237, or an amino acid sequence being at least 90% identical to the amino acid sequence of SEQ ID NO: 232 and SEQ ID NO: 237;
(y) [NI-607.649_G7] the amino acid sequence of SEQ ID NO: 242, or SEQ ID NO: 247, or an amino acid sequence being at least 90% identical to the amino acid sequence of SEQ ID NO: 242 and SEQ ID NO: 247;
(z) [NI-607.761_B7] the amino acid sequence of SEQ ID NO: 252, or SEQ ID NO: 257, or an amino acid sequence being at least 90% identical to the amino acid sequence of SEQ ID NO: 252 and SEQ ID NO: 257;
(za) [NI-607.791_B10] the amino acid sequence of SEQ ID NO: 262, or SEQ ID NO: 267, or an amino acid sequence being at least 90% identical to the amino acid sequence of SEQ ID NO: 262 and SEQ ID NO: 267;
(zb) [NI-607.531_E3] the amino acid sequence of SEQ ID NO: 272, or SEQ ID NO: 277, or an amino acid sequence being at least 90% identical to the amino acid sequence of SEQ ID NO: 272 and SEQ ID NO: 277;
(zc) [NI-607.820_B6] the amino acid sequence of SEQ ID NO: 282, or SEQ ID NO: 287, or an amino acid sequence being at least 90% identical to the amino acid sequence of SEQ ID NO: 282 and SEQ ID NO: 287; and
(zd) [NI-607.820_B7] the amino acid sequence of SEQ ID NO: 292, or SEQ ID NO: 297, or an amino acid sequence being at least 90% identical to the amino acid sequence of SEQ ID NO: 292 and SEQ ID NO: 297.

12. An antibody or antigen-binding fragment thereof, wherein the antibody competes with (i) antibody NI-607.531_C8 for binding to the RBD of SARS-CoV-2 or (ii) antibody NI-607.649_B11 for binding to the RBD of SARS-CoV-2, and optionally for binding to the RBD of SARS-CoV.

13. The antibody or antigen-binding fragment thereof of claim 11 or 12, wherein the antibody or antigen-binding fragment thereof binds to at least a first epitope, a second epitope and/or a third epitope of a discontinuous epitope of the RBD of the SARS-CoV-2, wherein the RBD of the SARS-CoV-2 comprises or consists of the amino acid sequence of SEQ ID NO: 301,
wherein the first epitope comprises or consists of amino acid residues 112 to 120 (SEQ ID NO: 302) of SEQ ID NO: 301, the second epitope comprises or consists of amino acid residues 140 to 144 (SEQ ID NO: 303) of SEQ ID NO: 301 and the third epitope comprises or consists of amino acid residues 151 to 177 (SEQ ID NO: 304) of SEQ ID NO: 301, as determined by Cross-linking Mass Spectrometry.

14. The antibody or antigen-binding fragment thereof of any one of claims 11 to 13, wherein the antibody or antigen-binding fragment thereof binds to at least a first epitope and/or a second epitope of a discontinuous epitope of the RBD of the SARS-CoV-2, as determined by HDX-MS,
wherein the RBD of SARS-CoV-2 comprises or consists of an amino acid sequence SEQ ID NO: 301,
wherein the first epitope comprises or consists of amino acid residues 105 to 135 (SEQ ID NO: 305) of SEQ ID NO: 301, preferably wherein the first epitope comprises or consists of amino acid residues 105 to 134 (SEQ ID NO: 306), 106 to 135 (SEQ ID NO: 307) or 106 to 134 (SEQ ID NO: 308) of SEQ ID NO: 301,
wherein the second epitope comprises or consists of amino acid residues 153 to 178 (SEQ ID NO: 309) of SEQ ID NO: 301, preferably wherein the second epitope comprises or consists of amino acid residues 155 to 178 (SEQ ID NO: 310) of SEQ ID NO: 301 or amino acid residues 164 to 176 (SEQ ID NO: 311).

15. The antibody or antigen-binding fragment thereof of claim 11 or 12, wherein the antibody binds to at least a first epitope, a second epitope and/or a third epitope of the RBD of the SARS-CoV-2,
wherein the RBD of the SARS-CoV-2 comprises or consists of the amino acid sequence of SEQ ID NO: 301,
wherein the first epitope comprises or consists of amino acid residues 51 to 68 (SEQ ID NO: 312) of SEQ ID NO: 301, the second epitope comprises or consists of amino acid residues 112 to 120 (SEQ ID NO: 302) of SEQ ID NO: 301 and the third epitope comprises or consists of amino acid residues 139 to 152 (SEQ ID NO: 313) of SEQ ID NO: 301, as determined by Cross-linking Mass Spectrometry.

16. The antibody or antigen-binding fragment thereof of any one of claims 11, 12 or 15, wherein the antibody or antigen-binding fragment thereof binds to at least a first epitope and/or a second epitope of a discontinuous epitope of the RBD of the SARS-CoV-2, as determined by HDX-MS,
wherein the RBD of SARS-CoV-2 comprises or consists of an amino acid sequence SEQ ID NO: 301,
wherein the first epitope comprises or consists of amino acid residues 55 to 65 (SEQ ID NO: 314) of SEQ ID NO: 301, preferably wherein the first epitope comprises or consists of amino acid residues 56 to 65 (SEQ ID NO: 315) of SEQ ID NO: 301, and
wherein the second epitope comprises or consists of amino acid residues 69 to 73 (SEQ ID NO: 316) of SEQ ID NO: 301, preferably wherein the second epitope comprises or consists of amino acid residues 70 to 73 (SEQ ID NO: 317) of SEQ ID NO 301.

17. The antibody or antigen-binding fragment thereof of any one of claims 11 to 16, wherein the antibody binds to at least one epitope of the RBD of the SARS-CoV-2 comprising or consisting of the amino acid sequence of SEQ ID NO: 302 and/or to an epitope comprising or consisting of the amino acid sequence of SEQ ID NO: 313, as determined by Cross-linking Mass Spectrometry.

18. The antibody or antigen-binding fragment thereof of any one of claims 11 to 17, wherein the binding of the antibody to SARS-CoV-2 and its RBD, respectively, involves interaction with one or more, preferably all amino acid residues: 112 T, 120 S, 140 K, 144 K, 151 S of the RBD (SEQ ID NO: 301) as determined by Cross-linking Mass Spectrometry.

19. The antibody or antigen-binding fragment thereof of any one of claims 1 to 18, wherein the antibodies do not substantially bind to the corresponding RBD of Middle East Respiratory Syndrome Coronavirus (MERS-CoV).

20. The antibody or the antigen-binding fragment thereof of any one of claims 1 to 19, capable of:
(i) inhibiting binding of the RBD to the human angiotensin converting enzyme 2 (ACE2) at an IC₅₀ of <3000 pM, preferably <200 pM;
(ii) neutralizing vesicular stomatitis virus (VSV) pseudoviruses expressing the SARS-CoV-2 Spike (S) protein at an IC₅₀ of ≤100 μg/mL, preferably ≤15 μg/mL, more preferably ≤300 ng/mL;
(iii) neutralizing SARS-CoV-2 at a concentration of ≤15 μg/mL, preferably ≤5 μg/mL; and/or
(iv) neutralizing SARS-CoV-2-GFP at a concentration of ≤40 μg/mL, preferably ≤μg/mL.

21. The antibody of any one of claims 1 to 20, wherein the antibody is a chimeric antibody, a bispecific antibody, and/or is an IgG, or wherein the antibody fragment is a recombinant scFv (single chain fragment variable) antibody, Fab fragment, F(ab′)₂ fragment, a disulfide-linked Fv (sdFv), an Fd, or Fv fragment.

22. The antibody of any one of claims 1 to 21, wherein the antibody is an IgG, or a recombinant IgG antibody or antibody fragment comprising an Fc portion mutated to alter (eliminate or enhance) FcR interactions, such as a LALA, N297, GASD/ALIE, or is glycan modified to alter (eliminate or enhance) FcR interactions, such as enzymatic or chemical addition or removal of glycans, or genetic modification of a glycosylation pattern, or comprises an Fc portion mutated to alter FcRn interactions to increase in vivo half-life and in vivo protection, such as a YTE or LS mutation.

23. The antibody of any one of claims 1 to 22, wherein the antibody is IgA or has been derived from an IgA and class-switched to an IgG.

24. The antibody of any one of claims 21 to 23, wherein said antibody is an IgG1 or IgG4.

25. The antibody of any one of claims 21 to 23, wherein said antibody is an IgG1 variant L234A, L235A, P329G (LALA-PG) or an IgG4 variant S228P.

26. The antibody or antigen-binding fragment thereof of any one of claims 1 to 25, wherein
the antibody binds to the RBD of SARS-CoV-2 with an EC₅₀ of <100 pM;
the antibody does not substantially bind to the corresponding RBD of MERS-CoV;
the antibody is capable of:
(i) inhibiting binding of the RBD to human ACE2 at an IC₅₀ of <200 pM;
(ii) neutralizing vesicular stomatitis virus (VSV) pseudoviruses expressing the SARS-CoV-2 Spike (S) protein at an IC₅₀ of ≤15 μg/mL;
(iii) neutralizing SARS-CoV-2 at a concentration of ≤15 μg/mL, and/or
(iv) neutralizing SARS-CoV-2-GFP at a concentration of ≤40 μg/mL; and
the antibody binds to the RBD of SARS-CoV with an EC₅₀ which is in the same [NI-607.649_B11], at least one [NI-607.529_B9] or two [NI-607.531_C8] order of magnitude(s) higher than the EC₅₀ for its binding to the RBD of SARS-CoV-2.

27. A composition comprising one or more antibodies or antigen-binding fragments thereof of claims 1 to 26.

28. An antibody cocktail comprising or consisting of at least the antibody of any one of claims 12 to 18.

29. The antibody cocktail comprising or consisting of at least two antibodies or antigen-binding fragments thereof of any one of claims 1 to 26, wherein the antibodies or antigen-binding fragments thereof compete with each other for binding to the RBD of SARS-CoV-2 and/or which do not compete with each other for binding to the RBD of SARS-CoV-2.

30. One or more polynucleotide(s) encoding the antibody or antigen-binding fragment of any one of claims 1 to 26 or an immunoglobulin VH and VL chain of any one thereof.

31. The polynucleotide(s) of claim 30, wherein the polynucleotide is a cDNA.

32. The polynucleotide(s) of claim 30 or 31, wherein the polynucleotide(s) is operably linked to a heterologous nucleic acid.

33. The polynucleotide(s) of claim 32, wherein the heterologous sequence is one or more expression control sequences.

34. One or more vector(s) comprising the polynucleotide(s) of claims 30 to 33.

35. A host cell comprising the polynucleotide(s) of claims 30 to 33 or the vector(s) of claim 34.

36. A method for producing an anti-SARS-CoV-2 antibody, antigen-binding fragment or immunoglobulin chain(s) thereof, said method comprising:
(a) culturing the cell of claim 35, and
(b) isolating the antibody, antigen-binding fragment or immunoglobulin chain(s) thereof from the culture; or
(c) expressing a polynucleotide encoding the antibody or antigen-binding fragment thereof of any one of claims 1 to 26 within a cell or cell-free expression system.

37. An antibody, antigen-binding fragment or immunoglobulin chain(s) thereof encoded by the polynucleotide(s) of any one of claims 30 to 33 or obtainable by the method of claim 36.

38. The antibody or antigen-binding fragment thereof of any one of claims 1 to 26 or the antibody cocktail of claim 28 or 29, wherein the antibody or antigen-binding fragment thereof
(i) is detectably labeled with a label selected from the group consisting of an enzyme, a radioisotope, a fluorescent compound, a chemiluminescent compound, a bioluminescent compound, a tag, a flag and a heavy metal;
(ii) is attached to a drug;
(iii) comprises polyethylene glycol;
(iv) comprises a brain targeting entity; and/or
(v) is contained in or conjugated to a vehicle such as an exosome or nanoparticle.

39. A composition comprising at least the antibody or antigen-binding fragments thereof of any one of claims 1 to 26, 37, or 38 the antibody cocktail of any one of claims 28, 29 or 38, the polynucleotide(s) of any one of claims 30 to 33, the vector(s) of claim 34 or the cell of claim 35.

40. The composition of claim 39, wherein the composition is:
(i) a pharmaceutical composition and further comprises a pharmaceutically acceptable carrier; or
(ii) a diagnostic composition and designed as a kit.

41. The composition of claim 40(ii), further comprising reagents conventionally used in immuno-based diagnostic methods.

42. A composition comprising at least the antibody or antigen-binding fragments thereof of any one of claims 1 to 26, 37 or 38, the antibody cocktail of any one of claims 28, 29 or 38 for use in:
(i) the prophylactic or therapeutic treatment of Covid-19 and infection with SARS-CoV-2, or
(ii) monitoring the progression of infection with SARS-CoV-2, or the response to a treatment of COVID-19 disease in a subject,
wherein the disease and infection, respectively, affects the respiratory tract, kidneys, liver, heart, brain, pancreas, adrenal glands, or lymphatic system.

43. The composition for use according to claim 42, wherein the composition is designed for topical mucosal and/or pulmonary delivery of the antibody.

44. The composition for use according to claim 43, wherein the composition is designed as aerosol.

45. The composition of claim 44, wherein the composition is designed as aerosol together with a system for aerosol drug delivery such as nebulizer, metered dose inhaler (MDI), or dry powder inhalers (DPI).

46. The antibody or antigen-binding fragments thereof of any one of claims 1 to 26, 37 or 38, or the antibody cocktail of claim 28, 29 or 38 for use in the prophylactic and/or therapeutic treatment of COVID-19 disease, wherein the treatment is characterized by parenteral injection or infusion of the antibody to a subject in need thereof.

47. The composition for use according to any one of claims 42 to 45, the antibody or the antigen-binding fragment thereof, or the antibody cocktail for use according to claim 46, wherein the prophylactic treatment is characterized by administering the antibody about one day before the expected exposure of the subject to SARS-CoV-2 and the therapeutic treatment is characterized by administering the antibody about two days after infection with SARS-CoV-2.

48. The composition for use according to any one of claims 42 to 45 or 47, the antibody or antigen-binding fragment thereof or antibody cocktail for use according to claim 46, wherein the antibody is administered by intravenous (IV) infusion or subcutaneous (SC) injection.

49. The composition, antibody or antigen-binding fragment thereof or antibody cocktail for use according to claim 48, wherein the antibody is administered at a single dose of 1 to 2 mg/kg, 8 to 10 mg/kg, 40 to 45 mg/kg or 50 mg/kg.

50. The composition, antibody or antigen-binding fragment thereof or antibody cocktail for use according to any one of claim 48 or 49, wherein the antibody is administered before the subject is at risk to be exposed to SARS-CoV-2.

51. The composition, antibody or antigen-binding fragment thereof or antibody cocktail for use according to any one of claims 48 to 50, wherein the subject is working or living in environment of potential SARS-CoV-2 infection, for example Hospital, clinic, nursing home, retirement home, school, or COVID-19 hot spot.


SEQUENCE LISTING (319 sequences)

SEQ ID NO: 1 - DNA, 360 bp, Homo sapiens
  CDS (1)..(360)
caggtgcagctggtggagtctgggggaggcagggtccagcctgggaggtccctgagactctcctgtgcagcctctggattcaccttcagtactcatggcatgcactgggtccgccaggttccaggcaagggtctggactggattacaggtatcacatatgatggaagtgatcagttttatgcagactccgtgaagggccgcttcaccatctccagagacaattcaaagaatatgctatatctacaaatgaacagcctgagagctgaggacacggctgtgtatttctgtgcgaaagatctggagtggttaacaaactatcttgaccactggggccagggaaccctggtcaccgtctcctca

SEQ ID NO: 2 - protein, 120 aa, Homo sapiens
QVQLVESGGGRVQPGRSLRLSCAASGFTFSTHGMHWVRQVPGKGLDWITGITYDGSDQFYADSVKGRFTISRDNSKNMLYLQMNSLRAEDTAVYFCAKDLEWLTNYLDHWGQGTLVTVSS

SEQ ID NO: 3 - protein, 7 aa, Homo sapiens
GFTFSTH

SEQ ID NO: 4 - protein, 6 aa, Homo sapiens
TYDGSD

SEQ ID NO: 5 - protein, 11 aa, Homo sapiens
DLEWLTNYLDH

SEQ ID NO: 6 - DNA, 318 bp, Homo sapiens
  CDS (1)..(318)
gatgttgtgatgactcagtctccaggcaccctgtctgtgtctccaggggaaagagccaccctctcctgcagggccagtcagaatattaactacaacttagcctggtaccagcagaagcctggccaggctcccaggcttctcatcttcgatgcgtccaccagggccactggtatcccagccagattcactggcagtgggtctgggacagagttcactctcaccatcagcagcctgcagtctgaagattttgcagtttattactgtcagcagtctaatgcctggctcactttcggcggagggaccaaggtggaaatcaaa

SEQ ID NO: 7 - protein, 106 aa, Homo sapiens
DVVMTQSPGTLSVSPGERATLSCRASQNINYNLAWYQQKPGQAPRLLIFDASTRATGIPARFTGSGSGTEFTLTISSLQSEDFAVYYCQQSNAWLTFGGGTKVEIK

SEQ ID NO: 8 - protein, 11 aa, Homo sapiens
RASQNINYNLA

SEQ ID NO: 9 - protein, 7 aa, Homo sapiens
DASTRAT

SEQ ID NO: 10 - protein, 8 aa, Homo sapiens
QQSNAWLT

SEQ ID NO: 11 - DNA, 360 bp, Homo sapiens
  CDS (1)..(360)
gaggtgcagctggtggagtctgggggaggcatggtccagcctgggaggtccctgagactctcctgtgcaggctctggattcaccttcagcactcatggcatgcactgggtccgccaggctccaggcaagggtctggagtggattacaggtatcacatatgatggaagtgatgaattttatgcagactccgtgaagggccgattcaccatcgccagagacaattcaaagaatatgctatatctacaaatgaacagcctgagagctgaggacacggctgtgtatttctgtgcgaaagatctggagtggttaacaaactatgttgaccactggggccagggaaccctggtcaccgtctcctca

SEQ ID NO: 12 - protein, 120 aa, Homo sapiens
EVQLVESGGGMVQPGRSLRLSCAGSGFTFSTHGMHWVRQAPGKGLEWITGITYDGSDEFYADSVKGRFTIARDNSKNMLYLQMNSLRAEDTAVYFCAKDLEWLTNYVDHWGQGTLVTVSS

SEQ ID NO: 13 - protein, 7 aa, Homo sapiens
GFTFSTH

SEQ ID NO: 14 - protein, 6 aa, Homo sapiens
TYDGSD

SEQ ID NO: 15 - protein, 11 aa, Homo sapiens
DLEWLTNYVDH

SEQ ID NO: 16 - DNA, 318 bp, Homo sapiens
  CDS (1)..(318)
gatattgtgatgactcagtctccaggcaccctgtctgtgtctccaggggaaagagccaccctctcctgcagggccagtcagagtgttaactacaacttagcctggtaccagcagaagcctggccaggctcccaggcttctcatcttcgatgcgtccaccagggccactggtatcccagccagattcactggcagtgggtctgggacagagttcactctcaccatcagcagcctgcagtctgaagattttgcagtttattactgtcagcagtctaatgcctggctcactttcggcggagggaccaagctggagatcaaa

SEQ ID NO: 17 - protein, 106 aa, Homo sapiens
DIVMTQSPGTLSVSPGERATLSCRASQSVNYNLAWYQQKPGQAPRLLIFDASTRATGIPARFTGSGSGTEFTLTISSLQSEDFAVYYCQQSNAWLTFGGGTKLEIK

SEQ ID NO: 18 - protein, 11 aa, Homo sapiens
RASQSVNYNLA

SEQ ID NO: 19 - protein, 7 aa, Homo sapiens
DASTRAT

SEQ ID NO: 20 - protein, 8 aa, Homo sapiens
QQSNAWLT

SEQ ID NO: 21 - DNA, 351 bp, Homo sapiens
  CDS (1)..(351)
caggtgcagctggtggagtctgggggaggcttggtccagcctggggggtccctgagactctcctgtgcagcctctggattcaccgtcagtagcaactacatgagctgggtccgccaggctccagggaaggggctggagtgggtctcagttatttatagcggtggtagcacatactacgcagactccgtgaagggcagattcaccatctccagagacaattccaagaacacgctgtatcttcaaatgaacagcctgagagccgaggacacggctgtgtattactgtgcgagagattttggggattactactttgactactggggccagggaaccctggtcaccgtctcctca

SEQ ID NO: 22 - protein, 117 aa, Homo sapiens
QVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCARDFGDYYFDYWGQGTLVTVSS

SEQ ID NO: 23 - protein, 7 aa, Homo sapiens
GFTVSSN

SEQ ID NO: 24 - protein, 5 aa, Homo sapiens
YSGGS

SEQ ID NO: 25 - protein, 9 aa, Homo sapiens
DFGDYYFDY

SEQ ID NO: 26 - DNA, 324 bp, Homo sapiens
  CDS (1)..(324)
gatattgtgttgacgcagtctccagccaccctgtctttgtctccaggggaaagagccaccctctcctgcggggccagtcagagtgttagcagcagctacttagcctggtaccagcagaaacctggcctggcgcccaggctcctcatctatgatgcatccagcagggccactggcatcccagacaggttcagtggcagtgggtctgggacagacttcactctcaccatcagcagactggagcctgaagattttgcagtgtattactgtcagcagtatggtagctcacctaggacttttggccaggggaccaagctggagatcaaa

SEQ ID NO: 27 - protein, 108 aa, Homo sapiens
DIVLTQSPATLSLSPGERATLSCGASQSVSSSYLAWYQQKPGLAPRLLIYDASSRATGIPDRFSGSGSGTDFTLTISRLEPEDFAVYYCQQYGSSPRTFGQGTKLEIK

SEQ ID NO: 28 - protein, 12 aa, Homo sapiens
GASQSVSSSYLA

SEQ ID NO: 29 - protein, 7 aa, Homo sapiens
DASSRAT

SEQ ID NO: 30 - protein, 9 aa, Homo sapiens
QQYGSSPRT

SEQ ID NO: 31 - DNA, 384 bp, Homo sapiens
  CDS (1)..(384)
gaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccatgtattactgtgcgagagaggggtgtatagtgtgggagatagcgggggcactttgctactacggtatggacgtctggggccaagggaccacggtcaccgtctcctca

SEQ ID NO: 32 - protein, 128 aa, Homo sapiens
EVQLVQSGAEVKKPGASVKVSCKASGYTFTGYYMHWVRQAPGQGLEWMGWINPNSGGTNYAQKFQGRVTMTRDTSISTAYMELSRLRSDDTAMYYCAREGCIVWEIAGALCYYGMDVWGQGTTVTVSS

SEQ ID NO: 33 - protein, 7 aa, Homo sapiens
GYTFTGY

SEQ ID NO: 34 - protein, 6 aa, Homo sapiens
NPNSGG

SEQ ID NO: 35 - protein, 19 aa, Homo sapiens
EGCIVWEIAGALCYYGMDV

SEQ ID NO: 36 - DNA, 324 bp, Homo sapiens
  CDS (1)..(324)
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggtcaccttcggccaagggacacgactggagattaaa

SEQ ID NO: 37 - protein, 108 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPPVTFGQGTRLEIK

SEQ ID NO: 38 - protein, 11 aa, Homo sapiens
RASQSVSSYLA

SEQ ID NO: 39 - protein, 7 aa, Homo sapiens
DASNRAT

SEQ ID NO: 40 - protein, 10 aa, Homo sapiens
QQRSNWPPVT

SEQ ID NO: 41 - DNA, 363 bp, Homo sapiens
  CDS (1)..(363)
gaggtgcagctggtggagtctgggggaggcttggtacagcctggggggtccctgagactctcctgtgcagcctctggattcaccttcagtagctacgacatgcactgggtccgccaagctacaggaaaaggtctggagtgggtctcagctattggtactgctggtgacacatactatccaggctccgtgaagggccgattcaccatctccagagaaaatgccaagaactccttgtatcttcaaatgaacagcctgagagccgaggacacagccgtgtattactgtgcaagagcatcctttctagactactcgtggctctactttgactactggggccagggaaccctggtcaccgtctcctca

SEQ ID NO: 42 - protein, 121 aa, Homo sapiens
EVQLVESGGGLVQPGGSLRLSCAASGFTFSSYDMHWVRQATGKGLEWVSAIGTAGDTYYPGSVKGRFTISRENAKNSLYLQMNSLRAEDTAVYYCARASFLDYSWLYFDYWGQGTLVTVSS

SEQ ID NO: 43 - protein, 7 aa, Homo sapiens
GFTFSSY

SEQ ID NO: 44 - protein, 5 aa, Homo sapiens
GTAGD

SEQ ID NO: 45 - protein, 13 aa, Homo sapiens
ASFLDYSWLYFDY

SEQ ID NO: 46 - DNA, 324 bp, Homo sapiens
  CDS (1)..(324)
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacactcaaccagaggggacgttcggccaagggaccaaagtggatatcaaa

SEQ ID NO: 47 - protein, 108 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYTQPEGTFGQGTKVDIK

SEQ ID NO: 48 - protein, 11 aa, Homo sapiens
RASQSISSYLN

SEQ ID NO: 49 - protein, 7 aa, Homo sapiens
AASSLQS

SEQ ID NO: 50 - protein, 10 aa, Homo sapiens
QQSYTQPEGT

SEQ ID NO: 51 - DNA, 360 bp, Homo sapiens
  CDS (1)..(360)
caggtgcagctggtggagtctgggggaggcttggtccagcctggggggtccctgagactctcctgtgcagcctctggattcacctttagtagctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgcgagaggactagttgggcagtggctgggggttgactactggggccagggaaccctggtcaccgtctcctca

SEQ ID NO: 52 - protein, 120 aa, Homo sapiens
QVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARGLVGQWLGVDYWGQGTLVTVSS

SEQ ID NO: 53 - protein, 7 aa, Homo sapiens
GFTFSSY

SEQ ID NO: 54 - protein, 6 aa, Homo sapiens
KQDGSE

SEQ ID NO: 55 - protein, 11 aa, Homo sapiens
GLVGQWLGVDY

SEQ ID NO: 56 - DNA, 330 bp, Homo sapiens
  CDS (1)..(330)
cagtctgtgctgactcagccccactctgtgtcggagtctccggggaagacggtaaccatctcctgcaccggcagcagtggcagcattgccagcaactatgtgcagtggtaccagcagcgcccgggcagtgcccccaccactgtgatctatgaggataaccaaagaccctctggggtccctgatcggttctctggctccatcgacagctcctccaactctgcctccctcaccatctctggactgaagactgaggacgaggctgactactactgtcagtcttatgatagcagcaattgggtgttcggcggagggaccaagctgaccgtccta

SEQ ID NO: 57 - protein, 110 aa, Homo sapiens
QSVLTQPHSVSESPGKTVTISCTGSSGSIASNYVQWYQQRPGSAPTTVIYEDNQRPSGVPDRFSGSIDSSSNSASLTISGLKTEDEADYYCQSYDSSNWVFGGGTKLTVL

SEQ ID NO: 58 - protein, 13 aa, Homo sapiens
TGSSGSIASNYVQ

SEQ ID NO: 59 - protein, 7 aa, Homo sapiens
EDNQRPS

SEQ ID NO: 60 - protein, 9 aa, Homo sapiens
QSYDSSNWV

SEQ ID NO: 61 - DNA, 360 bp, Homo sapiens
  CDS (1)..(360)
caggtgcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagataatcgggcggggtgggaccaactggactactggggccagggaaccctggtcaccgtctcctca

SEQ ID NO: 62 - protein, 120 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDNRAGWDQLDYWGQGTLVTVSS

SEQ ID NO: 63 - protein, 7 aa, Homo sapiens
GYTFTSY

SEQ ID NO: 64 - protein, 6 aa, Homo sapiens
SAYNGN

SEQ ID NO: 65 - protein, 11 aa, Homo sapiens
DNRAGWDQLDY

SEQ ID NO: 66 - DNA, 321 bp, Homo sapiens
  CDS (1)..(321)
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttacccgtacacttttggccaggggaccaagctggagatcaaa

SEQ ID NO: 67 - protein, 107 aa, Homo sapiens
DIQMTQSPSTLSASVGDRVTITCRASQSISSWLAWYQQKPGKAPKLLIYKASSLESGVPSRFSGSGSGTEFTLTISSLQPDDFATYYCQQYNSYPYTFGQGTKLEIK

SEQ ID NO: 68 - protein, 11 aa, Homo sapiens
RASQSISSWLA

SEQ ID NO: 69 - protein, 7 aa, Homo sapiens
KASSLES

SEQ ID NO: 70 - protein, 9 aa, Homo sapiens
QQYNSYPYT

SEQ ID NO: 71 - DNA, 384 bp, Homo sapiens
  CDS (1)..(384)
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagagaggggtgtatagtgtgggagatagcgggggcactttgctactacggtatggacgtctggggccaagggaccacggtcaccgtctcctca

SEQ ID NO: 72 - protein, 128 aa, Homo sapiens
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAREGCIVWEIAGALCYYGMDVWGQGTTVTVSS

SEQ ID NO: 73 - protein, 7 aa, Homo sapiens
GYTFTGY

SEQ ID NO: 74 - protein, 6 aa, Homo sapiens
NPNSGG

SEQ ID NO: 75 - protein, 19 aa, Homo sapiens
EGCIVWEIAGALCYYGMDV

SEQ ID NO: 76 - DNA, 324 bp, Homo sapiens
  CDS (1)..(324)
gaaatagtgatgacgcagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggtcaccttcggccaaggaacacgactggagattaaa

SEQ ID NO: 77 - protein, 108 aa, Homo sapiens
EIVMTQSPATLSLSPGERATLSCRASQSVSSYLAWYQQKPGQAPRLLIYDASNRATGIPARFSGSGSGTDFTLTISSLEPEDFAVYYCQQRSNWPPVTFGQGTRLEIK

SEQ ID NO: 78 - protein, 11 aa, Homo sapiens
RASQSVSSYLA

SEQ ID NO: 79 - protein, 7 aa, Homo sapiens
DASNRAT

SEQ ID NO: 80 - protein, 10 aa, Homo sapiens
QQRSNWPPVT

SEQ ID NO: 81 - DNA, 357 bp, Homo sapiens
  CDS (1)..(357)
gaggtgcagctggtggaatctgggggaggcttggtccagcctggggggtccctgagactctcctgtgcagcctctggattcacctttagtagctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgcgagccgaggacacggccgtgtattactgtgcgagcttattttggagacggggctcgttcgacccctggggccagggaaccctggtcaccgtctcctca

SEQ ID NO: 82 - protein, 119 aa, Homo sapiens
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCASLFWRRGSFDPWGQGTLVTVSS

SEQ ID NO: 83 - protein, 7 aa, Homo sapiens
GFTFSSY

SEQ ID NO: 84 - protein, 6 aa, Homo sapiens
KQDGSE

SEQ ID NO: 85 - protein, 10 aa, Homo sapiens
LFWRRGSFDP

SEQ ID NO: 86 - DNA, 333 bp, Homo sapiens
  CDS (1)..(333)
cagtctgtgctgactcagccgcactctgtgtcggagtctccggggaagacggtaaccatctcctgcacccgcagcactggcagcattgccagcaactatgtgcagtggtaccagcagcgcccgggcagtgcccccaccactgtgatctatgaggataaccaaagaccctctggggtccctgatcggttctctggctccatcgacagctcctccaactctgcctccctcaccatctctggactgaagactgaggacgaggctgactactactgtcagtcttatgataccagctcctattgggtgttcggcggagggaccaagctgaccgtccta

SEQ ID NO: 87 - protein, 111 aa, Homo sapiens
QSVLTQPHSVSESPGKTVTISCTRSTGSIASNYVQWYQQRPGSAPTTVIYEDNQRPSGVPDRFSGSIDSSSNSASLTISGLKTEDEADYYCQSYDTSSYWVFGGGTKLTVL

SEQ ID NO: 88 - protein, 13 aa, Homo sapiens
TRSTGSIASNYVQ

SEQ ID NO: 89 - protein, 7 aa, Homo sapiens
EDNQRPS

SEQ ID NO: 90 - protein, 10 aa, Homo sapiens
QSYDTSSYWV

SEQ ID NO: 91 - DNA, 351 bp, Homo sapiens
  CDS (1)..(351)
caggtgcagctggtggagtctgggggaggcttggtccagcctggggggtccctgagactctcctgtgcagcctctggattcaccgtcagtagcaactacatgagctgggtccgccaggctccagggaaggggctggagtgggtctcagttatttatagcggtggtagcacattctacgcagactccgtgaagggcagattcaccatctccagagacaattccaagaacacgctgtatcttcaaatgaacagcctgagagccgaggacacggctgtgtattactgtgcgagagatttgcaggagtccggtatggacgtttggggccaagggaccacggtcaccgtctcctca

SEQ ID NO: 92 - protein, 117 aa, Homo sapiens
QVQLVESGGGLVQPGGSLRLSCAASGFTVSSNYMSWVRQAPGKGLEWVSVIYSGGSTFYADSVKGRFTISRDNSKNTLYLQMNSLRAEDTAVYYCARDLQESGMDVWGQGTTVTVSS

SEQ ID NO: 93 - protein, 7 aa, Homo sapiens
GFTVSSN

SEQ ID NO: 94 - protein, 5 aa, Homo sapiens
YSGGS

SEQ ID NO: 95 - protein, 9 aa, Homo sapiens
DLQESGMDV

SEQ ID NO: 96 - DNA, 318 bp, Homo sapiens
  CDS (1)..(318)
gacatccagttgacccagtctccatcttccgtgtctgcatctgtaggagacagagtcaccatcacttgtcgggcgagtcagggtattagcagctggttagcctggtatcagcagaagccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttccccaccttcggccaagggacacgactggagatcaaa

SEQ ID NO: 97 - protein, 106 aa, Homo sapiens
DIQLTQSPSSVSASVGDRVTITCRASQGISSWLAWYQQKPGKAPKLLIYAASSLQSGVPSRFSGSGSGTDFTLTISSLQPEDFATYYCQQANSFPTFGQGTRLEIK

SEQ ID NO: 98 - protein, 11 aa, Homo sapiens
RASQGISSWLA

SEQ ID NO: 99 - protein, 7 aa, Homo sapiens
AASSLQS

SEQ ID NO: 100 - protein, 8 aa, Homo sapiens
QQANSFPT

SEQ ID NO: 101 - DNA, 357 bp, Homo sapiens
  CDS (1)..(357)
caggtgcagctgcaggagtcgggcccaggactggtgaagccttcggagaccctgtccctcacctgcactgtctctggtggctccatcagcagtagtagttactactggggctggatccgccagcccccagggaaggggctggagtggattgggaatatctattatagtgggagcacctactacaacccgtccctcaagagtcgagtcaccatatccgtagacacgtccaagaaccagttctccctgaagctgaactctgtgaccgccgcagacacggctgtgtatttctgtgcgagactactatggttacgcgggagctactattggggccagggaaccctggtcaccgtctcctca

SEQ ID NO: 102 - protein, 119 aa, Homo sapiens
QVQLQESGPGLVKPSETLSLTCTVSGGSISSSSYYWGWIRQPPGKGLEWIGNIYYSGSTYYNPSLKSRVTISVDTSKNQFSLKLNSVTAADTAVYFCARLLWLRGSYYWGQGTLVTVSS

SEQ ID NO: 103 - protein, 9 aa, Homo sapiens
GGSISSSSY

SEQ ID NO: 104 - protein, 5 aa, Homo sapiens
YYSGS

SEQ ID NO: 105 - protein, 9 aa, Homo sapiens
LLWLRGSYY

SEQ ID NO: 106 - DNA, 330 bp, Homo sapiens
  CDS (1)..(330)
tcctatgagctgactcagccccactctgtgtcggagtctccggggaagacggtaaccatctcctgcaccggcagcagtggcagcattgccagcaactatgtgcagtggtaccagcagcgcccgggcagtgcccccaccactgtgatctatgaggataaccaaagaccctctggggtccctgatcggttctctggctccatcgacagctcctccaactctgcctccctcaccatctctggactgaagactgaggacgaggctgactactactgtcagtcttatgatagcagcaattgggtgttcggcggagggaccaagctgaccgtccta

SEQ ID NO: 107 - protein, 110 aa, Homo sapiens
SYELTQPHSVSESPGKTVTISCTGSSGSIASNYVQWYQQRPGSAPTTVIYEDNQRPSGVPDRFSGSIDSSSNSASLTISGLKTEDEADYYCQSYDSSNWVFGGGTKLTVL

SEQ ID NO: 108 - protein, 13 aa, Homo sapiens
TGSSGSIASNYVQ

SEQ ID NO: 109 - protein, 7 aa, Homo sapiens
EDNQRPS

SEQ ID NO: 110 - protein, 9 aa, Homo sapiens
QSYDSSNWV

SEQ ID NO: 111 - DNA, 381 bp, Homo sapiens
  CDS (1)..(381)
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgagagcctccctctattactctgatagtagtggttattcacacccgtactactttgactactggggccagggaaccctggtcaccgtctcctca

SEQ ID NO: 112 - protein, 127 aa, Homo sapiens
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARASLYYSDSSGYSHPYYFDYWGQGTLVTVSS

SEQ ID NO: 113 - protein, 7 aa, Homo sapiens
GGSFSGY

SEQ ID NO: 114 - protein, 5 aa, Homo sapiens
NHSGS

SEQ ID NO: 115 - protein, 19 aa, Homo sapiens
ASLYYSDSSGYSHPYYFDY

SEQ ID NO: 116 - DNA, 333 bp, Homo sapiens
  CDS (1)..(333)
cagcctgtgctgacgcagccgccctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcatgtcagcctccctggccatcactgggctccaggctgaggatgaggctgattattactgccagtcctatgacagcagcctgagtggttgggtgttcggcggagggaccaagctgaccgtccta

SEQ ID NO: 117 - protein, 111 aa, Homo sapiens
QPVLTQPPSVSGAPGQRVTISCTGSSSNIGAGYDVHWYQQLPGTAPKLLIYGNSNRPSGVPDRFSGSKSGMSASLAITGLQAEDEADYYCQSYDSSLSGWVFGGGTKLTVL

SEQ ID NO: 118 - protein, 14 aa, Homo sapiens
TGSSSNIGAGYDVH

SEQ ID NO: 119 - protein, 7 aa, Homo sapiens
GNSNRPS

SEQ ID NO: 120 - protein, 11 aa, Homo sapiens
QSYDSSLSGWV

SEQ ID NO: 121 - DNA, 372 bp, Homo sapiens
  CDS (1)..(372)
gaggtgcagctggtggaatctgggggaggcgtggtccagcctgggaggtccctgagactctcctgtacagcctctggattcaccttcagtttctattctgtgcactgggtccgccaggctccaggcaaggggctggagtgggtggcagttatttcatatgatggaagcactaaatactacgcagactccgtgaagggccgattcaccatctccagagacaattccaagaatacgctgtatctgcaaatggacagcctgagaactgaggacacggctgtctattactgtgcgagtccccccatctcccccatacctgcagctggtacgccccttgcctactggggccagggaaccctggtcaccgtctcctca

SEQ ID NO: 122 - protein, 124 aa, Homo sapiens
EVQLVESGGGVVQPGRSLRLSCTASGFTFSFYSVHWVRQAPGKGLEWVAVISYDGSTKYYADSVKGRFTISRDNSKNTLYLQMDSLRTEDTAVYYCASPPISPIPAAGTPLAYWGQGTLVTVSS

SEQ ID NO: 123 - protein, 7 aa, Homo sapiens
GFTFSFY

SEQ ID NO: 124 - protein, 6 aa, Homo sapiens
SYDGST

SEQ ID NO: 125 - protein, 15 aa, Homo sapiens
PPISPIPAAGTPLAY

SEQ ID NO: 126 - DNA, 339 bp, Homo sapiens
  CDS (1)..(339)
gacatccagttgacccagtctccagactccctggctgtgtctctgggcgagagggccaccatcaactgcaagtccagccagaatattttatacagctccagcaataagaactacttggcttggtaccagcagaaaccaggacagcctcctaagctgctcattgactgggcatctacccgggaatccggggtccctgaccgattcagtggcagcgggtctgggacagatttcactctcaccatcagcagcctgcaggctgaagatgtggcagtttattactgtcaccaatattatagtactccattcactttcggccctgggaccaaggtggaaatcaaa

SEQ ID NO: 127 - protein, 113 aa, Homo sapiens
DIQLTQSPDSLAVSLGERATINCKSSQNILYSSSNKNYLAWYQQKPGQPPKLLIDWASTRESGVPDRFSGSGSGTDFTLTISSLQAEDVAVYYCHQYYSTPFTFGPGTKVEIK

SEQ ID NO: 128 - protein, 17 aa, Homo sapiens
KSSQNILYSSSNKNYLA

SEQ ID NO: 129 - protein, 7 aa, Homo sapiens
WASTRES

SEQ ID NO: 130 - protein, 9 aa, Homo sapiens
HQYYSTPFT

SEQ ID NO: 131 - DNA, 363 bp, Homo sapiens
  CDS (1)..(363)
gaggtgcagctggtggagtctgggggaggcttggtacagcctggcaggtccctgagactctcctgtgcagcctctggatttatatttgatgattatgccatgcactgggtccggcaagctccagggaagggcctggagtgggtctcaggtattagttggaatagtgatagcataggctatgcggactctgtgaagggccgattcaccatctccagagacaacgccaagaactccctgtatctgcaaatgaacagtctgagagctgaggacacggccttgtattactgtgcaaaaggcagccgcagtgactggtcaaacccatcggactactggggccagggaaccttggtcaccgtctcttca

SEQ ID NO: 132 - protein, 121 aa, Homo sapiens
EVQLVESGGGLVQPGRSLRLSCAASGFIFDDYAMHWVRQAPGKGLEWVSGISWNSDSIGYADSVKGRFTISRDNAKNSLYLQMNSLRAEDTALYYCAKGSRSDWSNPSDYWGQGTLVTVSS

SEQ ID NO: 133 - protein, 7 aa, Homo sapiens
GFIFDDY

SEQ ID NO: 134 - protein, 6 aa, Homo sapiens
SWNSDS

SEQ ID NO: 135 - protein, 12 aa, Homo sapiens
GSRSDWSNPSDY

SEQ ID NO: 136 - DNA, 324 bp, Homo sapiens
  CDS (1)..(324)
tcctatgagctgactcagccaccctcggtgtcagtggccccaggaaagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatccggtggttttcggcggagggaccaagctgaccgtccta

SEQ ID NO: 137 - protein, 108 aa, Homo sapiens
SYELTQPPSVSVAPGKTARITCGGNNIGSKSVHWYQQKPGQAPVLVVYDDSDRPSGIPERFSGSNSGNTATLTISRVEAGDEADYYCQVWDSSSDPVVFGGGTKLTVL

SEQ ID NO: 138 - protein, 11 aa, Homo sapiens
GGNNIGSKSVH

SEQ ID NO: 139 - protein, 7 aa, Homo sapiens
DDSDRPS

SEQ ID NO: 140 - protein, 11 aa, Homo sapiens
QVWDSSSDPVV

SEQ ID NO: 141 - DNA, 357 bp, Homo sapiens
  CDS (1)..(357)
caggtgcagctggtgcagtctggggctgaggtgaagaagcctggggcctcagtgaaggtctcctgcaaggcttctggatacaccttcaccagttatgatatcaactgggtgcgacaggccactggacaagggcttgagtggatgggatggatgaaccctaacagtggtaacacaggctatgcacagaagttccagggcagagtcaccatgaccaggaacacctccataagcacagcctacatggagctgagcagcctgagatctgaggacacggccgtgtattactgtgcgagagtcgtatctttgactggcttttttgactactggggccagggaaccctggtcaccgtctcctca

SEQ ID NO: 142 - protein, 119 aa, Homo sapiens
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCARVVSLTGFFDYWGQGTLVTVSS

SEQ ID NO: 143 - protein, 7 aa, Homo sapiens
GYTFTSY

SEQ ID NO: 144 - protein, 6 aa, Homo sapiens
NPNSGN

SEQ ID NO: 145 - protein, 10 aa, Homo sapiens
VVSLTGFFDY

SEQ ID NO: 146 - DNA, 324 bp, Homo sapiens
  CDS (1)..(324)
tcctatgagctgactcagccaccctcggtgtcagtggccccaggaaagacggccaggattacctgtgggggaaacaacattggaagtaaaaatgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatccttgggtgttcggcggagggaccaagctgaccgtccta

SEQ ID NO: 147 - protein, 108 aa, Homo sapiens
SYELTQPPSVSVAPGKTARITCGGNNIGSKNVHWYQQKPGQAPVLVVYDDSDRPSGIPERFSGSNSGNTATLTISRVEAGDEADYYCQVWDSSSDPWVFGGGTKLTVL

SEQ ID NO: 148 - protein, 11 aa, Homo sapiens
GGNNIGSKNVH

SEQ ID NO: 149 - protein, 7 aa, Homo sapiens
DDSDRPS

SEQ ID NO: 150 - protein, 11 aa, Homo sapiens
QVWDSSSDPWV

SEQ ID NO: 151 - DNA, 351 bp, Homo sapiens
  CDS (1)..(351)
caggtgcagctggtggagtctggaggaggcttgatccagcctggggggtccctgagactctcctgtgcagcctctgggtttaccgtcagtagcaactacatgagctgggtccgccaggctccagggaaggggctggagtgggtctcagttatttatagcggtggtagcacatactacgcagactccgtgaagggccgattcaccatctccagagacaattccaagaacacgctgtttcttcaaatgaacagcctgagagccgaggacacggccgtgtattactgtgcgagagatcttcagtactacggtatggacgtctggggccaagggaccacggtcaccgtctcctca

SEQ ID NO: 152 - protein, 117 aa, Homo sapiens
QVQLVESGGGLIQPGGSLRLSCAASGFTVSSNYMSWVRQAPGKGLEWVSVIYSGGSTYYADSVKGRFTISRDNSKNTLFLQMNSLRAEDTAVYYCARDLQYYGMDVWGQGTTVTVSS

SEQ ID NO: 153 - protein, 7 aa, Homo sapiens
GFTVSSN

SEQ ID NO: 154 - protein, 5 aa, Homo sapiens
YSGGS

SEQ ID NO: 155 - protein, 9 aa, Homo sapiens
DLQYYGMDV

SEQ ID NO: 156 - DNA, 327 bp, Homo sapiens
  CDS (1)..(327)
gacatccagatgacccagtctccatccttcctgtctgcatctgtaggagacagagtcaccatcacttgccgggccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacagcttgatagttaccctccgcgggacacttttggccaggggaccaaggtggaaatcaaa

SEQ ID NO: 157 - protein, 109 aa, Homo sapiens
DIQMTQSPSFLSASVGDRVTITCRASQGISSYLAWYQQKPGKAPKLLIYAASTLQSGVPSRFSGSGSGTEFTLTISSLQPEDFATYYCQQLDSYPPRDTFGQGTKVEIK

SEQ ID NO: 158 - protein, 11 aa, Homo sapiens
RASQGISSYLA

SEQ ID NO: 159 - protein, 7 aa, Homo sapiens
AASTLQS

SEQ ID NO: 160 - protein, 11 aa, Homo sapiens
QQLDSYPPRDT

SEQ ID NO: 161 - DNA, 372 bp, Homo sapiens
  CDS (1)..(372)
caggtgcagctggtggagtctgggggaggcttggtaaagccagggcggtccctgagactctcctgtacagcttctggattcacctttggtgattatgctatgagctggttccgccaggctccagggaaggggctggagtgggtaggtttcattagaagcaaagcttatggtgggacaacagaatacgccgcgtctgtgaaaggcagattcagcatctcaagagatgattccaaaagcatcgcctatctgcaaatgaacagcctgaaaaccgaggacacagccgtgtattactgtactagaggttggatagtagtggttatcgaggatgcttttgatatctggggccaagggacaatggtcaccgtctcttca

SEQ ID NO: 162 - protein, 124 aa, Homo sapiens
QVQLVESGGGLVKPGRSLRLSCTASGFTFGDYAMSWFRQAPGKGLEWVGFIRSKAYGGTTEYAASVKGRFSISRDDSKSIAYLQMNSLKTEDTAVYYCTRGWIVVVIEDAFDIWGQGTMVTVSS

SEQ ID NO: 163 - protein, 7 aa, Homo sapiens
GFTFGDY

SEQ ID NO: 164 - protein, 8 aa, Homo sapiens
RSKAYGGT

SEQ ID NO: 165 - protein, 13 aa, Homo sapiens
GWIVVVIEDAFDI

SEQ ID NO: 166 - DNA, 324 bp, Homo sapiens
  CDS (1)..(324)
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgctcactttcggcggagggaccaaggtggaaatcaaa

SEQ ID NO: 167 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIK

SEQ ID NO: 168 - protein, 12 aa, Homo sapiens
RASQSVSSSYLA

SEQ ID NO: 169 - protein, 7 aa, Homo sapiens
GASSRAT

SEQ ID NO: 170 - protein, 9 aa, Homo sapiens
QQYGSSPLT

SEQ ID NO: 171 - DNA, 363 bp, Homo sapiens
  CDS (1)..(363)
gaggtgcagctggtgcagtctggggctgaggtgaagaggcctggggcctcagtgaaggtctcctgcaaggcttctggataccacttcaccgactactatatacactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagggtttattactatgatagtagtagttctgctgactactggggccagggaaccctggtcaccgtctcctca

SEQ ID NO: 172 - protein, 121 aa, Homo sapiens
EVQLVQSGAEVKRPGASVKVSCKASGYHFTDYYIHWVRQAPGQGLEWMGWINPNSGGTNYAQKFQGRVTMTRDTSISTAYMELSRLRSDDTAVYYCARVYYYDSSSSADYWGQGTLVTVSS

SEQ ID NO: 173 - protein, 7 aa, Homo sapiens
GYHFTDY

SEQ ID NO: 174 - protein, 6 aa, Homo sapiens
NPNSGG

SEQ ID NO: 175 - protein, 12 aa, Homo sapiens
VYYYDSSSSADY

SEQ ID NO: 176 - DNA, 327 bp, Homo sapiens
  CDS (1)..(327)
gacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagacattgcaacatattactgtcaacagtatgataatctccctccggcgctcactttcggcggagggaccaaggtggaaatcaaa

SEQ ID NO: 177 - protein, 109 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQYDNLPPALTFGGGTKVEIK

SEQ ID NO: 178 - protein, 11 aa, Homo sapiens
QASQDISNYLN

SEQ ID NO: 179 - protein, 7 aa, Homo sapiens
DASNLET

SEQ ID NO: 180 - protein, 11 aa, Homo sapiens
QQYDNLPPALT

SEQ ID NO: 181 - DNA, 363 bp, Homo sapiens
  CDS (1)..(363)
caggtgcagctggtgcagtctggggctgaggtgaagaggcctggggcctcagtgaaggtctcctgcaaggcttctggataccacttcaccgactactatatacactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagggtttattactatgatagtagtagttttgctgactactggggccagggaaccctggtcaccgtctcctca

SEQ ID NO: 182 - protein, 121 aa, Homo sapiens
QVQLVQSGAEVKRPGASVKVSCKASGYHFTDYYIHWVRQAPGQGLEWMGWINPNSGGTNYAQKFQGRVTMTRDTSISTAYMELSRLRSDDTAVYYCARVYYYDSSSFADYWGQGTLVTVSS

SEQ ID NO: 183 - protein, 7 aa, Homo sapiens
GYHFTDY

SEQ ID NO: 184 - protein, 6 aa, Homo sapiens
NPNSGG

SEQ ID NO: 185 - protein, 12 aa, Homo sapiens
VYYYDSSSFADY

SEQ ID NO: 186 - DNA, 327 bp, Homo sapiens; identical to SEQ ID NO: 176
  CDS (1)..(327)

SEQ ID NO: 187 - protein, 109 aa, Homo sapiens; identical to SEQ ID NO: 177

SEQ ID NO: 188 - protein, 11 aa, Homo sapiens
QASQDISNYLN

SEQ ID NO: 189 - protein, 7 aa, Homo sapiens
DASNLET

SEQ ID NO: 190 - protein, 11 aa, Homo sapiens
QQYDNLPPALT

SEQ ID NO: 191 - DNA, 384 bp, Homo sapiens
  CDS (1)..(384)
gaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaacaaggagggggccaatattgtagtggtggtagctgctactctggctactttgactactggggccagggaaccctggtcaccgtctcctca

SEQ ID NO: 192 - protein, 128 aa, Homo sapiens
EVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKQGGGQYCSGGSCYSGYFDYWGQGTLVTVSS

SEQ ID NO: 193 - protein, 7 aa, Homo sapiens
GFTFSSY

SEQ ID NO: 194 - protein, 6 aa, Homo sapiens
SYDGSN

SEQ ID NO: 195 - protein, 19 aa, Homo sapiens
QGGGQYCSGGSCYSGYFDY

SEQ ID NO: 196 - DNA, 318 bp, Homo sapiens
  CDS (1)..(318)
gacatccagttgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttatccgacgttcggccaagggaccaaagtggatatcaaa

SEQ ID NO: 197 - protein, 106 aa, Homo sapiens
DIQLTQSPSTLSASVGDRVTITCRASQSISSWLAWYQQKPGKAPKLLIYKASSLESGVPSRFSGSGSGTEFTLTISSLQPDDFATYYCQQYNSYPTFGQGTKVDIK

SEQ ID NO: 198 - protein, 11 aa, Homo sapiens
RASQSISSWLA

SEQ ID NO: 199 - protein, 7 aa, Homo sapiens
KASSLES

SEQ ID NO: 200 - protein, 8 aa, Homo sapiens
QQYNSYPT

SEQ ID NO: 201 - DNA, 366 bp, Homo sapiens
  CDS (1)..(366)
gaggtgcagctggtggagtctggaggaaacttgatccagcctggggggtccctgagactctcctgtgcagcctctgggttcaccgtcagtagcaactacatgagctgggtccgccaggctccagggaaggggctggagtgggtctcagttatttatagcggtggtagcacatactacgcagactccgtgaagggccgattcaccatctccagagacaattccaggaacacgctgtatcttcaaatgaacagcctgagagccgaggacacggccgtgtatttctgtgcgagagatagggtggttcggggagttagagggaacggtatggacgtctggggccaagggaccacggtcaccgtctcctca

SEQ ID NO: 202 - protein, 122 aa, Homo sapiens
EVQLVESGGNLIQPGGSLRLSCAASGFTVSSNYMSWVRQAPGKGLEWVSVIYSGGSTYYADSVKGRFTISRDNSRNTLYLQMNSLRAEDTAVYFCARDRVVRGVRGNGMDVWGQGTTVTVSS

SEQ ID NO: 203 - protein, 7 aa, Homo sapiens
GFTVSSN

SEQ ID NO: 204 - protein, 5 aa, Homo sapiens
YSGGS

SEQ ID NO: 205 - protein, 14 aa, Homo sapiens
DRVVRGVRGNGMDV

SEQ ID NO: 206 - DNA, 321 bp, Homo sapiens
  CDS (1)..(321)
gccatccggatgacccagtctccatcctccctgtctgcatctgtaggagacagagtcaccatcacttgccaggcgagtcaggacattaacatctatttaaattggtatcagcagagaccagggaaagcccctaagctcctgatctacgatgcatccaatttggaaacaggggtcccatcaaggttcagtggaagtggatctgggacagattttaccttcaccatcagcagcctgcagcctgaagatattgcaacatattactgtcaccagtatgataatctccctcggacgttcggccaagggaccaaagtggatatcaaa

SEQ ID NO: 207 - protein, 107 aa, Homo sapiens
AIRMTQSPSSLSASVGDRVTITCQASQDINIYLNWYQQRPGKAPKLLIYDASNLETGVPSRFSGSGSGTDFTFTISSLQPEDIATYYCHQYDNLPRTFGQGTKVDIK

SEQ ID NO: 208 - protein, 11 aa, Homo sapiens
QASQDINIYLN

SEQ ID NO: 209 - protein, 7 aa, Homo sapiens
DASNLET

SEQ ID NO: 210 - protein, 9 aa, Homo sapiens
HQYDNLPRT

SEQ ID NO: 211 - DNA, 384 bp, Homo sapiens
  CDS (1)..(384)
caggttcagctggtgcagtctggggctgaggtgaagaagcctgggtcctcggtgaaggtctcctgcaaggcttctggaggcaccttcagcaactatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgggagatagatggtactatgatagtagtggttatccaacaccagatgagtacttccagcactggggccagggcacccaggtcaccgtctcctca

SEQ ID NO: 212 - protein, 128 aa, Homo sapiens
QVQLVQSGAEVKKPGSSVKVSCKASGGTFSNYAISWVRQAPGQGLEWMGGIIPIFGTANYAQKFQGRVTITADESTSTAYMELSSLRSEDTAVYYCAGDRWYYDSSGYPTPDEYFQHWGQGTQVTVSS

SEQ ID NO: 213 - protein, 7 aa, Homo sapiens
GGTFSNY

SEQ ID NO: 214 - protein, 6 aa, Homo sapiens
IPIFGT

SEQ ID NO: 215 - protein, 19 aa, Homo sapiens
DRWYYDSSGYPTPDEYFQH

SEQ ID NO: 216 - DNA, 333 bp, Homo sapiens
  CDS (1)..(333)
cagtctgtgctgacgcagccgccctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagcctgagtggtgtggtattcggcggagggaccaagctgaccgtccta

SEQ ID NO: 217 - protein, 111 aa, Homo sapiens
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGVVFGGGTKLTVL

SEQ ID NO: 218 - protein, 14 aa, Homo sapiens
TGSSSNIGAGYDVH

SEQ ID NO: 219 - protein, 7 aa, Homo sapiens
GNSNRPS

SEQ ID NO: 220 - protein, 11 aa, Homo sapiens
QSYDSSLSGVV

SEQ ID NO: 221 - DNA, 372 bp, Homo sapiens
  CDS (1)..(372)
gaagtgcagctggtggagtctgggggaggcgtggtccagcctgggaggtccctgagactctcctgtgcagcctctggattcaccttcaatttctattctatgcactgggtccgccaggttccaggcaaggggctggaatgggtggcagttatatcgttcgatgggagcactaaacactacgcagagtccctgaggggccgatttgccgtctccagagacaattccaagagtaccctgtatctccaaatgaacagcctgagacctgaggacacggctgtgtattactgtgcgagtccccccctctcccccataccagctgctggtacgccccttgactactggggccagggaaccctggtcaccgtctcctca

SEQ ID NO: 222 - protein, 124 aa, Homo sapiens
EVQLVESGGGVVQPGRSLRLSCAASGFTFNFYSMHWVRQVPGKGLEWVAVISFDGSTKHYAESLRGRFAVSRDNSKSTLYLQMNSLRPEDTAVYYCASPPLSPIPAAGTPLDYWGQGTLVTVSS

SEQ ID NO: 223 - protein, 7 aa, Homo sapiens
GFTFNFY

SEQ ID NO: 224 - protein, 6 aa, Homo sapiens
SFDGST

SEQ ID NO: 225 - protein, 15 aa, Homo sapiens
PPLSPIPAAGTPLDY

SEQ ID NO: 226 - DNA, 339 bp, Homo sapiens
  CDS (1)..(339)
gaaatagtgatgacgcagtctccagactccctggctgtgtctctgggcgagagggccaccatcaactgcaagtccagccagagtattttctacagatccgacgataaggacttcttagcttggtaccagcagaaaccaggacagcctcctaagttgctcatttcctgggcatctacccgggaatccggggtccctgaccgattcagtggcagcgggtctgggacagatttcactctcaccatcagcagcctgcaggctgaagatgtggcagtttattactgtcagcaattttactatactccattcactttcggccctgggaccaagctggagatcaaa

SEQ ID NO: 227 - protein, 113 aa, Homo sapiens
EIVMTQSPDSLAVSLGERATINCKSSQSIFYRSDDKDFLAWYQQKPGQPPKLLISWASTRESGVPDRFSGSGSGTDFTLTISSLQAEDVAVYYCQQFYYTPFTFGPGTKLEIK

SEQ ID NO: 228 - protein, 17 aa, Homo sapiens
KSSQSIFYRSDDKDFLA

SEQ ID NO: 229 - protein, 7 aa, Homo sapiens
WASTRES

SEQ ID NO: 230 - protein, 9 aa, Homo sapiens
QQFYYTPFT

SEQ ID NO: 231 - DNA, 372 bp, Homo sapiens
  CDS (1)..(372)
gaggtgcagctggtgcagtctggagcagaggtgaaaaagcccggggaatctctgaagatctcatgtaagggttttggatacatctttagcaacgactggatcggctgggtgcgccagaagcccgggaaaggcctggagtggatgggcattatctatcctggtgactctgacacccgatacagcccgtccttccgaggccaggtcaccatctcagccgacaagtccgtcagcaccgcctatctgcagtgggacagcctgaaggcctcggacaccgccgtctattattgtacaagaccccgatacactacaatgatagtgggatcgaatccctttgatgtctggggccaggggactacggtcaccgtctcttca

SEQ ID NO: 232 - protein, 124 aa, Homo sapiens
EVQLVQSGAEVKKPGESLKISCKGFGYIFSNDWIGWVRQKPGKGLEWMGIIYPGDSDTRYSPSFRGQVTISADKSVSTAYLQWDSLKASDTAVYYCTRPRYTTMIVGSNPFDVWGQGTTVTVSS

SEQ ID NO: 233 - protein, 7 aa, Homo sapiens
GYIFSND

SEQ ID NO: 234 - protein, 6 aa, Homo sapiens
YPGDSD

SEQ ID NO: 235 - protein, 15 aa, Homo sapiens
PRYTTMIVGSNPFDV

SEQ ID NO: 236 - DNA, 339 bp, Homo sapiens
  CDS (1)..(339)
cagtctgtgctgacgcagccgccctcagtgtctggggccccagggcagagggtcaccatctcctgcactgggagcagctccaacatcgggggaggctatgatgtacactggtaccagaagcttccaggaacagcccccaaactcctcatctttgataatgacaatcggccctcaggggtccctgaccgattctctggctccaagtctgacacttcagcctccctgaccatcactggactccaggctgaggatgaggctgattatttttgtcagtcctatgacagcagcctgagtggctcgggagtggtattcggcggagggaccaagctgaccgtcctg

SEQ ID NO: 237 - protein, 113 aa, Homo sapiens
QSVLTQPPSVSGAPGQRVTISCTGSSSNIGGGYDVHWYQKLPGTAPKLLIFDNDNRPSGVPDRFSGSKSDTSASLTITGLQAEDEADYFCQSYDSSLSGSGVVFGGGTKLTVL

SEQ ID NO: 238 - protein, 14 aa, Homo sapiens
TGSSSNIGGGYDVH

SEQ ID NO: 239 - protein, 7 aa, Homo sapiens
DNDNRPS

SEQ ID NO: 240 - protein, 13 aa, Homo sapiens
QSYDSSLSGSGVV

SEQ ID NO: 241 - DNA, 360 bp, Homo sapiens
  CDS (1)..(360)
caggtgcagctggtggagtctgggggaggcttggtacagcctggggggtccctgagactctcctgtgcagcctctggattcacctttagccgctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaattaacagcctgagagccgaggacacggccgtatattactgtgcgaaaggggttaggtggggagtcgactactttgactactggggccagggaaccctggtcaccgtctcttca

SEQ ID NO: 242 - protein, 120 aa, Homo sapiens
QVQLVESGGGLVQPGGSLRLSCAASGFTFSRYAMSWVRQAPGKGLEWVSAISGSGGSTYYADSVKGRFTISRDNSKNTLYLQINSLRAEDTAVYYCAKGVRWGVDYFDYWGQGTLVTVSS

SEQ ID NO: 243 - protein, 7 aa, Homo sapiens
GFTFSRY

SEQ ID NO: 244 - protein, 6 aa, Homo sapiens
SGSGGS

SEQ ID NO: 245 - protein, 11 aa, Homo sapiens
GVRWGVDYFDY

SEQ ID NO: 246 - DNA, 324 bp, Homo sapiens
  CDS (1)..(324)
gccatccggatgacccagtctccttccaccctgtctgcatctgtaggagacagagtcaccatcacttgccgggccagtcagagtattagtagctggttggcctggtatcagcagaaaccagggaaagcccctaagctcctgatctatgatgcctccagtttggaaagtggggtcccatcaaggttcagcggcagtggatctgggacagaattcactctcaccatcagcagcctgcagcctgatgattttgcaacttattactgccaacagtataatagttattcttcgtggacgttcggccaagggaccaagctggagatcaaa

SEQ ID NO: 247 - protein, 108 aa, Homo sapiens
AIRMTQSPSTLSASVGDRVTITCRASQSISSWLAWYQQKPGKAPKLLIYDASSLESGVPSRFSGSGSGTEFTLTISSLQPDDFATYYCQQYNSYSSWTFGQGTKLEIK

SEQ ID NO: 248 - protein, 11 aa, Homo sapiens
RASQSISSWLA

SEQ ID NO: 249 - protein, 7 aa, Homo sapiens
DASSLES

SEQ ID NO: 250 - protein, 10 aa, Homo sapiens
QQYNSYSSWT

SEQ ID NO: 251 - DNA, 366 bp, Homo sapiens
  CDS (1)..(366)
gaggtgcagctggtggagtctgggggaggcgtggtccagcctgggaggtccctgagactctcctgtgcagcgtctggattcaccttcactaggtatggcatgcactgggtccgccaggctccaggcaaggggctggagtgggtggcagttatatggtatgatggaaataataaaaactatgaagactccgtgaggggccgattcaccatctccagagacaattccaagaacacgctgtttctgcaaatgaacagcctgagagccgaagacacggctgtgtattactgtgcgagaggccgccgcggtggatacagctatggcctctttgactactggggccagggaatcctggtcaccgtctcctca

SEQ ID NO: 252 - protein, 122 aa, Homo sapiens
EVQLVESGGGVVQPGRSLRLSCAASGFTFTRYGMHWVRQAPGKGLEWVAVIWYDGNNKNYEDSVRGRFTISRDNSKNTLFLQMNSLRAEDTAVYYCARGRRGGYSYGLFDYWGQGILVTVSS

SEQ ID NO: 253 - protein, 7 aa, Homo sapiens
GFTFTRY

SEQ ID NO: 254 - protein, 6 aa, Homo sapiens
WYDGNN

SEQ ID NO: 255 - protein, 13 aa, Homo sapiens
GRRGGYSYGLFDY

SEQ ID NO: 256 - DNA, 321 bp, Homo sapiens
  CDS (1)..(321)
gccatccggatgacccagtctccatcctccctgtctgcatctgtaggggacagagtcaccatcacttgccaggcgagtcaggacattagcaactatttaaattggtatcagcagaaaccagggaaagcccctaagctcctgatctacgatgcatccaatttggagacaggggtcccatcaaggttcagtggaagtggatctgggacagattttactttcaccatcagcagcctgcagcctgaagatgttgcaacatattcctgtcaacagtatgataatctcccgctcactttcggcggggggaccaaggtggaaatcaaa

SEQ ID NO: 257 - protein, 107 aa, Homo sapiens
AIRMTQSPSSLSASVGDRVTITCQASQDISNYLNWYQQKPGKAPKLLIYDASNLETGVPSRFSGSGSGTDFTFTISSLQPEDVATYSCQQYDNLPLTFGGGTKVEIK

SEQ ID NO: 258 - protein, 11 aa, Homo sapiens
QASQDISNYLN

SEQ ID NO: 259 - protein, 7 aa, Homo sapiens
DASNLET

SEQ ID NO: 260 - protein, 9 aa, Homo sapiens
QQYDNLPLT

SEQ ID NO: 261 - DNA, 363 bp, Homo sapiens
  CDS (1)..(363)
caggtgcagctacagcagtggggcgcaggactgttgaagccttcggagaccctgtcccgcacctgcgctgtctatggtgggtccttcagtgattactactggagctggatccgccagcccccagggaagggactggagtggattggcgaagtcaatcataggggtgtcaccaactacaacccgtccctcaagagtcgagtcaccatatcaatagacacgtctaagagccagttctccctggaactgaggtctgtgaccgacgcggacacggctctctatttctgtgcgagaagccccttctggacgatagcggctcgtccgtttgactactggggccggggaaccctggtcaccgtctcctca

SEQ ID NO: 262 - protein, 121 aa, Homo sapiens
QVQLQQWGAGLLKPSETLSRTCAVYGGSFSDYYWSWIRQPPGKGLEWIGEVNHRGVTNYNPSLKSRVTISIDTSKSQFSLELRSVTDADTALYFCARSPFWTIAARPFDYWGRGTLVTVSS

SEQ ID NO: 263 - protein, 7 aa, Homo sapiens
GGSFSDY

SEQ ID NO: 264 - protein, 5 aa, Homo sapiens
NHRGV

SEQ ID NO: 265 - protein, 13 aa, Homo sapiens
SPFWTIAARPFDY

SEQ ID NO: 266 - DNA, 324 bp, Homo sapiens
  CDS (1)..(324)
gaaatagtgatgacgcagtctccaggcaccctgtctttgtctccaggggaaagagccaccctctcctgcagggccagtcagagtgttatcagcaactacttagcctggtaccagcagaaacctggccaggctcccaggttcctcatctatgctgcatccaccagggccagaggcatcccagacaggttcagtggcagtgggtctgggacagacttcactctcaccatcagcagactggaggctgaagattttgcagtttattactgtcagcaatatggtaactcaccgctcactttcggcggagggaccaagctggagatcaaa

SEQ ID NO: 267 - protein, 108 aa, Homo sapiens
EIVMTQSPGTLSLSPGERATLSCRASQSVISNYLAWYQQKPGQAPRFLIYAASTRARGIPDRFSGSGSGTDFTLTISRLEAEDFAVYYCQQYGNSPLTFGGGTKLEIK

SEQ ID NO: 268 - protein, 12 aa, Homo sapiens
RASQSVISNYLA

SEQ ID NO: 269 - protein, 7 aa, Homo sapiens
AASTRAR

SEQ ID NO: 270 - protein, 9 aa, Homo sapiens
QQYGNSPLT

SEQ ID NO: 271 - DNA, 366 bp, Homo sapiens
  CDS (1)..(366)
gaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaacaaatcagtggctacaattacccctactactttgactactggggccagggaaccctggtcaccgtctcctca

SEQ ID NO: 272 - protein, 122 aa, Homo sapiens
EVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKQISGYNYPYYFDYWGQGTLVTVSS

SEQ ID NO: 273 - protein, 7 aa, Homo sapiens
GFTFSSY

SEQ ID NO: 274 - protein, 6 aa, Homo sapiens
SYDGSN

SEQ ID NO: 275 - protein, 13 aa, Homo sapiens
QISGYNYPYYFDY

SEQ ID NO: 276 - DNA, 321 bp, Homo sapiens
  CDS (1)..(321)
gacatccagttgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataatctcccgctcactttcggcggagggaccaagctggagatcaaa

SEQ ID NO: 277 - protein, 107 aa, Homo sapiens
DIQLTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKLEIK

SEQ ID NO: 278 - protein, 11 aa, Homo sapiens
QASQDISNYLN

SEQ ID NO: 279 - protein, 7 aa, Homo sapiens
DASNLET

SEQ ID NO: 280 - protein, 9 aa, Homo sapiens
QQYDNLPLT

SEQ ID NO: 281 - DNA, 384 bp, Homo sapiens
  CDS (1)..(384)
gaggtgcagctgcaggagtcgggcccaggactggtgaagccttcggagaccctgtccctcacctgcactgtctctggtggctccgtcagcagtggtagttattactggagctggatccggcagcccccagggaagggactggagtggattgggtatatctattacagtgggagcaccaactacaacccctccctcaagagtcgagtcaccatatcagtagacacgtccaagaaccagttctccctgaaggtgagctctgtgaccgctgcggacacggccgtgtattactgtgcgagagattctattgtagtagtaccagctgctagaccgctttactatggtatggacgtctggggccaagggaccacggtcaccgtctcctca

SEQ ID NO: 282 - protein, 128 aa, Homo sapiens
EVQLQESGPGLVKPSETLSLTCTVSGGSVSSGSYYWSWIRQPPGKGLEWIGYIYYSGSTNYNPSLKSRVTISVDTSKNQFSLKVSSVTAADTAVYYCARDSIVVVPAARPLYYGMDVWGQGTTVTVSS

SEQ ID NO: 283 - protein, 9 aa, Homo sapiens
GGSVSSGSY

SEQ ID NO: 284 - protein, 5 aa, Homo sapiens
YYSGS

SEQ ID NO: 285 - protein, 18 aa, Homo sapiens
DSIVVVPAARPLYYGMDV

SEQ ID NO: 286 - DNA, 333 bp, Homo sapiens
  CDS (1)..(333)
cagtctgtgctgacgcagccaccctcagcgtctgggacccccgggcagagggtcaccatctcttgttctggaagcagctccaacatcggaagtaattctgtagactggtaccagcagctcccaggaacggcccccaaactcctcatctatagtactaatcagcggccctcaggggtccctgaccgattctctggctccaagtctggcacctcagcctccctggccatcagtgggctccagtctgaggatgaggctgattattactgtgcagcatgggatgacagcctgaatgtccgttatgtcttcggaactgggaccaaggtcaccgtccta

SEQ ID NO: 287 - protein, 111 aa, Homo sapiens
QSVLTQPPSASGTPGQRVTISCSGSSSNIGSNSVDWYQQLPGTAPKLLIYSTNQRPSGVPDRFSGSKSGTSASLAISGLQSEDEADYYCAAWDDSLNVRYVFGTGTKVTVL

SEQ ID NO: 288 - protein, 13 aa, Homo sapiens
SGSSSNIGSNSVD

SEQ ID NO: 289 - protein, 7 aa, Homo sapiens
STNQRPS

SEQ ID NO: 290 - protein, 12 aa, Homo sapiens
AAWDDSLNVRYV

SEQ ID NO: 291 - DNA, 384 bp, Homo sapiens
  CDS (1)..(384)
gaggtgcagctggtggagtctgggggaggcttggtacagcctggcaggtccctgagactctcctgtgcagcctccggatttacttttgatgattttgacatgcactgggtccggcaagctccagggaagggcctggaatgggtctcaggcgttacttggaacagtggaatcatagggtatgcggactctgtgaagggccgattcaccatctccagagacaacgccaagaactccctgtatctgcaaatgaacagtctgagagctgaggacacggccttgtattactgtgcaaaagaattctatccccgttatggttcgggtattaggtcccactacttcggtatggacgtctggggccaagggaccacggtcaccgtctcctca

SEQ ID NO: 292 - protein, 128 aa, Homo sapiens
EVQLVESGGGLVQPGRSLRLSCAASGFTFDDFDMHWVRQAPGKGLEWVSGVTWNSGIIGYADSVKGRFTISRDNAKNSLYLQMNSLRAEDTALYYCAKEFYPRYGSGIRSHYFGMDVWGQGTTVTVSS

SEQ ID NO: 293 - protein, 7 aa, Homo sapiens
GFTFDDF

SEQ ID NO: 294 - protein, 6 aa, Homo sapiens
TWNSGI

SEQ ID NO: 295 - protein, 19 aa, Homo sapiens
EFYPRYGSGIRSHYFGMDV

SEQ ID NO: 296 - DNA, 330 bp, Homo sapiens
  CDS (1)..(330)
cagtctgtgctgactcagtcaccctcagcgtctgggacccccgggcagagggtcaccatctcttgttctggcagcagctccaacgtcggaagtaatgctgtaaactggtaccagcagctcccaggaacggcccccaaactcctcatctatagtaataatcggcggccctcaggggtcccgaaccgattctctggctccaagtctggcacctcagcctccctggccatcagtggactacagtctgacgatgaggctgattattactgtgcagcatgggatgacagcctggatggccccacattcggcggagggaccaagctgaccgtccta

SEQ ID NO: 297 - protein, 110 aa, Homo sapiens
QSVLTQSPSASGTPGQRVTISCSGSSSNVGSNAVNWYQQLPGTAPKLLIYSNNRRPSGVPNRFSGSKSGTSASLAISGLQSDDEADYYCAAWDDSLDGPTFGGGTKLTVL

SEQ ID NO: 298 - protein, 13 aa, Homo sapiens
SGSSSNVGSNAVN

SEQ ID NO: 299 - protein, 7 aa, Homo sapiens
SNNRRPS

SEQ ID NO: 300 - protein, 11 aa, Homo sapiens
AAWDDSLDGPT

SEQ ID NO: 301 - protein, 223 aa, SARS-CoV-2 (severe acute respiratory syndrome coronavirus type 2)
  PEPTIDE (1)..(223): Reference sequence: NCBI: YP_009724390.1 (Arg319-Phe541) RBD only
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 302 - protein, 9 aa, SARS-CoV-2 (severe acute respiratory syndrome coronavirus type 2)
  PEPTIDE (1)..(9): aa 112 to 120
TGCVIAWNS

SEQ ID NO: 303 - protein, 5 aa, SARS-CoV-2 (severe acute respiratory syndrome coronavirus type 2)
  PEPTIDE (1)..(5): aa 140 to 144
KSNLK

SEQ ID NO: 304 - protein, 27 aa, SARS-CoV-2 (severe acute respiratory syndrome coronavirus type 2)
  PEPTIDE (1)..(27): aa 151 to 177
STEIYQAGSTPCNVGEGFNCYFPLQSY

SEQ ID NO: 305 - protein, 31 aa, SARS-CoV-2 (severe acute respiratory syndrome coronavirus type 2)
  PEPTIDE (1)..(31): aa 105 to 135
YKLPDDFTGCVIAWNSNNLDSKVGGNYNYLY

SEQ ID NO: 306 - protein, 30 aa, SARS-CoV-2 (severe acute respiratory syndrome coronavirus type 2)
  PEPTIDE (1)..(30): aa 105 to 134
YKLPDDFTGCVIAWNSNNLDSKVGGNYNYL

SEQ ID NO: 307 - protein, 30 aa, SARS-CoV-2 (severe acute respiratory syndrome coronavirus type 2)
  PEPTIDE (1)..(30): aa 106 to 135
KLPDDFTGCVIAWNSNNLDSKVGGNYNYLY

SEQ ID NO: 308 - protein, 29 aa, SARS-CoV-2 (severe acute respiratory syndrome coronavirus type 2)
  PEPTIDE (1)..(29): aa 106 to 134
KLPDDFTGCVIAWNSNNLDSKVGGNYNYL

SEQ ID NO: 309 - protein, 26 aa, SARS-CoV-2 (severe acute respiratory syndrome coronavirus type 2)
  PEPTIDE (1)..(26): aa 153 to 178
EIYQAGSTPCNGVEGFNCYFPLQSYG

SEQ ID NO: 310 - protein, 24 aa, SARS-CoV-2 (severe acute respiratory syndrome coronavirus type 2)
  PEPTIDE (1)..(24): aa 155 to 178
YQAGSTPCNGVEGFNCYFPLQSYG

SEQ ID NO: 311 - protein, 13 aa, SARS-CoV-2 (severe acute respiratory syndrome coronavirus type 2)
  PEPTIDE (1)..(13): aa 164 to 176
GVEGFNCYFPLQS

SEQ ID NO: 312 - protein, 18 aa, SARS-CoV-2 (severe acute respiratory syndrome coronavirus type 2)
  PEPTIDE (1)..(18): aa 51 to 68
YNSASFSTFKCYGVSPTK

SEQ ID NO: 313 - protein, 14 aa, SARS-CoV-2 (severe acute respiratory syndrome coronavirus type 2)
  PEPTIDE (1)..(14): aa 139 to 152
RKSNLKPFERDIST

SEQ ID NO: 314 - protein, 11 aa, SARS-CoV-2 (severe acute respiratory syndrome coronavirus type 2)
  PEPTIDE (1)..(11): aa 55 to 65
SFSTFKCYGVS

SEQ ID NO: 315 - protein, 10 aa, SARS-CoV-2 (severe acute respiratory syndrome coronavirus type 2)
  PEPTIDE (1)..(10): aa 56 to 65
FSTFKCYGVS

SEQ ID NO: 316 - protein, 5 aa, SARS-CoV-2 (severe acute respiratory syndrome coronavirus type 2)
  PEPTIDE (1)..(5): aa 69 to 73
LNDLC

SEQ ID NO: 317 - protein, 4 aa, SARS-CoV-2 (severe acute respiratory syndrome coronavirus type 2)
  PEPTIDE (1)..(4): aa 70 to 73
NDLC

SEQ ID NO: 318 - protein, 222 aa, SARS-CoV (severe acute respiratory syndrome coronavirus)
  PEPTIDE (1)..(222): Reference sequence: NCBI: AAX16192.1 (Arg306-Phe527) RBD only
RVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNF

SEQ ID NO: 319 - protein, 224 aa, Artificial Sequence
  SARS CoV-2 RBD wt -N-terminus modified
  PEPTIDE (1)..(224): SARS CoV-2 RBD wt -N-terminus modified
MRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF